(19) United States
(12) Patent Application Publication
(10) Pub. No.: US 2024/0295554 A1
(43) Pub. Date: Sep. 5, 2024

(54) SYSTEM, METHOD, APPARATUS AND DIAGNOSTIC TEST FOR PLASMODIUM VIVAX

(71) Applicant: The Walter and Eliza Hall Institute of Medical Research, Parkville (AU)

(72) Inventors: Ivo Mueller, Mount Macedon (AU); Takafumi Tsuboi, Ehime Prefecture (JP); Michael White, Melbourne (AU); Rhea Longley, Brunswick West (AU)

(73) Assignee: The Walter and Eliza Hall Institute of Medical Research, Parkville (AU)

(21) Appl. No.: 18/378,736

(22) Filed: Oct. 11, 2023

Related U.S. Application Data
(63) Continuation of application No. 16/472,269, filed on Jun. 21, 2019, now Pat. No. 11,835,520.
(60) Provisional application No. 62/438,963, filed on Dec. 23, 2016.

(51) Int. Cl. G01N 33/569; C07K 14/415; C07K 16/20; G01N 33/68

(52) U.S. Cl. CPC G01N 33/56905; C07K 14/415; C07K 16/205; G01N 33/6857; G01N 2333/445

(57) ABSTRACT

A system, method, apparatus and diagnostic test for Plasmodium vivax, to determine a likelihood of a specific timing of infection by P. vivax in a subject, and hence identify individuals with a high probability of being infected with otherwise undetectable liver-stage hypnozoites. The system, method, apparatus and diagnostic test relate to the identification of hypnozoites (“dormant” liver-stages), or at least of the likelihood of the subject being so infected. Optionally and preferably, the specific timing relates to recent infections, for example within the last 9 months.

### CROSS REFERENCE TO RELATED APPLICATIONS

The present invention is a continuation of U.S. application Ser. No. 16/472,269 filed Jun. 21, 2019, which is a national stage application which claims priority from PCT Application No. PCT/IB2017/001776 filed Dec. 21, 2017, and U.S. Application No. 62/438,963 filed Dec. 23, 2016. Applicants claim the benefits of 35 U.S.C. § 120 as to the said priority U.S. and PCT applications, and priority under 35 U.S.C. § 119 as to the said U.S. provisional application, and the entire disclosures of all applications are incorporated herein by reference in their entireties.

### SEQUENCE LISTING

This application contains a Sequence Listing, submitted in, XML format via PatentCenter and hereby incorporated by reference in its entirety. The XML copy, created on May 7, 2024, is named “2762-9_PCT_US_CON_2024-05-07.xml” and is 248,294 bytes in size.

### FIELD OF THE INVENTION

The present invention is of a system, method, apparatus and diagnostic test for relapsing Plasmodium species (i.e Plasmodium vivax and Plasmodium ovale), and in particular, to such a system, method, apparatus and diagnostic test for Plasmodium vivax for characterizing at least one aspect of infection in a subject or a population of subjects.

### BACKGROUND OF THE INVENTION

Plasmodium vivax (P. vivax) is one of five species of parasites that cause malaria in humans. This disease is marked by severe fever and pain, and can be fatal. The symptoms are caused by the parasite's infection, and destruction, of red blood cells in the subject. Infection of new subjects occurs when infectious mosquitoes take a blood meal from humans and inoculate parasites with their saliva.

Like one other species that infects humans, P. ovale, P. vivax has the ability to “hide” in the liver of a subject and remain dormant—and asymptomatic—before (re-)emerging to cause renewed bloodstage infections and malarial symptoms. Transmission from humans to mosquitoes can only occur when the sexual stages of the parasite (gametocytes) are circulating in the blood. Liver-stage infection with hypnozoites is completely undetectable and asymptomatic, and transmission to mosquitoes is not possible. P. falciparum and P. knowlesi do not have this ability. P. malariae can cause recurrent infections but it remains unclear if these infections persist in the bloodstream, the liver or another organ. This ability to hide from the immune system in the liver for prolonged periods makes P. vivax and P. ovale particularly difficult to detect and treat.

FIG. 1 shows the overall life cycle of the P. vivax parasite (see Mueller, I. et al. Key gaps in the knowledge of Plasmodium vivax, a neglected human malaria parasite. Lancet Infectious Diseases 9, 555-566 (2009)). During a blood meal, a malaria-infected female Anopheles mosquito inoculates sporozoites into the human host (1). Sporozoites infect liver cells (2) and either enter a dormant hypnozoite state or mature into schizonts (3), which rupture and release merozoites (4). After this initial replication in the liver (exo-erythrocytic schizogony A), the parasites undergo asexual multiplication in the erythrocytes (erythrocytic schizogony B). Merozoites infect red blood cells (5). The ring stage trophozoites mature into schizonts, which rupture releasing further merozoites into the blood stream (6). Some parasites differentiate into sexual erythrocytic stages (gametocytes) (7). Blood stage parasites are responsible for the clinical manifestations of the disease.

The gametocytes, male (microgametocytes) and female (macrogametocytes), are ingested by an Anopheles mosquito during a blood meal (8). The parasites' multiplication in the mosquito is known as the sporogonic cycle (C). While in the mosquito's stomach, the microgametes penetrate the macrogametes generating zygotes (9). The zygotes in turn become motile and elongated (ookinetes) (10) which invade the midgut wall of the mosquito where they develop into oocysts (11). The oocysts grow, rupture, and release sporozoites (12), which make their way to the mosquito's salivary glands. Inoculation of the sporozoites (1) into a new human host perpetuates the malaria life cycle.

Diagnosis of subjects with P. vivax infections is of paramount importance to reducing or even eliminating transmission in a population. Such diagnosis would also significantly help individual subjects to receive proper treatment, including those that have only silent liverstage infections. Given the high degree of population mobility today, particularly in response to natural disasters or human conflicts, accurate and rapid diagnosis of all P. vivax infections has become even more important to controlling the disease. In addition, as transmission in countries decreases (as each population approaches elimination of the disease), population-level surveillance becomes increasingly important. This surveillance will aid in determining residual areas of transmission within a country, and can also be used to provide evidence for the absence of transmission indicating that elimination has been achieved.

Some proteins have been very well studied and characterized for diagnostic purposes. For example, merozoite surface protein 1 (MSP1), in particular certain C-terminal MSP1-19 fragments and the N-terminal Pv200L fragments have been described as suitable diagnostic antigens. Some examples of prior publications related to this protein include U.S. Pat. No. 6,958,235, which focuses on a fragment of this protein for diagnostic purposes; WO9208795A1, which focuses on this protein for diagnosis; and US20100119539. Merozoite surface protein 3 (MSP3) is described with regard to a diagnostic tool in U.S. Pat. No. 7,488,489. MSP3.10 [merozoite surface protein 3 alpha (MSP3a)] is described as part of the family of merozoite surface protein 3 like proteins for diagnostic and other purposes in US20070098738. Rhoptry associated membrane antigen is described with regard to a diagnostic tool in EP0372019 B1. Many other proteins were described in relation to their immunogenicity and hence their therapeutic utility as part of a vaccine. Some non-limiting examples are given below.

| UniProt | Annotation¹ | Patent information |
|---|---|---|
| A5K3N8 | rhoptry neck protein 2, | Vaccine including this protein (US20160158332); |
| | putative (RON2) | specifically described and claimed for diagnosis in |
| | | EP2520585, no family members, abandoned in 2013 |
| A5KBS6 | hypothetical protein, | WO2015091734 (vaccine) |
| | conserved (PvLSA3^{d}) | |
| A5K4Z2 | apical merozoite | U.S. Pat. No. 9,364,525 (one of a list of antigens |
| | antigen 1 (PvAMA1) | for a vaccine, downloaded as US20100150998); |
| | | WO2006037807-structure of this antigen; U.S. Pat. |
| | | No. 7,150,875-vaccine specifically directed |
| | | at this antigen |
| A5K0N7 | translocon component | US20140348870-Especially preferred antigens are |
| | PTEX150, putative | post-challenge immunity associated antigens that |
| | (PTEX150) | are identified via pre-infection suppressive |
| | | treatment, controlled sub-symptomatic infection to |
| | | develop immunity, and comparative proteomic |
| | | differential analysis. WO2010127398-more focused |
| | | on treatment |
| A5KBL6 | merozoite surface | WO2014186798-immune stimulation (1 of a long |
| | protein 5 | list of diseases and antigens); U.S. Pat. No. |
| | | 8,350,019 (focuses on this protein for diagnostic |
| | | use); WO2015031904-use of this protein to |
| | | determine if an individual is protected against |
| | | malaria; WO2016030292-focused on treatment; |
| | | US20110020387-malaria vaccine |
| A5K800 | MSP7 [merozoite surface | EP2990059-therapeutic but mentions MSP7 |
| | protein 7 (MSP7)] | specifically |
| A5K736 | reticulocyte binding | U.S. Pat. No. 8,703,147-treatment and prevention |
| | protein 2b (RBP2b) | of malaria |
| A5KAV2 | merozoite surface | EP2223937-prevention and treatment of malaria; |
| | protein 3 (MSP3.3) | describes the gene family that includes this protein |
| | | for diagnosis and treatment-EP1689866 |
| A5KAU1 | merozoite surface | US20140348870-identified this protein as |
| | protein 8, putative | immunogenic |
| A5K806 | thrombospondin-related | Immunogenic, part of a vaccine: US20100272745, |
| | anonymous protein | U.S. Pat. No. 7,790,186, U.S. Pat. No. 7,150,875, |
| | (PvTRAP/SSP2) | WO2013142278, WO2015091734 |
| A5KDR7 | Duffy receptor | mentioned as immunogenic protein, part of a |
| | precursor (DBP) | vaccine: U.S. Pat. No. 7,790,186 |
| A5KAW0 | MSP3.10 [merozoite | US20070098738-describes entire protein family; |
| | surface protein | US707129-describes various members of this |
| | 3 alpha (MSP3a)] | family as being immunogenic |

Still other proteins have barely been described or characterized in the literature. In some cases, these proteins have not yet been described with regard to their stage in the P. vivax life cycle. In other cases, an initial determination of the stage has been made but their diagnostic or therapeutic utility is not known. A non-limiting list of some of these proteins is provided below. A further list is provided with regard to Appendix I, although optionally any annotated proteins from P. vivax in Uniprot (http://www.uniprot.org/uniprot/) or another suitable protein database could be included.

| Uniprot | Protein name |
|---|---|
| A5K7E7 | hypothetical protein, conserved |
| A5K482 | hypothetical protein, conserved |
| A5K0Q6 | hypothetical protein, conserved |
| A5K4N0 | hypothetical protein, conserved |
| A5KAP7 | hypothetical protein, conserved |
| A5K4I6 | hypothetical protein, conserved |
| A5K659 | hypothetical protein, conserved |
| A5KB45 | hypothetical protein, conserved |

Very few attempts have been made to characterize the life cycle of the parasite within the body for diagnostic purposes, in terms of the dynamics of the proteins or antibody responses to specific proteins present in the blood. For example, an assay for determining a state of protective immunity is described in US20160216276. However, the disclosure relates to diagnostic assays for identifying individuals that are protected against Plasmodium falciparum caused malaria. As noted above, P. falciparum does not have a dormant liver stage with long-latency giving rise to relapses. This patent application does not mention P. vivax.

Other prior art disclosures for diagnostics focus only on the blood stage of P. vivax, which again prevents a complete picture of the dynamics of the infection in the subject from being determined. U.S. Pat. No. 6,231,861 and US20090117602 both suffer from this deficiency.

In other cases, where a plurality of antigens were examined for malarial diagnostics of P. vivax, the results still did not provide a complete picture of the dynamics of the infection in the subject. For example, “Genome-Scale Protein Microarray Comparison of Human Antibody Responses in Plasmodium vivax Relapse and Reinfection” (Chuquiyauri et al; Am. J. Trop. Med. Hyg., 93(4), 2015, pp. 801-809) suffered from the following drawbacks:
i) It only features antibody signatures that differentiate between blood-stage infections that are thought to stem either from direct infections or relapsing infections;
ii) The phenotypes are of poor quality because they are focused on genotyping with only one gene, so may overestimate the number of new infections vs relapses;
iii) They are only looking at the presence and titer of antigens at one timepoint (i.e. at the time of infection).

In another example, “Serological markers to measure recent changes in malaria at population level in Cambodia” (Kerkhof et al; Malaria Journal, 15 (1), 2016, pp. 529, the authors calculated estimated antibody half-lives to 19 Plasmodium proteins, including 5 P. vivax proteins. These 5 proteins are well-known vaccine candidates (CSP, AMA1, EBP, DBP and MSP1), and the work included no formal antigen down-selection. A major limitation of this study is that individuals were only assessed for malaria prevalence every 6 months, and hence the estimated half-lives are not a true biological reflection of what occurs in the absence of reinfection. The authors only identified one P. vivax antigen, EBP, that had an estimated antibody half-life of less than 2 years.

### BRIEF SUMMARY OF THE INVENTION

The present invention, in at least some embodiments, is of a system, method, apparatus and diagnostic test for Plasmodium vivax, to determine a likelihood of a specific timing of infection by P. vivax in a subject, and hence identify individuals with a high probability of being infected with otherwise undetectable liver-stage hypnozoites. According to at least some embodiments, the system, method, apparatus and diagnostic test relate to the identification of hypnozoites (“dormant” liver-stages), or at least of the likelihood of the subject being so infected. Optionally and preferably, the specific timing relates to recent infections, for example within the last 9 months. Without wishing to be limited by a closed list, the present invention is able to identify such recent infections, and not just current infections.

Non-limiting examples of elapsed time periods since an infection include time since infection ranging from 0 to 12 months, and each time period in between, by month, by week, and/or by day. Optionally and preferably a particular time period is determined as a binary decision of a more recent or an older infection, with each time point as a cut-off. As a non-limiting example, one such cut off could determine whether an infection in a subject was within the past 9 months or later than the past 9 months.

Optionally the timing of such an infection may also be determined, such that one or more of the following parameters may be determined. One such parameter is optionally whether the infection is a first infection in the patient, of P. vivax generally or of a particular strain of P. vivax. As there is no sterilizing immunity in malaria, immunity to one strain does not necessarily confer immunity to another, different strain. However, as described in greater detail below with regard to the examples, the present invention was tested by using samples from three different regions (including Brazil, Thailand and the Solomon Islands). These three populations are genetically highly diverse and represent the major part of the global genetic variation in P. vivax. Consequently, the present inventors believe, without wishing to be limited by a single hypothesis, that it will work anywhere in the world. Other parameters relate to time elapsed from the previous infection.

According to at least some embodiments, the antibody measurements may optionally be used to provide an estimation of elapsed time since last infection. An estimate of the time since last P. vivax blood-stage infection-depending on the available calibration data—can be defined either as the time since last PCR-detectable blood-stage parasitemia, or as the time since last infective mosquito bite. Time since last infection can be estimated continuously or categorically. Concurrent estimation of uncertainty will be important.

According to at least some embodiments, the antibody measurements may optionally be used to provide a determination of medium-term serological exposure, for example a frequency of infections during a particular time period and/or time since last infection.

According to at least some embodiments, there is provided a system, method, apparatus and diagnostic test for detection of a “silent” (asymptomatic or presymptomatic) infection by P. vivax.

According to at least some embodiments, there is provided a system, method, apparatus and diagnostic test for detection of a dormant infection, in which P. vivax is present in the liver but is not present at detectable levels in the blood. As described herein, detection of a dormant infection optionally comprises prediction from an indirect measurement of an antibody level.

During the life cycle of P. vivax, blood-stage forms of the parasite can initially be present at the same time as arrested liver forms, as described in the Background of the Invention. Even after the blood-stage infection has cleared, hypnozoites can still be present in the liver, and the parasite may still be indirectly detected via persisting antibody responses against the primary blood-stage infection. According to at least some embodiments, there is provided a system, method, apparatus and diagnostic test for detection of antibodies to malarial proteins that are present in the blood that indicate a high degree of probability of liver-stage infection.

According to at least some embodiments, there is provided a system, method, apparatus and diagnostic test for determination of the progression of infection by P. vivax in a population of a plurality of subjects. Optionally, it is possible to determine the rate of propagation of a particular Plasmodium species in a population not previously exposed to that species.

With regard to the diagnostic test, in at least some embodiments, there is provided a plurality of antibodies that bind to a plurality of antigens in a blood sample taken from the subject. Optionally any suitable tissue biological sample from a subject may be used for detecting a presence and/or level of a plurality of antibodies.

According to at least some embodiments, the dynamics of the measured antibodies preferably include a combination of short-lived and long-lived antibodies. Without wishing to be limited by a single hypothesis or a closed list, such a combination is useful to reduce measurement error.

Optionally the level of antibodies is measured at one time point or a plurality of time points.

Optionally, the presence of the actual antibodies in the blood of the subject is measured at a plurality of time points to determine decay in the level of the antibody in the blood. Such a decay in the level is then optionally and preferably fitted to a suitable model as described herein, in order to determine at least one of the infection parameters as described above. More preferably, decay of the level of a plurality of different antibodies is measured. Optionally and more preferably, the different antibodies are selected to have a range of different half-lives. Optionally, a maximum number of different antibodies is measured, which is optionally up to 20 or as few as two, or any integral number in between. According to at least some embodiments, the number of antibodies is preferably 4 or 8.

According to at least some embodiments, the level is measured of at least one antibody to a protein selected from the group consisting of: PVX_099980, PVX_112670, PVX 087885, PVX 082650, PVX_088860, PVX 112680, PVX 112675, PVX 092990, PVX_091710, PVX_117385, PVX_098915, PVX_088820, PVX_117880, PVX_121897, PVX 125728, PVX 001000, PVX_084340, PVX 090330, PVX_125738, PVX_096995, PVX_097715, PVX_094830, PVX_101530, PVX_090970, PVX_084720, PVX_003770, PVX 112690, PVX 003555, PVX_094255, PVX 090265, PVX_099930, PVX_123685, PVX_002550, PVX_082700, PVX_097680, PVX_097625, PVX_082670, PVX_082735, PVX 082645, PVX 097720, PVX 000930, PVX 094350, PVX 099930, PVX_114330, PVX_088820, PVX_080665, PVX_092995, PVX_087885, PVX_003795, PVX_087110, PVX_087670, PVX_081330, PVX_122805, RBP1b (P7), RBP2a (P9), RBP2b (P25), RBP2cNB (M5), RBP2-P2 (P55), PvDBP R3-5, PvGAMA, PvRipr, PvCYRPA, Pv DBPII (AH), PvEBP, RBP1a (P5) and Pv DBP (SacI).

Preferably, the level is measured of at least one antibody to a protein selected from the group consisting of PVX_099980, PVX_112670, PVX_087885, PVX_082650, PVX 088860, PVX 112680, PVX_112675, PVX_092990, PVX_091710, PVX_117385, PVX 098915, PVX 088820, PVX_117880, PVX_121897, PVX_125728, PVX_001000, PVX 084340, PVX 090330, PVX 125738, PVX_096995, PVX 097715, PVX_094830, PVX 101530, PVX_090970, PVX_084720, PVX_003770, PVX_112690, PVX_003555, PVX 094255, PVX_090265, PVX_099930 and PVX 123685.

More preferably, the level is measured of at least one antibody to a protein selected from the group consisting of PVX_099980, PVX_112670, PVX_087885, PVX 082650, PVX_096995, PVX 097715, PVX_094830, PVX_101530, PVX_090970, PVX 084720, PVX_003770, PVX_112690, PVX_003555, PVX_094255, PVX_090265, PVX 099930 and PVX 123685.

Most preferably, the level is measured of at least one antibody to a protein selected from the group consisting of PVX_099980, PVX_112670, PVX_087885 and PVX 082650.

According to at least some embodiments, preferably the level is measured of at least one antibody to a protein selected from the group consisting of RBP2b, L01, L31, X087885, PvEBP, L55, PvRipr, L54, L07, L30, PvDBPII, L34, X092995, L12, RBP1b, L23, L02, L32, L28, L19, L36, L41, X088820 and PvDBP . . . SacI.

More preferably the level is measured of at least one antibody to a protein selected from the group consisting of RBP2b, L01, L31, X087885, PvEBP, L55, PvRipr, L54, L07, L30, PvDBPII, L34, X092995, L12 and RBP1b.

Also more preferably the level is measured of at least one antibody to a protein selected from the group consisting of RBP2b, L01, L31, X087885, PvEBP, L55, PvRipr and L54.

Most preferably the level is measured of at least one antibody to a protein selected from the group consisting of RBP2b and L01.

A table containing additional proteins against which antibodies may optionally be measured is provided herein in Appendix I, as described in greater detail below, such that the level of one or more of these antibodies may optionally be measured.

Appendix II gives a list of preferred proteins against which antibodies may be measured, while Appendix III shows a complete set of data for antibodies against the proteins in Appendix II. Appendix III is given in two parts, due to the size of the table: Appendix IIIA and Appendix IIIB. The references to gene identifiers in Appendix II are the common ones used for Plasmodium—from PlasmoDB website: http://plasmodb.org/plasmo/.

For any protein described herein, optionally a fragment and/or variant may be used for detecting the presence and/or level of one or more antibodies in a biological sample taken from a subject.

According to at least some embodiments, a biologically-motivated model of the decay of antibody titers over time is used to determine a statistical inference of the time since last infection. The model preferably uses previously determined decay rates of a plurality of different antibodies to determine a likelihood that infection in the subject occurred within a particular time period. Optionally such previously determined decay rates may be achieved through estimation of antibody decay rates from longitudinal data, or estimation of decay rates from cross-sectional antibody measurements.

With regard to estimation of antibody decay rates from longitudinal data, preferably such an estimation is performed as described in equation (1), which is a mixed-effects linear regression model:

$$log ( {A}_{ijk} ) \sim ( {α}_{k}^{0} + {α}_{ik} ) + ( {r}_{k}^{0} + {r}_{ik} ) {t}_{j} + {ε}_{k} \qquad Equation 1$$

$${α}_{ik} \sim N ( 0 , {σ}_{α , k} )$$

$${r}_{ik} \sim N ( 0 , {σ}_{r , k} )$$

$${ε}_{ik} \sim N ( 0 , {σ}_{m , k} )$$

For the above equation to be true, the following assumptions were made. We assume that for individual i we have measurements of antibody titer Aᵢⱼₖ at time j to antigen k. We assume that at time 0, antibody titers are Normally distributed5 with mean αₖ⁰ and standard deviation σ_{α,k} on a log-scale. We assume that an individual's rate of antibody decay is drawn from a Normal distribution with mean rₖ⁰ and standard deviation σ_{r,k}.

According to at least some embodiments, the plurality of different antibodies selected maximizes probability of determining at least one of the infection parameters as described above. A method for such a selection process is described below in Example 3. Optionally the plurality of antibodies is selected for determining an answer to a binary determinant, such as for example, whether an individual was infected before x months ago or after as previously described.

According to at least some embodiments, the model for determining at least one parameter about the infection in the subject may optionally comprise one or more of the following algorithms: linear discriminant analysis (LDA), quadratic discriminant analysis (QDA), combined antibody dynamics (CAD), decision trees, random forests, boosted trees and modified decision trees.

According to at least some embodiments, the levels of antibody in a blood-sample can be measured and summarized in a variety of ways, for input to the above described model.

### a) Continuous Measurement

A continuous measurement that has a monotonic relationship with antibody titer. It can be compared with a titration curve to produce an estimate of antibody titer.

### b) Binary Classification

Assesses whether antibody levels are greater or less than some threshold

### c) Categorical Classification

Assigns antibody levels to one of a set of pre-defined categories, e.g. low, medium, high. A categorical classification can be generated via a series of binary classifications.

According to at least some embodiments, antibody levels may optionally be measured in a subject in a number of different ways, including but not limited to, bead-based assays (e.g. AlphaScreen® or Luminex® technology), the enzyme linked immunosorbent assay (ELISA), protein microarrays and the luminescence immunoprecipitation system (LIPS). All the aforementioned methods generate a continuous measurement of antibody.

### BRIEF DESCRIPTION OF THE DRAWINGS

FIG. 1 shows a background art description of the lifecycle of P. vivax (see Mueller, I. et al. Key gaps in the knowledge of Plasmodium vivax, a neglected human malaria parasite. Lancet Infectious Diseases 9, 555-566 (2009)).

FIG. 2 shows a method for data processing and down-selection of candidate serological markers.

FIG. 3 shows an example of two differing antibody kinetic profiles. Antibody responses at the four time-points measured in the AlphaScreen® assay are shown for two proteins, PVX_099980 and PVX_122680. An arbitrary positivity cut-off is marked at 0.94 (the average of the wheat germ extract control well+6× standard deviation). Data is generated from 32 individuals in Thailand.

FIG. 4 shows characteristics of the top 55 protein constructs. (A) Length of the estimated antibody half-lives, note for 4 proteins the classification was different between Thailand and Brazil. (B)-(F) Details of protein characteristics as determined by PlasmoDB release 25 or published literature: (B) predicted expression stage, (C) presence of a signal peptide sequence, (D) presence of transmembrane domain/s, (E) presence of a GPI anchor, (F) annotation. TM=transmembrane domains, MSPs=merozoite surface proteins, RBPs=reticulocyte binding proteins.

FIG. 5 shows correlation between antibody measurements in Thailand and Brazil. Correlation of data from the antigen discovery study generated using the AlphaScreen® assay. Correlations are shown for the 55 down-selected candidate serological markers. (A) Comparison of the proportion of individuals defined as positive at time of P. vivax infection (antibody value above the lower point of the standard curve, i.e. 0). (B) Comparison of the geometric mean antibody titers (GMT). (C) Comparison of the estimated antibody half-lives. Spearman correlation coefficients, r, are shown. Data was generated from 32 individuals in Thailand and 33 in Brazil.

FIG. 6A shows optimization of Luminex® bead-array assay for the first 17 proteins. Log-linear standard curves were achieved for all proteins, using the amounts of protein shown for one bulk reaction of 500 μl beads.

FIGS. 6B-6D show additional development and optimization of the Luminex bead-array assay for all 65 proteins assessed in the validation study as follows. FIG. 6B shows 40 down-selected proteins. FIG. 6C shows the remaining 25 proteins. Log-linear standard curves were achieved for all proteins. The amount of protein for one bulk reaction of 500 ul beads is shown in FIG. 6D, with the line indicating the median (1 and 1.08 ug, respectively).

FIG. 6E provides a key to FIG. 6B. FIG. 6F provides a key to FIG. 6C.

FIG. 7 shows the association of antibody levels with current P. vivax infections in the Thai validation cohort. Antibody responses were measured at the last time-point of the Thai cohort against the first 17 proteins assessed, using the Luminex® bead-array assay. The association between antibody responses and current infection was assessed using a logistic regression model, adjusting for age, sex and occupation. Odds ratios are shown, with 95% confidence intervals. Associations for all antibodies were significant (p<0.05). The estimate of antibody half-life shown is based on the antigen discovery dataset (AlphaScreen®).

FIG. 8 shows association of antibody levels with past P. vivax exposure in the Thai validation cohort. Antibody responses were measured at the last time-point of the Thai cohort against the first 17 proteins assessed, using the Luminex® bead-array assay. The association between antibody responses and total exposure over the past year was assessed using a generalised linear model, adjusting for age, sex, occupation and current infection status. Exponentiated coefficients are shown, with 95% confidence intervals. Associations for all antibodies, except PVX_09070, were significant (p<0.05). The estimate of antibody half-life shown is based on the antigen discovery dataset (AlphaScreen®).

FIG. 9 shows the association of antibody levels with current P. vivax infections in the Brazilian validation cohort. Antibody responses were measured at the last time-point of the Brazilian cohort against the first 17 proteins assessed, using the Luminex® bead-array assay. The association between antibody responses and current infection was assessed using a logistic regression model, adjusting for age, sex and occupation. Odds ratios are shown, with 95% confidence intervals. Associations for all antibodies, except PVX_088860, were significant (p<0.05). The estimate of antibody half-life shown is based on the antigen discovery dataset (AlphaScreen®).

FIG. 10 shows the association of antibody levels with past P. vivax exposure in the Brazilian validation cohort. Antibody responses were measured at the last time-point of the Brazilian cohort against the first 17 proteins assessed, using the Luminex® bead-array assay. The association between antibody responses and total exposure over the past year was assessed using a generalised linear model, adjusting for age, sex, occupation and current infection status. Exponentiated coefficients are shown, with 95% confidence intervals. Associations for 10 of the 17 antibodies were significant (p<0.05). The estimate of antibody half-life shown is based on the antigen discovery dataset (AlphaScreen®).

FIG. 11 shows longitudinal antibody dynamics of 4 antigens from 8 Thai participants in the antigen discovery cohort. For each blood sample antibody titers were measured in triplicate, using the AlphaScreen® assay. Each colour corresponds to antibodies to a different antigen. The lines represent the fit of the mixed-effects regression model described below.

FIG. 12 shows the relationship between antibody titers to 8 P. vivax antigens and time since last PCR-detectable in individuals from a malaria-endemic region of Thailand (validation study, antibodies measured via Luminex® bead-array assay). The grey bars denote individuals with current infection (n=25); infection within the last 9 months (n=47); infection 9-14 months ago (n=25); and no infection detected within the last 14 months (n=732). The orange bars show the antibody titers from three different panels of negative controls.

FIG. 13 presents the association between measured antibody titer xᵢₖ and time since infection t. (a) There are three sources of variation in the antibody titer xᵢₖ measured at time t since last infection: (i) variation in initial antibody titer; (ii) between individual variation in antibody decay rate; and (iii) measurement error. (b) Given estimates of the sources of variation, we can estimate the distribution of the time since last infection. The maximum likelihood estimate and the 95% confidence intervals of our estimate are indicated in blue.

FIG. 14 shows the dynamics of multiple antibodies. The variance in initial antibody titer, antibody decay rates and measurement error are now described by covariance matrices, which account for the correlations between antibodies.

FIG. 15 shows an example of QDA classification for participants from the Thai validation cohort. Antibody measurements were made using the Luminex® bead-array assay. Each point corresponds to a measurement from a single individual with log(anti-L01 antibody titer) on the x-axis and log(anti-L22 antibody titer) on the y-axis. The blue ellipse represents the multivariate Gaussian fitted to data from individuals with ‘old’ infections and the red ellipse represents the multivariate Gaussian fitted to data from individuals with ‘new’ infections. The dashed lack line represents the boundary for classifying individuals according to whether or not they have had a recent infection.

FIG. 16 shows receiver operator characteristic (ROC) curves estimated via cross-validation for LDA (blue) and QDA (black) classification algorithms, using the Thai validation data measured via the Luminex® bead-array assay.

FIG. 17 shows an example of a decision tree for classifying old versus new infections using measurements of antibodies to 6 P. vivax antigens, using the Thai validation data measured via the Luminex® bead-array assay.

FIG. 18 shows ROC curve demonstrating the association between sensitivity and specificity for a decision tree algorithm, using the Thai validation data measured via the Luminex® bead-array assay. These curves have been generated through cross-validation by splitting the data into training and testing sets. The algorithm is formulated using the training data set and the sensitivity and specificity evaluated on the testing data set. The colours correspond to different subsets of antigens. Notably, we can obtain nearly 80% sensitivity with specificity >95%.

FIG. 19 shows a random forest variable importance plot of the contribution of antibodies to 17 antigens towards correct classification of infections into ‘new’ versus ‘old’, using the Thai validation data measured via the Luminex® bead-array assay. Antigens with greater values of ‘MeanDecreaseAccuracy’ are considered the most informative. Therefore L01 provides the most information for classification purposes.

FIG. 20 shows an example of antigen down-selection using the simulated annealing algorithm. Data comes from the antigen discovery study using the AlphaScreen® assay. (A) Including additional antigens increases the likelihood that infection times will be correctly classified, but with diminishing returns. (B) Each column of the heatmap denotes one of K=98 antigens. The y-axis denotes the maximum number of antigens that can be included in a panel. Red antigens are more likely to be included in a panel of a given size. (C) Example of predicting time since last infection in 4 individuals using a panel of 15 antigens. The vertical dashed line at 6 months represents an infection occurring 6 months ago. The solid black curve denotes the estimated distribution of the time since last infection. The green point denotes the maximum likelihood estimate of the model, and the vertical green bars denote the 95% confidence intervals. The red, shaded area denotes infection within the last 9 months. If more than 50% of the probability mass of the distribution is in this region, then the infection will be classified as having occurred within the last 9 months.

FIG. 21 shows comparison of age-stratified prevalence of PCR detectable blood-stage infection within the last 9 months;

FIG. 22 shows measured antibody titers to four P. vivax antigens from Thailand, Brazil and the Solomon Islands, and from three panels of negative controls. The box plots show the median, interquartile range and 95% range of measured antibody titers. The horizontal dashed lines represent the lower and upper limits of detection;

FIGS. 23A-23C show an overview of cross-validated random forests classification algorithms. The classifiers were trained on data from either Thailand, Brazil or The Solomon Islands; and

FIG. 24 shows an exemplary network visualization of combinations of 4 antigens. The size of the node represents the probability that an antigen appears in the best performing combinations. The width and darkness of the edges represents the probability that two antigens are selected together in the best performing combinations. Red denotes proteins purified at high yield by CellFree Sciences (the 40 down selected proteins, the results for which are shown in FIG. 6B). Blue denotes vaccine candidate antigens. Green denotes proteins expressed in wheat-germ by Ehime University. Blue and green proteins are the 25 additional proteins, the results for which are shown in FIG. 6C.

FIG. 25 shows cross-validated Receiver Operating Characteristic (ROC) curves from linear discriminant analysis (LDA) classifiers trained and tested using combinations of four antigens from Thailand, Brazil and The Solomon Islands.

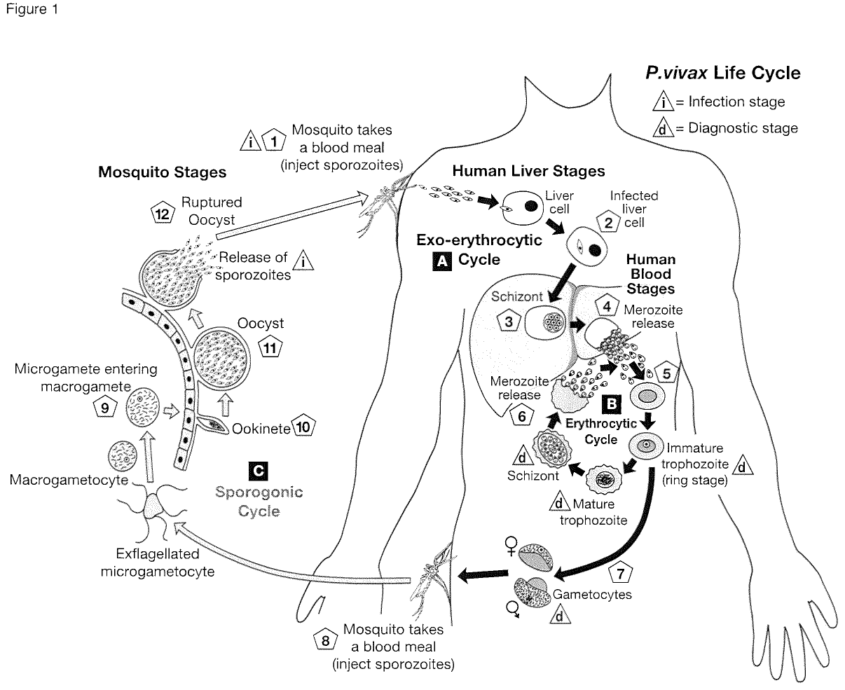

### DESCRIPTION OF AT LEAST SOME EMBODIMENTS

The present invention, in at least some embodiments, is of a system, method, apparatus and diagnostic test for at least Plasmodium vivax, and optionally other species such as P. ovale, to determine a likelihood of a concurrent or the specific timing of a recent past infection by P. vivax in a subject, and hence identify individuals with a high probability of being infected with otherwise undetectable liver-stage hypnozoites. According to at least some embodiments, the system, method, apparatus and diagnostic test relate to the identification of hypnozoites (“dormant” liver-stages), or at least of the likelihood of the subject being so infected. Optionally and preferably, the specific timing relates to recent infections, for example within the last 9 months. Without wishing to be limited by a closed list, the present invention is able to identify such recent infections, and not just current infections.

According to at least some embodiments, the antibody measurements may optionally be used to provide an estimation of elapsed time since last infection. An estimate of the time since last P. vivax blood-stage infection-depending on the available calibration data, the time since last infection can be defined either as the time since last PCR-detectable blood-stage parasitemia, or as the time since last infected mosquito bite. Time since last infection can be estimated continuously or categorically. Concurrent estimation of uncertainty will be important.

According to at least some embodiments, the antibody measurements may optionally be used to provide a determination of medium-term serological exposure, for example a frequency of infections during a particular time period and/or time since last infection.

According to at least some embodiments, there is provided a system, method, apparatus and diagnostic test for detection of a “silent” (asymptomatic or presymptomatic) infection by P. vivax.

### Protein Nomenclature

Throughout the below experiments, simplified names have been used for the proteins assessed. In the antigen discovery experiments using the AlphaScreen® assay, 342 proteins were assessed. These proteins were given codes consisting of single letters followed by 2 numbers in most instances, and on occasion 3 numbers.

In the validation experiments using the multiplexed assay (Luminex® technology), 40 proteins (out of the 53 potential candidates down-selected) were assessed. These proteins have been given codes beginning with ‘L’ followed by 2 numbers. These proteins were supplemented by an additional 25 proteins expressed in a variety of systems. These proteins have been given codes beginning with ‘V’ or ‘X’ followed by 2 numbers. The codes used for the tested candidates are outlined below, as well as in Appendix II, in reference to their PlasmoDB gene ID (plasmodb.org).

| PlasmoDB ID | AlphaScreen | Luminex |
|---|---|---|
| PVX_099980 | D10 | L01 |
| PVX_096995 | J12 | L02 |
| PVX_088860 | L19 | L03 |
| PVX_097715 | N17 | L07 |
| PVX_112680 | K21 | L06 |
| PVX_094830 | N13 | L10 |
| PVX_112675 | B19 | L11 |
| PVX_112670 | G21 | L12 |
| PVX_101530 | D21 | L05 |
| PVX_090970 | E10 | L14 |
| PVX_084720 | B8 | L18 |
| PVX_003770 | P17 | L19 |
| PVX_092990 | H14 | L20 |
| PVX_112690 | K10 | L21 |
| PVX_091710 | F13 | L22 |
| PVX_087885 | N9 | L23 |
| PVX_003555 | O21 | L24 |

A complete list of all sequences considered, plus the sequences themselves, may be found in Appendices I and II combined. These sequences include the reference to the amino acid and nucleic acid sequence records of the relevant antigens, plus actual sequences generated for testing. The actual amino acid sequences generated for testing include a methionine at the start (N-terminus) and a His-tag at the end (C-terminus) as non-limiting examples only. The nucleic acid sequences so generated correspond to these amino acid sequences. It should be noted that the sequences listed are intended as non-limiting examples only, as different sequences and/or different antigens may optionally be used with the present invention, additionally or alternatively. The amino acid sequences for the specific proteins referred to herein may optionally be obtained from Uniprot or another suitable protein database.

### Example 1—Testing of Antigens

This non-limiting Example relates to testing of antibody responses to various P. vivax proteins, present in the blood, as potential antigens for a diagnostic test.

### Materials and Methods

### Ethics Statement.

The relevant local ethics committees approved all field studies and all patients gave informed consent or assent. The Ethics Committee of the Faculty of Tropical Medicine, Mahidol University, Thailand approved the Thai antigen discovery and validation studies (MUTM 2014-025-01 and 02, and MUTM 2013-027-01, respectively). The Ethics Review Board of the Fundação de Medicina Tropical Dr. Heitor Vieira Dourado (FMT-HVD) (957.875/2014) approved the Brazilian antigen discovery study. The samples used from Brazil for the validation study were approved by the FMT-HVD (51536/2012), by the Brazilian National Committee of Ethics (CONEP) (349.211/2013) and by the Ethics Committee of the Hospital Clinic, Barcelona, Spain (2012/7306). The National Health Research and Ethics Committee of the Solomon Islands Ministry of Health and Medical Services (HRC12/022) approved collection of the samples used from the Solomon Islands for the validation study. The Human Research Ethics Committee at WEHI approved samples for use in Melbourne (#14/02).

### Field Sites and Sample Collection: Antigen Discovery Study.

Samples from two longitudinal cohorts, located in Thailand and Brazil, were used for the antigen discovery studies. The longitudinal study in Thailand was conducted from April 2014 to September 2015, as previously described (Longley et al., Am J Trop Med Hyg. 2016 Nov. 2; 95(5): 1086-1089). Briefly, 57 symptomatic P. vivax patients were enrolled from either the Tha Song Yang malaria clinic or hospital. Patients with glucose-6-phosphate dehydrogenase (G6PD) deficiency and those aged younger than 7 years or more than 80 years were excluded. Patients were treated with chloroquine (25 mg base/kg body weight, administered over 3 days) and primaquine (15 mg daily, for 14 days) according to the standard Thai treatment regimen. Anti-malarial drugs were given under directly-observed treatment in order to reduce the likelihood of treatment failure and the presence of recurrent infections during follow-up. Volunteers were followed for 9-months following enrolment, with finger-prick blood samples collected at enrolment and week 1, then every 2 weeks for 6 months, then every month until the end of the study. Blood was separated into packed red cells and plasma at the field site. All blood samples were analysed by both light microscopy and quantitative PCR (qPCR) for the presence of blood-stage parasites. A sub-set of volunteers, n=32, were selected for use in the antigen discovery project. These volunteers had no detectable recurrent infections during 9-months follow-up, and were the first to complete follow-up.

The longitudinal study in Brazil followed the same format as in Thailand. The study was conducted from May 2014 to May 2015. 91 malaria patients at Fundação de Medicina Tropical Doutor Heitor Vieira Dourado in Manaus aged between 7 and 70 years were enrolled. Individuals with G6PD deficiency or chronic diseases were not enrolled. Patients were treated according to the guidelines of the Brazilian Ministry of Health (3 days chloroquine, 7 days primaquine). Follow-up intervals with finger-prick blood sample collection were as in the Thai study. A sub-set of volunteers, n=33, whom had no detectable recurrent infections during 9-months follow-up, were selected for use in the antigen discovery project.

### Field Sites and Sample Collection: Validation Study.

For the validation studies, samples collected from four observational longitudinal cohort studies, conducted in Thailand, Brazil and the Solomon Islands, were used (data from the Solomon Islands not shown). Samples were collected from a cohort of volunteers every month for 1 year. Plasma samples from the final cohort time-point were used in the validation study, n=829 Thailand, n=925 Brazil, and n=751 Solomon Islands.

The Thailand observational cohort was conducted from May 2013 to June 2014 in the Kanchanaburi and Ratchaburi provinces of western Thailand. The design of this study has been published (Longley et al, Clin Vaccine Immunol. 2015 Dec. 9; 23(2): 117-24). Briefly, a total of 999 volunteers were enrolled (aged 1-82 years, median 23 years). Volunteers were sampled every month over the yearlong cohort, with 14 active case detection visits performed in total. A total of 609 volunteers attended all visits, with 829 attending the final visit. At each visit, volunteers completed a brief survey outlining their health over the past month (to determine the possibility of missed malarial infections), in addition to travel history and bed net usage. A finger-prick blood sample was also taken and axillary temperature recorded. Blood samples were separated into packed red blood cells, for detection of malaria parasites, and plasma, for antibody measurements, at the field sites. In addition to the monthly active case detection visits, passive case detection was also performed routinely by local malaria clinics.

The Brazilian observational cohort was conducted from April 2013 to April 2014 in three neighbouring communities located on the outskirts of Manaus, Amazonas State. Briefly, a total of 1274 residents of all age groups were enrolled (range 0-102 years, median 25 years). Volunteers were sampled every month over the yearlong period, with 13 active case detection visits performed in total. At each visit a finger-prick blood sample was collected, with the exception of children aged less than one in which blood was collected from the heel or big toe. As per the Thai cohort study, at each visit body temperature was also recorded and a questionnaire undertaken outlining the participants' health, bed net usage and travel history. Passive case detection was performed routinely by local health services. Blood samples were processed as per the Thai cohort. Plasma samples from 925 volunteers were available from the final visit.

The Solomon Islands observational cohort was conducted from May 2013 to May 2014 in 20 villages on the island of Ngella, Solomon Islands. 1111 children were initially enrolled, and after exclusion of children who withdrew, had inconsistent attendance or failed to meet other inclusion criteria, 860 remained (Quah & Waltmann, in preparation). The age of the children ranged from 6 months to 12 years, with a median age of 5.6 years. Over the yearlong cohort, children were visited approximately monthly, with 11 active case detection visits in total. Of the 860 children, 751 attended the final visit. At each visit, a brief survey was conducted as per the Thai cohort, temperature recorded and a finger-prick blood sample taken. Blood was separated into packed red cells for qPCR and plasma for antibody measurements. In addition to the monthly active case detection visits, local health clinics and centres also performed passive case detection routinely.

### Negative Control Samples: Melbourne and Thai Red Cross, Melbourne Blood Donors

Three panels of control samples were collected from individuals with no known previous exposure to malaria. The first panel was collected from the Volunteer Blood Donor Registry (VBDR) at the Walter and Eliza Hall of Medical Research in Melbourne, Australia. These individuals are not screened for diseases but a record of their past travel, medical history and current drug use is recorded. 102 volunteers from the VBDR were utilized (median age 39 years, range 19-68). The second panel was collected from the Australian Red Cross (Melbourne, Australia). 100 samples were utilized (median age 52 years, range 18-77), and these individuals were screened as per the standard conditions of the Australian Red Cross. Finally, another control panel was collected from the Thai Red Cross (Bangkok, Thailand). Samples from 72 individuals were utilized, but no demographic data was available (hence the age range is unknown). Standard Thai Red Cross screening procedures exclude individuals from donating blood if they had a past malaria infection with symptoms within the last three years, and individuals who have travelled to malaria-endemic regions within the past year.

All studies (antigen discovery and validation) detected malaria parasites by quantitative PCR as previously described (2, 3).

### Protein Expression.

Proteins were preferably expressed as full-length proteins, to ensure that any possible antibody recognition site was covered. For very large proteins, fragments were expressed that together cover the entire protein. These were treated as individual constructs in the down-selection process. The proteins were first produced at a small-scale with a biotin tag for the antigen discovery study, at Ehime University. A panel of 342 P. vivax proteins were assessed, including well-known P. vivax proteins such as potential vaccine candidates (i.e. MSP1, AMA1, CSP), orthologs of immunogenic P. falciparum proteins and proteins with a predicted signal peptide (SP) and/or 1-3 transmembrane domains (TM) (4). The genes were amplified by PCR and cloned into the pEU_E01 vector with N-terminal His-bls tag (CellFree Sciences, Matsuyama, Japan). P. vivax genes were obtained either from parent clones (4), using SAL-1 cDNA, or commercially synthesized from Genscript (Japan). The recombinant proteins were expressed without codon optimization using the wheat germ cell-free (WGCF) system as previously described (5). WGCF synthesis of the P. vivax protein library was based on the previously described bilayer diffusion system (6). For biotinylation of proteins, 500 nM D-biotin (Nacalai Tesque, Kyoto, Japan) was added to both the translation and substrate layers. Crude WGCF expressed BirA (1 μl) was added to the translation layer. In vitro transcription and cell-free protein synthesis for the P. vivax protein library were carried out using the GenDecoder 1000 robotic synthesizer (CellFree Sciences) as previously described (7, 8). Expression of the proteins was confirmed by western blot using HRP-conjugated streptavidin.

Large-scale protein expression for the down-selected candidates was then performed (CellFree Sciences Tokyo, Japan). Genes were synthesized by GenScript (Japan) and the products cloned into the pEU-E01-MCS expression vector. The sequence of all gene synthesis products and their correct insertion into the expression vector was confirmed by full-length sequencing of the vector inserts. Transcription was performed utilizing SP6 RNA polymerase (80 U/μl) and the SP6 promoter in the pEU-E01-MCS expression vector. For large-scale expression, a dialysis-based refeeding assay was used, with protein expression and solubility first tested on a 50 μl scale. The test results then enabled production on a 3 ml scale (maintained for up to 72 hours, 15° C.) to produce at least 300 μg of each target protein, using the wheat germ extract WEPRO7240H. The proteins were manually purified one-time on an affinity matrix (Ni Sepharose 6 Fast Flow from GE Healthcare, Chalfont, United Kingdom) using a batch method (all proteins were expressed with a His-tag at the C terminus). The purified proteins were stored and shipped in the following buffer: 20 mM Na-phosphate pH 7.5, 0.3 M NaCl, 500 mM imidazole and 10% (v/v) glycerol. Protein yields and purity were determined using 15% SDS page followed by Coomassie Brilliant Blue staining using standard laboratory methods. In addition, proteins were also analyzed by Western Blot using an anti-His-tag antibody.

An additional 25 proteins were also used in the validation study. 12 proteins were produced using the wheat-germ cell free system described above at Ehime University, and were selected based on associations with past exposure in preliminary work conducted in a PNG cohort. The remaining 13 proteins were produced using standard E. coli methods, and were selected based on their predicted high immunogenicity (due to their status as potential vaccine candidates). References can be found in Appendix II.

### AlphaScreen® Assay for the Antigen Discovery Study.

The AlphaScreen® assay was used to measure antibody responses in the antigen discovery study. Plasma samples from the sub-set of volunteers (n=32 Thailand, n=33 Brazil) were used from four time-points, enrolment (week 0) and weeks 12, 24 and 36. Responses were measured against 342 P. vivax proteins. The assay was conducted as previously reported (9), with slight modifications. The protocol was automated by use of the JANUS Automated Workstation (PerkinElmer Life and Analytical Science, Boston, MA). Reactions were carried out in 25 μl of reaction volume per well in 384-well OptiPlate microtiter plates (PerkinElmer). First, 0.1 μl of the translation mixture containing a recombinant P. vivax biotinylated protein was diluted 50-fold (5 μl), mixed with 10 μl of 4000-fold diluted plasma in reaction buffer (100 mM Tris-HCL [pH 8.0], 0.01% [v/v] Tween-20 and 0.1% [w/v] bovine serum albumin), and incubated for 30 min at 26° C. to form an antigen-antibody complex. Subsequently, a 10 μl suspension of streptavidin-coated donor-beads and acceptor-beads (PerkinElmer) conjugated with protein G (Thermo Scientific, Waltham, MA) in the reaction buffer was added to a final concentration of 12 μg/ml of both beads. The mixture was incubated at 26° C. for one hour in the dark to allow the donor and acceptor-beads to optimally bind to biotin and human IgG, respectively. Upon illumination of this complex, a luminescence signal at 620 nm was detected by the En Vision plate reader (PerkinElmer) and the result was expressed as AlphaScreen counts. A translation mixture of WGCF without template mRNA was used as a negative control. Each assay plate contained a standard curve of total biotinylated rabbit IgG. This enabled standardisation between plates using a 5-parameter logistic standard curve. All samples were run in triplicate. Reading the plates was conducted in a randomized manner to avoid biases.

### Multiplexed Bead-Based Assay for the Validation Study.

For validation of the down-selected candidate serological markers, IgG levels were measured in plasma collected from the last time-point of the longitudinal observation studies. IgG measurements were performed using a multiplexed bead-based assay as previously described (10). In brief, 2.5×10⁶ COOH microspheres (Bio-Rad, USA) were prepared for protein coupling by incubation for 20 minutes at room temperature in 100 mM monobasic sodium phosphate (pH 6.2), 50 mg/ml N-Hydroxysulfosuccinimide sodium salt and 50 mg/ml N-(3-Dimethylaminopropyl)-N′-ethylcarbodiimide hydrochloride. Proteins were then added and incubated overnight at 4° C. Optimal amounts of protein were determined experimentally, in order to achieve a log-linear standard curve when using a positive control plasma pool generated from hyper-immune PNG donors. Each assay plate subsequently included this 2-fold serial dilution standard curve (1/50 to 1/25600), to enable standardisation between plates.

The assay was run by incubating 50 μl of the protein-coupled microspheres (500 microspheres/well) with 50 μl test plasma (at 1/100 dilution) in 96-well multiscreen filter plates (Millipore, USA) for 30 minutes at room temperature, on a plate shaker. Plates were washed 3 times and then incubated for a further 15 minutes with the detector antibody, PE-conjugated anti-human IgG (1/100 dilution, Jackson ImmunoResearch, USA). The plates were once again washed and then assayed on a Luminex 200™ instrument. All median fluorescent intensity (MFI) values were converted to relative antibody unites using the plate-specific standard curve (five-parameter logistic function, as previously described in detail (10)).

### Statistical Modelling.

The models are described in greater detail below (see Example 3).

### Statistical Analysis.

All data manipulation and statistical analyses were performed in either R version 3.2.3 (11), Prism version 6 (GraphPad, USA) or Stata version 12.1 (StataCorp, USA).

### Results

### Down-Selection of Candidate Serological Markers.

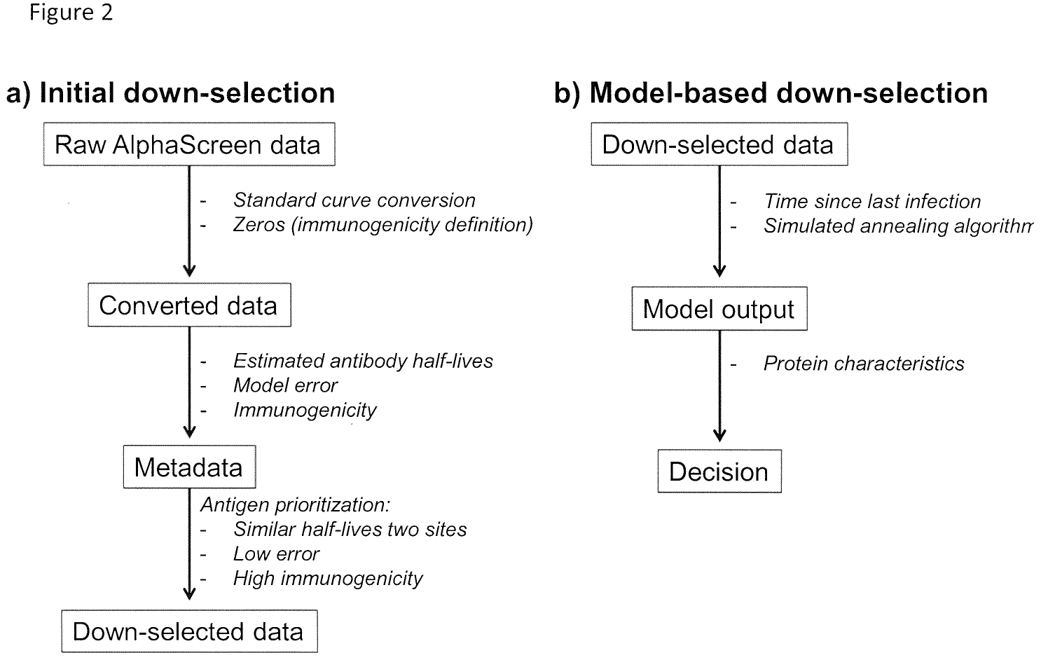

The data were processed and candidate serological markers down-selected following the pipeline shown in FIG. 2. The raw AlphaScreen data was converted based on the plate-specific standard curve, resulting in relative antibody units ranging from 0-400. Using the converted data, seropositivity was defined as a relative antibody unit greater than 0. For proteins that were defined as immunoreactive (more than 10% individuals seropositive at baseline, time of P. vivax infection), an estimated antibody half-life was determined using a mixed-effects linear regression model, described in detail below (see Statistical modelling). Using the metadata on immunoreactivity and half-life, an initial round of antigen down-selection was performed, with prioritisation of antigens that had similar estimated half-lives in both the Thai and Brazilian datasets (neither site more than double the other), high levels of seropositivity at baseline (more than 50% individuals seropositive, i.e. relative antibody units above 0), and low levels of error estimated in the model. Three rounds of initial down-selection were performed, resulting in approximately 100 antigens for the next round of model-based down-selection.

The model-based down-selection was performed in two stages: first, by calculating the estimated time since last infection based on antibody levels at 0, 3, 6 and 9 months (and comparing this with the known time since infection), and second, by determining the best combination of antigens for accurately detecting the time since last infection.

In more detail, FIG. 2 shows a pipeline for down-selection of candidate serological markers. As shown in the process of FIG. 2A, antigens were first down-selected based on prioritization of metadata characteristics such as similar levels of estimated antibody longevity in Thailand and Brazil, high levels of immunogenicity at the time of infection and low levels of error estimated in the model. As shown in the process of FIG. 2B, using the initial down-selected antigens, further modelling was performed to identify the optimal combination of antigens able to accurately estimate the time since last infection. A final decision on candidate serological markers was made using output from this modelling and other protein characteristics, as detailed above.

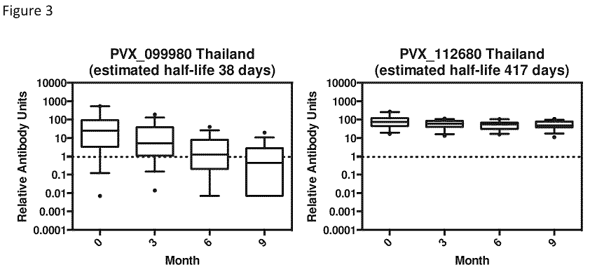

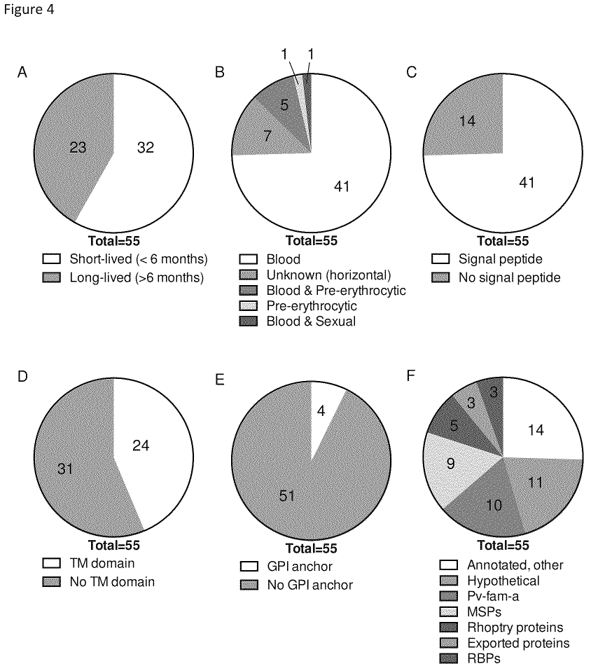

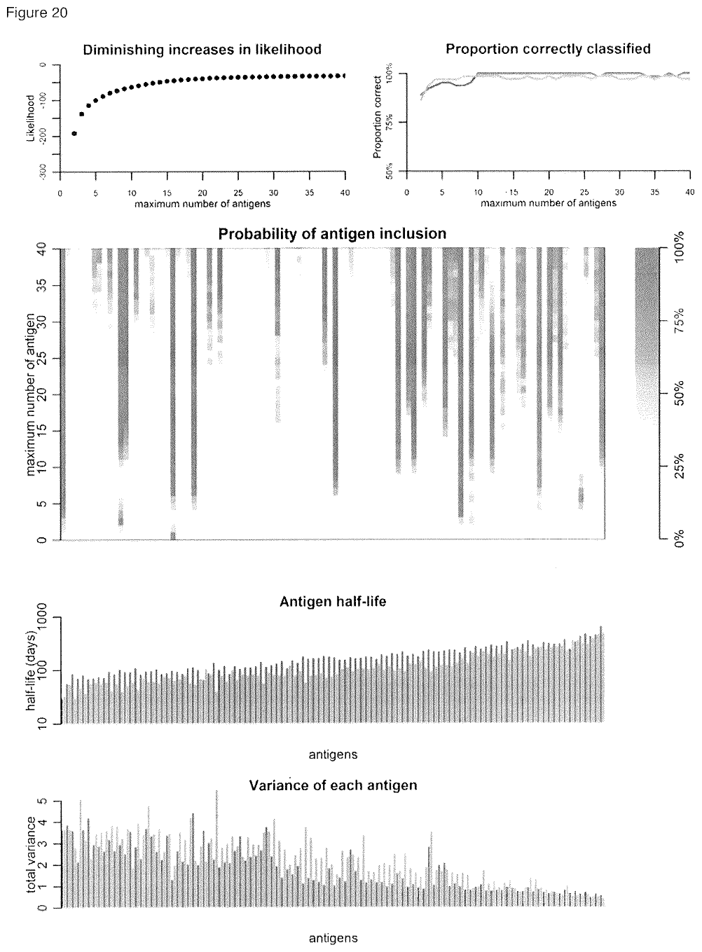

As expected, different antibody kinetic profiles over 9-months were observed for different proteins (see FIG. 3 for an example). Antigen down-selection was performed as described in detail in the Materials and Methods, essentially by prioritizing antigens with high levels of immunogenicity, similar estimated half-lives between Thailand and Brazil and low levels of error estimated in the model. The initial down-selection was followed by further model-based down selection. The model-based down-selection was used to determine the ability of various proteins to predict the time since last infection, utilizing the same datasets from Thailand and Brazil, and to determine the best combination of proteins to do so successfully (see for example FIG. 20 and its accompanying description). Antigens were excluded from selection if they had less than a 40% probability of inclusion in a 40-antigen panel that was able to accurately determine the time since last infection. Remaining antigens were then ranked according to a high probability of inclusion in a successful 20-antigen panel. When required, ranking in 30 and 40-antigen panels was also considered. Antigens were excluded if they had unfavorable protein production characteristics, such as low-yield in the small-scale WGCF expression or presence of aggregates. Three rounds of selection were performed: the first resulted in 12 proteins, the second in a further 12, and the third in an additional 31 candidates. A final list of 55 protein constructs (53 unique proteins) representing candidate serological markers of recent exposure to P. vivax infection was generated (two fragments were included from two different antigens). Characteristics of these proteins are highlighted in FIG. 4.

### Validation of Candidate Serological Markers.

Geographical validation (that is validation across different regions) was performed as follows.

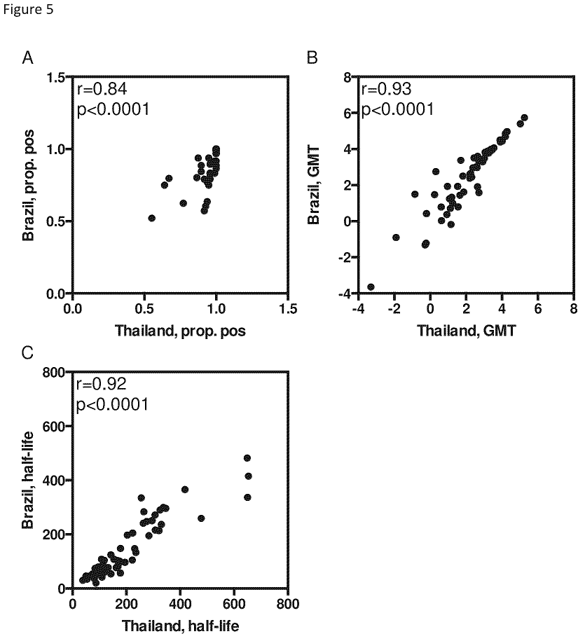

The down-selected markers were chosen based on antibody data from individuals in Thailand, Brazil and the Solomon Islands, three discrete geographical areas. Despite this, there was a strong correlation between the antibody responses measured, in terms of both immunogenicity (seropositivity rates) and antibody level at time of P. vivax infection, as well as the estimated antibody half-lives calculated from consecutive time-points. This is shown in FIG. 5.

Validation in association with recent and past infection was performed as well.

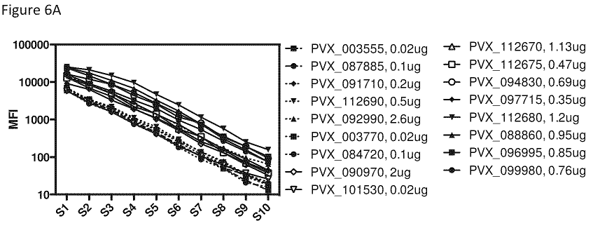

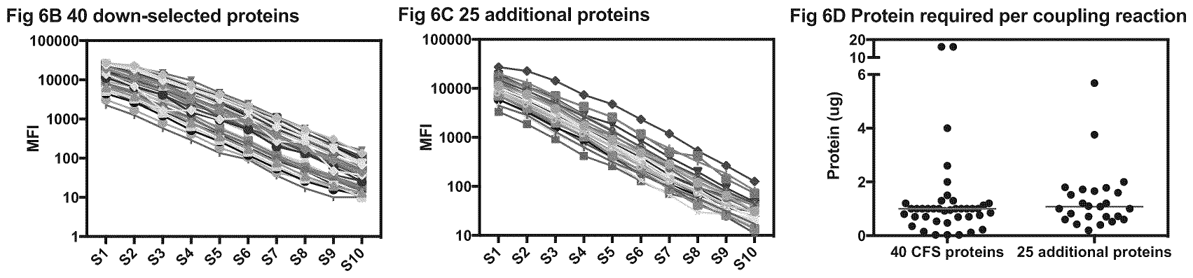

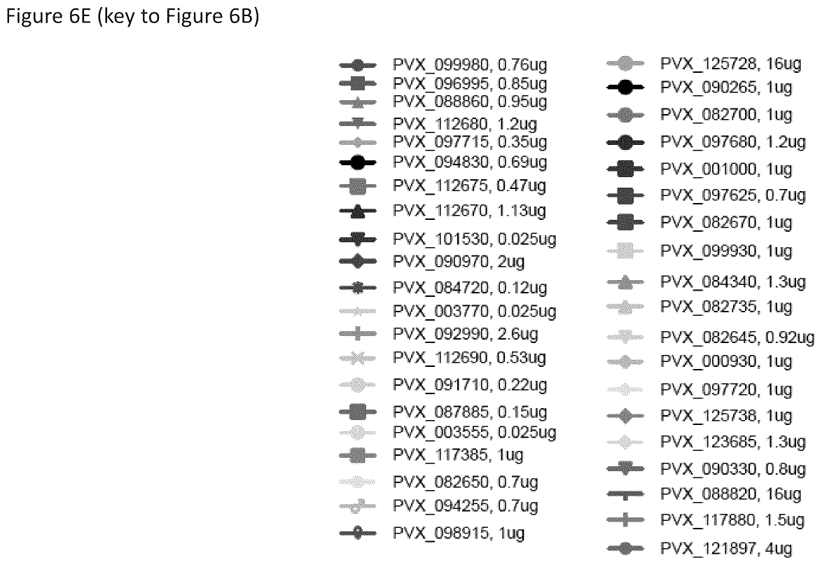

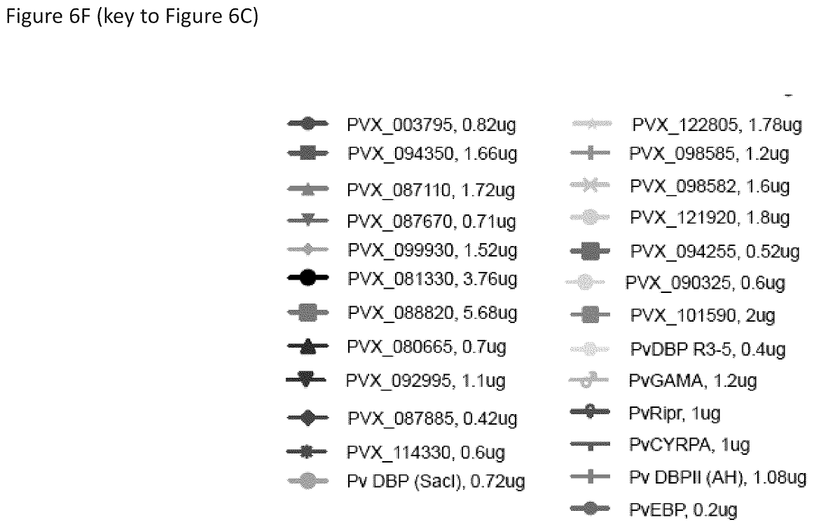

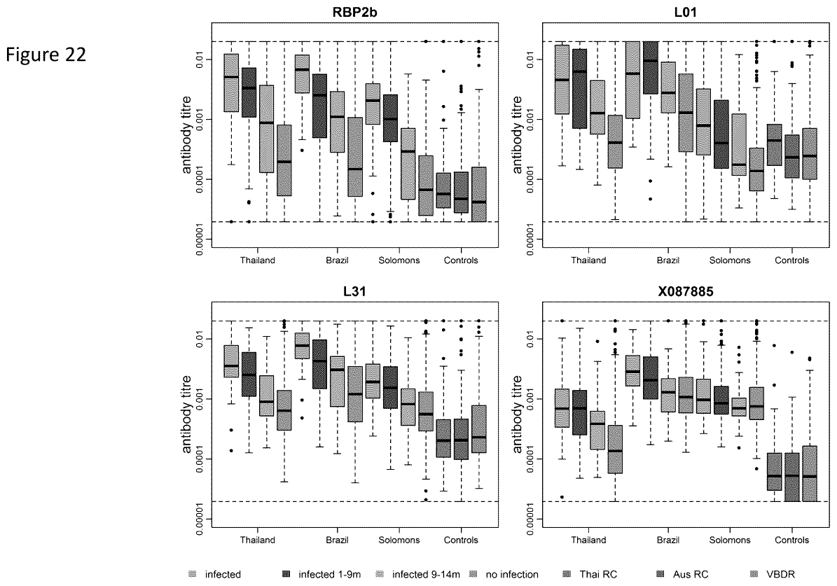

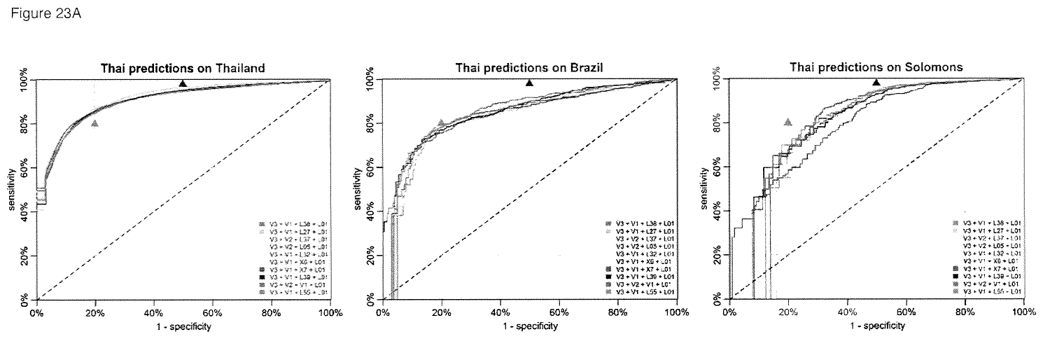

The Luminex® bead-array assay has been successfully established for 40 of the 55 proteins identified in the antigen discovery study (FIG. 6) as well as for the additional 25 proteins (65 total). Plasma samples from three observational cohorts (final time-point) have been screened against these 65 proteins, Thai (n=829), Brazilian (n=925) and Solomon Islands (n=751), in addition to 3 sets of non-exposed (malaria) controls (two panels from Australia and one panel from Thailand). An example of the responses in these cohorts, with relation to time since last infection, to 4 of 65 proteins is shown in FIG. 22, described with regard to Example 4 below.

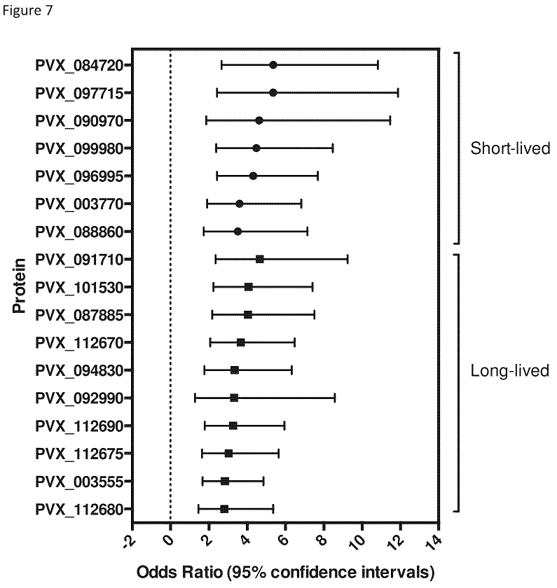

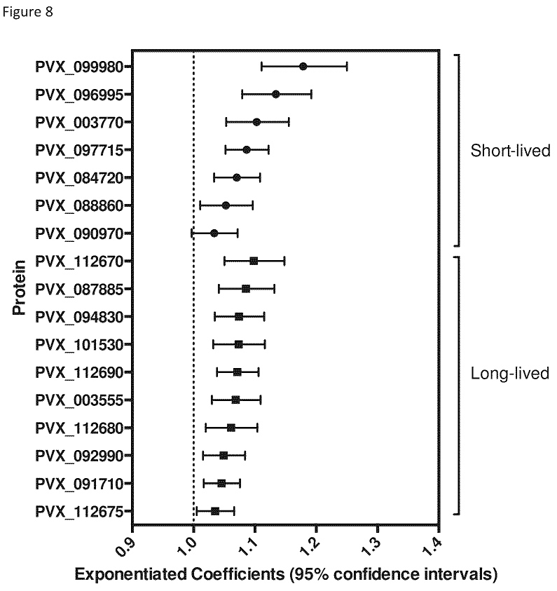

In the Thai cohort, antibody levels measured to all 17 proteins, selected for performing the first set of tests, were strongly associated with the presence of current P. vivax infections (logistic regression model, odds ratios of 2.8-5.4, p<0.05) (FIG. 7). In addition, antibody levels to 16 of 17 proteins at the last visit of the cohort study were positively and significantly associated with past exposure to P. vivax infections based on PCR results during the yearlong assessment period (measured as the molecular force of blood-stage infection, (molFOI) (generalised linear model, exponentiated coefficients of 1.03-1.18, p<0.05) (FIG. 8). The exception was for PVX_090970, exponentiated coefficient 1.03, p=0.073.

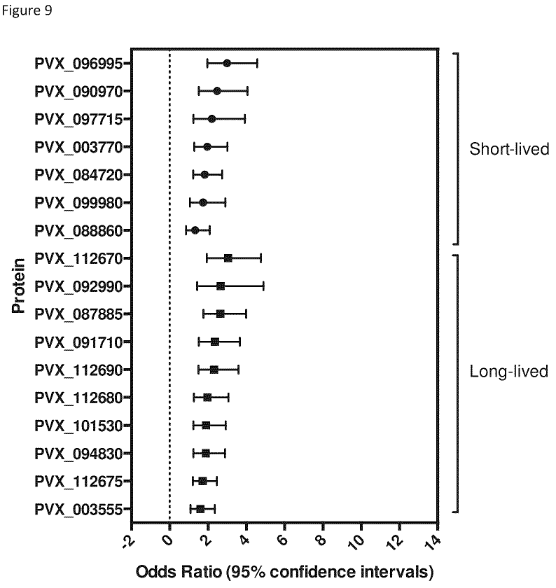

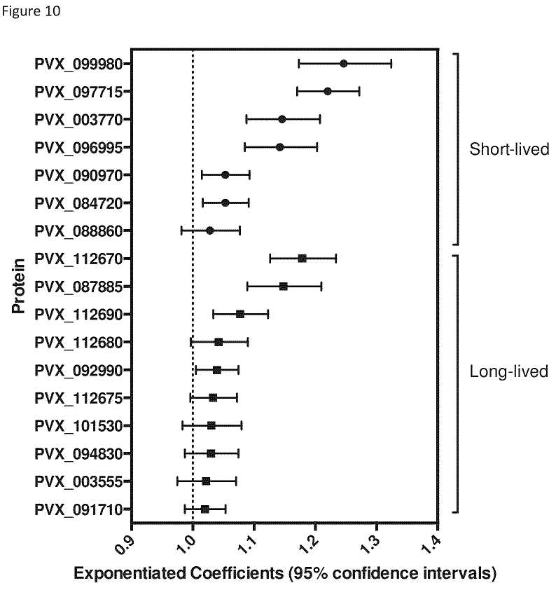

In the Brazilian cohort, the effect size, overall, was not as great as for Thailand. Nevertheless, antibody levels to 16 of 17 proteins were strongly associated with the presence of current P. vivax infections (logistic regression model, odds ratios of 1.59-3.04, p<0.05) (FIG. 9). The exception was for PVX_088860, with an odds ratio of 1.33 (p=0.21). Antibody levels to 10 of 17 proteins at the last visit of the cohort were positively and significantly associated with past exposure to P. vivax (molFOI) (generalised linear model, exponentiated coefficients of 1.04-1.18, p<0.05) (FIG. 10). Of the antibodies with estimated ‘short’ half-lives (less than 6 months), there was one exception, PVX_088860, with an exponentiated coefficient of 1.03 (p=0.24). Of the antibodies with estimated ‘long’ half-lives (more than 6 months), 6 of 10 were not associated with past exposure (exponentiated coefficients of 1.02-1.04, p>0.05).

Various statistical methods can be used to test the association between antibody level to certain proteins and past (recent) or current exposure to P. vivax infections. For most proteins, there was a clear significant association with both past and current P. vivax infections, which is promising for the use of these antigens as serological markers. For others, there was a trend towards an association, which did not reach significance. In a final test, it will be an antibody signature that is used for classification of recent infection, made up of antibody responses to a multitude of proteins. Therefore the lack of significance for some individual proteins does not imply that they will not be useful in the final classification algorithm.

These analyses show that 16 of 17 proteins generate antibodies that are strongly associated with both current infections and 10 of 17 with past P. vivax exposure in both Thailand and Brazil, demonstrating that a majority of these antigens have the potential to detect both concurrent and recent past P: vivax infections.

### References

1. Longley R J, Reyes-Sandoval A, Montoya-Diaz E, Dunachie S, Kumpitak C, Nguitragool W, Mueller I, Sattabongkot J. 2015. Acquisition and longevity of antibodies to Plasmodium vivax pre-erythrocytic antigens in western Thailand. Clin Vaccine Immunol doi: 10.1128/cvi.00501-15.
2. Wampfler R, Mwingira F, Javati S, Robinson L, Betuela I, Siba P, Beck H P, Mueller I, Felger I. 2013. Strategies for detection of Plasmodium species gametocytes. PLOS One 8:e76316.
3. Rosanas-Urgell A, Mueller D, Betuela I, Barnadas C, Iga J, Zimmerman P A, del Portillo H A, Siba P, Mueller I, Felger I. 2010. Comparison of diagnostic methods for the detection and quantification of the four sympatric Plasmodium species in field samples from Papua New Guinea. Malar J 9:361.
4. Lu F, Li J, Wang B, Cheng Y, Kong D H, Cui L, Ha K S, Sattabongkot J, Tsuboi T, Han ET. 2014. Profiling the humoral immune responses to Plasmodium vivax infection and identification of candidate immunogenic rhoptry-associated membrane antigen (RAMA). J Proteomics 102:66-82.
5. Sawasaki T, Ogasawara T, Morishita R, Endo Y. 2002. A cell-free protein synthesis system for high-throughput proteomics. Proc Natl Acad Sci USA 99:14652-14657.
6. Sawasaki T, Hasegawa Y, Tsuchimochi M, Kamura N, Ogasawara T, Kuroita T, Endo Y. 2002. A bilayer cell-free protein synthesis system for high-throughput screening of gene products. FEBS Lett 514: 102-105.
7. Sawasaki T, Morishita R, Gouda M D, Endo Y. 2007. Methods for high-throughput materialization of genetic information based on wheat germ cell-free expression system. Methods Mol Biol 375:95-106.
8. Sawasaki T, Gouda M D, Kawasaki T, Tsuboi T, Tozawa Y, Takai K, Endo Y. 2005. The wheat germ cell-free expression system: methods for high-throughput materialization of genetic information. Methods Mol Biol 310:131-144.
9. Matsuoka K, Komori H, Nose M, Endo Y, Sawasaki T. 2010. Simple screening method for autoantigen proteins using the N-terminal biotinylated protein library produced by wheat cell-free synthesis. J Proteome Res 9:4264-4273.
10. Franca C T, Hostetler J B, Sharma S, White M T, Lin E, Kiniboro B, Waltmann A, Darcy A W, Li Wai Suen C S, Siba P, King C L, Rayner J C, Fairhurst R M, Mueller I. 2016. An Antibody Screen of a Plasmodium vivax Antigen Library Identifies Novel Merozoite Proteins Associated with Clinical Protection. PLOS Negl Trop Dis 10:e0004639.
11. Team RC. 2015. R: A language and environment for statistical computing, R Foundation for Statistical Computing, Vienna, Austria. https://www R-project.org/.

### Example 2—Illustrative Diagnostic Test

A diagnostic test according to at least some embodiments of the present invention could optionally include any of bead-based assays previously described (AlphaScreen® assay and multiplexed assay using Luminex® technology).

In addition to the ability to measure antibody responses using the bead-based assays previously described, other methods could also be used, including, but not limited to, the enzyme linked immunosorbent assay (ELISA) (1), protein microarray (2) and the luminescence immunoprecipitation system (LIPs) (3).

Antibody measurements via ELISA rely on coating of specialised plates with the required antigen, followed by incubation with the plasma sample of interest. IgG levels are detected by incubation with a conjugated secondary antibody followed by substrate, for example a horseradish peroxidase-conjugated anti-IgG and ABTS [2,2=-azinobis(3-ethylbenzothiazo-line-6-sulfonic acid)-diammonium salt].

Protein microarray platforms offer a high-throughput system for measuring antibody responses. Proteins of interest are spotted onto microarray chips then probed with plasma samples. The arrays are then further incubated with a labeled anti-immunoglobulin and analysed using a microarray scanner.

The LIPs assay utilizes cell lysate containing the expressed antigen fused to a Renilla luciferase reporter protein. Plasma samples are incubated with a defined amount of this lysate, with protein A/G beads used to capture the antibody. The amount of antibody-bound antigen-luciferase is measured by the addition of a coelenterazine substrate, and the light emitted measured using a luminometer.

Any of these assays may optionally be combined with a reader and if necessary, an analyzer device, to form an apparatus according to at least some embodiments of the present invention. The reader would read the test results and the analyzer would then analyze them according to any of the previously described algorithms and software.

### References

1. Longley R J, Reyes-Sandoval A, Montoya-Diaz E, Dunachie S, Kumpitak C, Nguitragool W, Mueller I, Sattabongkot J. 2015. Acquisition and longevity of antibodies to Plasmodium vivax pre-erythrocytic antigens in western Thailand. Clin Vaccine Immunol doi: 10.1128/cvi.00501-15.
2 Finney O C, Danziger S A, Molina D M, Vignali M, Takagi A, Ji M, Stanisic D I, Siba PM, Liang X, Aitchison J D, Mueller I, Gardner M J, Wang R. 2014. Predicting anti-disease immunity using proteome arrays and sera from children naturally exposed to malaria. Mol Cell Proteomics doi:10.1074/mcp.M113.036632.
3. Longley R J, Salman A M, Cottingham M G, Ewer K, Janse C J, Khan S M, Spencer A J, Hill A V. 2015. Comparative assessment of vaccine vectors encoding ten malaria antigens identifies two protective liver-stage candidates. Sci Rep 5:11820.

### Example 3—Illustrative Software Process for Diagnosis

This Examples relates to processes for estimation of time since last P. vivax infection using measurements of antibody titers, which may optionally be provided through software.

a. Section 1 relates to calibration and validation of the input data, as well as non-limiting examples of models and algorithms which may optionally be used to analyze the data. Section 2 provides additional information on the algorithms utilized.

### Section 1—Overview of Calibration Data and Algorithms

### Calibration and Validation Data

Both the down-selection of antigens for incorporation into a diagnostic test, and the calibration and validation of algorithms for providing classifications of recent P. vivax infection given blood samples, will depend on the available epidemiological data. Data will be required on the demography of the populations under investigation, serological measurements, and monitoring for parasitemia and clinical episodes. Table 1 provides an overview of the data sets that are used.

### Algorithm Inputs and Outputs

A diagnostic test will take a blood sample as input and provide data to inform a decision making process as output. The type of data generated will depend on the technological specifications of the diagnostic platform. The outputted data can then be used as input for some algorithm to inform a decision making process. The following factors need to be taken into consideration when defining the inputs and outputs of a decision making algorithm:

### 1) Number of Antigens

The number of antigens to which antibodies can be measured will be restricted by the technological specifications of the diagnostic platform under consideration. Measurement of antigens to a greater number of antibodies will in theory provide more data as input for an algorithm, potentially increasing predictive power.

TABLE 1
Overview of data sets used for antigen down-selection and algorithm calibration and validation.

| demographic data | | | serological data | | | parasitological data | | |
|---|---|---|---|---|---|---|---|---|
| | | | number of | samples | | samples | PCR | |
| region | number | age | antigens | per person | platform | per person | positive | clinical |

Antigen down-selection

| | | | | | | | | |
|---|---|---|---|---|---|---|---|---|
| Thailand | 32 | 29 (7, 71) | 342 | 4 | AlphaScreen | 17 | enrolment | enrolment |
| Brazil | 33 | | 342 | 4 | AlphaScreen | 17 | enrolment | enrolment |

Algorithm calibration and validation

| | | | | | | | | |
|---|---|---|---|---|---|---|---|---|
| Thailand | 829 | 25 (2, 79) | 65 | 1 | Luminex | 14 | 97/829 | 25/829 |
| Brazil | 928 | 25 (0, 102) | 65 | 1 | Luminex | 13 | 236/928 | 80/928 |
| Solomon | 860 | 5.5 (0.5, 12.7) | 65 | 1 | Luminex | 11 | 294/860 | 35/860 |
| Islands | | | | | | | | |

Negative controls

| | | | | | | | | |
|---|---|---|---|---|---|---|---|---|
| Australian | 100 | 52 (18, 77) | 65 | 1 | Luminex | 1 | no | no |
| Red Cross | | | | | | | | |
| Thai Red | 72 | | 65 | 1 | Luminex | 1 | no | no |
| Cross | | | | | | | | |
| Australian | 102 | 39 (19, 68) | 65 | 1 | Luminex | 1 | no | no |
| donors | | | | | | | | |

### 2) Measurement of Antibody Levels

The levels of antibody in a blood-sample can be measured and summarised in a variety of ways.

### a) Continuous Measurement

A continuous measurement that has a monotonic relationship with antibody titer. It can be compared with a titration curve to produce an estimate of antibody titer.

### b) Binary Classification

Assesses whether antibody levels are greater or less than some threshold.

### c) Categorical Classification

Assigns antibody levels to one of a set of pre-defined categories, e.g. low, medium, high. A categorical classification can be generated via a series of binary classifications.

### 3) Decision Making Requirements

The result of a diagnostic test and accompanying algorithm can be used to inform a decision on whether or not to treat, as well as to inform surveillance systems.

### a) Classification of Recent Infection

A binary output corresponding to whether or not there was an infection with P. vivax blood-stage parasites in the past 9 months. This can be presented as a binary classification, or as a probabilistic classification. This can be adjusted for a range of different temporal thresholds: 3 months, 6 months, 12 months, 18 months.

### b) Estimation of Time Since Last Infection

An estimate of the time since last P. vivax blood-stage infection-depending on the available calibration data the time since last infection can be defined either as the time since last PCR-detectable blood-stage parasitemia, or as the time since last mosquito bite. Time since last infection can be estimated continuously or categorically. Concurrent estimation of uncertainty will be important.

### c) Medium-Term Serological Exposure

Given sufficient calibration data, the algorithms described here can be modified to provide extended measurements of an individual's recent to medium term P. vivax exposure, e.g. how many infections in the last 2 years?

### 4) Computational and Analytic Capabilities

An algorithm's complexity will be restricted by the analytic resources accompanying the diagnostic platform. In a low resource setting, we may require a decision to be made given a sequence of binary outputs from a rapid diagnostic test (sero-negative or sero-positive) without any access to computational devices. At the other extreme, in a high resource setting we may have continuous measurements of antibodies to multiple antigens accompanied with algorithms encoded in computational software.
a) No access to computational devices. Algorithms implemented via ‘easy to follow’ instructions on paper charts.
b) Algorithm implemented in software that can be installed on a portable computation device such as a smartphone or tablet. May require the manual entry of output from the diagnostic platform.
c) Computational software with encoded algorithms integrated within the diagnostic platform.

### Algorithms

There is a wide range of algorithms for classification and regression in the statistical inference and machine learning literature (Hastie, Tibshirani & Friedman³). A classification algorithm can take a diverse range of input data and provide some binary or categorical classification as output. A regression algorithm can take similar input, but provides a continuous prediction as output. Table 2 provides an overview of some algorithms that can be used for classification problems. Four of these have been regularly described in the statistical learning literature: linear discriminant analysis (LDA); quadratic discriminant analysis (QDA); decision trees; and random forests. One of these has been specifically developed for the application at hand: combined antibody dynamics (CAD). The candidate algorithms are classified according to a number of factors. The degree of transparency describes the straightforwardness and reproducibility of an algorithm. A decision tree is considered very transparent as it can be followed by a moderately well-informed individual, as it requires answering a sequence of questions in response to measured data. This simple, logical structure makes decision trees particularly popular with doctors. Because of the transparency and ease of use, decision trees are sometimes referred to as glass box algorithms. At the other extreme, algorithms such as random forests are considered to be black box algorithms where there may be no obvious association between the inputs and outputs.

TABLE 2
Overview of algorithms suitable for classification of recent P. vivax
infection or estimation of time since last P. vivax infection.

| algorithm | data needs | transparent | stochastic | time predicted | comments |
|---|---|---|---|---|---|
| linear | continuous | + | no | no | The assumption of |
| discriminant | | | | | common covariance for |
| analysis | | | | | each category may be too |
| (LDA) | | | | | restrictive. |
| quadratic | continuous | + | no | no; categorical | There is an approximate |
| discriminant | | | | estimation | equivalence between the |
| analysis | | | | possible, | QDA classification space |
| (QDA) | | | | incorporation of | and that predicted by the |
| | | | | uncertainty | CAD algorithm. |
| | | | | challenging | |
| decision | binary | +++ | no | no; possible via | Very transparent and |
| trees | | | | regression trees or | simple to implement in |
| | | | | categorical | low technology settings. |
| | | | | estimation | |
| random | continuous | −− | yes | no; possible via | Potentially very powerful |
| forests | | | | regression trees or | but requires considerable |
| | | | | categorical | computational resources. |
| | | | | estimation | |
| combined | continuous | ++ | no | yes; with | A biologically motivated |
| antibody | | | | uncertainty | representation of |
| dynamics | | | | | antibodies following |
| (CAD) | | | | | infection; prior |
| | | | | | information on decay |
| | | | | | rates can be incorporated. |

### Section 2—Expanded Details of Algorithms

Here we provide an overview of classification algorithms such as LDA, QDA, decision trees and random forests which have already been described extensively elsewhere (Hastie, Tibshirani & Friedman³). We also provide an extended description of the combined antibody dynamics (CAD) algorithm.

### Linear and Quadratic Discriminant Analysis

The theory of linear discriminant analysis (LDA) and quadratic discriminant analysis (QDA) is described in detail in “The Elements of Statistical Learning: Data Mining, Inference and Prediction” by Hastie, Tibshirani & Friedman⁶. We provide a brief overview of how these methods may be applied. A key assumption for LDA and QDA classification algorithms is that individuals who have similar antibody titers are likely to have the same classification. It is convenient to compare individuals with different antibody profiles via Euclidean distance of log antibody titers. An LDA or QDA classifier can be implemented by fitting multivariate Gaussian distributions to the clusters of data points representing ‘old’ and ‘new’ infections. Assume we have measurements of p antibodies. Denote k∈{new,old} to represent the classes of training individuals with new and old infections. These can be modelled as multivariate Gaussians:

$${f}_{k} ( x ) = \frac{1}{{( 2 π )}^{p / 2} {\vert {\sum }_{k} \vert}^{1 / 2}} {e}^{- \frac{1}{2} {( x - {μ}_{k} )}^{T} {\sum }_{k}^{- 1} ( x - {μ}_{k} )}$$

where μₖ and Σₖ are the mean and p*p covariance matrix of the training data of each class.

In the case of LDA, all classes are assumed to have the same covariance matrix (Σ_{new}=Σ_{old}=Σ), and the classification between new and old infections can be evaluated via the log ratio:

$$log ( \frac{P ( new \vert X = x )}{P ( old \vert X = x )} ) = - \frac{1}{2} {( {μ}_{new} + {μ}_{old} )}^{T} {\sum }^{- 1} ( {μ}_{new} + {μ}_{old} ) + {x}^{T} {\sum }^{- 1} ( {μ}_{new} + {μ}_{old} )$$

which is linear in x. The two categories are therefore separated by a hyperplane in p-dimensional space.

In QDA, the restriction that Σ_{new}=Σ_{old}=Σ is relaxed and it can be shown that the classification boundary is described by a conic section in p-dimensional space.

LDA and QDA have consistently been shown to provide robust classification for a wide range of problems. The predictive power of these algorithms can be assessed through cross-validation whereby the data is split into training and testing data sets. The algorithm is calibrated using the training data set and subsequently validated using the test data set. An important method for assessing an algorithm's predictive power is to evaluate the sensitivity and specificity. In this context, we define sensitivity to be the proportion of recent infections correctly classified as recent infections, and we define specificity to be the proportion of old infections correctly classified as old infections.

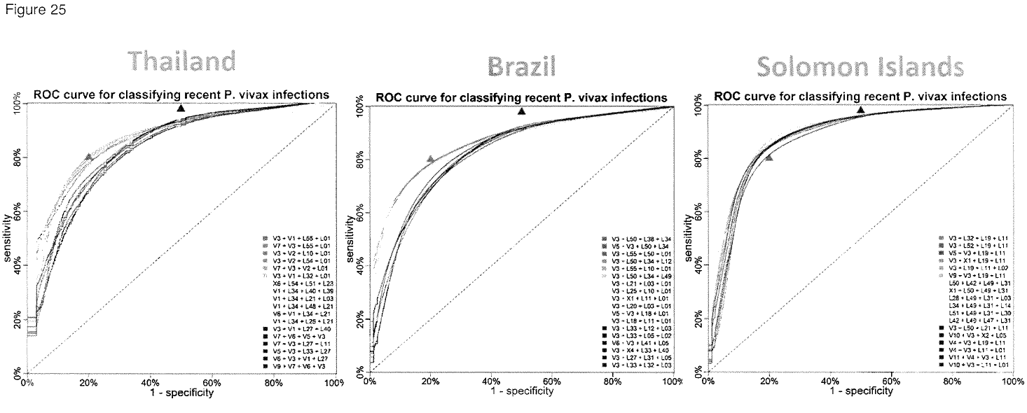

A receiver operating characteristic (ROC) curve allows for detailed investigation of the association between sensitivity and specificity. At one extreme, we could obtain 100% sensitivity and 0% specificity by simply classifying all blood samples as new infections. At the other extreme, we could obtain 100% specificity and 0% sensitivity by classifying all blood samples as old infections. FIG. 25 shows ROC curves describing the classification performance of LDA algorithms for combinations of 4 antigens in Thailand, Brazil and the Solomon Islands.

### Decision Trees and Random Forests

Tree-based algorithms partition the space spanned by the data into a set of rectangles with a unique classification applied to each rectangle. Similarly to the LDA and QDA classification algorithms, a great deal of theoretical information is supplied in the book “The Elements of Statistical Learning: Data Mining, Inference and Prediction”.

There are several powerful methods for extending decision tree classifiers including bagging (bootstrapp aggregating), boosting and random forests³. These methods can lead to substantially improved classifiers but typically require more computation and more data. In addition to providing powerful classifiers, these algorithms can provide important diagnostics for investigating the association between the signal in the input and the output.

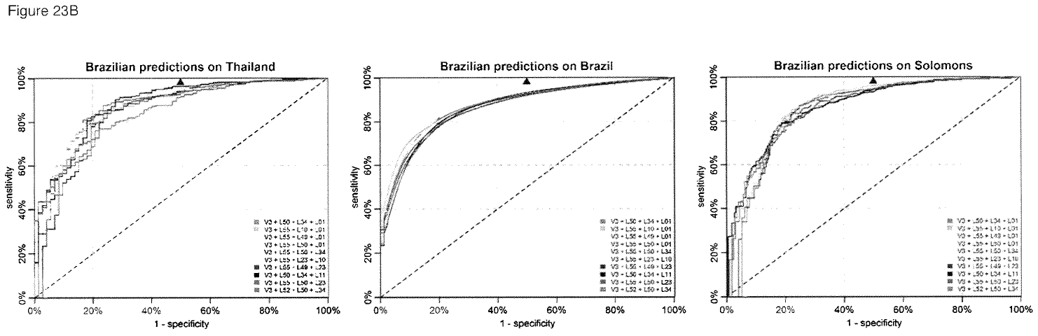

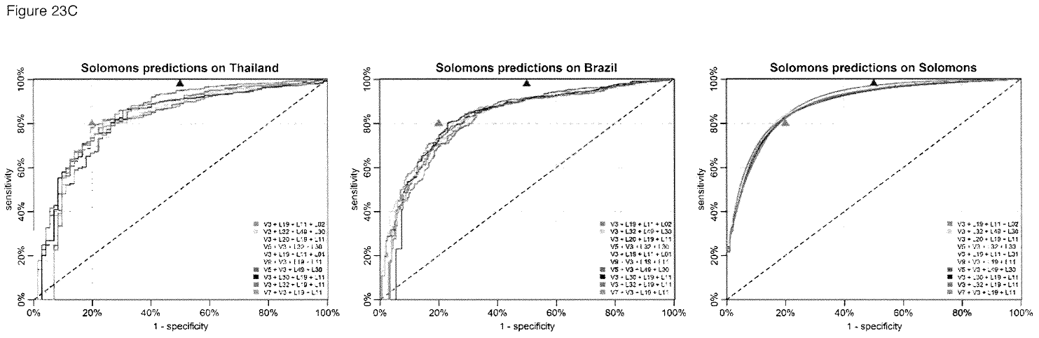

FIG. 23A-C shows the ROC curves for cross-validated random forests classifiers applied to data sets from Thailand, Brazil and Solomon Islands. Notably, when the training and testing data sets are from the same region, there are many combinations of four antigens that allow sensitivity >80% and specificity >80%. When training and testing data sets are from different regions, it was still possible to obtain combinations of four antigens with sensitivity >80% and specificity >80%.

### Modelling of Antibody Dynamics

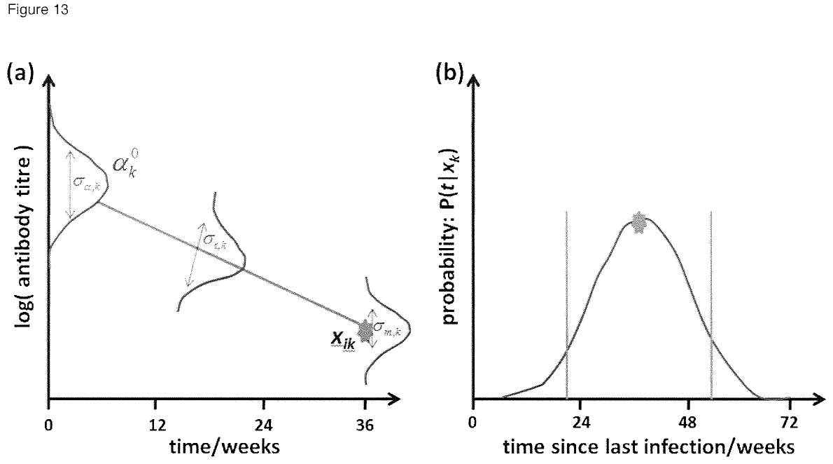

A key premise of the proposed diagnostic test is that following infection with P. vivax blood-stage parasites, an antibody response will be generated that will change predictably over time (FIG. 13). Here we present a subset of the data that demonstrates how antibodies to P. vivax antigens change over time.

Longitudinal Antibody Titers Following Clinical P. vivax

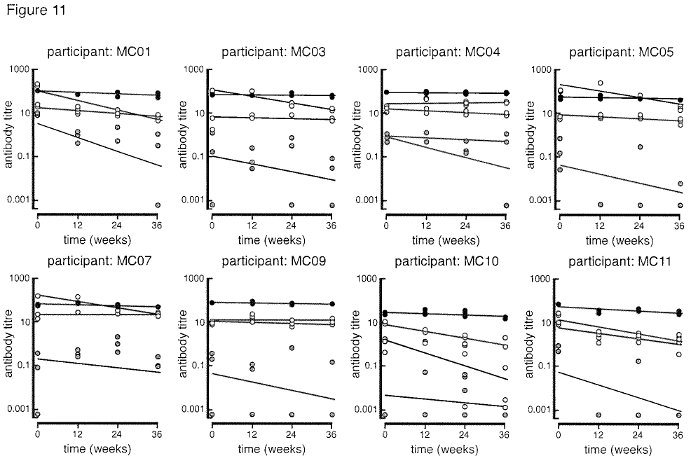

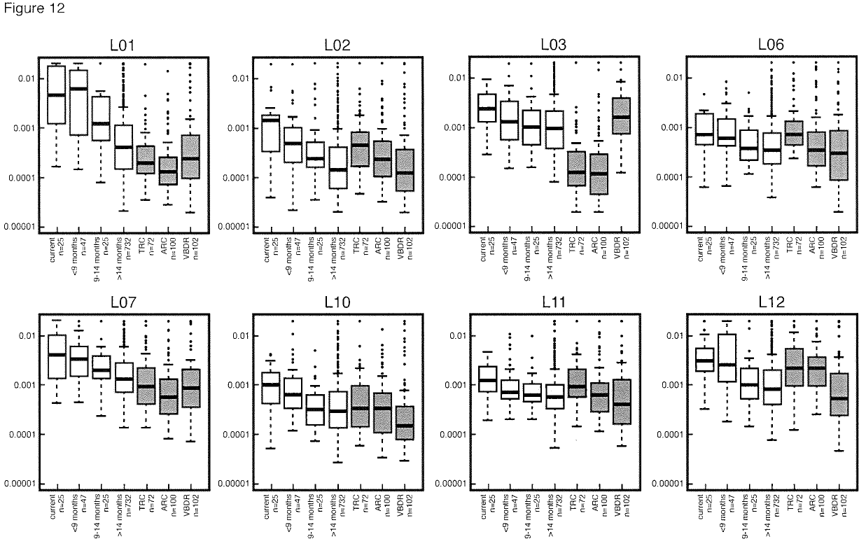

We have data from longitudinal cohorts in Thailand and Brazil where participants were followed for up to 36 weeks after a symptomatic clinical episode of P. vivax (see also Table 1/Materials and Methods in Example 1, antigen discovery cohorts). Participants were treated with primaquine, and blood samples were frequently tested to ensure they remained free from reinfection. Antibody levels to a wide range of antigens were measured at 12 week intervals to investigate the changing antibody dynamics. The sample data in FIG. 11 illustrates that antibodies exhibit a range of different half-lives—a pattern consistent with the rest of the data (see also FIG. 3). Another important general feature of the data is exhibited here: rapidly decaying antibodies (short half-life) exhibit much more measurement error than slowly decaying antibodies (long-lived half-life).

The decay of anti-malaria antibodies following infection can be described by an exponential or a bi-phasic exponential distribution⁴. Because of the sampling frequency (every 12 weeks) we assume that antibodies decay exponentially. Exponential decay equates to linear decay on a log scale. Therefore we utilise linear regression models. In particular, we utilise a mixed-effects linear regression framework so that we can estimate both the mean rate of antibody decay as well as the standard deviation.

We assume that for individual i we have measurements of antibody titer Aᵢⱼₖ at time j to antigen k. We assume that at time 0, antibody titers are Normally distributed⁵ with mean αₖ⁰ and standard deviation σ_{α,k} on a log-scale. We assume that an individual's rate of antibody decay is drawn from a Normal distribution with mean rₖ⁰ and standard deviation σ_{r,k}. The antibody dynamics in the population can therefore be described by the following mixed-effects linear regression model:

$$log ( {A}_{ijk} ) \sim ( {α}_{k}^{0} + {α}_{ik} ) + ( {r}_{k}^{0} + {r}_{ik} ) {t}_{j} + {ε}_{k} \qquad ( 1 )$$

$${α}_{ik} \sim N ( 0 , {σ}_{α , k} )$$

$${r}_{ik} \sim N ( 0 , {σ}_{r , k} )$$

$${ε}_{k} \sim N ( 0 , {σ}_{m , k} )$$

This model can be fitted to data using the lmer package in R. FIG. 11 shows a sample of the fitted profiles of antibody dynamics.

### Estimation Using Antibodies to a Single Antigen

Here we describe an algorithm that uses a biologically-motivated model of the decay of antibody titers over time to facilitate statistical inference of the time since last infection. A key requirement of this algorithm is that it requires some prior knowledge of the decay rates of antibodies. This can be achieved either through estimation of antibody decay rates from longitudinal data as described in equation (1), or estimation of decay rates from cross-sectional antibody measurements as presented in FIG. 12.

The linear regression model for the decay of antibody titers described in equation (1) has three sources of variation: (i) variation in initial antibody titer following infection; (ii) between individual variation in antibody decay rate; and (iii) measurement error. Notably, all these sources of variations are described by Normal distributions (FIG. 13a) so their combined variation will also be described by a Normal distribution. Therefore, the expected log antibody titer to antigen k in individual i at time t can be described by the following distribution.

$${x}_{ik} ∼ N ( {α}_{k}^{2} + {r}_{k} t , {σ}_{α , k}^{2} + {t}^{2} {σ}_{r , k}^{2} + {σ}_{m , k}^{2} ) \qquad ( 2 )$$

The probability distribution of the expected antibody titer to antigen k in individual i at time t is given by the following distribution:

$$P ( {x}_{ik} \vert t ) = \frac{1}{\sqrt{2 π ( {σ}_{α , k}^{2} + {t}^{2} {σ}_{r , k}^{2} + {σ}_{m , k}^{2}} )} {e}^{- \frac{{( {x}_{ik} - {α}_{k}^{0} - {r}_{k}^{0} t )}^{2}}{2 ( {σ}_{α , k}^{2} + {t}^{2} {σ}_{r , k}^{2} + {σ}_{m , k}^{2} )}} \qquad ( 3 )$$

Note that we have xᵢₖ∈(−∞, +∞), as xᵢₖ denotes the log antibody titer and measurements of antibody titer are assumed to be positive. The probability distribution for the time since infection/given measured antibody titer xᵢₖ can be calculated by inverting equation (3) using Bayes rule³.

$$P ( t \vert {x}_{ik} ) - \frac{P ( {x}_{ik} \vert t ) P ( t )}{P ( {x}_{ik} )} \qquad ( 4 )$$

The time since last infection will have a lower bound of zero. We can choose to impose an upper bound of either the individual's age ‘a’ or positive infinity. Choosing positive infinity allows us to better handle the case where an individual was never infected—the low measured antibody titers will be consistent with a very large time since last infection, possibly greater than the age of the individual. Therefore we should only restrict t to the interval (0, a) if we know for certain that the individual has been infected. In practice, we choose some large time tₘₐₓ for our upper bound. We assume P(t) denotes a uniform distribution on the interval (0, tₘₐₓ). P(xᵢₖ) is a normalising constant which is calculated via numerical integration to ensure that P(t|xᵢₖ) denotes a probability distribution.

Equation (4) provides a probability distribution for the time since last infection. For the purposes of a diagnostic test we may be more interested in obtaining a binary classification, e.g. was the individual infected within the last 9 months. It is usually not possible to definitively make such a categorisation, but we can instead calculate their probabilities as follows:

$${P}_{0 - 9 m} ( {x}_{ik} ) = {\int }_{0}^{9} P ( t \vert {x}_{ik} ) dt \qquad ( 5 )$$

$${P}_{9 m +} ( {x}_{ik} ) = {\int }_{9}^{{t}_{max}} P ( t \vert {x}_{ik} ) dt$$

### Combined Antibody Dynamics: Estimation Using Antibodies to Multiple Antigens

Previously, we described how the antibody titer to a single antigen can be used to estimate the time since last infection. However, in practice there is too much noise to make an accurate estimate of time since last infection with a single antigen. Increasing the number of measured antibodies can increase the information content in our data allowing us to obtain more accurate estimates of time since last infection. In particular, selecting antibodies with a range of half-lives may increase our power to resolve infection times more accurately.

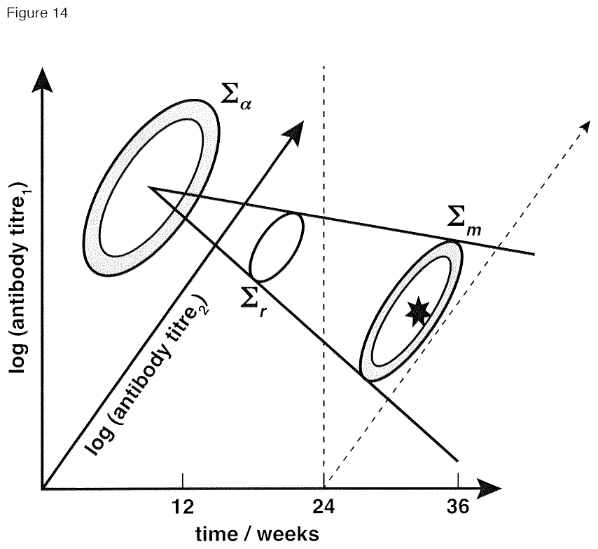

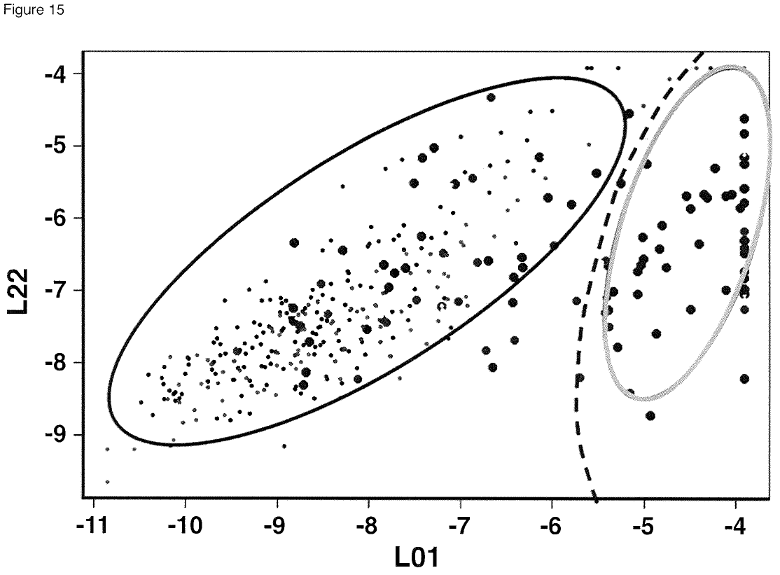

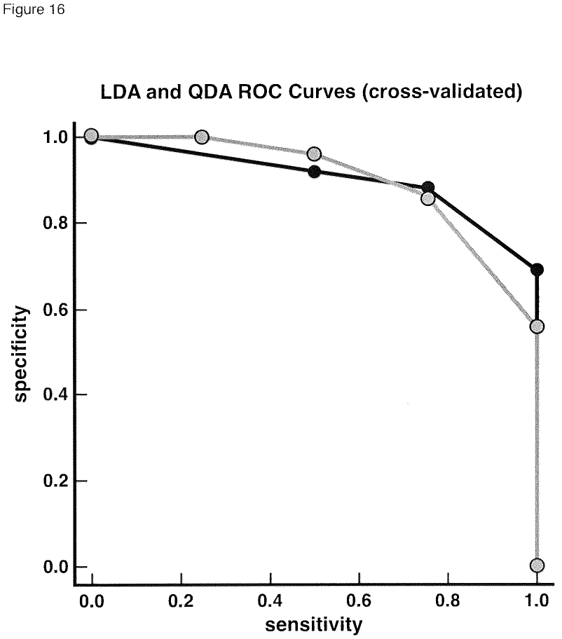

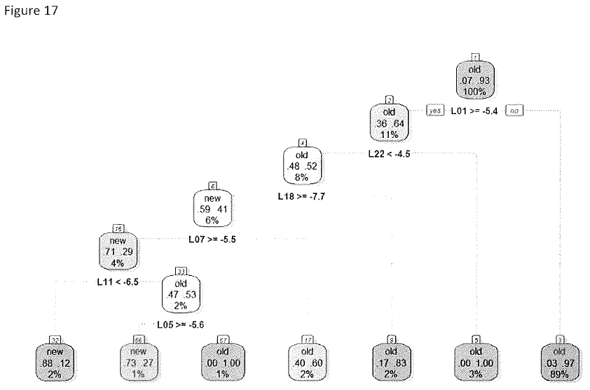

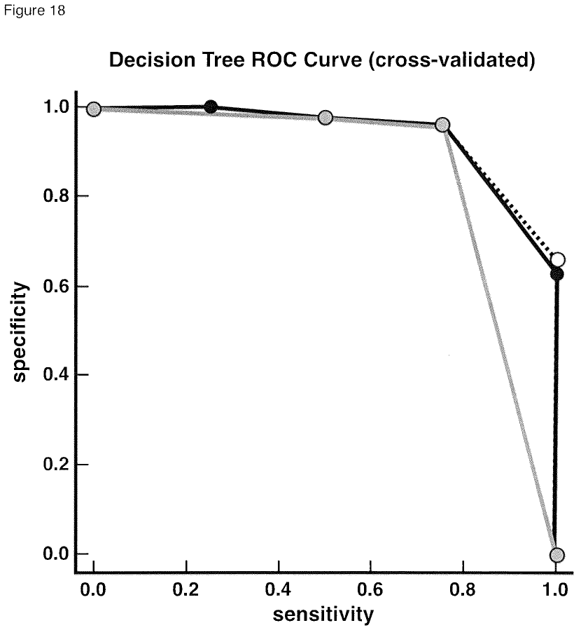

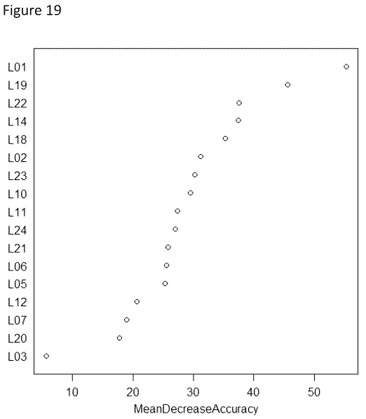

FIG. 14 shows a schematic of the dynamics of antibodies to two antigens. We have rapidly decaying antibody 1 and slowly decaying antibody 2. At baseline, antibody titers are likely to be correlated, so we assume that initial titer following infection is described by a multivariate Normal distribution with covariance matrix Σ_{α}. The between individual rates of antibody decay may also be correlated (i.e. all antibody titers may decay particularly quickly in some individuals) so we also assume that decay rates are described by a multivariate Normal distribution with covariance matrix Σᵣ. Finally, there will be measurement error associated with each antibody. In particular, we assume the measurement errors between different antibodies are independent so that the total measurement error can be described by a multivariate Normal distribution with diagonal covariance matrix Σₘ.

$$P ( {x}_{i} \vert t ) = {( 2 π )}^{- \frac{k}{2}} {\vert {\sum }_{α} + {t}^{2} {\sum }_{r} + {\sum }_{m} \vert}^{- \frac{1}{2}} {e}^{- \frac{1}{2} {( {x}_{i} - {α}^{0} - {r}^{0} t )}^{T} {( {\sum }_{α} + {t}^{2} {\sum }_{r} + {\sum }_{m} )}^{- 1} ( {x}_{i} - {α}^{0} - {r}^{0} t )} \qquad ( 6 )$$

The method for estimating the time since last infection given the multivariate probability distribution for the measured vector of antibody titers xᵢ is the same as described in equation (4).

### Selecting Optimal Combinations of Antigens

Machine learning algorithms take data from a large number of streams and identify which data streams have the most signal for classifying output. Such methods typically involve a greedy algorithm which will provide a good but not necessarily optimal solution. Greedy algorithms take the next best step, i.e. including the next antigen that gives the biggest increase in predictive power. As such they may provide a locally optimal solution but not necessarily a globally optimal solution. Simulated annealing algorithms provide an alternative to greedy algorithms that provide a higher likelihood of obtaining a globally optimal solution⁷.

Here we describe how a simulated annealing algorithm can be applied to the combined antibody dynamics (CAD) classifier to select a combination of antigens that provides optimal predictive power. Assume that P measurements of antibodies are available. We want to select some subset of these that maximises predictive power. Denote y to be a vector of 0's and 1's indicating whether the p^{th} antibody is included in our panel. Thus for example we may have

$$y = ( 0 , 0 , 1 , 1 , 0 , 1 , 0 , 0 , 1 ) \qquad ( 7 )$$

The vector of binary states depicted in equation (7) will correspond to a vector of antibody measurements as follows:

$${x}_{i} = ( {x}_{i , 1} , {x}_{i , 2} , {x}_{i , 3} , {x}_{i , 4} ) \qquad ( 8 )$$

Given data from I individuals on measured antibody responses, we can calculate the probability that the individual was infected within the last 9 months P_{0-9 m} (xᵢ) or greater than 9 months ago P_{9 m+} (xᵢ). Let zᵢ be an indicator denoting whether individual I was infected in the last 9 months (zᵢ=1) or not (zᵢ=0). We can then write down the likelihood of the data as follows:

$$L ( y ) = {\prod }_{i = 1}^{I} {{P}_{0 - 9 m} ( {x}_{i} )}^{{z}_{i}} {{P}_{9 m +} ( {x}_{i} )}^{1 - {z}_{i}} \qquad ( 9 )$$

The challenge is to select a binary vector y corresponding to a combination of antigens that maximises the likelihood in equation (9) and thus has the highest likelihood of correctly classifying infections according to whether they occurred in the last 9 months.

If we have P antigens, there are 2^{P} combinations of antigens. For P >15 it is not computationally feasible to test all possible combinations. We therefore utilise a simulated annealing algorithm for exploring the state space of combinations and identifying the optimal combinations subject to various constraints (e.g. enforcing a maximum of 10 antigens to a panel). FIG. 20 shows the results, and this contributed to the initial down-selected of antigens as described in Example 1.

### References

1 White. N. J. Determinants of relapse periodicity in Plasmodium vivax malaria. Malaria Journal 10, doi:29710.1186/1475-2875-10-297 (2011).
2 Mueller, I. et al. Key gaps in the knowledge of Plasmodium vivax, a neglected human malaria parasite. Lancet Infectious Diseases 9, 555-566 (2009).
3 Hastie. T., Tibshirani, R. & Friedman, J. The elements of statistical learning: Data mining, inference, and prediction. Second edn, (Springer, 2009).
4 White, M. T. et al. Dynamics of the Antibody Response to Plasmodium falciparum Infection in African Children. Journal of Infectious Diseases 210. 1115-1122, doi: 10.1093/infdis/jiu219 (2014).
5 Yman, V. et al. Antibody acquisition models: A new tool for serological surveillance of malaria transmission intensity. Scientific Reports 6. doi: 10.1038/srep19472 (2016).
6 The Elements of Statistical Learning: Data Mining, Inference and Prediction” by Hastie, Tibshirani & Friedman; 2001, Springer.
7 Kirkpatrick, S., Gelatt Jr, C. D. & Vecchi, M. P. Optimization by simulated annealing. Science 220, 671-680 (1983).

### Example 4—Additional Testing of Antigens

This non-limiting Example relates to additional testing of antibody responses to various P. vivax proteins, present in the blood, as potential antigens for a diagnostic test. It further relates to selection of Plasmodium vivax antigens for classification of samples with past blood-stage infections.

The blood collection and laboratory work was generally performed according to the materials and methods described in Example 1.

### Overview of Epidemiological Cohorts

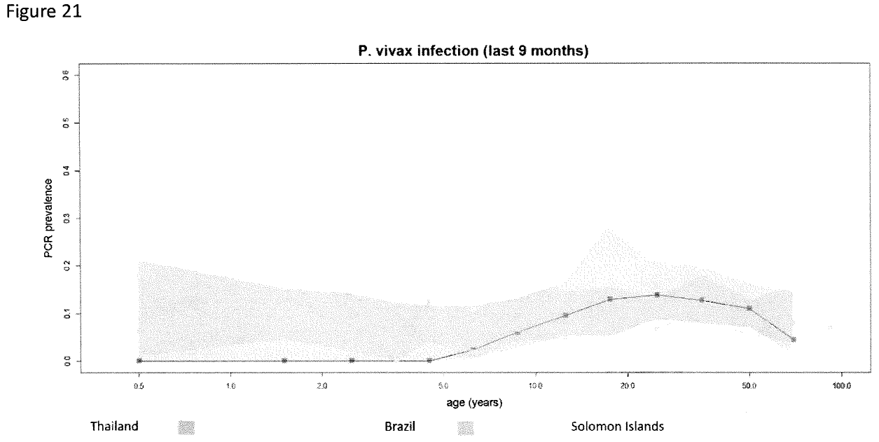

Data was obtained from longitudinal cohorts in three different regions of the P. vivax endemic world. In each cohort, approximately 1,000 individuals were followed over time for approximately 1 year, with active case detection samples taken every month. These samples were supplemented by passive case detection samples from individuals experiencing clinical episodes of P. vivax or P. falciparum. An overview of the data collected is shown in Table 3, and age-stratified prevalence of PCR detectable blood-stage infection within the last 9 months is shown in FIG. 21.

In addition data was obtained from three cohorts of negative controls who were highly to have ever been exposed to malaria. These cohorts consisted of 102 individuals from the Victorian Blood Donor Registry (VBDR), 100 individuals from the Australian Red Cross, and 72 individuals from the Thai Red Cross (residents of Bangkok with no reported history of malaria).

TABLE 3
Epidemiological overview of cohorts analysed for the association
between P. vivax antibody titers and time since last
PCR detectable infection. Number of samples per individual
and age are shown as median with range.

| | | | | | Solomon | |
|---|---|---|---|---|---|---|
| | Thailand | | Brazil | | Islands | |
| number of | 829 | | 928 | | 860 | |
| individuals | | | | | | |
| samples per | 14 | (4, 18) | 13 | (4, 16) | 10 | (6, 11) |
| individual | | | | | | |
| Female | 454 | (54.8%) | 471 | (50.7%) | 416 | (48.4%) |
| age (years) | 24 | (1, 78) | 25 | (0, 103) | 5.5 | (0.5, 12.7) |
| PCR infection | 97 | (11.7%) | 236 | (25.4%) | 294 | (34.2%) |
| during study | | | | | | |
| PCR infection in | 72 | (8.7%) | 205 | (22.1%) | 265 | (30.8%) |
| last 9 months | | | | | | |
| PCR infection in | 44 | (5.3%) | 119 | (12.8%) | 156 | (18.1%) |
| last 3 months | | | | | | |
| PCR infection at | 25 | (3.0%) | 40 | (4.3%) | 93 | (10.8%) |
| last final time | | | | | | |
| point | | | | | | |

### Measured Antibody Responses

In each of the three longitudinal cohorts, antibody responses were measured at the final time point to allow investigation of the association between antibody response and time since last infection. The antibody responses to 65 antigens were measured. 40 of these antigens were selected following a previously published down-selection procedure from a starting panel of 342 wheat-germ expressed proteins. These 40 proteins were supplemented by another 25 purified P. vivax proteins obtained from collaborators. These P. vivax antigens were coupled to COOH micro-beads, and a multiplexed Luminex assay was used to measure Mean Fluorescence Intensity (MFI) for each antigen in each sample. MFI measurements were converted to antibody titers by calibrated to measurements from a hyper-immune pool of Papua New Guinean adults. FIG. 22 shows the measured response from 4 of the 65 antigens, and the variation with time since last infection.

### Selection of Optimal Combinations of Antigens for Classification

### Initial Investigation of Combinations of Parameters

Of the 65 P. vivax proteins considered, 5 were excluded because of poor immunogenicity which resulted in missing data from a large proportion of samples. This resulted in a panel of 60 antigens for detailed investigation and further down-selection. The aim is to identify combinations of up to 5 antigens that can provide accurate classification within a single cohort, and identify combinations of 8-15 antigens that can accurately across multiple cohorts with a wide range of transmission intensities and age ranges.

Without wishing to be limited by a single hypothesis, selection optimized for three classification targets:
1 Surveillance target. Select combinations of antigens such that both sensitivity and specificity are given equal weight in optimisation. This is done by maximising the area under the curve (AUC) of a receiver operating characteristic (ROC) curve.
2. Serological Screen and Treat (SSAT) target. Select combinations of antigens that maximise sensitivity (e.g. >95%) while enforcing a lower bound on specificity (e.g. >50%).
3 Surveillance target. Select combinations of antigens that maximise specificity (e.g. >95%) while enforcing a lower bound on sensitivity (e.g. >50%).

The first step is to identify combinations of antigens for which there is a strong signal enabling classification. This was done by using a linear discriminant analysis (LDA) classifier to test all combinations of antigen of size up to 5. Above size 5, it was not computationally feasible to evaluate all possible combinations. Therefore for n>5, combinations of size n+1 were evaluated by identifying the optimal 500 combinations of size n antigens and including all positive individually.

### Optimisation of Algorithms Given Most Likely Parameter Combinations

Given a subset of n antigens, a range of classification algorithms were considered: LDA, quadratic discriminant analysis (QDA), decision trees, and random forests. For a given algorithm and subset of antigens classification performance was assessed through cross-validation. The key to cross-validation is to use disjoint training and testing data sets to assess classification of performance. For each cohort, this is done by randomly selecting ⅔ of the data as the training set and testing the algorithm on the remaining ⅓. This is repeated 200 times and the average of the cross-validated ROC curves is calculated.

FIGS. 23A-23C show cross-validated ROC curves for assessing the classification performance of random forests algorithms (determined according to the randomForests library in R). In cases where algorithms were trained and tested on data from the same region, many different combinations of 4 antigens resulted in sensitivity and specificity greater than 80%. Even when an algorithm was trained on data from one region and then tested on data from another region of the world, it was still possible to obtain combinations of antigens with both sensitivity and specificity greater than 80%, with the exception of algorithms trained on data from Thailand and tested on data from the Solomon Islands.

### Ranking of Antigens

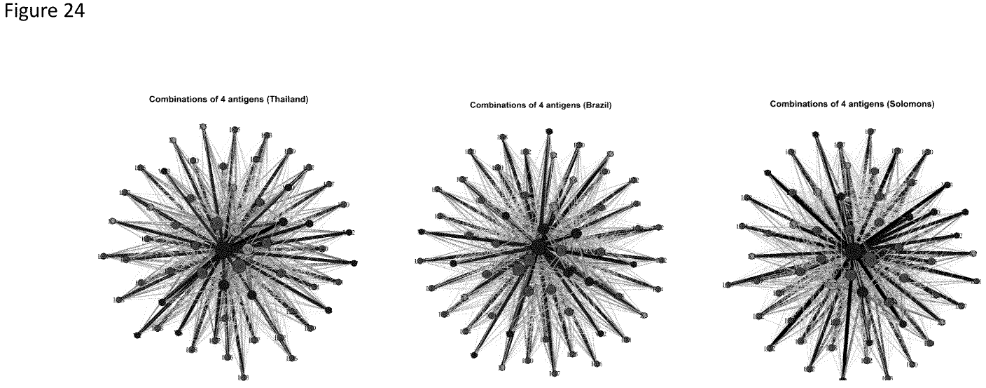

Multiple factors determine whether or not an antigen will contribute to classification of recent infection. These include but are not limited to: antibody dynamics; immunogenicity of recent infections compared to old infections and measurements from control samples; area under the ROC curve when considering one antigen at a time; frequency of selection in top combinations of antigens. FIG. 24 shows a network visualisation of how combinations of 4 antigens are selected. The size of each node represents the likelihood that an antigen is selected, and the width and colour of an edge represents the probability that a pair of antigens are selected in combination. Therefore, the most commonly selected antigens are biggest and cluster in the centre of the network. There was a high degree of consistency in the antigens that were selected in each of the three cohorts, with the most strongly identified antigens being RBP2b (V3), L01, L31, X087885 (X7), PvEBP (V11), L55, PvRipr (V8) and L54.

Table 4 shows a ranking of antigens according to a range of criteria. The top two antigens, RBP2b and L01, are preferred candidates. The next six antigens are likely candidates. The next seven antigens are possible candidates. Also included are an additional nine antigens worth further consideration.

TABLE 4
List of antigens ranked according to their contribution to classification of
individuals with PCR detectable blood-stage P. vivax in the last 9 months. The area under the
curve (AUC) is based on using antibody titers to a single antigen for classification. Combinations
of antigens were investigated by assessing classification performance of linear discriminant
analysis (LDA) for all combination of 4 antigens from the initial panel of 60 antigens. Recent
infection sero-positivity shows the proportion of individuals with PCR detectable P. vivax in the
last 9 months, with the threshold of sero-positivity defined as the geometric mean titer (GMT)
plus two standard deviations of the negative controls.

| | Area Under Curve | | | Top 1% of combination | | | Recent infection | | |
|---|---|---|---|---|---|---|---|---|---|
| | (1 antigen) | | | (4 antigens) | | | sero-positivity | | |
| antigen | Thailand | Brazil | Solomons | Thailand | Brazil | Solomons | Thailand | Brazil | Solomons |
| RBP2b | 0.849 | 0.818 | 0.868 | 89.7% | 98.5% | 100.0% | 70.8% | 64.4% | 45.7% |
| (V3) | | | | | | | | | |
| L01 | 0.812 | 0.787 | 0.697 | 43.5% | 23.9% | 4.3% | 51.4% | 56.6% | 14.3% |
| L31 | 0.805 | 0.762 | 0.766 | 5.0% | 2.7% | 3.7% | 25.0% | 38.0% | 7.4% |
| X087885 | 0.807 | 0.748 | 0.697 | 20.3% | 9.2% | 14.6% | 41.7% | 81.0% | 50.9% |
| (X7) | | | | | | | | | |
| PvEBP | 0.794 | 0.739 | 0.707 | 5.0% | 2.4% | 3.1% | 55.6% | 41.0% | 7.8% |
| (V11) | | | | | | | | | |
| L55 | 0.79 | 0.781 | 0.643 | 17.2% | 20.9% | 2.6% | 38.9% | 29.8% | 3.5% |
| PvRipr | 0.754 | 0.772 | 0.646 | 3.0% | 9.1% | 3.1% | 31.9% | 29.3% | 4.8% |
| (V8) | | | | | | | | | |
| L54 | 0.79 | 0.727 | 0.654 | 5.6% | 4.4% | 3.1% | 26.4% | 19.0% | 2.2% |
| L07 | 0.747 | 0.765 | 0.599 | 3.1% | 5.3% | 2.8% | 27.8% | 41.5% | 3.9% |
| L30 | 0.732 | 0.61 | 0.609 | 2.3% | 3.8% | 5.4% | 47.2% | 11.7% | 9.6% |
| PvDBPII | 0.74 | 0.773 | 0.639 | 1.7% | 2.6% | 4.0% | 20.8% | 47.3% | 3.5% |
| (V10) | | | | | | | | | |
| L34 | 0.767 | 0.746 | 0.67 | 4.5% | 16.6% | 2.2% | 12.5% | 19.0% | 3.9% |
| X092995 | 0.792 | 0.703 | 0.642 | 11.5% | 1.9% | 5.6% | 15.3% | 34.1% | 10.0% |
| (X6) | | | | | | | | | |
| L12 | 0.755 | 0.731 | 0.637 | 3.5% | 6.1% | 2.9% | 16.7% | 15.1% | 3.0% |
| RBP1b | 0.533 | 0.578 | 0.525 | 24.1% | 4.7% | 2.5% | 0.0% | 0.0% | 0.0% |
| (V1) | | | | | | | | | |
| L23 | 0.759 | 0.753 | 0.658 | 4.0% | 14.8% | 2.9% | 12.5% | 19.5% | 5.7% |
| L02 | 0.746 | 0.724 | 0.677 | 2.7% | 3.7% | 3.9% | 15.3% | 13.7% | 2.6% |
| L32 | 0.705 | 0.651 | 0.493 | 3.7% | 1.9% | 30.2% | 4.2% | 3.9% | 0.4% |
| L28 | 0.759 | 0.744 | 0.667 | 3.8% | 2.5% | 2.4% | 45.8% | 33.2% | 9.1% |
| L19 | 0.753 | 0.67 | 0.664 | 2.6% | 2.3% | 6.5% | 33.3% | 19.5% | 10.9% |
| L36 | 0.727 | 0.698 | 0.662 | 3.2% | 1.8% | 2.8% | 36.1% | 22.0% | 10.4% |
| L41 | 0.702 | 0.66 | 0.636 | 2.55 | 1.7% | 3.3% | 29.2% | 17.6% | 8.3% |
| X088820 | 0.723 | 0.666 | 0.638 | 4.0% | 1.8% | 6.7% | 15.3% | 35.6% | 14.8% |
| (X4) | | | | | | | | | |
| PvDBP.. | 0.716 | 0.761 | 0.616 | 1.7% | 2.6% | 7.2% | 16.7% | 36.6% | 1.3% |
| Sacl (V13) | | | | | | | | | |

FIG. 25 shows Receiver Operating Characteristic (ROC) curves for assessing the trade-off between sensitivity and specificity for a cross-validated linear discriminant analysis (LDA) classifier applied to data from Thailand, Brazil and the Solomon Islands.

Appendix I

| | | Pro- | Insert aa | |
|---|---|---|---|---|
| | | tein | sequence (add | |
| | Protein | Refer- | M as start/His- | Insert DNA sequence (Start from ATG to |
| No. | Name | ence | tag at C-term) | His-tag stop codon) |
| 1 | merozoite | PVX_ | MNESKEILSQLLNVQTQLLTMSSEH | ATGAACGAGTCCAAGGAGATCCTCAGCCAACTCCTGAACGTGCAAACC |
| | surface | 099980 | TCIDTNVPDNAACYRYLDGTEEWRC | CAGCTCCTGACCATGTCCAGCGAGCACACCTGCATCGACACCAACGTCC |
| | protein 1 | | LLTFKEEGGKCVPASNVTCKDNNG | CAGACAACGCCGCCTGCTACAGGTACCTGGACGGCACCGAGGAGTGG |
| | (MSP1), | | GCAPEAECKMTDSNKIVCKCTKEGS | CGCTGCCTCCTGACCTTCAAGGAAGAGGGCGGCAAGTGCGTGCCAGCC |
| | MSP1-19 | | EPLFEGVFCSHHHHHH | TCCAACGTCACCTGCAAGGACAACAACGGCGGCTGCGCTCCAGAGGCT |
| | | | (SEQ ID NO: 1) | GAGTGCAAGATGACCGACAGCAACAAGATCGTGTGCAAGTGCACCAA |
| | | | | GGAAGGCTCCGAGCCACTCTTCGAGGGCGTCTTCTGCAGCCACCACCA |
| | | | | CCACCACCACTGA (SEQ ID NO: 2) |
| 2 | trypto- | PVX_ | MKTETVTSRSNPHQAIEYANQGPS | ATGAAGACCGAGACGGTGACCTCCAGGAGCAACCCACACCAAGCCATC |
| | phan- | 096995 | RDKVEEWKRNAWTDWMVQLDDD | GAGTACGCCAACCAGGGCCCATCCAGGGACAAGGTGGAGGAGTGGAA |
| | rich | | WKDFNAQIEEEKKAWIEEKEGDWV | GCGCAACGCCTGGACCGACTGGATGGTCCAACTCGACGACGACTGGA |
| | antigen | | ILLKHLQNKWLHFNPNLDAEYQTD | AGGACTTCAACGCCCAGATCGAGGAAGAGAAGAAGGCCTGGATTGAG |
| | (Pv-fam-a) | | MLAKSETWDERQWKMWISTEGKQ | GAGAAGGAAGGCGACTGGGTCATCCTCCTGAAGCACCTCCAAAACAA |
| | | | LLEMDLKKWFTNNEMIYCKWTMDE | GTGGCTGCACTTCAACCCAAACCTCGACGCCGAGTACCAGACCGACAT |
| | | | WNEWKNEKIKEWVTSEWKESEDQ | GCTGGCCAAGTCCGAGACGTGGGACGAGAGGCAGTGGAAGATGTGG |
| | | | YWSKYDDATIQTLTVAERNQWFKW | ATCAGCACCGAGGGCAAGCAGCTCCTGGAGATGGACCTCAAGAAGTG |
| | | | KERIYREGIEWKNWIAIKESKFVNA | GTTCACCAACAACGAGATGATCTACTGCAAGTGGACCATGGACGAGTG |
| | | | NWNSWSEWKNEKRLEFNDWIEAF | GAACGAGTGGAAGAACGAGAAGATCAAGGAGTGGGTGACCTCCGAGT |
| | | | VEKWIRQKQWLIWTDERKNFANRQ | GGAAGGAGAGCGAGGACCAATACTGGTCCAAGTACGACGACGCCACC |
| | | | KAAPGGVAAAPGVFAPRPAFGAPS | ATCCAAACCCTGACCGTCGCCGAGCGCAACCAGTGGTTCAAGTGGAAG |
| | | | GFAPRPGFAAPSQPPRYSFAAASG | GAGAGGATCTACCGCGAGGGCATCGAGTGGAAGAACTGGATCGCCAT |
| | | | YVAPSATSEAAPATSEAPASAEATT | CAAGGAGAGCAAGTTCGTGAACGCCAACTGGAACTCCTGGTCTGAGTG |
| | | | ALSSETTTPVNPEETAASPEAATPV | GAAGAACGAGAAAAGGCTGGAGTTCAACGACTGGATCGAGGCCTTCG |
| | | | NPEETAASSETTTVNPEATPVNPEA | TCGAGAAGTGGATCCGCCAAAAGCAGTGGCTGATCTGGACCGACGAG |
| | | | PVAEPEKKEEEPAAEPLLAIEPAQT | AGGAAGAACTTCGCCAACCGCCAAAAGGCTGCTCCAGGCGGCGTGGC |
| | | | EPAALEAAPSTSAHHHHHH | TGCCGCCCCAGGCGTCTTCGCCCCACGCCCAGCCTTCGGCGCCCCATCC |
| | | | (SEQ ID NO: 3) | GGCTTCGCCCCAAGGCCAGGCTTCGCTGCTCCAAGCCAGCCACCACGC |
| | | | | TACTCCTTCGCTGCCGCCAGCGGCTACGTGGCTCCATCCGCTACCAGCG |
| | | | | AGGCTGCTCCAGCCACCTCCGAGGCCCCAGCCAGCGCCGAGGCTACCA |
| | | | | CCGCTCTCTCCAGCGAGACGACCACCCCAGTCAACCCAGAGGAGACGG |
| | | | | CTGCTAGCCCGGAGGCTGCTACCCCAGTGAACCCGGAGGAGACGGCT |
| | | | | GCCTCCAGCGAGACGACGACGGTCAACCCAGAGGCCACCCCGGTGAA |
| | | | | CCCAGAGGCTCCAGTGGCTGAGCCAGAGAAGAAGGAAGAGGAGCCA |
| | | | | GCTGCTGAGCCACTGCTCGCTATCGAGCCAGCTCAAACCGAGCCAGCT |
| | | | | GCTCTGGAGGCTGCTCCATCCACCAGCGCCCACCACCACCACCACCACT |
| | | | | GA (SEQ ID NO: 4) |
| 3 | sporozoite | PVX_ | MQLELEPAPDYESTSPTVPVRLLLH | ATGCAGCTGGAGCTGGAGCCAGCCCCAGACTACGAGTCCACCAGCCCA |
| | invasion- | 088860 | DDYAPNAEDMFGPEASQVMTNLYE | ACCGTGCCAGTCAGGCTCCTGCTCCACGACGACTACGCCCCAAACGCC |
| | associated | | TIDEDGTTTDGYQNGSDDDQSNQS | GAGGACATGTTCGGCCCAGAGGCCTCCCAAGTGATGACCAACCTCTAC |
| | protein | | DSNDDAVMLNYLSNETDSFDELIDEI | GAGACGATCGACGAGGACGGCACCACCACCGACGGCTACCAAAACGG |
| | 2, | | DNHKKKKKIYSPLRKPVLKRSDSSD | CTCCGACGACGACCAAAGCAACCAGTCCGACAGCAACGACGACGCCGT |
| | putative | | SLSDYELDEVLRQTENEPEEDEDLD | CATGCTCAACTACCTGTCCAACGAGACGGACAGCTTCGACGAGCTCATC |
| | (SIAP2) | | LSLEDSFEVINYPWKDILESSPYSTD | GACGAGATCGACAACCACAAGAAGAAGAAGAAGATCTACTCCCCACTC |
| | | | HTNEEDFSSLEELELEDPVQEMNFG | AGGAAGCCAGTGCTGAAGCGCAGCGACTCCAGCGACTCCCTGAGCGA |
| | | | KLKFFEIGDPDLLIRKTPITPNTKTKS | CTACGAGCTCGACGAGGTCCTGCGCCAGACCGAGAACGAGCCAGAGG |
| | | | GLEKNGNNTEASNINQHEKEKMDK | AAGACGAGGACCTGGACCTCTCCCTGGAGGACAGCTTCGAGGTCATCA |
| | | | RKRRTHKQFKNPIENFSVTTTYDDF | ACTACCCATGGAAGGACATCCTGGAGTCCAGCCCATACAGCACCGACC |
| | | | LKQNGLRDHPSKHQKDSSEPFVLD | ACACCAACGAGGAAGACTTCTCCAGCCTGGAGGAGCTGGAGCTGGAG |
| | | | QYNYRNAKFKNVRFYILRMLYDNIK | GACCCAGTCCAAGAGATGAACTTCGGCAAGCTGAAGTTCTTCGAGATC |
| | | | DIGLKEFQYLKSHKYEVEEFIKNILRN | GGCGACCCAGACCTGCTCATCAGGAAGACCCCAATCACCCCAAACACC |
| | | | NLICLTFSQEDHLFNDAHLLIEKASIK | AAGACCAAGTCCGGCCTGGAGAAGAACGGCAACAACACCGAGGCCAG |
| | | | SEHHHHHH (SEQ ID NO: 5) | CAACATCAACCAGCACGAGAAGGAGAAGATGGACAAGCGCAAGAGGC |
| | | | | GCACCCACAAGCAATTCAAGAACCCAATCGAGAACTTCTCCGTGACCAC |
| | | | | CACCTACGACGACTTCCTCAAGCAAAACGGCCTGAGGGACCACCCAAG |
| | | | | CAAGCACCAGAAGGACTCCAGCGAGCCATTCGTGCTCGACCAATACAA |
| | | | | CTACCGCAACGCCAAGTTCAAGAACGTCAGGTTCTACATCCTCCGCATG |
| | | | | CTGTACGACAACATCAAGGACATCGGCCTCAAGGAGTTCCAGTACCTG |
| | | | | AAGTCCCACAAGTACGAGGTCGAGGAGTTCATCAAGAACATCCTCAGG |
| | | | | AACAACCTCATCTGCCTGACCTTCAGCCAAGAGGACCACCTGTTCAACG |
| | | | | ACGCCCACCTGCTCATCGAGAAGGCCTCCATCAAGAGCGAGCACCACC |
| | | | | ACCACCACCACTGA (SEQ ID NO: 6) |
| 4 | rhoptry | PVX_ | MNAGDGQGVYGGNGINNPLVYHVQ | ATGAACGCTGGCGACGGCCAAGGCGTGTACGGCGGAAACGGCATCAA |
| | neck | 117880 | HGVNIPNSNSDKKASDHTPDEDEDT | CAACCCACTCGTGTACCACGTCCAGCACGGCGTCAACATCCCAAACTCC |
| | protein 2, | | YGRTRNKRYMHRNPGEKYKGSNSP | AACAGCGACAAGAAGGCCAGCGACCACACCCCAGACGAGGACGAGGA |
| | putative | | HDSNDDSGDTEYELNEGDVKRLTP | CACCTACGGCAGGACCCGCAACAAGAGGTACATGCACCGCAACCCAG |
| | (RON2) | | KNKKGATTEEVDTYPYGKKTNGSEF | GCGAGAAGTACAAGGGCTCCAACAGCCCACACGACTCCAACGACGACA |
| | | | PRMNGSETGHYGYNNTGSGGHND | GCGGCGACACCGAGTACGAGCTGAACGAGGGCGACGTGAAGAGGCTC |
| | | | ENGYTPIIVKYDNTHAKNRANEIEEN | ACCCCAAAGAACAAGAAGGGCGCCACCACCGAGGAAGTGGACACCTA |
| | | | LNKGEYSRIKMAKGKKGQKSGGYE | CCCATACGGCAAGAAGACCAACGGCAGCGAGTTCCCACGCATGAACG |
| | | | SDGEDSDVDSSNVFYVDNGQDMLI | GCTCCGAGACGGGCCACTACGGCTACAACAACACCGGCAGCGGCGGC |
| | | | KEKMSRSEGPDEMSEEGLNVKYKA | CACAACGACGAGAACGGCTACACCCCAATCATCGTGAAGTACGACAAC |
| | | | QRGPVNYHFSNYMNLDKRNTLSSN | ACCCACGCCAAGAACAGGGCCAACGAGATCGAGGAGAACCTCAACAA |
| | | | EIELQKMIGPKFSEEVNKYCRLNEPS | GGGCGAGTACTCCCGCATCAAGATGGCCAAGGGCAAGAAGGGCCAAA |
| | | | SKKGEFLNVSFEYSRALEELRSEMI | AGTCCGGCGGCTACGAGAGCGACGGCGAGGACTCCGACGTCGACTCC |
| | | | NELQKRKAVGSNYYNNILNAIYTSM | AGCAACGTGTTCTACGTCGACAACGGCCAGGACATGCTGATCAAGGAG |
| | | | NRKNANFGRDAYEDKSFISEANSFR | AAGATGTCCAGGAGCGAGGGCCCAGACGAGATGAGCGAGGAAGGCC |
| | | | NEEMQPLSAKYNKILROYLCHVFVG | TCAACGTGAAGTACAAGGCCCAAAGGGGCCCAGTCAACTACCACTTCT |
| | | | NPGVNQLERLYFHNLALGELIEPIRR | CCAACTACATGAACCTGGACAAGCGCAACACCCTCTCCAGCAACGAGA |
| | | | KYNKLASSSVGLNYEIYIASSSNIYLM | TCGAGCTCCAGAAGATGATCGGCCCAAAGTTCAGCGAGGAAGTGAAC |
| | | | GHLLMLSLAYLSYNSYFVQGLKPFY | AAGTACTGCAGGCTGAACGAGCCATCCAGCAAGAAGGGCGAGTTCCTC |
| | | | SLETMLMANSDYSFFMYNEVCNVY | AACGTCTCCTTCGAGTACAGCAGGGCCCTGGAGGAGCTGAGGTCCGA |
| | | | YHPKGTFNKDITFIPIESRPGRHSTY | GATGATCAACGAGCTGCAAAAGCGCAAGGCCGTGGGCAGCAACTACT |
| | | | VGERKVTCDLLELILNAYTLINVHEIQ | ACAACAACATCCTCAACGCCATCTACACCTCCATGAACAGGAAGAACGC |
| | | | KVFNTSEAYGYENSISFGHNAVRIFS | CAACTTCGGCCGCGACGCCTACGAGGACAAGTCCTTCATCAGCGAGGC |
| | | | QVCPRDDAKNTFGCDFEKSTLYNS | CAACAGCTTCAGGAACGAGGAGATGCAACCACTCTCCGCCAAGTACAA |
| | | | KVLKMDEGDKENQRSLKRAFDMLR | CAAGATCCTGCGCCAGTACCTCTGCCACGTGTTCGTCGGCAACCCAGGC |
| | | | TFAEIESTSHLGDPSPNYISLIFEQNL | GTGAACCAACTGGAGCGCCTGTACTTCCACAACCTCGCCCTGGGCGAG |
| | | | YTDFYKYLFWYDNRELINVQIRNAG | CTGATCGAGCCAATCAGGCGCAAGTACAACAAGCTGGCCTCCAGCTCC |
| | | | RRKKGKKVKFVYDEFVKRGKQLKD | GTCGGCCTCAACTACGAGATCTACATCGCCAGCTCCAGCAACATCTACC |
| | | | KLIKIDAKYNARSKALLVFYALVDKYA | TCATGGGCCACCTCCTGATGCTCAGCCTGGCCTACCTGTCCTACAACAG |
| | | | NIFRKSENVRKFFLNDVSSIRHHLYL | CTACTTCGTGCAGGGCCTCAAGCCATTCTACTCCCTCGAAACCATGCTC |
| | | | NSVLTKSPKSNLDSMKKTLEELQSL | ATGGCCAACTCCGACTACAGCTTCTTCATGTACAACGAGGTGTGCAACG |
| | | | TNAPLKFIVRGNNLKFLNNVAKFENL | TCTACTACCACCCAAAGGGCACCTTCAACAAGGACATCACCTTCATCCC |
| | | | FYVNLFIMSSLSRKHHHHHH | AATCGAGAGCAGGCCAGGCAGGCACTCCACCTACGTGGGCGAGAGGA |
| | | | (SEQ ID NO: 7) | AGGTCACCTGCGACCTCCTGGAGCTCATCCTGAACGCCTACACCCTGAT |
| | | | | CAACGTGCACGAGATCCAAAAGGTCTTCAACACCAGCGAGGCCTACGG |
| | | | | CTACGAGAACTCCATCAGCTTCGGCCACAACGCCGTGAGGATCTTCTCC |
| | | | | CAGGTCTGCCCACGCGACGACGCCAAGAACACCTTCGGCTGCGACTTC |
| | | | | GAGAAGAGCACCCTGTACAACTCCAAGGTGCTCAAGATGGACGAGGG |
| | | | | CGACAAGGAGAACCAGAGGTCCCTGAAGCGCGCCTTCGACATGCTCCG |
| | | | | CACCTTCGCCGAGATCGAGTCCACCAGCCACCTCGGCGACCCAAGCCC |
| | | | | AAACTACATCTCCCTGATCTTCGAGCAAAACCTCTACACCGACTTCTACA |
| | | | | AGTACCTGTTCTGGTACGACAACAGGGAGCTCATCAACGTGCAGATCC |
| | | | | GCAACGCCGGCAGGCGCAAGAAGGGCAAGAAGGTGAAGTTCGTCTAC |
| | | | | GACGAGTTCGTCAAGAGGGGCAAGCAACTGAAGGACAAGCTCATCAA |
| | | | | GATCGACGCCAAGTACAACGCCCGCAGCAAGGCCCTCCTGGTGTTCTA |
| | | | | CGCCCTGGTCGACAAGTACGCCAACATCTTCAGGAAGTCCGAGAACGT |
| | | | | GCGCAAGTTCTTCCTCAACGACGTCTCCAGCATCAGGCACCACCTCTAC |
| | | | | CTGAACAGCGTGCTGACCAAGTCCCCAAAGAGCAACCTCGACAGCATG |
| | | | | AAGAAGACCCTGGAGGAGCTGCAGTCCCTCACCAACGCCCCACTGAAG |
| | | | | TTCATCGTCAGGGGCAACAACCTGAAGTTCCTCAACAACGTGGCCAAG |
| | | | | TTCGAGAACCTGTTCTACGTGAACCTCTTCATCATGTCCAGCCTCTCCCG |
| | | | | CAAGCACCACCACCACCACCACTGA (SEQ ID NO: 8) |
| 5 | Plasmodium | PVX_ | MNVNKKSSGEENNTKQALGLRVSR | ATGAACGTCAACAAGAAGTCCAGCGGCGAGGAGAACAACACCAAGCA |
| | exported | 101530 | TLAKDGANENAEEGLSEEEEEAVEE | AGCTCTGGGCCTGAGGGTGTCCCGCACCCTCGCTAAGGACGGCGCCAA |
| | protein, | | GEEEAVEEGEEEVVEEEGEEVVEG | CGAGAACGCCGAGGAAGGCCTCAGCGAGGAAGAGGAAGAGGCCGTC |
| | unknown | | EEEEVVEGEEEVVEDEEVVEGEEYA | GAGGAAGGCGAGGAAGAGGCCGTGGAGGAAGGCGAGGAAGAGGTG |
| | function | | EGEEPVEGEEYAEGEEPVEGEEPV | GTCGAGGAAGAGGGCGAGGAAGTGGTCGAGGGCGAGGAAGAGGAA |
| | | | VEEYAEGEEPVEGEEYAEGEEPV | GTGGTGGAGGGGGAGGAAGAGGTGGTGGAGGATGAGGAAGTGGTG |
| | | | EGEEVVEGEEVVEGEEVAEGEEVA | GAGGGCGAGGAGTACGCTGAGGGCGAGGAGCCGGTGGAGGGGGAG |
| | | | EGEEVAEGEEAVEGEEVAEGEEVA | GAGTACGCCGAGGGGGAGGAGCCAGTGGAGGGCGAGGAGCCAGTGG |
| | | | EGEEVAEGEEAAEEGAAEEGATEE | AGGTGGAGGAGTACGCGGAGGGGGAGGAGCCGGTGGAAGGTGAGG |
| | | | GATEEGATKEEATEKAAEGEETAES | AGTACGCCGAGGGCGAGGAGCCTGTCGAGGGGGAGGAAGTGGTGGA |
| | | | EKPAEEQPTTFVETVEKKVEPVSKP | AGGCGAGGAAGTGGTGGAAGGTGAGGAAGTGGCTGAGGGCGAGGA |
| | | | PFKPLFPVDEKYLETLEDIAQSFLKE | AGTGGCCGAGGGGGAGGAAGTGGCCGAGGGCGAGGAAGCCGTGGA |
| | | | FQEAEGKRKQKKVKKRAKKITKKLA | GGGCGAGGAAGTGGCGGAGGGGGAGGAAGTGGCGGAAGGCGAGGA |
| | | | KEYAKKFKSKKKHHHHHH | AGTGGCCGAAGGCGAGGAAGCCGCTGAGGAAGGCGCTGCCGAGGAA |
| | | | (SEQ ID NO: 9) | GGCGCCACGGAGGAAGGCGCTACCGAGGAAGGCGCCACCAAGGAAG |
| | | | | AGGCCACCGAGAAGGCTGCTGAGGGCGAGGAGACGGCTGAGTCCGA |
| | | | | GAAGCCAGCTGAGGAGCAACCAACCACCTTCGTGGAGACGGTCGAGA |
| | | | | AGAAGGTGGAGCCAGTCAGCAAGCCACCATTCAAGCCACTCTTCCCAG |
| | | | | TCGACGAGAAGTACCTCGAAACCCTGGAGGACATCGCCCAATCCTTCCT |
| | | | | GAAGGAGTTCCAAGAGGCCGAGGGCAAGAGGAAGCAGAAGAAGGTG |
| | | | | AAGAAGCGCGCCAAGAAGATCACCAAGAAGCTCGCCAAGGAGTACGC |
| | | | | CAAGAAGTTCAAGTCCAAGAAGAAGCACCACCACCACCACCACTGA |
| | | | | (SEQ ID NO: 10) |
| 6 | trypto- | PVX_ | MPKPDQKNLKGGVKNAPLQQRKGS | ATGCCAAAGCCAGACCAAAAGAACCTCAAGGGCGGCGTGAAGAACGC |
| | phan/ | 112680 | VPINPPKPVNDKLKDGSNKTETKNA | CCCACTGCAACAGAGGAAGGGCTCCGTGCCAATCAACCCACCAAAGCC |
| | threo- | | KNTLSKPPMQVTDKSKDEAKKTPLQ | AGTCAACGACAAGCTCAAGGACGGCAGCAACAAGACCGAGACGAAGA |
| | nine- | | STPKLTPKTKEVPKESNMEMWLKDT | ACGCCAAGAACACCCTGTCCAAGCCACCAATGCAAGTGACCGACAAGA |
| | rich | | KDEYENLKCQYRTCLYDWFRKINDE | GCAAGGACGAGGCCAAGAAGACCCCACTCCAGTCCACCCCAAAGCTGA |
| | antigen | | YNELLNKLEEKWAKFPNDPKNKDVF | CCCCAAAGACCAAGGAAGTGCCAAAGGAGAGCAACATGGAGATGTGG |
| | | | DNLKTSSLKNDEKKAQWMRKNLKD | CTCAAGGACACCAAGGACGAGTACGAGAACCTCAAGTGCCAGTACAG |
| | | | LMREQVDEWLEGKKKIYEGMSPTY | GACCTGCCTGTACGACTGGTTCCGCAAGATCAACGACGAGTACAACGA |
| | | | WDAWEKKIAKGLMGAAWYKMNSS | GCTCCTGAACAAGCTGGAGGAGAAGTGGGCCAAGTTCCCAAACGACC |
| | | | GRTKEWDKLRNELETRYNKKIKSLW | CAAAGAACAAGGACGTGTTCGACAACCTCAAGACCTCCAGCCTGAAGA |
| | | | GGFHRDVYFRFKEWIEEVFNKWIEN | ACGACGAGAAGAAGGCCCAGTGGATGAGGAAGAACCTCAAGGACCTG |
| | | | KQIDTWMNSGKKHHHHHH | ATGAGGGAGCAGGTGGACGAGTGGCTGGAGGGCAAGAAGAAGATCT |
| | | | (SEQ ID NO: 11) | ACGAGGGCATGTCCCCAACCTACTGGGACGCCTGGGAGAAGAAGATC |
| | | | | GCTAAGGGCCTGATGGGCGCTGCTTGGTACAAGATGAACTCCTCCGGC |
| | | | | AGGACCAAGGAGTGGGACAAGCTCAGGAACGAGCTCGAAACCCGCTA |
| | | | | CAACAAGAAGATCAAGTCCCTCTGGGGCGGCTTCCACAGGGACGTGTA |
| | | | | CTTCCGCTTCAAGGAGTGGATCGAGGAAGTGTTCAACAAGTGGATCGA |
| | | | | GAACAAGCAAATCGACACCTGGATGAACAGCGGCAAGAAGCACCACC |
| | | | | ACCACCACCACTGA (SEQ ID NO: 12) |
| 7 | hypothe- | PVX_ | MQYSIVKNEITKRRKPKIRNESPPDG | ATGCAATACTCCATCGTGAAGAACGAGATCACCAAGAGGCGCAAGCCA |
| | tical | 097715 | NSPGGGKNNAAGNNGGGDNNAKN | AAGATCAGGAACGAGTCCCCACCAGACGGCAACAGCCCAGGCGGCGG |
| | protein | | KAANKAANNAANKAANNAANNAAN | CAAGAACAACGCTGCTGGCAACAACGGCGGCGGCGACAACAACGCCA |
| | | | NAANNAANNAANNAANNAANNAAN | AGAACAAGGCTGCTAACAAGGCTGCTAACAACGCCGCCAACAAGGCC |
| | | | NAANNAANNANEQNGNKKKKGKPK | GCCAACAACGCTGCTAACAACGCCGCGAACAACGCCGCCAACAACGCC |
| | | | KEEADLPVQAQNENDRNKIEDIADE | GCCAACAACGCAGCTAACAACGCCGCTAACAACGCGGCCAACAACGCC |
| | | | AELFAEEAKMLADLASKRSKEVEQIL | GCGAACAACGCGGCGAACAACGCTGCCAACAACGCCAACGAGCAAAA |
| | | | SSIPENKFGSEPKEDAIFAAKDAVRA | CGGCAACAAGAAGAAGAAGGGCAAGCCAAAGAAGGAAGAGGCCGAC |
| | | | SEDAMKAAQKARAAETVTQANEEK | CTCCCAGTGCAAGCCCAGAACGAGAACGACAGGAACAAGATCGAGGA |
| | | | DKAKTAKELAERSAQIVKKNAVEALK | CATCGCTGACGAGGCTGAGCTGTTCGCTGAGGAAGCCAAGATGCTCGC |
| | | | EFGKIAEAAEMEAIKIPIPENLKPKKK | CGACCTGGCCTCCAAGCGCAGCAAGGAAGTGGAGCAGATCCTCTCCAG |
| | | | VKQPRAAAQKVEPTQATAHKVVPP | CATCCCAGAGAACAAGTTCGGCTCCGAGCCAAAGGAAGACGCCATCTT |
| | | | PAEPPRAPSPPPPPAKPEAAPPAKE | CGCTGCTAAGGACGCCGTGAGGGCTAGCGAGGACGCCATGAAGGCTG |
| | | | VAPAVTTPEAPKEEAPKADAAPAAP | CTCAAAAGGCCAGGGCCGCTGAGACGGTCACCCAGGCCAACGAGGAG |
| | | | QPAAESKVAKEPTDQSAENQSDSL | AAGGACAAGGCTAAGACCGCTAAGGAGCTGGCTGAGAGGTCCGCTCA |
| | | | YKETNIKEGTEEAGTGQEQKQEPEL | AATCGTGAAGAAGAACGCCGTCGAGGCCCTGAAGGAGTTCGGCAAGA |
| | | | QNLLEQQMNIFYILVQFFKSKIKALIK | TCGCCGAGGCCGCCGAGATGGAGGCCATCAAGATCCCAATCCCAGAG |
| | | | FLLILVSHHHHHH | AACCTGAAGCCAAAGAAGAAGGTGAAGCAACCAAGGGCCGCCGCCCA |
| | | | (SEQ ID NO: 13) | AAAGGTGGAGCCAACCCAAGCTACCGCTCACAAGGTGGTGCCACCACC |
| | | | | AGCTGAGCCACCACGCGCCCCATCCCCACCACCACCACCAGCTAAGCCA |
| | | | | GAGGCTGCCCCACCAGCTAAGGAAGTGGCTCCAGCTGTCACCACCCCA |
| | | | | GAGGCTCCAAAGGAAGAGGCCCCAAAGGCTGACGCTGCTCCAGCTGC |
| | | | | CCCACAGCCAGCCGCCGAGTCCAAGGTCGCCAAGGAGCCAACCGACCA |
| | | | | GAGCGCCGAGAACCAATCCGACAGCCTCTACAAGGAGACGAACATCAA |
| | | | | GGAAGGCACCGAGGAAGCCGGCACCGGCCAAGAGCAGAAGCAAGAG |
| | | | | CCAGAGCTCCAAAACCTCCTGGAGCAACAGATGAACATCTTCTACATCC |
| | | | | TGGTGCAGTTCTTCAAGTCCAAGATCAAGGCCCTCATCAAGTTCCTCCT |
| | | | | GATCCTGGTCAGCCATCACCACCACCACCACTGA |
| | | | | (SEQ ID NO: 14) |
| 8 | 41K blood | PVX_ | MDENTGWPIDYEFNSKTLPSIEVKLS | ATGGACGAGAACACCGGCTGGCCAATCGACTACGAGTTCAACTCCAAG |
| | stage | 084420 | PPENPLPQVAAEIKLLESARLKLEEG | ACCCTGCCAAGCATCGAGGTGAAGCTCTCCCCACCAGAGAACCCACTG |
| | antigen | | MMQKLEDEYNKSLSSAKIKIQDTVE | CCACAAGTCGCCGCCGAGATCAAGCTCCTGGAGAGCGCCCGCCTCAAG |
| | precursor | | KSLSIFNDPNMLGSVISNSVKMLRSE | CTCGAAGAGGGCATGATGCAGAAGCTGGAGGACGAGTACAACAAGTC |
| | 41-3, | | NVKKRTENVQAKHNLKKMQTVNQA | CCTGTCCAGCGCCAAGATCAAGATCCAAGACACCGTGGAGAAGTCCCT |
| | putative | | KSGPLPPPELRKHTSFLEQNYVNRV | CAGCATCTTCAACGACCCAAACATGCTGGGCTCCGTGATCTCCAACAGC |
| | | | LPSVKISLSELTEPSVEIKEKIEEMEQ | GTCAAGATGCTCAGGAGCGAGAACGTGAAGAAGCGCACCGAGAACGT |
| | | | YRTDEEVAMFEMAISEFSILTDITILE | CCAGGCCAAGCACAACCTCAAGAAGATGCAGACCGTCAACCAAGCCAA |
| | | | LEKQIQLQLNPFLVDKKVVHRALTKE | GAGCGGCCCACTCCCACCACCAGAGCTGCGCAAGCACACCTCCTTCCTG |
| | | | LKELEQREEKQKIKENFQRQSSFIEA | GAGCAAAACTACGTGAACAGGGTCCTGCCATCCGTGAAGATCTCCCTC |
| | | | GEDEDTGNILNVKISQTDYGYPTVD | AGCGAGCTGACCGAGCCAAGCGTCGAGATCAAGGAGAAGATCGAGGA |
| | | | ELVMQMQKRRDISEKLERQKILDLQ | GATGGAGCAGTACAGGACCGACGAGGAAGTGGCCATGTTCGAGATGG |
| | | | MKLLKAQSEMIKDALHFALSKVIAQY | CCATCTCCGAGTTCAGCATCCTCACCGACATCACCATCCTGGAGCTGGA |
| | | | SPLVETMKLESMRMLHHHHHH | GAAGCAAATCCAGCTCCAACTGAACCCATTCCTCGTCGACAAGAAGGT |
| | | | (SEQ ID NO: 15) | GGTCCACAGGGCCCTGACCAAGGAGCTCAAGGAGCTGGAGCAGCGCG |
| | | | | AGGAGAAGCAAAAGATCAAGGAGAACTTCCAGAGGCAATCCAGCTTC |
| | | | | ATCGAGGCTGGCGAGGACGAGGACACCGGCAACATCCTCAACGTGAA |
| | | | | GATCTCCCAGACCGACTACGGCTACCCAACCGTGGACGAGCTCGTCAT |
| | | | | GCAGATGCAAAAGAGGCGCGACATCTCCGAGAAGCTGGAGCGCCAGA |
| | | | | AGATCCTCGACCTGCAGATGAAGCTCCTGAAGGCCCAGAGCGAGATGA |
| | | | | TCAAGGACGCCCTCCACTTCGCCCTGTCCAAGGTCATCGCCCAATACAG |
| | | | | CCCACTCGTCGAGACGATGAAGCTGGAGAGCATGAGGATGCTCCACCA |
| | | | | CCACCACCACCACTGA (SEQ ID NO: 16) |
| 9 | rhoptry- | PVX_ | MSSDGKSSASAKSGSKSGSKYGGS | ATGAGCAGCGACGGCAAGTCCAGCGCTTCCGCTAAGTCCGGCAGCAA |
| | associ- | 085930 | SYSDYSAYDSGSASSVGSREFENE | GTCCGGCAGCAAGTACGGCGGCTCCAGCTACTCCGACTACAGCGCCTA |
| | ated | | MYEFALQHPMEKLTKEMDILKNDYT | CGACTCCGGCAGCGCCTCCAGCGTGGGCAGCCGCGAGTTCGAGAACG |
| | protein 1, | | KVKEEEGKILDEEHKEIEEKRKEERL | AGATGTACGAGTTCGCCCTGCAACACCCGATGGAGAAGCTCACCAAGG |
| | putative | | KMLAEGDVEKNKGDEEINFIKHDYT | AGATGGACATCCTGAAGAACGACTACACCAAGGTGAAGGAAGAGGAA |
| | (RAP1) | | DTRIRGGFTEFLSNLNPFKKEIKPMK | GGCAAGATCCTCGACGAGGAGCACAAGGAGATCGAGGAGAAGAGGA |
| | | | KEISLITYIPDKIVNKEKIMRDLGISHK | AGGAAGAGCGCCTCAAGATGCTGGCCGAGGGCGACGTGGAGAAGAA |
| | | | YEPYQQSILYTCPNSVFFFDSMENL | CAAGGGCGACGAGGAGATCAACTTCATCAAGCACGACTACACCGACAC |
| | | | RKELDKNHEKEAITNKILDHNKECLK | CAGGATCCGCGGCGGCTTCACCGAGTTCCTCTCCAACCTGAACCCATTC |
| | | | NFGLFDFELPDNKTKLGNVIGSIGEY | AAGAAGGAGATCAAGCCGATGAAGAAGGAGATCTCCCTCATCACCTAC |
| | | | HVRLYEIENDLLKYQPSLDYMTLAD | ATCCCAGACAAGATCGTCAACAAGGAGAAGATCATGCGCGACCTGGG |
| | | | DYKLVKNDVNTLENVNFCLLNPKTL | CATCTCCCACAAGTACGAGCCATACCAACAGAGCATCCTCTACACCTGC |
| | | | EDFLKKKEIMELMGEDPIAYEEKFTK | CCAAACTCCGTGTTCTTCTTCGACAGCATGGAGAACCTCAGGAAGGAG |
| | | | YMEESINCHLESLIYEDLDSSQDTKI | CTGGACAAGAACCACGAGAAGGAAGCCATCACCAACAAGATCCTCGAC |
| | | | VLKNVKSKLYLLONGLTYKSKKLINK | CACAACAAGGAGTGCCTCAAGAACTTCGGCCTGTTCGACTTCGAGCTCC |
| | | | LFNEIQKNPEPIFEKLTWIYENMYHL | CAGACAACAAGACCAAGCTGGGCAACGTCATCGGCTCCATCGGCGAGT |
| | | | KRDYTFLAFKTVCDKYVSHNSIYTSL | ACCACGTGAGGCTCTACGAGATCGAGAACGACCTCCTGAAGTACCAAC |
| | | | QGMTSYIIEYTRLYGACFKNITIYNAV | CAAGCCTGGACTACATGACCCTCGCCGACGACTACAAGCTGGTGAAGA |
| | | | ISGIHEQMKNLMKLMPRSGLLSDVH | ACGACGTCAACACCCTGGAGAACGTGAACTTCTGCCTCCTGAACCCAA |
| | | | FEALLHKENKKITRTDYVLNDYDPSV | AGACCCTGGAGGACTTCCTCAAGAAGAAGGAGATCATGGAGCTGATG |
| | | | KAYALTQVERLPMVSVINSFFEAKKK | GGCGAGGACCCAATCGCCTACGAGGAGAAGTTCACCAAGTACATGGA |
| | | | ALSKMLAQMKLDLFTLTNEDLKIPND | GGAGTCCATCAACTGCCACCTGGAGAGCCTGATCTACGAGGACCTCGA |
| | | | KGANSKLTAKLISIYKAEIKKYFKEMR | CTCCAGCCAAGACACCAAGATCGTGCTCAAGAACGTCAAGTCCAAGCT |
| | | | DDYVFLIKARYKGHYKKNYLLYKRLE | GTACCTCCTGCAGAACGGCCTCACCTACAAGAGCAAGAAGCTCATCAA |
| | | | HHHHHH (SEQ ID NO: 17) | CAAGCTGTTCAACGAGATCCAGAAGAACCCAGAGCCAATCTTCGAGAA |
| | | | | GCTCACCTGGATCTACGAGAACATGTACCACCTGAAGCGCGACTACAC |
| | | | | CTTCCTCGCCTTCAAGACCGTGTGCGACAAGTATGTGTCCCACAACAGC |
| | | | | ATCTACACCTCCCTGCAAGGCATGACCAGCTACATCATCGAGTACACCA |
| | | | | GGCTCTACGGCGCCTGCTTCAAGAACATCACCATCTACAACGCCGTCAT |
| | | | | CTCCGGCATCCACGAGCAGATGAAGAACCTCATGAAGCTGATGCCAAG |
| | | | | GTCCGGCCTCCTGAGCGACGTGCACTTCGAGGCCCTCCTGCACAAGGA |
| | | | | GAACAAGAAGATCACCCGCACCGACTACGTGCTCAACGACTACGACCC |
| | | | | ATCCGTCAAGGCCTACGCCCTGACCCAAGTGGAGAGGCTCCCAATGGT |
| | | | | GTCCGTCATCAACAGCTTCTTCGAGGCCAAGAAGAAGGCCCTCAGCAA |
| | | | | GATGCTGGCCCAGATGAAGCTCGACCTGTTCACCCTGACCAACGAGGA |
| | | | | CCTCAAGATCCCAAACGACAAGGGCGCCAACTCCAAGCTCACCGCCAA |
| | | | | GCTGATCAGCATCTACAAGGCCGAGATCAAGAAGTACTTCAAGGAGAT |
| | | | | GAGGGACGACTACGTCTTCCTGATCAAGGCCCGCTACAAGGGGCACTA |
| | | | | CAAGAAGAACTACCTCCTGTACAAGCGCCTGGAGCACCACCACCACCA |
| | | | | CCACTGA (SEQ ID NO: 18) |
| 10 | hypothe- | PVX_ | MNTRASKFANSKRKRNGNAMRENK | ATGAACACCAGGGCCTCCAAGTTCGCCAACAGCAAGAGGAAGCGCAA |
| | tical | 094830 | LNNDDVDHYSFLSLRTANEEKAATE | CGGCAACGCCATGCGCGAGAACAAGCTCAACAACGACGACGTGGACC |
| | protein, | | NDSNNAKKEGEENTNGNEKKNEEN | ACTACTCCTTCCTCAGCCTGAGGACCGCTAACGAGGAGAAGGCTGCTA |
| | conserved | | GSGNEKRNEENNANEKKNEQTNDQ | CCGAGAACGACTCCAACAACGCCAAGAAGGAAGGCGAGGAGAACACC |
| | | | SNGQSNSQTNIPKKNEAVPPEKKIN | AACGGCAACGAGAAGAAGAACGAGGAGAACGGCAGCGGCAACGAGA |
| | | | KENLLEYGTHDKDGHFIPSYKTLTDE | AGCGCAACGAGGAGAACAACGCTAACGAGAAGAAGAACGAGCAAACC |
| | | | ILSTNNSLERASSFLKIACSHIMKIVE | AACGACCAGTCCAACGGCCAATCCAACAGCCAGACCAACATCCCAAAG |
| | | | FIPESKLSSQYIKVESKNVYIKDITSE | AAGAACGAGGCCGTCCCACCAGAGAAGAAGATCAACAAGGAGAACCT |
| | | | CQNIFFSLEKLTMTMIVLNSKMNKLV | CCTGGAGTACGGCACCCACGACAAGGACGGCCACTTCATCCCAAGCTA |
| | | | YVQDKHHHHHH | CAAGACCCTCACCGACGAGATCCTGTCCACCAACAACAGCCTGGAGAG |
| | | | (SEQ ID NO: 19) | GGCCTCCAGCTTCCTGAAGATCGCCTGCTCCCACATCATGAAGATCGTG |
| | | | | GAGTTCATCCCAGAGTCCAAGCTGTCCAGCCAATACATCAAGGTGGAG |
| | | | | AGCAAGAACGTCTACATCAAGGACATCACCTCCGAGTGCCAGAACATC |
| | | | | TTCTTCAGCCTGGAGAAGCTGACCATGACCATGATCGTCCTCAACAGCA |
| | | | | AGATGAACAAGCTGGTCTACGTGCAAGACAAGCACCACCACCACCACC |
| | | | | ACTGA (SEQ ID NO: 20) |
| 11 | trypto- | PVX_ | MPKPAQNLKGGVKKPSLQQTKSPL | ATGCCAAAGCCAGCCCAAAACCTCAAGGGCGGCGTGAAGAAGCCATC |
| | phan-rich | 112675 | PSKPPKPVNDKLKDDSNKTETKDAK | CCTCCAACAGACCAAGTCCCCACTGCCAAGCAAGCCACCAAAGCCAGT |
| | antigen | | NGLNKPPKNINDKVKDGENKTPSQD | CAACGACAAGCTCAAGGACGACAGCAACAAGACCGAGACGAAGGACG |
| | (Pv-fam-a) | | LNEPSFKLPMRQKASSWDAWLKGT | CCAAGAACGGCCTGAACAAGCCACCAAAGAACATCAACGACAAGGTG |
| | | | KKDYENLKCFAKGNLYDWLCSVRD | AAGGACGGCGAGAACAAGACCCCATCCCAAGACCTCAACGAGCCAAG |
| | | | SFELYLOSLESKWTSCSDNTTTVFL | CTTCAAGCTGCCAATGAGGCAAAAGGCCTCCAGCTGGGACGCTTGGCT |
| | | | CECLAESSGWGDPQWESWVKKEL | CAAGGGCACCAAGAAGGACTACGAGAACCTGAAGTGCTTCGCCAAGG |
| | | | KEQLKTEAQAWISTKKKDFDGLTSK | GCAACCTCTACGACTGGCTGTGCTCCGTCCGCGACAGCTTCGAGCTCTA |
| | | | YFSLWKDHRRKELEEEAWKTKASS | CCTGCAATCCCTGGAGAGCAAGTGGACCTCCTGCAGCGACAACACCAC |
| | | | GGLSEWEELTDKMNTRYTNNLDNM | CACCGTGTTCCTCTGCGAGTGCCTCGCTGAGTCCAGCGGCTGGGGCGA |
| | | | WSNYSGDLLFRFDEWSPEVLEKWI | CCCACAGTGGGAGTCCTGGGTCAAGAAGGAGCTCAAGGAGCAACTGA |
| | | | ESKQWNQWVKKVRKHHHHHH | AGACCGAGGCCCAGGCCTGGATCAGCACCAAGAAGAAGGACTTCGAC |
| | | | (SEQ ID NO: 21) | GGCCTCACCTCCAAGTACTTCAGCCTGTGGAAGGACCACAGGCGCAAG |
| | | | | GAGCTGGAGGAAGAGGCCTGGAAGACCAAGGCCTCCAGCGGCGGCCT |
| | | | | CTCCGAGTGGGAGGAGCTGACCGACAAGATGAACACCAGGTACACCA |
| | | | | ACAACCTCGACAACATGTGGTCCAACTACAGCGGCGACCTCCTGTTCCG |
| | | | | CTTCGACGAGTGGTCCCCAGAGGTGCTGGAGAAGTGGATCGAGAGCA |
| | | | | AGCAGTGGAACCAGTGGGTGAAGAAGGTCAGGAAGCACCACCACCAC |
| | | | | CACCACTGA (SEQ ID NO: 22) |
| 12 | trypto- | PVX_ | MVTEGGDNLDDDLGGDLEGLLGDD | ATGGTGACCGAGGGCGGCGACAACCTCGACGACGACCTCGGCGGCGA |
| | phan-rich | 112670 | AEGGAAGGEGAAAAASAEGLSGEV | CCTGGAGGGCCTCCTGGGCGACGACGCTGAGGGCGGCGCCGCCGGCG |
| | antigen | | ENELLYVKEDDDDAPAATPDEKPST | GCGAGGGCGCTGCCGCCGCCGCCTCCGCCGAGGGCCTGAGCGGCGAG |
| | (Pv-fam-a) | | SGEETPAAFVDLVNETVPPPAKAPL | GTGGAGAACGAGCTCCTCTACGTGAAGGAAGACGACGACGACGCTCC |
| | | | PLQTKAPQGPKIKDWNQWMKQAKK | AGCTGCTACCCCAGACGAGAAGCCATCCACCAGCGGCGAGGAGACGC |
| | | | DFSGYKGTMHTQRHEWTKEKEDEL | CAGCTGCTTTCGTGGACCTCGTCAACGAGACGGTGCCACCACCAGCTA |
| | | | QKFCKYLEKRWMNYTGNIDRECRS | AGGCCCCACTCCCACTGCAAACCAAGGCCCCACAGGGCCCAAAGATCA |
| | | | DFLKSTQNWNESQWNKWVKSEGK | AGGACTGGAACCAGTGGATGAAGCAGGCCAAGAAGGACTTCTCCGGC |
| | | | HHMNKQFQKWLDYNKYKLQDWTN | TACAAGGGCACCATGCACACCCAAAGGCACGAGTGGACCAAGGAGAA |
| | | | TEWNKWKTTVKEQLDDEEWKKKEA | GGAAGACGAGCTGCAGAAGTTCTGCAAGTACCTGGAGAAGCGCTGGA |
| | | | AGKTKEWIKCTDKMEKKCLKKTKKH | TGAACTACACCGGCAACATCGACAGGGAGTGCCGCTCCGACTTCCTGA |
| | | | CKNWEKKANSSFKKWEGDFTKKWT | AGAGCACCCAAAACTGGAACGAGTCCCAGTGGAACAAGTGGGTGAAG |
| | | | SNKQWNSWCKELEKHHHHHH | AGCGAGGGCAAGCACCACATGAACAAGCAATTCCAGAAGTGGCTGGA |
| | | | (SEQ ID NO: 23) | CTACAACAAGTACAAGCTCCAAGACTGGACCAACACCGAGTGGAACAA |
| | | | | GTGGAAGACCACCGTCAAGGAGCAGCTGGACGACGAGGAGTGGAAG |
| | | | | AAGAAGGAAGCCGCCGGCAAGACCAAGGAGTGGATCAAGTGCACCGA |
| | | | | CAAGATGGAGAAGAAGTGCCTCAAGAAGACCAAGAAGCACTGCAAGA |
| | | | | ACTGGGAGAAGAAGGCCAACTCCAGCTTCAAGAAGTGGGAGGGCGAC |
| | | | | TTCACCAAGAAGTGGACCTCCAACAAGCAGTGGAACAGCTGGTGCAAG |
| | | | | GAGCTGGAGAAGCACCACCACCACCACCACTGA |
| | | | | (SEQ ID NO: 24) |
| 13 | Hyp, huge | PVX_ | mAVEVVQEAADEVLEEEKIEEPLEIV | ATGGCTGTGGAGGTGGTCCAAGAGGCCGCTGACGAGGTGCTCGAAGA |
| | list of | 002550 | EEEPVQVAAEEPVEEVLEEVVQEAA | GGAGAAGATCGAGGAGCCACTGGAGATCGTGGAGGAAGAGCCAGTG |
| | orthologs, | | DEVMEEEKIEEPLEIVAEEPLEIVAEE | CAAGTCGCCGCCGAGGAGCCAGTCGAGGAAGTGCTCGAAGAGGTGGT |
| | paralogs, | | PVQVAAEEVLVEKEEVNENILNIVEEI | GCAAGAGGCCGCCGACGAGGTCATGGAGGAAGAGAAGATCGAGGAG |
| | synteny | | KESIVDKLEANEEASEEGNEDLLESA | CCTCTGGAGATCGTCGCTGAAGAACCTCTGGAGATCGTGGCTGAGGAG |
| | with | | EEAAEEVAEEAVDTTTEADVVETVE | CCTGTGCAGGTGGCTGCCGAGGAAGTGCTGGTCGAGAAGGAAGAGGT |
| | PyLSA3 | | EEAANATTEVSAEESLEVSTEAPEE | GAACGAGAACATCCTCAACATCGTGGAGGAGATCAAGGAGAGCATCG |
| | (PyLSA3syn- | | TTESESHETFEEDILKNLEENKEANE | TCGACAAGCTGGAGGCCAACGAGGAAGCCAGCGAGGAAGGCAACGA |
| | 2) | | NALEDIKEMKEEFLDYVEQRVEDNE | GGACCTCCTGGAGTCCGCTGAGGAAGCCGCTGAGGAAGTGGCTGAGG |
| | | | NVLVDLLQHLERNAHVNESVLEDLE | AAGCCGTGGACACCACCACCGAGGCTGACGTGGTGGAGACGGTGGAG |
| | | | EIKEDLLANIQMAEETRKEVTDASAE | GAAGAGGCCGCTAACGCTACCACCGAGGTGTCCGCTGAGGAGAGCCT |
| | | | SAEEVEEPVEVSAEVAAEEPVEVAA | GGAGGTGTCCACCGAGGCTCCAGAGGAGACGACCGAGTCCGAGAGCC |
| | | | EEPVEVTAEEPVEVTAEEPVEIPTEE | ACGAGACGTTCGAGGAAGACATCCTGAAGAACCTGGAGGAGAACAAG |
| | | | NIFDVIEEIKEKVLENLEETTAESVAE | GAAGCCAACGAGAACGCCCTGGAGGACATCAAGGAGATGAAGGAAG |
| | | | SVGEGADENALDVLKEMQESLLENF | AGTTCCTCGACTACGTGGAGCAAAGGGTCGAGGACAACGAGAACGTG |
| | | | GQKIEANENILASVLENIQEKVELNK | CTGGTCGACCTCCTGCAGCACCTGGAGCGCAACGCCCACGTGAACGAG |
| | | | SVLVDVLAELKEEAVSQRETAQEVA | AGCGTCCTGGAGGACCTGGAGGAGATCAAGGAAGACCTCCTGGCCAA |
| | | | AELVEEAAEVPAVEPVEEEVVEPAV | CATCCAAATGGCCGAGGAGACGAGGAAGGAAGTGACCGACGCTTCCG |
| | | | EVVEEPVEEEVVEPVVDVIEEPAVE | CTGAGAGCGCTGAGGAAGTGGAGGAGCCCGTCGAGGTGTCCGCTGAG |
| | | | VVEVPVEETVEEPVEVTAEEPVEVT | GTGGCTGCTGAGGAGCCTGTCGAGGTGGCCGCCGAGGAGCCAGTGGA |
| | | | AEEPVEETVEEPVVEVVEEPVEEPV | GGTCACCGCTGAGGAGCCTGTTGAGGTGACGGCTGAGGAGCCAGTGG |
| | | | VEAIEEPVVEPVVEPAVEVIEDATEE | AGATCCCAACCGAGGAGAACATCTTCGACGTGATCGAGGAGATCAAG |
| | | | PVEEAAEEPDVEVAEGSAIESVEEA | GAGAAGGTCCTGGAGAACCTGGAGGAGACGACCGCTGAGAGCGTGG |
| | | | FEQIIEDAAQVIAEESVEETAEQILEQ | CTGAGTCCGTGGGCGAGGGCGCTGACGAGAACGCCCTGGACGTGCTC |
| | | | ATQAVTEEAADAADVADAEEAVGTA | AAGGAGATGCAAGAGAGCCTCCTGGAGAACTTCGGCCAGAAGATCGA |
| | | | QVVTEESVAEAIEDTVEEISAEPIQAT | GGCCAACGAGAACATCCTGGCCAGCGTGCTGGAGAACATCCAGGAGA |
| | | | IEGIVGEVVESVEENIEAVEEAIKDIV | AGGTCGAGCTGAACAAGTCCGTGCTCGTCGACGTGCTGGCCGAGCTCA |
| | | | EGAVEGAPELSLEEMIEDVMVGTVA | AGGAAGAGGCCGTGTCCCAAAGGGAGACGGCTCAAGAGGTGGCTGCT |
| | | | EEDSAKEAAEETVEEVVQEDAAEEE | GAGCTGGTGGAGGAAGCCGCTGAGGTCCCAGCTGTGGAGCCAGTCGA |
| | | | AAKEAAEETVEEAEREATQEAVEET | GGAAGAGGTGGTGGAGCCAGCTGTGGAGGTGGTGGAGGAGCCTGTG |
| | | | VEDVVEEVSAEAVEEIVLETPEGTSD | GAGGAAGAGGTGGTCGAGCCAGTGGTCGACGTGATCGAGGAGCCTGC |
| | | | ESVETVVEHAVEDSLGETIATIVDDV | CGTGGAGGTCGTGGAGGTCCCAGTGGAGGAGACGGTCGAGGAGCCT |
| | | | AEETTEKSEESVVDNLGVKVEEVLD | GTGGAGGTTACCGCGGAGGAGCCTGTGGAGGTCACGGCCGAGGAGCC |
| | | | VDVEEVAQEAADDVIMRVSENESEG | TGTCGAGGAGACGGTGGAGGAGCCAGTGGTCGAGGTGGTCGAGGAG |
| | | | ESGAESGEEVEELESALFEVEKDIKK | CCAGTTGAGGAGCCTGTGGTCGAGGCCATCGAGGAGCCCGTCGTCGA |
| | | | KVLDMFSGNVEFDEKESEKLALDLQ | GCCAGTGGTCGAGCCAGCCGTCGAGGTCATCGAGGACGCTACGGAGG |
| | | | KNLLShhhhhh | AGCCCGTGGAGGAAGCCGCCGAGGAGCCGGACGTGGAGGTGGCTGA |
| | | | (SEQ ID NO: 25) | GGGCAGCGCTATCGAGTCCGTGGAGGAAGCCTTCGAGCAAATCATCG |
| | | | | AGGACGCCGCCCAAGTGATCGCTGAGGAGAGCGTGGAGGAGACGGCT |
| | | | | GAGCAAATCCTGGAGCAAGCCACCCAGGCCGTGACCGAGGAAGCCGC |
| | | | | TGACGCTGCTGACGTGGCTGACGCTGAGGAAGCCGTGGGCACCGCTC |
| | | | | AAGTCGTCACCGAGGAGAGCGTGGCTGAGGCTATCGAGGACACCGTC |
| | | | | GAGGAGATCTCCGCCGAGCCAATCCAGGCCACCATCGAGGGCATCGTG |
| | | | | GGCGAGGTCGTCGAGTCCGTCGAGGAGAACATCGAGGCCGTGGAGGA |
| | | | | AGCCATCAAGGACATCGTGGAGGGCGCTGTGGAGGGCGCTCCAGAGC |
| | | | | TCAGCCTGGAGGAGATGATCGAGGACGTCATGGTGGGCACCGTGGCT |
| | | | | GAGGAAGACTCCGCTAAGGAAGCCGCTGAGGAGACGGTGGAGGAAG |
| | | | | TGGTGCAAGAGGACGCTGCTGAGGAAGAGGCCGCCAAGGAAGCCGCC |
| | | | | GAAGAGACGGTGGAGGAAGCCGAGAGGGAGGCTACCCAAGAGGCCG |
| | | | | TCGAGGAGACGGTTGAGGACGTGGTCGAGGAAGTGTCCGCTGAGGCT |
| | | | | GTGGAGGAGATCGTCCTCGAAACCCCGGAGGGCACCTCCGACGAGAG |
| | | | | CGTGGAGACGGTGGTGGAGCACGCTGTGGAGGACTCCCTGGGCGAGA |
| | | | | CGATCGCCACCATCGTGGACGACGTCGCCGAGGAGACGACCGAGAAG |
| | | | | TCCGAGGAGAGCGTGGTCGACAACCTGGGCGTCAAGGTGGAGGAAGT |
| | | | | GCTCGACGTCGACGTGGAGGAAGTGGCCCAAGAGGCCGCCGACGACG |
| | | | | TGATCATGCGCGTCAGCGAGAACGAGTCCGAGGGCGAGAGCGGCGCT |
| | | | | GAGTCCGGCGAGGAAGTGGAGGAGCTGGAGAGCGCCCTCTTCGAGGT |
| | | | | GGAGAAGGACATCAAGAAGAAGGTCCTCGACATGTTCAGCGGCAACG |
| | | | | TGGAGTTCGACGAGAAGGAGTCCGAGAAGCTCGCCCTGGACCTCCAG |
| | | | | AAGAACCTCCTGTCCCACCACCACCACCACCACTGA |
| | | | | (SEQ ID NO: 26) |
| 14 | conserved | PVX_ | mTYMLMKDDDSHDDKDDENEEKKK | ATGACCTACATGCTCATGAAGGACGACGACTCCCACGACGACAAGGAC |
| | Plasmodium | 090970 | KEGKTNKDTNKIIKGESMTREDLLQL | GACGAGAACGAGGAGAAGAAGAAGAAGGAAGGCAAGACCAACAAGG |
| | protein, | | LNEMLKLQTDMKNIVKDLIVVAKKNS | ACACCAACAAGATCATCAAGGGCGAGAGCATGACCAGGGAGGACCTC |
| | unknown | | YDFMSVYNVAKTYNTVDPLGKYQIE | CTGCAACTCCTGAACGAGATGCTCAAGCTGCAGACCGACATGAAGAAC |
| | function | | MPEFDKVVENYHFDPEVKETVSKLM | ATCGTCAAGGACCTCATCGTGGTCGCCAAGAAGAACTCCTACGACTTCA |
| | | | SSQENYYANMSETATLNVDKIIEIHH | TGAGCGTGTACAACGTCGCCAAGACCTACAACACCGTGGACCCACTGG |
| | | | FMLNELYKIDPEFKKIPNKHELDPKLI | GCAAGTACCAAATCGAGATGCCAGAGTTCGACAAGGTGGTCGAGAAC |
| | | | ALVIQSIVSAKVEEEFNLTSEDVEASI | TACCACTTCGACCCAGAGGTGAAGGAGACGGTGTCCAAGCTCATGTCC |
| | | | ANQQYALTSNMEFARVNIQMQTIMN | AGCCAGGAGAACTACTACGCCAACATGAGCGAGACGGCCACCCTGAA |
| | | | KFMGDhhhhhh (SEQ ID NO: 27) | CGTCGACAAGATCATCGAGATCCACCACTTCATGCTCAACGAGCTGTAC |
| | | | | AAGATCGACCCAGAGTTCAAGAAGATCCCAAACAAGCACGAGCTGGAC |
| | | | | CCAAAGCTCATCGCCCTCGTGATCCAATCCATCGTGAGCGCCAAGGTCG |
| | | | | AGGAAGAGTTCAACCTCACCTCCGAGGACGTCGAGGCCAGCATCGCCA |
| | | | | ACCAACAGTACGCCCTGACCTCCAACATGGAGTTCGCCCGCGTGAACA |
| | | | | TCCAAATGCAGACCATCATGAACAAGTTCATGGGCGACCACCACCACC |
| | | | | ACCACCACTGA (SEQ ID NO: 28) |
| 15 | conserved | PVX_ | mAGGVSEEAIKKLKEIKKLELDILKDF | ATGGCCGGCGGCGTCAGCGAGGAAGCCATCAAGAAGCTCAAGGAGAT |
| | Plasmodium | 084815 | MKQDAGHADLYKKYHCIASDYISGN | CAAGAAGCTGGAGCTGGACATCCTGAAGGACTTCATGAAGCAAGACG |
| | protein, | | PKGSSAEGPNLAKKGEKSKKGEKH | CCGGCCACGCCGACCTCTACAAGAAGTACCACTGCATCGCCAGCGACT |
| | unknown | | QNGEKPQNGEKPKKSFIEKIASFVSI | ACATCTCCGGCAACCCAAAGGGCTCCAGCGCTGAGGGCCCAAACCTGG |
| | function | | FSYNNVSKIYSEHVORIFPKARDHA | CCAAGAAGGGCGAGAAGAGCAAGAAGGGCGAGAAGCACCAAAACGG |
| | | | GDGSAGDAIYPDDKIETGKKQNQSS | CGAGAAGCCACAGAACGGCGAGAAGCCAAAGAAGTCCTTCATCGAGA |
| | | | YVQLSALNLMKRNMFLGGKDKSSE | AGATCGCCTCCTTCGTGAGCATCTTCTCCTACAACAACGTCAGCAAGAT |
| | | | HFEVGNLGSFYMIFGARNTDYPWA | CTACTCCGAGCACGTGCAAAGGATCTTCCCAAAGGCCCGCGACCACGC |
| | | | CSCDPLQLIDYKEKKRNYVLCSNQV | TGGCGACGGCAGCGCCGGCGACGCCATCTACCCAGACGACAAGATCG |
| | | | DMSIQNADLFCNPKhhhhhh | AGACGGGCAAGAAGCAAAACCAGTCCAGCTACGTCCAGCTCTCCGCCC |
| | | | (SEQ ID NO: 29) | TCAACCTGATGAAGCGCAACATGTTCCTGGGCGGCAAGGACAAGTCCA |
| | | | | GCGAGCACTTCGAAGTGGGCAACCTCGGCAGCTTCTACATGATCTTCG |
| | | | | GCGCCAGGAACACCGACTACCCATGGGCCTGCTCCTGCGACCCACTCC |
| | | | | AGCTGATCGACTACAAGGAGAAGAAGCGCAACTACGTGCTCTGCAGCA |
| | | | | ACCAAGTCGACATGTCCATCCAGAACGCCGACCTGTTCTGCAACCCAAA |
| | | | | GCACCACCACCACCACCACTGA (SEQ ID NO: 30) |
| 16 | trypto- | PVX_ | mVSCTSLCLYIIYSLFLLNNVSLSIQV | ATGGTGTCCTGCACCAGCCTCTGCCTGTACATCATCTACAGCCTCTTCCT |
| | phan- | 090270 | KTNEIKNGONGSVQLKEKGGGVNL | CCTGAACAACGTGTCCCTGAGCATCCAAGTCAAGACCAACGAGATCAA |
| | rich | | APKVGTNITQKRDTKMAKKTVTKVA | GAACGGCCAAAACGGCTCCGTCCAGCTCAAGGAGAAGGGCGGCGGCG |
| | antigen | | KKKVTKVAEKTGTKVADKTGTKVAD | TGAACCTGGCTCCAAAGGTCGGCACCAACATCACCCAGAAGAGGGACA |
| | (Pv-fam-a) | | KTGTKVADKTGTKVAEKTGTKVADK | CCAAGATGGCCAAGAAGACCGTGACCAAGGTCGCCAAGAAGAAGGTC |
| | | | TGTKVAEKTGTNISQKEDEKGPPKE | ACGAAGGTCGCCGAGAAGACCGGCACCAAGGTGGCCGACAAGACCGG |
| | | | DTQGTQKADAKAIQQADAQVSEKW | CACCAAGGTCGCTGATAAGACGGGGACGAAGGTCGCTGATAAGACCG |
| | | | KKKEWKEWIKKAESDLDIFNALMDN | GGACGAAGGTGGCTGAGAAGACGGGGACGAAGGTTGCTGATAAGAC |
| | | | EKEKKWYSEKEKEWNKWIKGVEKK | GGGGACCAAGGTGGCTGAGAAGACCGGCACCAACATCAGCCAAAAGG |
| | | | WMHYNKNIYVEYRSLVFWVGLKWV | AAGACGAGAAGGGCCCACCAAAGGAAGACACCCAAGGCACCCAGAAG |
| | | | ESQWEKWILSDGLEFLVMDWKKWI | GCCGACGCCAAGGCCATCCAACAGGCCGACGCCCAGGTGAGCGAGAA |
| | | | KENKSNFDEWLKSEWDTWTNSQM | GTGGAAGAAGAAGGAGTGGAAGGAGTGGATCAAGAAGGCCGAGTCC |
| | | | EEWKSSNWKLNEDKRWEMWENDK | GACCTCGACATCTTCAACGCCCTGATGGACAACGAGAAGGAGAAGAA |
| | | | KWIKWLYLKDWINCSKWKKRIQKES | GTGGTACAGCGAGAAGGAGAAGGAGTGGAACAAGTGGATCAAGGGC |
| | | | KEWLRWTKLKEEMYhhhhhh | GTGGAGAAGAAGTGGATGCACTACAACAAGAACATCTACGTCGAGTA |
| | | | (SEQ ID NO: 31) | CAGGTCCCTCGTGTTCTGGGTCGGCCTGAAGTGGGTGGAGTCCCAATG |
| | | | | GGAGAAGTGGATCCTCAGCGACGGCCTGGAGTTCCTGGTCATGGACTG |
| | | | | GAAGAAGTGGATCAAGGAGAACAAGTCCAACTTCGACGAGTGGCTCA |
| | | | | AGAGCGAGTGGGACACCTGGACCAACTCCCAGATGGAGGAGTGGAAG |
| | | | | TCCAGCAACTGGAAGCTGAACGAGGACAAGCGCTGGGAGATGTGGGA |
| | | | | GAACGACAAGAAGTGGATCAAGTGGCTCTACCTGAAGGACTGGATCA |
| | | | | ACTGCAGCAAGTGGAAGAAGAGGATCCAAAAGGAGTCCAAGGAGTG |
| | | | | GCTCCGCTGGACCAAGCTGAAGGAAGAGATGTACCACCACCACCACCA |
| | | | | CCACTGA (SEQ ID NO: 32) |
| 17 | apical | PVX_ | mGEDAEVENAKYRIPAGRCPVFGK | ATGGGCGAGGACGCCGAGGTGGAGAACGCCAAGTACAGGATCCCAGC |
| | membrane | 092275 | GIVIENSDVSFLRPVATGDQKLKDG | TGGCAGGTGCCCAGTGTTCGGCAAGGGCATCGTCATCGAGAACTCCGA |
| | antigen | | GFAFPNANDHISPMTLANLKERYKD | CGTGAGCTTCCTCCGCCCAGTGGCTACCGGCGACCAAAAGCTGAAGGA |
| | 1, AMA1 | | NVEMMKLNDIALCRTHAASFVMAGD | CGGCGGATTCGCCTTCCCAAACGCCAACGACCACATCTCCCCAATGACC |
| | (Orthologs | | QNSSYRHPAVYDEKEKTCHMLYLS | CTCGCCAACCTGAAGGAGAGGTACAAGGACAACGTGGAGATGATGAA |
| | with Pf | | AQENMGPRYCSPDAQNRDAVFCFK | GCTCAACGACATCGCTCTGTGCAGGACCCACGCTGCTAGCTTCGTGATG |
| | vaccine | | PDKNESFENLVYLSKNVRNDWDKK | GCTGGCGACCAGAACTCCAGCTACAGGCACCCAGCCGTCTACGACGAG |
| | candidates) | | CPRKNLGNAKFGLWVDGNCEEIPY | AAGGAGAAGACCTGCCACATGCTCTACCTGTCCGCCCAAGAGAACATG |
| | | | VKEVEAEDLRECNRIVFGASASDQP | GGCCCAAGGTACTGCTCCCCAGACGCTCAGAACAGGGACGCTGTCTTC |
| | | | TQYEEEMTDYQKIQQGFRQNNREM | TGCTTCAAGCCAGACAAGAACGAGTCCTTCGAGAACCTCGTGTACCTG |
| | | | IKSAFLPVGAFNSDNFKSKGRGFNW | AGCAAGAACGTCAGGAACGACTGGGACAAGAAGTGCCCACGCAAGAA |
| | | | ANFDSVKKKCYIFNTKPTCLINDKNFI | CCTCGGCAACGCCAAGTTCGGCCTGTGGGTGGACGGCAACTGCGAGG |
| | | | ATTALSHPQEVDLEFPCSIYKDEIER | AGATCCCATACGTGAAGGAAGTGGAGGCCGAGGACCTCAGGGAGTGC |
| | | | EIKKQSRNMNLYSVDGERIVLPRIFIS | AACAGGATCGTCTTCGGCGCTTCCGCTAGCGACCAACCAACCCAGTAC |
| | | | NDKESIKCPCEPERISNSTCNFYVC | GAGGAAGAGATGACCGACTACCAAAAGATCCAACAGGGCTTCAGGCA |
| | | | NCVEKRAEIKENNQVVIKEEFRDYY | GAACAACCGCGAGATGATCAAGTCCGCCTTCCTCCCAGTGGGCGCCTT |
| | | | ENGEEKSNKQhhhhhh | CAACTCCGACAACTTCAAGAGCAAGGGCCGCGGCTTCAACTGGGCCAA |
| | | | (SEQ ID NO: 33) | CTTCGACAGCGTGAAGAAGAAGTGCTACATCTTCAACACCAAGCCAAC |
| | | | | CTGCCTGATCAACGACAAGAACTTCATCGCCACCACCGCCCTCTCCCAC |
| | | | | CCACAAGAGGTCGACCTGGAGTTCCCATGCAGCATCTACAAGGACGAG |
| | | | | ATCGAGAGGGAGATCAAGAAGCAGTCCCGCAACATGAACCTCTACAGC |
| | | | | GTGGACGGCGAGAGGATCGTCCTGCCACGCATCTTCATCTCCAACGAC |
| | | | | AAGGAGAGCATCAAGTGCCCATGCGAGCCAGAGAGGATCTCCAACAG |
| | | | | CACCTGCAACTTCTACGTGTGCAACTGCGTCGAGAAGAGGGCCGAGAT |
| | | | | CAAGGAGAACAACCAAGTGGTCATCAAGGAAGAGTTCAGGGACTACT |
| | | | | ACGAGAACGGCGAGGAGAAGTCCAACAAGCAGCACCACCACCACCAC |
| | | | | CACTGA (SEQ ID NO: 34) |
| 18 | hypothe- | PVX_ | mNGNRNLNIKPTCHKSGKNDKANG | ATGAACGGCAACAGGAACCTGAACATCAAGCCAACCTGCCACAAGAGC |
| | tical | 084720 | SDNIANKGGAQHAANGATGTPSGS | GGCAAGAACGACAAGGCCAACGGCTCCGACAACATCGCTAACAAGGG |
| | protein | | SNGKKGATTTSASAGQAGASGGMA | CGGCGCCCAACACGCTGCTAACGGCGCCACCGGCACCCCAAGCGGCTC |
| | | | APGMNPNFEQMMKPLNDMFKGNG | CAGCAACGGCAAGAAGGGCGCTACGACCACCAGCGCTTCCGCTGGCC |
| | | | EGLNIENIMNSDMFQNFFNSLMGGN | AAGCTGGCGCTTCCGGCGGCATGGCCGCCCCAGGCATGAACCCAAACT |
| | | | PHDGAGGGQEILFKDMLNAMNAQG | TCGAGCAGATGATGAAGCCACTGAACGACATGTTCAAGGGCAACGGC |
| | | | GGAPGAAATSGGANKDPNISVSPE | GAGGGCCTCAACATCGAGAACATCATGAACAGCGACATGTTCCAGAAC |
| | | | QLNKINQLKDKLENVLKNVGVDVEQ | TTCTTCAACTCCCTGATGGGCGGCAACCCACACGACGGCGCTGGCGGC |
| | | | LKENMQNENIMQNKDALRDLLANLP | GGCCAAGAGATCCTGTTCAAGGACATGCTCAACGCCATGAACGCCCAA |
| | | | MNPGMMQNMMAGKDGNMFNMDP | GGCGGCGGCGCCCCAGGCGCTGCCGCCACCTCCGGCGGCGCCAACAA |
| | | | NQMMNMFNQLSQGKMNMKDFGM | GGACCCAAACATCAGCGTCTCCCCAGAGCAGCTGAACAAGATCAACCA |
| | | | GDFMPPPVHANDQDAEDDSRGKAF | ACTCAAGGACAAGCTGGAGAACGTGCTCAAGAACGTGGGCGTCGACG |
| | | | VTNSSNNDINFAHKLNAFEYSNGPS | TGGAGCAGCTCAAGGAGAACATGCAAAACGAGAACATCATGCAGAAC |
| | | | EGMFQLYGMNNDDGVIDDGMSDSV | AAGGACGCTCTGAGGGACCTCCTGGCTAACCTCCCGATGAACCCAGGC |
| | | | GKNSALDVSGGSINRNLSDGDSAKE | ATGATGCAAAACATGATGGCCGGCAAGGACGGCAACATGTTCAACATG |
| | | | DSDESNANATSNSNATVPNKGGHE | GACCCAAACCAGATGATGAACATGTTCAACCAACTCAGCCAGGGCAAG |
| | | | GGSANEVYSNEEELITSSGSKGDAN | ATGAACATGAAGGACTTCGGCATGGGCGACTTCATGCCACCACCAGTC |
| | | | KLAGTGGYKNNNAFLDLNNLKKDAS | CACGCCAACGACCAAGACGCTGAGGACGACTCCCGCGGCAAGGCTTTC |
| | | | AAKYGKDNSGDKSNGGNSNGGNN | GTGACCAACTCCAGCAACAACGACATCAACTTCGCCCACAAGCTGAAC |
| | | | KVMNKRIGGKKKKTFKKKKNPGQIP | GCCTTCGAGTACAGCAACGGCCCATCCGAGGGCATGTTCCAGCTCTAC |
| | | | FKMETLQKLVKEYTNTSNQKIMEKII | GGCATGAACAACGACGACGGCGTCATCGACGACGGCATGAGCGACTC |
| | | | KKYVSMSNQSARGNSEEEDDEEEA | CGTCGGCAAGAACAGCGCTCTGGACGTGAGCGGCGGCTCCATCAACA |
| | | | EDEKSAKDKNSEKEAELNMNEFSVK | GGAACCTCAGCGACGGCGACTCCGCCAAGGAAGACAGCGACGAGTCC |
| | | | DIKKLISEGILTYEDLTEEELKKLAKP | AACGCCAACGCCACCAGCAACTCCAACGCCACCGTCCCAAACAAGGGC |
| | | | DDMFYELSPYANEEKDLSLNETSGV | GGCCACGAGGGCGGCAGCGCTAACGAGGTGTACTCCAACGAGGAAGA |
| | | | SNEQLNAFLRKNGSYHMSYDSKAID | GCTGATCACCTCCAGCGGCTCCAAGGGCGACGCTAACAAGCTGGCTGG |
| | | | YLKQKKAEKKEEEQEDDNFYDAYK | CACCGGCGGCTACAAGAACAACAACGCCTTCCTCGACCTGAACAACCT |
| | | | QIKNSYEGIPSNYYHDAPQLIGENYV | GAAGAAGGACGCCAGCGCCGCCAAGTACGGCAAGGACAACAGCGGC |
| | | | FTSVYDKKKELIDFLKRSNGATDSSN | GACAAGTCCAACGGCGGCAACTCCAACGGCGGCAACAACAAGGTCAT |
| | | | SSAGKDKGNSAESGTYKSKYYDKY | GAACAAGCGCATCGGCGGCAAGAAGAAGAAGACCTTCAAGAAGAAGA |
| | | | MKKLSEYRRREAFKILKKRRAQEKK | AGAACCCAGGCCAAATCCCATTCAAGATGGAGACGCTCCAGAAGCTGG |
| | | | MQKKQEMQNNSSNEVDYSEYFKKN | TCAAGGAGTACACCAACACCAGCAACCAAAAGATCATGGAGAAGATCA |
| | | | GFINSSNGTVKTFSKDQLDNMVKQF | TCAAGAAGTATGTGTCCATGTCCAACCAGAGCGCCAGGGGCAACTCCG |
| | | | NSDGDDIPSSSGAGADLGDNYSGV | AGGAAGAGGACGACGAGGAAGAGGCCGAGGACGAGAAGAGCGCCAA |
| | | | SGGGQFSPSGGSGNNPSGYVTFD | GGACAAGAACTCCGAGAAGGAAGCCGAGCTGAACATGAACGAGTTCA |
| | | | GQNIVGPNENEEEEPTEDVLNEDDD | GCGTCAAGGACATCAAGAAGCTCATCTCCGAGGGCATCCTGACCTACG |
| | | | NADDDDhhhhhh | AGGACCTCACCGAGGAAGAGCTCAAGAAGCTGGCCAAGCCAGACGAC |
| | | | (SEQ ID NO: 35) | ATGTTCTACGAGCTCAGCCCATACGCCAACGAGGAGAAGGACCTCTCC |
| | | | | CTGAACGAGACGAGCGGCGTGTCCAACGAGCAACTGAACGCCTTCCTC |
| | | | | CGCAAGAACGGCTCCTACCACATGAGCTACGACTCCAAGGCCATCGAC |
| | | | | TACCTGAAGCAAAAGAAGGCCGAGAAGAAGGAAGAGGAGCAAGAGG |
| | | | | ACGACAACTTCTACGACGCCTACAAGCAAATCAAGAACAGCTACGAGG |
| | | | | GCATCCCATCCAACTACTACCACGACGCCCCACAGCTCATCGGCGAGAA |
| | | | | CTACGTCTTCACCAGCGTGTACGACAAGAAGAAGGAGCTGATCGACTT |
| | | | | CCTCAAGAGGTCCAACGGCGCTACCGACTCCAGCAACTCCAGCGCTGG |
| | | | | CAAGGACAAGGGCAACAGCGCTGAGTCCGGCACCTACAAGAGCAAGT |
| | | | | ACTACGACAAGTACATGAAGAAGCTGTCCGAGTACAGGCGCAGGGAG |
| | | | | GCCTTCAAGATCCTCAAGAAGCGCAGGGCCCAGGAGAAGAAGATGCA |
| | | | | AAAGAAGCAGGAGATGCAAAACAACTCCAGCAACGAGGTGGACTACT |
| | | | | CCGAGTACTTCAAGAAGAACGGCTTCATCAACTCCAGCAACGGCACCG |
| | | | | TCAAGACCTTCAGCAAGGACCAACTGGACAACATGGTGAAGCAGTTCA |
| | | | | ACTCCGACGGCGACGACATCCCATCCAGCTCCGGCGCTGGCGCTGACC |
| | | | | TCGGCGACAACTACAGCGGCGTGTCCGGCGGCGGCCAATTCAGCCCAT |
| | | | | CCGGCGGCAGCGGCAACAACCCATCCGGCTACGTCACCTTCGACGGCC |
| | | | | AGAACATCGTGGGCCCAAACGAGAACGAGGAAGAGGAGCCAACCGA |
| | | | | GGACGTGCTCAACGAGGACGACGACAACGCCGACGACGACGACCACC |
| | | | | ACCACCACCACCACTGA (SEQ ID NO: 36) |
| 19 | merozoite | PVX_ | mPLEVSLWGQGNAHLGTQTSRLLR | ATGCCGCTGGAGGTGTCCCTGTGGGGCCAGGGCAACGCTCACCTCGGC |
| | surface | 003770 | ESGRNGQANRVNQADQADQVASP | ACCCAAACCTCCCGCCTGCTCAGGGAGTCCGGCAGGAACGGCCAGGCC |
| | protein 5 | | PISGKERRRGIGMTSNLQLLSGEDE | AACAGGGTGAACCAGGCTGACCAGGCTGACCAAGTGGCTTCCCCACCA |
| | | | KDSTSEEAPNLEGKDNADAGKDGE | ATCTCCGGCAAGGAGAGGCGCAGGGGCATCGGCATGACCTCCAACCTC |
| | | | KEPSEKQSGDVDPTVTDAERAKDE | CAACTCCTGAGCGGCGAGGACGAGAAGGACTCCACCAGCGAGGAAGC |
| | | | NASVSEEEQMKTLDSGEDHTDDGN | CCCAAACCTGGAGGGCAAGGACAACGCTGACGCTGGCAAGGATGGCG |
| | | | ADGGQGGGDGNDENQKGDGKEKE | AGAAGGAGCCATCCGAGAAGCAGAGCGGCGACGTGGACCCAACCGTC |
| | | | GGEEKKEDGKDDHEKGEKGSEGES | ACCGACGCTGAGAGGGCTAAGGACGAGAACGCTTCCGTCAGCGAGGA |
| | | | GEKDEAAPKGDAAEKDKKLESKTAD | AGAGCAGATGAAGACCCTGGACAGCGGCGAGGACCACACCGACGACG |
| | | | AKVSEHKADDANPGGNKDSPEGES | GCAACGCTGACGGCGGACAAGGCGGCGGCGACGGCAACGACGAGAA |
| | | | PKEGNPDDPSQKNPEAAGDDDSRL | CCAAAAGGGCGACGGCAAGGAGAAGGAAGGCGGCGAGGAGAAGAA |
| | | | HLDNLDDKVPHYSALRNNRVEKGVT | GGAAGACGGCAAGGACGACCACGAGAAGGGCGAGAAGGGCTCCGAG |
| | | | DTMVLNDIIGENAKSCSVDNGGCAD | GGCGAGAGCGGCGAGAAGGACGAGGCTGCTCCAAAGGGCGACGCTG |
| | | | DQICIRIDNIGIKCICKEGHLFGDKCIL | CCGAGAAGGACAAGAAGCTGGAGTCCAAGACCGCCGACGCCAAGGTG |
| | | | TKhhhhhh (SEQ ID NO: 37) | AGCGAGCACAAGGCTGACGACGCTAACCCAGGCGGCAACAAGGACTC |
| | | | | CCCAGAGGGCGAGAGCCCAAAGGAAGGCAACCCAGACGACCCATCCC |
| | | | | AGAAGAACCCGGAGGCTGCTGGCGACGACGACAGCCGCCTCCACCTG |
| | | | | GACAACCTCGACGACAAGGTCCCACACTACTCCGCCCTGCGCAACAAC |
| | | | | AGGGTGGAGAAGGGCGTCACCGACACCATGGTGCTGAACGACATCAT |
| | | | | CGGCGAGAACGCCAAGTCCTGCAGCGTGGACAACGGCGGCTGCGCTG |
| | | | | ACGACCAAATCTGCATCAGGATCGACAACATCGGCATCAAGTGCATCT |
| | | | | GCAAGGAAGGCCACCTCTTCGGCGACAAGTGCATCCTGACCAAGCACC |
| | | | | ACCACCACCACCACTGA (SEQ ID NO: 38) |
| 20 | TRAg (Pv- | PVX_ | mDVLQLVIPSEEDIQLDKPKKDELGS | ATGGACGTGCTCCAACTGGTCATCCCAAGCGAGGAAGACATCCAGCTC |
| | fam-a) | 092990 | GILSILDVHYQDVPKEFMEEEEETAV | GACAAGCCAAAGAAGGACGAGCTGGGCAGCGGCATCCTCTCCATCCTG |
| | | | YPLKPEDFAKEDSQSTEWLTFIQGL | GACGTGCACTACCAAGACGTCCCAAAGGAGTTCATGGAGGAAGAGGA |
| | | | EGDWERLEVSLNKARERWMEQRN | AGAGACGGCCGTGTACCCACTCAAGCCAGAGGACTTCGCCAAGGAAG |
| | | | KEWAGWLRLIENKWSEYSQISTKGK | ACTCCCAAAGCACCGAGTGGCTCACCTTCATCCAAGGCCTGGAGGGCG |
| | | | DPAGLRKREWSDEKWKKWFKAEV | ACTGGGAGAGGCTGGAGGTGTCCCTGAACAAGGCCAGGGAGCGCTGG |
| | | | KSQIDSHLKKWMNDTHSNLFKILVK | ATGGAGCAAAGGAACAAGGAGTGGGCTGGCTGGCTCAGGCTGATCGA |
| | | | DMSQFENKKTKEWLMNHWKKNER | GAACAAGTGGTCCGAGTACAGCCAGATCTCCACCAAGGGCAAGGACC |
| | | | GYGSESFEVMTTSKLLNVAKSREW | CGGCTGGCCTCAGGAAGCGCGAGTGGTCCGACGAAAAGTGGAAGAAG |
| | | | YRANPNINRERRELMKWFLLKENEY | TGGTTCAAGGCCGAGGTGAAGAGCCAAATCGACTCCCACCTGAAGAA |
| | | | LGQEWKKWTHWKKVKFFVFNSMC | GTGGATGAACGACACCCACAGCAACCTCTTCAAGATCCTGGTCAAGGA |
| | | | TTFSGKRLTKEEWNQFVNEIKVhhhh | CATGTCCCAGTTCGAGAACAAGAAGACCAAGGAGTGGCTCATGAACCA |
| | | | hh (SEQ ID NO: 39) | CTGGAAGAAGAACGAGAGGGGCTACGGCTCCGAGAGCTTCGAGGTCA |
| | | | | TGACCACCAGCAAGCTCCTGAACGTCGCCAAGTCCAGGGAGTGGTACC |
| | | | | GCGCCAACCCAAACATCAACCGCGAGAGGCGCGAGCTCATGAAGTGG |
| | | | | TTCCTCCTGAAGGAGAACGAGTACCTGGGCCAAGAGTGGAAGAAGTG |
| | | | | GACCCACTGGAAGAAGGTGAAGTTCTTCGTCTTCAACAGCATGTGCAC |
| | | | | CACCTTCTCCGGCAAGCGCCTGACCAAGGAAGAGTGGAACCAGTTCGT |
| | | | | GAACGAGATCAAGGTCCACCACCACCACCACCACTGA |
| | | | | (SEQ ID NO: 40) |
| 21 | unspeci- | PVX_ | mEAMPKFPQNNLKGGLKDSPLKQP | ATGGAGGCCATGCCAAAGTTCCCACAAAACAACCTCAAGGGCGGCCTG |
| | fied | 112690 | KSPLINGPPKPVNDKLKDDSNKTET | AAGGACTCCCCACTCAAGCAGCCAAAGAGCCCACTGATCAACGGCCCA |
| | product | | KDAKNGLNKPPKNINDKVKDGENKT | CCAAAGCCAGTGAACGACAAGCTCAAGGACGACTCCAACAAGACCGA |
| | | | PSQDLNEPSFKLPMRQKESSWYTW | GACGAAGGACGCCAAGAACGGCCTGAACAAGCCACCAAAGAACATCA |
| | | | LKGTKKDYETLKCFAKGNLYDWLCN | ACGACAAGGTCAAGGACGGCGAGAACAAGACCCCATCCCAAGACCTC |
| | | | VRESFDLYLQSLEKKWTTCSDSATT | AACGAGCCAAGCTTCAAGCTGCCAATGAGGCAGAAGGAGTCCAGCTG |
| | | | LFLCECFAESSGWNDSQWGNWMN | GTACACCTGGCTCAAGGGCACCAAGAAGGACTACGAGACGCTGAAGT |
| | | | NQLKEQLKTEAEAWISTKKKDFDGL | GCTTCGCCAAGGGCAACCTCTACGACTGGCTGTGCAACGTGCGCGAGT |
| | | | TSKYFSLWKDHRRKELDADEWKNK | CCTTCGACCTCTACCTGCAAAGCCTGGAGAAGAAGTGGACCACCTGCT |
| | | | VSSGGLSEWEELTNKMNTRYRNNL | CCGACAGCGCTACCACCCTCTTCCTGTGCGAGTGCTTCGCCGAGTCCAG |
| | | | DNMWSHFSRDLFFNFDEWAPQVLE | CGGCTGGAACGACTCCCAGTGGGGCAACTGGATGAACAACCAACTCAA |
| | | | KWIENKQWNRWVKKVRKhhhhhh | GGAGCAGCTGAAGACCGAGGCCGAGGCCTGGATCAGCACCAAGAAGA |
| | | | (SEQ ID NO: 41) | AGGACTTCGACGGCCTCACCTCCAAGTACTTCAGCCTGTGGAAGGACC |
| | | | | ACAGGCGCAAGGAGCTCGACGCCGACGAGTGGAAGAACAAGGTGTCC |
| | | | | AGCGGCGGCCTCAGCGAGTGGGAGGAGCTGACCAACAAGATGAACAC |
| | | | | CAGGTACCGCAACAACCTCGACAACATGTGGTCCCACTTCAGCAGGGA |
| | | | | CCTGTTCTTCAACTTCGACGAGTGGGCCCCACAAGTCCTGGAGAAGTG |
| | | | | GATCGAGAACAAGCAGTGGAACCGCTGGGTGAAGAAGGTCCGCAAGC |
| | | | | ACCACCACCACCACCACTGA (SEQ ID NO: 42) |
| 22 | petidase, | PVX_ | mQKAPNNGKNNYGLNDDELGAILF | ATGCAAAAGGCCCCAAACAACGGCAAGAACAACTACGGCCTCAACGAC |
| | M16 | 091710 | GLNYDSIAKNKDNLEKRKNVENESIF | GACGAGCTGGGCGCCATCCTCTTCGGCCTGAACTACGACAGCATCGCC |
| | family | | LRNFANEDTSKNTQSEKAQKEIKIET | AAGAACAAGGACAACCTGGAGAAGAGGAAGAACGTCGAGAACGAGT |
| | | | ETESVNSNEKEVATSQKSDTSNKNS | CCATCTTCCTGCGCAACTTCGCCAACGAGGACACCAGCAAGAACACCC |
| | | | SVENEKIELKNDELLGKNFEKDKVN | AATCCGAGAAGGCCCAGAAGGAGATCAAGATCGAGACGGAGACGGA |
| | | | KKGDNTNTTNNHDLTNSSEKQGVDI | GTCCGTCAACAGCAACGAGAAGGAAGTGGCCACCTCCCAGAAGAGCG |
| | | | RGSKNMNNYLQKTGDTNIEKSESLQ | ACACCTCCAACAAGAACTCCAGCGTCGAGAACGAGAAGATCGAGCTGA |
| | | | KDVNIKNHNEEANDAKRLDSAQTNN | AGAACGACGAGCTCCTGGGCAAGAACTTCGAGAAGGACAAGGTGAAC |
| | | | EKSKISKDTIDKDVQSNELTNLASNR | AAGAAGGGCGACAACACCAACACCACCAACAACCACGACCTCACCAAC |
| | | | SNKKSQGLAKKENELKSANLEENHN | TCCAGCGAGAAGCAAGGCGTCGACATCAGGGGCAGCAAGAACATGAA |
| | | | AKKDLLKKDQKREDGKKITHPENSN | CAACTACCTCCAAAAGACCGGCGACACCAACATCGAGAAGTCCGAGAG |
| | | | SDQYGVQVSLNDEEKNTNTKSVSH | CCTGCAGAAGGACGTGAACATCAAGAACCACAACGAGGAAGCCAACG |
| | | | SEDHSASYSGEKFGTHVSNSQKDM | ACGCCAAGAGGCTGGACAGCGCCCAGACCAACAACGAGAAGAGCAAG |
| | | | LKNIRPVQFDESAYGKLNGGSPEND | ATCTCCAAGGACACCATCGACAAGGACGTGCAATCCAACGAGCTCACC |
| | | | ENEILNKINKNNENNFSEKVALRKGT | AACCTGGCCAGCAACCGCTCCAACAAGAAGAGCCAGGGCCTCGCCAA |
| | | | KDRNEYEYFKLKSNDFKVLGIINKYS | GAAGGAGAACGAGCTCAAGTCCGCCAACCTGGAGGAGAACCACAACG |
| | | | SRGGFSISVDCGGYDDFDEVPGVS | CCAAGAAGGACCTCCTGAAGAAGGACCAAAAGAGGGAGGACGGCAA |
| | | | NLLQHAIFYKSEKRNTTLLSELGKYS | GAAGATCACCCACCCAGAGAACTCCAACAGCGACCAATACGGCGTGCA |
| | | | SEYNSCTSESSTSYYATAHSEDIYHL | AGTGTCCCTGAACGACGAGGAGAAGAACACCAACACCAAGTCCGTCA |
| | | | LNLFAENLFYPVFSEEHIQNEVKEIN | GCCACTCCGAGGACCACAGCGCTTCCTACAGCGGCGAGAAGTTCGGCA |
| | | | NKYISIENNLESCLKIASQYITNFKYS | CCCACGTCTCCAACAGCCAAAAGGACATGCTCAAGAACATCCGCCCAG |
| | | | KFFVNGNYTTLCENVLKNRLSIKNIL | TGCAGTTCGACGAGAGCGCTTACGGCAAGCTCAACGGCGGCTCCCCAG |
| | | | TEFHKKCYQPRNMSLTILLGNKVNT | AGAACGACGAGAACGAGATCCTGAACAAGATCAACAAGAACAACGAG |
| | | | ADHYNMKDVENMVVHIFGKIKNESY | AACAACTTCAGCGAGAAGGTGGCCCTCAGGAAGGGCACCAAGGACCG |
| | | | PIDGDVIGKRINRMESERVNLYGKK | CAACGAGTACGAGTACTTCAAGCTCAAGTCCAACGACTTCAAGGTCCT |
| | | | DSYNDANFIHIEGRNEKEAAFLQSM | GGGCATCATCAACAAGTACTCCAGCAGGGGCGGCTTCTCCATCAGCGT |
| | | | NELHYALDLNOKSRYVEIIKKEEWG | GGACTGCGGCGGATACGACGACTTCGACGAGGTGCCAGGCGTCTCCA |
| | | | DQLYLYWSSKTNAELCKKIEEFGSM | ACCTCCTGCAACACGCCATCTTCTACAAGAGCGAGAAGCGCAACACCA |
| | | | TFLREIFSDFRRNGLYYKISVENKYV | CCCTCCTGTCCGAGCTCGGCAAGTACTCCAGCGAGTACAACAGCTGCA |
| | | | YDLEVTSICNKYYLNFGILVKLTQRG | CCTCCGAGTCCAGCACCAGCTACTACGCCACCGCCCACTCCGAGGACAT |
| | | | RTNLAHLIHICNVFVNEIGKLFDRDSL | CTACCACCTCCTGAACCTCTTCGCCGAGAACCTGTTCTACCCAGTCTTCA |
| | | | DKGISKYILDYYREKALVTDLKFNSD | GCGAGGAGCACATCCAAAACGAGGTGAAGGAGATCAACAACAAGTAC |
| | | | NVNVSLDDLVIYSKRLLVHADDPSSL | ATCTCCATCGAGAACAACCTGGAGAGCTGCCTGAAGATCGCCTCCCAG |
| | | | LTIHSLIEDKHKNDFRNHIKIThhhhhh | TACATCACCAACTTCAAGTACAGCAAGTTCTTCGTCAACGGCAACTACA |
| | | | (SEQ ID NO: 43) | CCACCCTCTGCGAGAACGTGCTCAAGAACAGGCTGAGCATCAAGAACA |
| | | | | TCCTGACCGAGTTCCACAAGAAGTGCTACCAGCCACGCAACATGTCCCT |
| | | | | CACCATCCTCCTGGGCAACAAGGTCAACACCGCCGACCACTACAACAT |
| | | | | GAAGGACGTGGAGAACATGGTGGTCCACATCTTCGGCAAGATCAAGA |
| | | | | ACGAGTCCTACCCAATCGACGGCGACGTCATCGGCAAGAGGATCAACC |
| | | | | GCATGGAGAGCGAGAGGGTCAACCTCTACGGCAAGAAGGACTCCTAC |
| | | | | AACGACGCCAACTTCATCCACATCGAGGGCCGCAACGAGAAGGAAGC |
| | | | | CGCCTTCCTCCAAAGCATGAACGAGCTGCACTACGCCCTCGACCTGAAC |
| | | | | CAGAAGTCCCGCTACGTGGAGATCATCAAGAAGGAAGAGTGGGGCGA |
| | | | | CCAACTCTACCTGTACTGGTCCAGCAAGACCAACGCCGAGCTCTGCAA |
| | | | | GAAGATCGAGGAGTTCGGCAGCATGACCTTCCTCCGCGAGATCTTCTC |
| | | | | CGACTTCAGGCGCAACGGCCTGTACTACAAGATCAGCGTGGAGAACAA |
| | | | | GTATGTGTACGACCTGGAGGTGACCTCCATCTGCAACAAGTACTACCTG |
| | | | | AACTTCGGCATCCTCGTCAAGCTGACCCAAAGGGGCCGCACCAACCTC |
| | | | | GCTCACCTGATCCACATCTGCAACGTGTTCGTCAACGAGATCGGCAAGC |
| | | | | TCTTCGACAGGGACAGCCTGGACAAGGGCATCTCCAAGTACATCCTCG |
| | | | | ACTACTACCGCGAGAAGGCCCTCGTGACCGACCTGAAGTTCAACAGCG |
| | | | | ACAACGTGAACGTCTCCCTCGATGACCTGGTCATCTACAGCAAGAGGC |
| | | | | TCCTGGTGCACGCCGACGACCCATCCAGCCTCCTGACCATCCACTCCCT |
| | | | | CATCGAGGACAAGCATAAGAACGACTTCCGCAACCACATCAAGATCAC |
| | | | | CCACCACCACCACCACCACTGA (SEQ ID NO: 44) |
| 23 | rhoptry- | PVX_ | mKEAVKKGSKKAMKQPMHKPNLLE | ATGAAGGAAGCCGTGAAGAAGGGCTCCAAGAAGGCCATGAAGCAACC |
| | associated | 087885 | EEDFEEKESFSDDEMNGFMEESMD | AATGCACAAGCCAAACCTCCTGGAGGAAGAGGACTTCGAGGAGAAGG |
| | membrane | | ASKLDAKKAKTTLRSSEKKKTPTSG | AGTCCTTCAGCGACGACGAGATGAACGGCTTCATGGAGGAGTCCATGG |
| | antigen, | | MSGMSGSGATSAATEAATNMNATA | ACGCCAGCAAGCTGGACGCCAAGAAGGCCAAGACCACCCTCAGGTCC |
| | RAMA | | MNAAAKGNSEASKKQTDLSNEDLF | AGCGAGAAGAAGAAGACCCCAACCTCCGGCATGAGCGGCATGTCCGG |
| | | | NDELTEEVIADSYEEGGNVGSEEAE | CAGCGGCGCTACCAGCGCTGCTACCGAGGCCGCCACCAACATGAACGC |
| | | | SLTNAFDDKLLDQGVNENTLLNDNM | TACCGCCATGAACGCTGCCGCCAAGGGCAACTCCGAGGCTAGCAAGAA |
| | | | IYNVNMVPHKKRELYISPHKHTSAAS | GCAAACCGACCTCTCCAACGAGGACCTGTTCAACGACGAGCTCACCGA |
| | | | SKNGKHHAADADALDKKLRAHELLE | GGAAGTGATCGCCGACAGCTACGAGGAAGGCGGCAACGTGGGCTCCG |
| | | | LENGEGSNSVIVETEEVDVDLNGGK | AGGAAGCCGAGAGCCTGACCAACGCCTTCGACGACAAGCTCCTGGACC |
| | | | SSGSVSFLSSVVFLLIGLLCFTNhhhh | AGGGCGTGAACGAGAACACCCTCCTGAACGACAACATGATCTACAACG |
| | | | hh (SEQ ID NO: 45) | TGAACATGGTCCCACACAAGAAGAGGGAGCTCTACATCTCCCCACACA |
| | | | | AGCACACCAGCGCCGCCTCCAGCAAGAACGGCAAGCACCACGCTGCTG |
| | | | | ACGCTGACGCTCTGGACAAGAAGCTCAGGGCTCACGAGCTCCTGGAGC |
| | | | | TGGAGAACGGCGAGGGCTCCAACAGCGTGATCGTCGAGACGGAGGAA |
| | | | | GTGGACGTGGACCTGAACGGCGGCAAGTCCTCCGGCTCCGTCAGCTTC |
| | | | | CTCTCCAGCGTGGTCTTCCTCCTGATCGGCCTCCTGTGCTTCACCAACCA |
| | | | | CCACCACCACCACCACTGA (SEQ ID NO: 46) |
| 24 | HP, | PVX_ | mDDNGRRLPRKAAPPVDKAKQDVM | ATGGACGACAACGGCAGGCGCCTCCCAAGGAAGGCTGCCCCACCAGT |
| | conserved | 003555 | KDIVNYLSKNMLAFVRQKRNVSGKE | GGACAAGGCCAAGCAGGACGTGATGAAGGACATCGTCAACTACCTCTC |
| | | | GEAPTGPSGAQGGDSSQYASKFTF | CAAGAACATGCTGGCCTTCGTGAGGCAAAAGCGCAACGTCTCCGGCAA |
| | | | TDHSVDFSKYNKLDKEKFAAKDDLK | GGAAGGCGAGGCTCCAACCGGCCCAAGCGGCGCTCAAGGCGGCGACT |
| | | | SRLKNEVVASMLDTEGDILTEEFGYL | CCAGCCAGTACGCCAGCAAGTTCACCTTCACCGACCACTCCGTGGACTT |
| | | | LRNYFDKVKLEEKKSQEAESAKPAE | CAGCAAGTACAACAAGCTCGACAAGGAGAAGTTCGCCGCCAAGGACG |
| | | | QEEEAEEAPEQKEEATAEKATEETT | ACCTCAAGTCCAGGCTGAAGAACGAGGTGGTCGCCAGCATGCTCGACA |
| | | | EAATEETTEAATEETTEAATEETTEA | CCGAGGGCGACATCCTGACCGAGGAGTTCGGCTACCTCCTGCGCAACT |
| | | | ATEETTEAATEETTEAATEETTEAAT | ACTTCGACAAGGTCAAGCTGGAGGAGAAGAAGTCCCAAGAGGCCGAG |
| | | | EETTEAATEEATEGATEEGAEETTE | AGCGCTAAGCCAGCTGAGCAAGAGGAAGAGGCCGAGGAAGCCCCAG |
| | | | EATEEGAEEATEEGAEEATEEGAEE | AGCAAAAGGAAGAGGCCACCGCTGAGAAGGCTACCGAGGAGACGACC |
| | | | TTEEATEEGAEETTEETTEEGAEEE | GAGGCTGCCACGGAGGAGACGACGGAGGCCGCCACGGAGGAGACGA |
| | | | ATEEGAEETTEEGAEEAAEEGAEEG | CCGAGGCCGCCACCGAGGAGACGACGGAGGCTGCCACTGAAGAGACG |
| | | | AEAATEEATEEATEEATEEATEEAT | ACCGAGGCTGCGACGGAAGAGACGACCGAGGCCGCGACGGAAGAGA |
| | | | EEATEEATAEVAEAATPEKVTEEAT | CGACTGAGGCTGCCACTGAGGAGACGACGGAAGCTGCTACCGAGGAA |
| | | | EEATEEGDNEPAEQAAEKEEDVKG | GCCACCGAGGGCGCTACCGAGGAAGGCGCTGAGGAGACGACGGAGG |
| | | | GLMDNETYYNTLQELYEEIENDDKK | AAGCCACGGAGGAAGGCGCTGAGGAAGCCACCGAGGAAGGCGCCGA |
| | | | EKEKIQKAKEQEELEKKLFKESKKG | GGAAGCCACGGAGGAAGGCGCAGAGGAGACGACAGAGGAAGCCACG |
| | | | KKKEKKRRKKLCKMAKIVEKYAEEIP | GAGGAAGGCGCCGAAGAGACGACCGAAGAGACGACCGAGGAAGGCG |
| | | | KDSERSLRYDKEEHIDDPDEMDDLL | CGGAGGAAGAGGCCACTGAGGAAGGCGCCGAGGAGACGACTGAGGA |
| | | | FGEFKTLEKYGTHKTSTFYYEMTCF | AGGCGCAGAGGAAGCCGCTGAGGAAGGCGCTGAGGAAGGCGCTGAG |
| | | | DERLRDFEINTKLKEMEEVPEKWEL | GCCGCCACGGAGGAAGCCACCGAGGAAGCCACGGAGGAAGCCACGG |
| | | | LSLYWQSYRNERHKYLAVKKYLLEK | AGGAAGCCACAGAGGAAGCCACTGAGGAAGCCACAGAGGAAGCCAC |
| | | | FLELKTNQSTEALPKYNKKWKQCEE | AGCTGAGGTGGCTGAGGCTGCTACCCCAGAGAAGGTCACAGAGGAAG |
| | | | IVDNNFTKQHEHVNDVFYTFVAKEN | CCACAGAGGAAGCCACCGAGGAAGGCGACAACGAGCCAGCTGAGCAG |
| | | | LSRDEFKEILNDVRASWhhhhhh | GCTGCTGAGAAGGAAGAGGACGTGAAGGGCGGCCTCATGGACAACG |
| | | | (SEQ ID NO: 47) | AGACGTACTACAACACCCTCCAAGAGCTGTACGAGGAGATCGAGAACG |
| | | | | ACGACAAGAAGGAGAAGGAGAAGATCCAAAAGGCCAAGGAGCAAGA |
| | | | | GGAGCTGGAGAAGAAGCTGTTCAAGGAGTCCAAGAAGGGCAAGAAG |
| | | | | AAGGAGAAGAAGAGGCGCAAGAAGCTCTGCAAGATGGCCAAGATCGT |
| | | | | CGAGAAGTACGCCGAGGAGATCCCAAAGGACTCCGAGAGGAGCCTGC |
| | | | | GCTACGACAAGGAAGAGCACATCGACGACCCAGACGAGATGGACGAC |
| | | | | CTCCTGTTCGGCGAGTTCAAGACCCTGGAGAAGTACGGCACCCACAAG |
| | | | | ACCTCCACCTTCTACTACGAGATGACCTGCTTCGACGAGAGGCTCCGCG |
| | | | | ACTTCGAGATCAACACCAAGCTGAAGGAGATGGAGGAAGTGCCAGAG |
| | | | | AAGTGGGAGCTCCTGTCCCTCTACTGGCAGAGCTACAGGAACGAGCGC |
| | | | | CACAAGTACCTGGCCGTCAAGAAGTACCTCCTGGAGAAGTTCCTGGAG |
| | | | | CTGAAGACCAACCAAAGCACCGAGGCCCTGCCAAAGTACAACAAGAA |
| | | | | GTGGAAGCAGTGCGAGGAGATCGTCGACAACAACTTCACCAAGCAAC |
| | | | | ACGAGCACGTGAACGACGTCTTCTACACCTTCGTGGCCAAGGAGAACC |
| | | | | TCTCCAGGGACGAGTTCAAGGAGATCCTGAACGACGTCCGCGCCAGCT |
| | | | | GGCACCACCACCACCACCACTGA (SEQ ID NO: 48) |
| 25 | phosphati- | PVX_ | MRCCTKDAVNVESPKKVVVGETEE | ATGAGGTGCTGCACCAAGGACGCCGTCAACGTGGAGTCCCCAAAGAA |
| | dylinosi- | 117385 | DTREEENPYEDLPTVTVTLSDGSVY | GGTGGTCGTGGGCGAGACGGAGGAAGACACCAGGGAGGAAGAGAAC |
| | tol-4- | | TGTTKDNRVHGRGVLKYVNGDQYE | CCATACGAGGACCTCCCAACCGTCACCGTGACCCTGTCCGACGGCAGC |
| | phosphate- | | GEFVDGKKEGKGKWTDKENNTYEG | GTCTACACCGGCACCACCAAGGACAACAGGGTGCACGGCCGCGGCGT |
| | 5- | | DWVKDKRHGHGVYKTAEGFIFEGE | CCTCAAGTATGTGAACGGCGACCAATACGAGGGCGAGTTCGTCGACG |
| | kinase, | | FANNKREGKGTIITPEKTKYVCSFQD | GCAAGAAGGAAGGCAAGGGCAAGTGGACCGACAAGGAGAACAACAC |
| | putative | | DEEVGEVEFFFANGDHALGYIKDGY | CTACGAGGGCGACTGGGTCAAGGACAAGAGGCACGGCCACGGCGTGT |
| | | | LCQNGRYEFKNGDIYVGNFEKGLFH | ACAAGACCGCTGAGGGCTTCATCTTCGAGGGCGAGTTCGCCAACAACA |
| | | | GEGYYKWNNDANYTIYEGNYSEGK | AGCGCGAGGGCAAGGGCACCATCATCACCCCAGAGAAGACCAAGTAT |
| | | | KHGKGQLINKDGRILCGMFRDNNM | GTGTGCAGCTTCCAAGACGACGAGGAAGTGGGCGAGGTGGAGTTCTT |
| | | | DGEFLEISPQGNQTKVLYDKGFFVK | CTTCGCCAACGGCGACCACGCCCTCGGCTACATCAAGGACGGCTACCT |
| | | | VLDKIEENLDVQEFLKDSIIHTTIFSD | GTGCCAGAACGGCCGCTACGAGTTCAAGAACGGCGACATCTACGTGG |
| | | | PTTYKKLYEITEKKKPQFRLNLKRTQ | GCAACTTCGAGAAGGGCCTGTTCCACGGCGAGGGCTACTACAAGTGG |
| | | | PTShhhhhh (SEQ ID NO: 49) | AACAACGACGCCAACTACACCATCTACGAGGGCAACTACTCCGAGGGC |
| | | | | AAGAAGCACGGCAAGGGCCAACTCATCAACAAGGACGGCAGGATCCT |
| | | | | GTGCGGCATGTTCCGCGACAACAACATGGACGGCGAGTTCCTGGAGAT |
| | | | | CAGCCCACAAGGCAACCAGACCAAGGTCCTCTACGACAAGGGCTTCTT |
| | | | | CGTCAAGGTGCTGGACAAGATCGAGGAGAACCTCGACGTGCAGGAGT |
| | | | | TCCTGAAGGACTCCATCATCCACACCACCATCTTCAGCGACCCAACCAC |
| | | | | CTACAAGAAGCTGTACGAGATCACCGAGAAGAAGAAGCCACAATTCAG |
| | | | | GCTCAACCTGAAGCGCACCCAGCCAACCTCCCACCACCACCACCACCAC |
| | | | | TGA (SEQ ID NO: 50) |
| 26 | Plasmodium | PVX_ | mNKLGTSLVEDATANGEFGLRVQRL | ATGAACAAGCTGGGCACCAGCCTCGTGGAGGACGCTACCGCTAACGG |
| | exported | 113225 | LGGSRSSRDSIFADSFYDDDDDDDD | CGAGTTCGGCCTCCGCGTCCAAAGGCTGCTGGGCGGCTCCAGGTCCAG |
| | protein, | | NNDKLFDYDSDHKSRREVKDRHHR | CCGCGACAGCATCTTCGCCGACTCCTTCTACGATGATGACGACGACGAC |
| | unknown | | HRHSHSHRHKRRHSHKHRTSSRSR | GACGACAACAACGACAAGCTGTTCGACTACGACAGCGACCACAAGTCC |
| | function | | REKEESSTTNDDDDEVLSLSRFDVD | AGGCGCGAGGTGAAGGACAGGCACCACAGGCACAGGCACAGCCACTC |
| | | | DDKDDRSHSRYSVDYDDENDDEPS | CCACCGCCACAAGAGGCGCCACAGCCACAAGCACAGGACCTCCAGCCG |
| | | | SSRPASTDYDDIIDLTNARRSGSKYR | CTCCAGGCGCGAGAAGGAAGAGTCCAGCACCACCAACGACGACGACG |
| | | | ISSMDIELYPEHEDEYLFEGKRRSG | ACGAGGTGCTCAGCCTGTCCAGGTTCGACGTCGACGACGACAAGGAC |
| | | | GVLKKADNYCENKIFDALSALDKYK | GACAGGAGCCACTCCCGCTACAGCGTGGACTACGACGACGAGAACGA |
| | | | EYYGEERRVMKQAAYRKATKVFAIP | CGACGAGCCATCCAGCTCCAGGCCAGCCTCCACCGACTACGACGACAT |
| | | | GAAALSPLIITLFLTTSNVVALPLAAS | CATCGACCTCACCAACGCTAGGCGCAGCGGCTCCAAGTACCGCATCAG |
| | | | AVILGGILYKKSKDKSDYGRPHLKSI | CTCCATGGACATCGAGCTCTACCCAGAGCACGAGGACGAGTACCTGTT |
| | | | TYhhhhhh (SEQ ID NO: 51) | CGAGGGCAAGAGGCGCAGCGGCGGCGTCCTGAAGAAGGCTGACAACT |
| | | | | ACTGCGAGAACAAGATCTTCGACGCCCTCTCCGCCCTGGACAAGTACA |
| | | | | AGGAGTACTACGGCGAGGAGAGGCGCGTGATGAAGCAGGCCGCCTAC |
| | | | | AGGAAGGCCACCAAGGTCTTCGCTATCCCAGGCGCTGCCGCCCTCAGC |
| | | | | CCACTGATCATCACCCTCTTCCTGACCACCAGCAACGTGGTGGCTCTCC |
| | | | | CACTGGCTGCTTCCGCCGTCATCCTCGGCGGCATCCTGTACAAGAAGA |
| | | | | GCAAGGACAAGTCCGACTACGGCCGCCCACACCTCAAGTCCATCACCT |
| | | | | ACCACCACCACCACCACCACTGA (SEQ ID NO: 52) |
| 27 | trypto- | PVX_ | MEAARGVSGLVPSSNSLQEITLRYK | ATGGAGGCTGCCAGGGGCGTGTCCGGCCTCGTCCCATCCAGCAACAGC |
| | phan- | 090265 | DKLLNMDKEQMILTLGVTMIAITSAV | CTCCAAGAGATCACCCTGCGCTACAAGGACAAGCTCCTGAACATGGAC |
| | rich | | AFGVLATHGDINDFLGVESDEESEK | AAGGAGCAGATGATCCTCACCCTGGGCGTCACCATGATCGCTATCACCT |
| | antigen | | KKEIVEKSEEWKRKEWSNWLKKLE | CCGCTGTGGCTTTCGGCGTCCTGGCTACCCACGGCGACATCAACGACTT |
| | (Pv-fam-a) | | QDWKVFNEKLQNEKKTFLEEKEED | CCTGGGCGTCGAGTCCGACGAGGAGAGCGAGAAGAAGAAGGAGATC |
| | | | WNTWIKSVEKKWTHFNPNMDKEFH | GTGGAGAAGTCCGAGGAGTGGAAGAGGAAGGAGTGGAGCAACTGGC |
| | | | TNMMRRSINWTESQWREWIQTEGR | TCAAGAAGCTGGAGCAAGACTGGAAGGTCTTCAACGAGAAGCTCCAG |
| | | | LYLDIEWKKWFFENQSRLDELIVKK | AACGAGAAGAAGACCTTCCTGGAGGAGAAGGAAGAGGACTGGAACAC |
| | | | WIQWKKDKIINWLMSDWKRAEQEH | CTGGATCAAGTCCGTGGAGAAGAAGTGGACCCACTTCAACCCAAACAT |
| | | | WEEFEEKSWSSKFFQIFEKRNYEDF | GGACAAGGAGTTCCACACCAACATGATGAGGCGCTCCATCAACTGGAC |
| | | | KDRVSDEWEDWFEWVKRKDNIFIT | CGAGAGCCAATGGCGCGAGTGGATCCAGACCGAGGGCAGGCTCTACC |
| | | | NVLDQWIKWKEEKNLLYNNWADAF | TGGACATCGAGTGGAAGAAGTGGTTCTTCGAGAACCAAAGCAGGCTC |
| | | | VTNWINKKQWVVWVNERRNLAAKA | GACGAGCTGATCGTGAAGAAGTGGATCCAGTGGAAGAAGGACAAGAT |
| | | | KAALNKKKhhhhhh | CATCAACTGGCTCATGTCCGACTGGAAGCGCGCCGAGCAAGAGCACTG |
| | | | (SEQ ID NO: 53) | GGAGGAGTTCGAGGAGAAGAGCTGGTCCAGCAAGTTCTTCCAGATCTT |
| | | | | CGAGAAGCGCAACTACGAGGACTTCAAGGACCGCGTGAGCGACGAGT |
| | | | | GGGAGGACTGGTTCGAGTGGGTCAAGCGCAAGGACAACATCTTCATC |
| | | | | ACCAACGTGCTGGACCAGTGGATCAAGTGGAAGGAAGAGAAGAACCT |
| | | | | CCTGTACAACAACTGGGCCGACGCCTTCGTCACCAACTGGATCAACAA |
| | | | | GAAGCAGTGGGTGGTCTGGGTGAACGAGAGGCGCAACCTCGCTGCTA |
| | | | | AGGCTAAGGCTGCCCTGAACAAGAAGAAGCACCACCACCACCACCACT |
| | | | | GA (SEQ ID NO: 54) |
| 28 | MSP7 | PVX_ | mTKGPSGPPPNKKLNANALHFLRG | ATGACCAAGGGCCCATCCGGCCCACCACCAAACAAGAAGCTCAACGCC |
| | family | 082700 | KLELLNKISEEQVVSPDFKKNVELLK | AACGCCCTCCACTTCCTGAGGGGCAAGCTGGAGCTCCTGAACAAGATC |
| | | | KKIEELQGKAEKDKSKTDGEDTTPK | TCCGAGGAGCAAGTGGTCAGCCCAGACTTCAAGAAGAACGTCGAGCTC |
| | | | EQQEDQNVSQNGLEEQAPSDSNEG | CTCAAGAAGAAGATCGAGGAGCTCCAGGGCAAGGCCGAGAAGGACAA |
| | | | EAQEENTQVKNVIFTEKEEAVDEEA | GTCCAAGACCGACGGCGAGGACACCACCCCAAAGGAGCAACAAGAGG |
| | | | EKEDTAVISEKANFPNEESQGNDET | ACCAAAACGTGAGCCAGAACGGCCTGGAGGAGCAAGCTCCGTCCGAC |
| | | | QTQESIEGEASPGVVVDETDDSPEG | AGCAACGAGGGCGAGGCTCAAGAGGAGAACACCCAGGTCAAGAACGT |
| | | | EPLSGLETEGNSSAESAPNEPDVNT | GATCTTCACCGAGAAGGAAGAGGCCGTCGACGAGGAAGCCGAGAAG |
| | | | THTAVDTHMPADANIGVDTNMPFDT | GAAGACACCGCCGTGATCTCCGAGAAGGCCAACTTCCCAAACGAGGA |
| | | | PPHPSGENPGAPQETHLPSIDENAN | GAGCCAGGGCAACGACGAGACGCAAACCCAAGAGTCCATCGAGGGCG |
| | | | RRASRMKHMSSFLNGLLTNQSNNK | AGGCTAGCCCGGGCGTGGTGGTGGACGAGACGGACGACTCCCCGGAG |
| | | | KEIFFHPYYGPYFNHGGYYNYDPYY | GGCGAGCCACTCAGCGGCCTCGAAACCGAGGGCAACTCCAGCGCTGA |
| | | | NYAPAYNPFVSQARDYEVIKKLLDA | GTCCGCTCCAAACGAGCCAGACGTCAACACCACCCACACCGCTGTGGA |
| | | | CFNKGEGADPNVPCIIDIFKKVLDDE | CACCCACATGCCAGCTGACGCCAACATCGGCGTCGACACCAACATGCC |
| | | | RFRNELKTFMYDLYEFLKKNDVLSD | ATTCGACACCCCACCACACCCAAGCGGCGAGAACCCGGGCGCCCCACA |
| | | | DEKKNELMRFFFDNAFQLVNPMFYY | AGAGACGCACCTCCCATCCATCGACGAGAACGCCAACAGGCGCGCCAG |
| | | | hhhhhh (SEQ ID NO: 55) | CAGGATGAAGCACATGTCCAGCTTCCTGAACGGCCTCCTGACCAACCA |
| | | | | GTCCAACAACAAGAAGGAGATCTTCTTCCACCCATACTACGGCCCATAC |
| | | | | TTCAACCACGGCGGATACTACAACTACGACCCATACTACAACTACGCCC |
| | | | | CAGCCTACAACCCATTCGTCAGCCAAGCCCGCGACTACGAGGTCATCA |
| | | | | AGAAGCTCCTGGACGCCTGCTTCAACAAGGGCGAGGGCGCTGACCCA |
| | | | | AACGTCCCATGCATCATCGACATCTTCAAGAAGGTGCTCGACGACGAG |
| | | | | AGGTTCCGCAACGAGCTGAAGACCTTCATGTACGACCTCTACGAGTTCC |
| | | | | TGAAGAAGAACGACGTCCTCAGCGACGACGAGAAGAAGAACGAGCTG |
| | | | | ATGAGGTTCTTCTTCGACAACGCCTTCCAGCTCGTGAACCCAATGTTCT |
| | | | | ACTACCACCACCACCACCACCACTGA (SEQ ID NO: 56) |
| 29 | Hyp, huge | PVX_ | mFSGGVGDDEEEEEEEEGEEGESE | ATGTTCAGCGGCGGCGTGGGCGACGACGAGGAAGAGGAAGAGGAAG |
| | list of | 002550 | RDDSERDYAGRDDAGRDDAERND | AGGAAGGCGAGGAAGGCGAGAGCGAGAGGGACGACTCCGAGAGGG |
| | orthologs, | | AERDDAERNDAERDDAERDHAERD | ACTACGCTGGCAGGGACGATGCCGGCAGGGACGACGCCGAGAGGAA |
| | paralogs, | | HADKAESDRESSLEANENRLVKLSE | CGACGCCGAGCGCGATGATGCTGAGCGCAACGACGCCGAGCGCGACG |
| | synteny | | GGESEPALLEVEEDIKQTVLGMFSL | ACGCCGAGAGGGACCACGCCGAGCGCGACCACGCCGACAAGGCCGAG |
| | with Py | | KGEFDEAESEKLALDLQKNLLSMLS | TCCGACAGGGAGTCCAGCCTGGAGGCCAACGAGAACAGGCTGGTGAA |
| | LSA3 | | GNMEDNDDEYEDIDEEYEEVEEDY | GCTCAGCGAGGGCGGCGAGTCCGAGCCAGCTCTCCTGGAGGTGGAGG |
| | (PyLSA3syn- | | EEEKLGKPVEVVVEDATEEAVDEVV | AAGACATCAAGCAAACCGTCCTGGGCATGTTCAGCCTCAAGGGCGAGT |
| | 3) | | GVVQEPEEEGAEESDKDTGEVSEE | TCGACGAGGCCGAGTCCGAGAAGCTCGCCCTGGACCTCCAGAAGAACC |
| | | | EVAKEAADEVMEEEKKEEAGEPSV | TCCTGTCCATGCTCAGCGGCAACATGGAGGACAACGACGACGAGTACG |
| | | | VVEEPSVVVKEPSVVVKEPSVVVEE | AGGACATCGACGAGGAGTACGAGGAAGTGGAGGAAGACTACGAGGA |
| | | | PSVVVEEPSVVVEEPSVVVEEPAFT | AGAGAAGCTCGGCAAGCCAGTGGAGGTGGTCGTGGAGGACGCCACCG |
| | | | VEEPAFTVEEPAITVEEPAITVEEPVF | AGGAAGCCGTGGACGAGGTGGTGGGCGTCGTGCAAGAGCCAGAGGA |
| | | | TVEEPVFTVEEPAFTVEEPAFTVEEP | AGAGGGCGCTGAGGAGAGCGACAAGGACACCGGCGAGGTGTCCGAG |
| | | | AFTVEEPATTVEELVEEVLKVAEEEV | GAAGAGGTGGCCAAGGAAGCCGCCGACGAGGTCATGGAGGAAGAGA |
| | | | ATEAVEKDGEEAEEQVTEESVEEDE | AGAAGGAAGAGGCCGGCGAGCCATCCGTGGTGGTGGAGGAGCCAAG |
| | | | EESGEEEGEESEEEETEESAEEEVA | CGTGGTCGTGAAGGAGCCATCCGTCGTGGTCAAGGAGCCTTCCGTGGT |
| | | | KESVEEEVAKEAEESEESGEESAEE | CGTGGAGGAGCCTAGCGTCGTCGTCGAGGAGCCTTCCGTCGTGGTGG |
| | | | EKEKAEEPVAPVDEVLKEGMQKIEE | AGGAGCCCAGCGTGGTCGTCGAGGAGCCAGCCTTCACCGTGGAGGAG |
| | | | SVKEALGVVQEAVDKVAEEEQTEQ | CCTGCCTTCACCGTCGAGGAGCCAGCCATCACCGTGGAGGAGCCCGCT |
| | | | AQGPAEAGPVGVVKEPEEEEESEE | ATCACGGTGGAGGAGCCAGTGTTCACCGTGGAAGAACCCGTGTTCACC |
| | | | EGEEGEEGEEGEEEEEEESEEEES | GTGGAAGAGCCCGCCTTCACCGTTGAGGAGCCCGCCTTCACCGTAGAA |
| | | | EEGESEAGESEAGKSDAAESEVAE | GAGCCTGCCTTCACCGTTGAAGAACCAGCTACCACCGTGGAGGAGCTG |
| | | | SEAGEPAEDQAGMDAKMKDELLGM | GTGGAGGAAGTGCTCAAGGTGGCTGAGGAAGAGGTGGCTACCGAGG |
| | | | LSEKMKAEGKDLDKLPPEVKKNLLD | CTGTGGAGAAGGACGGCGAGGAAGCCGAGGAGCAAGTCACCGAGGA |
| | | | MLAGNMEMDDEEEEGEEEGEDLG | GAGCGTCGAGGAAGACGAGGAAGAGTCCGGCGAGGAAGAGGGCGA |
| | | | NEELDLQKNLLEMLSGKGGFNPNM | GGAGAGCGAGGAAGAGGAGACCGAGGAGTCCGCTGAGGAAGAGGTG |
| | | | LGNLKELEALQKSVPGLMGKAQGIS | GCGAAGGAGAGCGTGGAGGAAGAGGTGGCTAAGGAAGCCGAGGAGT |
| | | | PAEIESLKSMFSGAFDSRGFKGMPQ | CCGAGGAGAGCGGGGAGGAGAGCGCTGAGGAAGAGAAGGAGAAGG |
| | | | MKLPAELQSIMMPKKEEKGKPQGA | CCGAGGAGCCAGTGGCTCCAGTGGACGAGGTCCTGAAGGAAGGCATG |
| | | | QAKAKVPAKAGQVQKPKAQDIMPS | CAGAAGATCGAGGAGAGCGTGAAGGAAGCCCTGGGCGTGGTCCAAG |
| | | | RRIRDLFVLPKEIFGSLKNFKESALKF | AGGCCGTGGACAAGGTCGCCGAGGAAGAGCAGACCGAGCAGGCTCA |
| | | | ANHIGLNLETIKKHLTTVKNFLLRVDA | GGGCCCAGCTGAGGCTGGCCCAGTCGGCGTGGTCAAGGAGCCTGAGG |
| | | | VVDKEIGNIIEAGKSPQNVVQANEGF | AAGAGGAAGAGTCTGAGGAAGAGGGCGAGGAAGGCGAGGAAGGCG |
| | | | LDKMKRLVNKYKIFSIPFFAGMGSFG | AGGAAGGCGAGGAAGAGGAAGAGGAAGAGAGTGAGGAAGAGGAGT |
| | | | Fhhhhhh (SEQ ID NO: 57) | CTGAGGAAGGCGAGTCCGAGGCTGGGGAGAGCGAGGCTGGCAAGAG |
| | | | | CGACGCCGCCGAGTCCGAGGTGGCCGAGAGCGAGGCCGGCGAGCCG |
| | | | | GCTGAGGACCAAGCTGGCATGGACGCCAAGATGAAGGACGAGCTCCT |
| | | | | GGGCATGCTGAGCGAGAAGATGAAGGCCGAGGGCAAGGACCTGGAC |
| | | | | AAGCTCCCACCAGAGGTCAAGAAGAACCTCCTGGACATGCTCGCCGGC |
| | | | | AACATGGAGATGGACGATGAGGAAGAGGAAGGCGAGGAAGAGGGC |
| | | | | GAAGACCTGGGCAACGAGGAGCTCGACCTCCAGAAGAACCTCCTGGA |
| | | | | GATGCTCTCCGGCAAGGGCGGCTTCAACCCAAACATGCTGGGCAACCT |
| | | | | CAAGGAGCTGGAGGCCCTCCAAAAGAGCGTGCCAGGCCTGATGGGCA |
| | | | | AGGCTCAGGGCATCTCCCCAGCTGAGATCGAGTCCCTCAAGAGCATGT |
| | | | | TCTCCGGCGCCTTCGACAGCAGGGGCTTCAAGGGCATGCCACAGATGA |
| | | | | AGCTGCCAGCCGAGCTCCAGTCCATCATGATGCCAAAGAAGGAAGAG |
| | | | | AAGGGCAAGCCACAAGGCGCTCAAGCTAAGGCTAAGGTGCCAGCTAA |
| | | | | GGCTGGCCAAGTCCAGAAGCCAAAGGCCCAGGACATCATGCCAAGCA |
| | | | | GGCGCATCCGCGACCTGTTCGTGCTCCCAAAGGAGATCTTCGGCAGCC |
| | | | | TGAAGAACTTCAAGGAGTCCGCCCTCAAGTTCGCCAACCACATCGGCCT |
| | | | | GAACCTGGAGACCATCAAGAAGCACCTCACCACCGTGAAGAACTTCCT |
| | | | | CCTGAGGGTCGACGCCGTGGTCGACAAGGAGATCGGCAACATCATCG |
| | | | | AGGCCGGCAAGTCCCCACAAAACGTGGTCCAGGCCAACGAGGGCTTCC |
| | | | | TGGACAAGATGAAGCGCCTCGTGAACAAGTACAAGATCTTCAGCATCC |
| | | | | CATTCTTCGCCGGCATGGGCTCCTTCGGCTTCCACCATCACCACCATCAC |
| | | | | TGA (SEQ ID NO: 58) |
| 30 | MSP7-like | PVX_ | mQLGIQKKKKNLEQDAMHALMKKLE | ATGCAGCTCGGCATCCAAAAGAAGAAGAAGAACCTGGAGCAGGACGC |
| | protein | 082650 | SLYKLSATDNGEIFNKEIDALKKQID | CATGCACGCCCTCATGAAGAAGCTGGAGAGCCTGTACAAGCTCTCCGC |
| | | | QLHQHGGGNEGESLGHLLESEAAD | CACCGACAACGGCGAGATCTTCAACAAGGAGATCGACGCCCTGAAGA |
| | | | DSGKKTIFGVDEDDLDNYDADFIGQ | AGCAAATCGACCAGCTCCACCAACACGGCGGCGGAAACGAGGGCGAG |
| | | | SKGKIKGQADTTAVAKPPTGSGAGA | AGCCTGGGCCACCTCCTGGAGAGCGAGGCTGCTGACGACTCCGGCAA |
| | | | HGSHSPPKPSVLVVPGKSGKEDSV | GAAGACCATCTTCGGCGTGGACGAGGACGACCTGGACAACTACGACG |
| | | | ATLENGYESIHGEDEPREDSTSHDS | CCGACTTCATCGGCCAGTCCAAGGGCAAGATCAAGGGCCAGGCTGACA |
| | | | PPALPVGRSEGDSSASGGGTEGQQ | CCACCGCTGTGGCTAAGCCACCAACCGGCAGCGGCGCTGGCGCTCACG |
| | | | PDPASARGSQASGGRGGGDQTNT | GCAGCCACTCCCCACCAAAGCCATCCGTGCTCGTGGTCCCAGGCAAGA |
| | | | TQPAGGQQSSSAARSLQAPHAGDS | GCGGCAAGGAAGACTCCGTCGCCACCCTGGAGAACGGCTACGAGAGC |
| | | | QLPNAGGDPQSPAAAGHQQPPTSP | ATCCACGGCGAGGACGAGCCAAGGGAGGACAGCACCTCCCACGACTC |
| | | | PANNEGTTVTQESALAATPPKGTAD | CCCACCAGCTCTCCCAGTGGGCCGCAGCGAGGGCGACTCCAGCGCTTC |
| | | | SNDAKIKYLDKLYDEVLTTSDNTSGI | CGGCGGCGGCACCGAGGGCCAACAGCCAGACCCAGCTAGCGCCAGGG |
| | | | HVPDYHSKYNTIRQKYEYSMNPVEY | GCAGCCAGGCTTCCGGCGGCAGGGGCGGCGGCGACCAAACCAACACC |
| | | | EIVKNLFNVGFKNDGAASSDATPLV | ACCCAACCAGCTGGCGGCCAACAGTCCAGCTCCGCTGCTAGGAGCCTG |
| | | | DVFKKALADEKFQAEFDNFVHGLYG | CAGGCCCCACACGCTGGCGACAGCCAGCTCCCAAACGCCGGCGGCGA |
| | | | FAKRHSYLSEARMKDNKLYSDLLKN | CCCACAATCCCCAGCTGCCGCCGGCCACCAACAGCCACCAACCTCCCCA |
| | | | AISLMSTLQVShhhhhh | CCAGCCAACAACGAGGGCACCACCGTGACCCAAGAGTCCGCTCTGGCT |
| | | | (SEQ ID NO: 59) | GCTACCCCACCAAAGGGCACCGCCGACTCCAACGACGCCAAGATCAAG |
| | | | | TACCTGGACAAGCTCTACGACGAGGTGCTGACCACCAGCGACAACACC |
| | | | | TCCGGCATCCACGTCCCAGACTACCACAGCAAGTACAACACCATCCGCC |
| | | | | AAAAGTACGAGTACTCCATGAACCCAGTGGAGTACGAGATCGTCAAGA |
| | | | | ACCTCTTCAACGTGGGCTTCAAGAACGACGGCGCTGCCAGCTCCGACG |
| | | | | CTACCCCACTGGTGGACGTCTTCAAGAAGGCCCTCGCCGACGAGAAGT |
| | | | | TCCAGGCCGAGTTCGACAACTTCGTCCACGGCCTGTACGGCTTCGCCAA |
| | | | | GAGGCACAGCTACCTCTCCGAGGCCCGCATGAAGGACAACAAGCTGTA |
| | | | | CAGCGACCTCCTGAAGAACGCCATCAGCCTGATGTCCACCCTCCAAGTG |
| | | | | TCCCACCACCACCACCACCACTGA (SEQ ID NO: 60) |
| 31 | reticulo- | PVX_ | mAAYNTVLQIYKYSDDIVRKQEKCE | ATGGCCGCCTACAACACCGTGCTCCAAATCTACAAGTACTCCGACGACA |
| | cyte | 094255 | QLVKDGKDICLKFKSINEIKVMIQNSK | TCGTGAGGAAGCAAGAGAAGTGCGAGCAGCTGGTCAAGGACGGCAA |
| | binding | | GKESTLSAKVSHSFNKLSELNKIKCN | GGACATCTGCCTCAAGTTCAAGTCCATCAACGAGATCAAGGTCATGATC |
| | protein | | DESYDAILETPSREELNKLRSTFKQE | CAGAACAGCAAGGGCAAGGAGTCCACCCTCAGCGCCAAGGTGTCCCA |
| | 2b | | KDTIANQAKLSGYKTDFETHIGKLND | CAGCTTCAACAAGCTCAGCGAGCTGAACAAGATCAAGTGCAACGACGA |
| | (RBP2b) | | LAKIVDNLKASETLPKNIEEKKTSINLI | GAGCTACGACGCCATCCTCGAAACCCCATCCAGGGAGGAGCTCAACAA |
| | | | STKLETIEKEIESINSSFDQLLEKGKK | GCTGCGCAGCACCTTCAAGCAAGAGAAGGACACCATCGCCAACCAGGC |
| | | | CEMTKYKLVRDSLSTKINDHSAIIKD | CAAGCTCTCCGGCTACAAGACCGACTTCGAGACGCACATCGGCAAGCT |
| | | | NQKKATEYLTYIQNNHISIFKDIDMLN | CAACGACCTGGCCAAGATCGTGGACAACCTCAAGGCCAGCGAGACGCT |
| | | | ENLGEKSVSRYAIAKIEEANDLSAQL | GCCAAAGAACATCGAGGAGAAGAAGACCTCCATCAACCTCATCAGCAC |
| | | | TAAVSEYEAIANSIRKEFTNISDHTE | CAAGCTCGAAACCATCGAGAAGGAGATCGAGTCCATCAACTCCAGCTT |
| | | | MDTLENEAKMLKEHYDNLINKKNIIT | CGACCAACTCCTGGAGAAGGGCAAGAAGTGCGAGATGACCAAGTACA |
| | | | ELHNKINLIKLLEIRATSDKYVDIAELL | AGCTCGTCAGGGACTCCCTGAGCACCAAGATCAACGACCACTCCGCCA |
| | | | GEVVKDQKKKLQEAKNKLDTLKDHI | TCATCAAGGACAACCAAAAGAAGGCCACCGAGTACCTCACCTACATCC |
| | | | AVKEKELINHDSSFTLVSIKAFDEIYD | AGAACAACCACATCAGCATCTTCAAGGACATCGACATGCTCAACGAGA |
| | | | DIKYNVGQLHTLEVTNFDELKKGKT | ACCTGGGCGAGAAGTCCGTGAGCAGGTACGCCATCGCCAAGATCGAG |
| | | | YEENVTHLLNRRETLONDLHNYEEK | GAAGCCAACGACCTCTCCGCTCAACTCACCGCTGCCGTCAGCGAGTAC |
| | | | DKLKNTNIEMSNEENNQIRQTSEVIK | GAGGCTATCGCCAACTCCATCCGCAAGGAGTTCACCAACATCTCCGACC |
| | | | KLESEFQNLLKIIQQSNTLCSNDNIK | ACACCGAGATGGACACCCTGGAGAACGAGGCCAAGATGCTGAAGGAG |
| | | | QFISDILKKVETIRERFVKNFPEREKY | CACTACGACAACCTCATCAACAAGAAGAACATCATCACCGAGCTCCACA |
| | | | HQIEINYNEIKGIVKEVDTNPEISIFTE | ACAAGATCAACCTGATCAAGCTCCTGGAGATCCGCGCCACCAGCGACA |
| | | | KINTYIRQKIRSAHHLEDAQKIKDIIED | AGTATGTGGACATCGCCGAGCTCCTGGGCGAGGTGGTCAAGGACCAA |
| | | | VTSNYRKIKSKLSQVNNALDRIKIKK | AAGAAGAAGCTGCAAGAGGCCAAGAACAAGCTCGACACCCTGAAGGA |
| | | | SEMDTLFESLSKENANNYNSAKYFL | CCACATCGCCGTGAAGGAGAAGGAGCTGATCAACCACGACTCCAGCTT |
| | | | VDSDKIIKHLEDQVSKMSSLISYAER | CACCCTCGTCAGCATCAAGGCCTTCGACGAGATCTACGACGACATCAA |
| | | | EIKELEEKVYShhhhhh | GTACAACGTGGGCCAACTCCACACCCTGGAGGTCACCAACTTCGACGA |
| | | | (SEQ ID NO: 61) | GCTCAAGAAGGGCAAGACCTACGAGGAGAACGTGACCCACCTCCTGA |
| | | | | ACAGGCGCGAGACGCTCCAGAACGACCTGCACAACTACGAGGAGAAG |
| | | | | GACAAGCTCAAGAACACCAACATCGAGATGTCCAACGAGGAGAACAA |
| | | | | CCAAATCAGGCAGACCAGCGAGGTCATCAAGAAGCTGGAGTCCGAGT |
| | | | | TCCAAAACCTCCTGAAGATCATCCAACAGTCCAACACCCTCTGCAGCAA |
| | | | | CGATAACATCAAGCAGTTCATCAGCGACATCCTGAAGAAGGTGGAGAC |
| | | | | GATCAGGGAGCGCTTCGTCAAGAACTTCCCAGAGCGCGAGAAGTACCA |
| | | | | CCAAATCGAGATCAACTACAACGAGATCAAGGGCATCGTGAAGGAAG |
| | | | | TGGACACCAACCCAGAGATCTCCATCTTCACCGAGAAGATCAACACCTA |
| | | | | CATCAGGCAAAAGATCAGGAGCGCTCACCACCTGGAGGACGCTCAGA |
| | | | | AGATCAAGGACATCATCGAGGACGTGACCTCCAACTACAGGAAGATCA |
| | | | | AGTCCAAGCTGAGCCAAGTCAACAACGCCCTCGACCGCATCAAGATCA |
| | | | | AGAAGAGCGAGATGGACACCCTCTTCGAGTCCCTGAGCAAGGAGAAC |
| | | | | GCCAACAACTACAACAGCGCCAAGTACTTCCTGGTGGACTCCGACAAG |
| | | | | ATCATCAAGCACCTGGAGGACCAAGTGTCCAAGATGTCCAGCCTGATC |
| | | | | AGCTACGCCGAGCGCGAGATCAAGGAGCTGGAGGAGAAGGTCTACTC |
| | | | | CCACCACCACCACCACCACTGA (SEQ ID NO: 62) |
| 32 | MSP3.3 | PVX_ | mNVATRGEIVNLKNPNLRNGWSMK | ATGAACGTCGCCACCAGGGGCGAGATCGTGAACCTGAAGAACCCAAA |
| | [merozo- | 097680 | NLSAQNEENIVHSDGSDDVTDKEED | CCTCCGCAACGGCTGGAGCATGAAGAACCTGTCCGCCCAAAACGAGGA |
| | ite | | GEVLEGQKGSPKKSAEQKVHAQEE | GAACATCGTCCACTCCGACGGCAGCGACGACGTGACCGACAAGGAAG |
| | surface | | VNKESLKSKAQNAKAEAEKAAKAAE | AGGACGGCGAGGTGCTGGAGGGCCAGAAGGGCAGCCCAAAGAAGTC |
| | protein 3 | | SAKENTLDALEKVNVPTELNNEKNF | CGCCGAGCAAAAGGTCCACGCCCAAGAGGAAGTGAACAAGGAGTCCC |
| | beta | | AESAATEAKKQEKISTEAAEEVKEIE | TCAAGAGCAAGGCCCAAAACGCCAAGGCTGAGGCTGAGAAGGCTGCT |
| | (MSP3b)] | | VDGQLEKLKNEEEKTAKKARKQEIK | AAGGCTGCCGAGTCCGCCAAGGAGAACACCCTCGACGCCCTGGAGAA |
| | | | TEIAEQAAKAQAAKTEAETAQKDAT | GGTGAACGTCCCAACCGAGCTCAACAACGAGAAGAACTTCGCTGAGA |
| | | | TAKDEAIKETGKPKSQNTTKAVTMA | GCGCTGCTACCGAGGCCAAGAAGCAGGAGAAGATCTCCACCGAGGCC |
| | | | TEEEKKTKDEAQTASEKAGKTAEEA | GCCGAGGAAGTGAAGGAGATCGAGGTGGACGGCCAACTGGAGAAGC |
| | | | QKEVGKETADDDKEVSQLEEEIKEL | TGAAGAACGAGGAAGAGAAGACCGCCAAGAAGGCCAGGAAGCAGGA |
| | | | ERILKIVKDLASEASSASDNAKKAKL | GATCAAGACCGAGATCGCTGAGCAAGCTGCTAAGGCTCAGGCTGCTAA |
| | | | KTQIAAEVVKAEKARIEAEEAEKEAG | GACCGAGGCCGAGACGGCCCAAAAGGACGCCACCACCGCCAAGGACG |
| | | | EAKTKTEATEKEVLKISDESKAAKVK | AGGCCATCAAGGAGACGGGCAAGCCAAAGAGCCAGAACACCACCAAG |
| | | | KAVEKAKEAEKQAKSEAEKAKGMA | GCCGTCACCATGGCCACCGAGGAAGAGAAGAAGACCAAGGACGAGGC |
| | | | DDAGGKGTTNLEDVLTKLSEVLTSV | TCAAACCGCTTCCGAGAAGGCTGGCAAGACCGCTGAGGAAGCCCAGA |
| | | | KSLASNAEVASKNAKKEMTKAQIAA | AGGAAGTGGGCAAGGAGACGGCCGACGACGACAAGGAAGTGTCCCA |
| | | | EVAKAEKAKIEAENAKLLADTASKAA | ACTCGAAGAGGAGATCAAGGAGCTGGAGAGGATCCTCAAGATCGTGA |
| | | | ENIAKSSKAAKIANNVSTIAAEKSKVA | AGGACCTGGCTAGCGAGGCCTCCAGCGCTTCCGACAACGCCAAGAAG |
| | | | TEAADEAAKALDETENPESKIAEVTE | GCCAAGCTCAAGACCCAAATCGCTGCTGAGGTGGTCAAGGCTGAGAA |
| | | | KATKAVNAAEEAKKEKAKAEVAVEV | GGCTAGGATCGAGGCTGAGGAAGCCGAGAAGGAAGCCGGCGAGGCT |
| | | | AHAEVAKEKAQEAKEAAKQVADKS | AAGACCAAGACCGAGGCTACCGAGAAGGAAGTGCTGAAGATCTCCGA |
| | | | KLEKAIQAADKASEKANEASKLAEEA | CGAGAGCAAGGCCGCCAAGGTCAAGAAGGCCGTGGAGAAGGCCAAG |
| | | | LSNLESLEKETGEIVEKVNAIEQKVQ | GAAGCCGAGAAGCAAGCCAAGTCCGAGGCTGAGAAGGCTAAGGGCAT |
| | | | TAKNAAIEAHKEKTKAEIAVEVAKAE | GGCTGACGACGCCGGCGGCAAGGGCACCACCAACCTGGAGGACGTGC |
| | | | EAKKEADNAKVAAEKAKETAEKIAKT | TCACCAAGCTGAGCGAGGTCCTGACCTCCGTGAAGTCCCTGGCTTCCAA |
| | | | SKSTEKITEEVRKATEFAKTAGDETT | CGCTGAGGTGGCTTCCAAGAACGCCAAGAAGGAGATGACCAAGGCTC |
| | | | LAATKAESEIPSEEKNQKELLDSIKQ | AGATCGCTGCTGAGGTGGCTAAGGCTGAGAAGGCCAAGATCGAGGCC |
| | | | KAESAFQASQEAIKAKTEAENFLEIA | GAGAACGCCAAGCTGCTGGCTGACACCGCTAGCAAGGCTGCCGAGAA |
| | | | KEVPKAEAAKEEAQKAATAAEEAKT | CATCGCCAAGTCCAGCAAGGCCGCCAAGATCGCCAACAACGTCAGCAC |
| | | | EVLKIAEEVNKSDASESEKKKIETAA | CATCGCCGCCGAGAAGTCCAAGGTGGCTACCGAGGCTGCTGACGAGG |
| | | | NETAGEAEKAATFAKEAADAAKDTN | CTGCCAAGGCCCTCGACGAGACGGAGAACCCAGAGTCCAAGATCGCC |
| | | | KAVTLAVAKEKVEKALKAAKEAKKA | GAGGTGACCGAGAAGGCTACCAAGGCTGTGAACGCTGCTGAGGAAGC |
| | | | NEKASYALIRTKKQYALEPLEITSEA | CAAGAAGGAGAAGGCCAAGGCTGAGGTGGCTGTGGAGGTGGCTCAC |
| | | | GYNITEKEEQVKEEIEEQDDKASEE | GCTGAGGTGGCTAAGGAGAAGGCCCAAGAGGCCAAGGAAGCCGCCA |
| | | | EEEDTQQIDQTQIDEVDISVDNEEEE | AGCAGGTGGCCGACAAGAGCAAGCTGGAGAAGGCCATCCAAGCCGCC |
| | | | EGAAEEQIEGEKDTPTKEAKEEQTS | GACAAGGCCAGCGAGAAGGCCAACGAGGCCTCCAAGCTCGCCGAGGA |
| | | | GEKILDDKEAHKTLAEKFKDSNTAKT | AGCCCTCAGCAACCTGGAGTCCCTGGAGAAGGAGACGGGCGAGATCG |
| | | | GGVEFLETLISDVGEDTLKNLQQDL | TCGAGAAGGTGAACGCCATCGAGCAAAAGGTGCAGACCGCCAAGAAC |
| | | | HQYFKGKhhhhhh | GCCGCCATCGAGGCCCACAAGGAGAAGACCAAGGCTGAGATCGCTGT |
| | | | (SEQ ID NO: 63) | GGAGGTCGCCAAGGCCGAGGAAGCCAAGAAGGAAGCCGACAACGCC |
| | | | | AAGGTGGCTGCTGAGAAGGCTAAGGAGACGGCCGAGAAGATCGCCAA |
| | | | | GACCTCCAAGAGCACCGAGAAGATCACCGAGGAAGTGAGGAAGGCTA |
| | | | | CCGAGTTCGCTAAGACCGCTGGCGACGAGACGACCCTGGCTGCTACCA |
| | | | | AGGCTGAGAGCGAGATCCCATCCGAGGAGAAGAACCAAAAGGAGCTC |
| | | | | CTGGACAGCATCAAGCAGAAGGCCGAGAGCGCCTTCCAAGCCTCCCAA |
| | | | | GAGGCCATCAAGGCCAAGACCGAGGCCGAGAACTTCCTGGAGATCGC |
| | | | | CAAGGAAGTGCCAAAGGCCGAGGCCGCCAAGGAAGAGGCCCAAAAG |
| | | | | GCTGCTACGGCCGCTGAGGAAGCCAAGACCGAGGTCCTCAAGATCGCC |
| | | | | GAGGAAGTGAACAAGTCCGACGCCTCCGAGAGCGAGAAGAAGAAGAT |
| | | | | CGAGACGGCTGCTAACGAGACGGCTGGCGAGGCCGAGAAGGCCGCTA |
| | | | | CCTTCGCTAAGGAAGCCGCTGACGCTGCTAAGGACACCAACAAGGCCG |
| | | | | TCACCCTGGCCGTGGCCAAGGAGAAGGTCGAGAAGGCCCTCAAGGCC |
| | | | | GCCAAGGAAGCCAAGAAGGCCAACGAGAAGGCCAGCTACGCCCTGAT |
| | | | | CCGCACCAAGAAGCAGTACGCCCTGGAGCCACTGGAGATCACCTCCGA |
| | | | | GGCCGGCTACAACATCACCGAGAAGGAAGAGCAAGTGAAGGAAGAG |
| | | | | ATCGAGGAGCAGGACGACAAGGCCAGCGAGGAAGAGGAAGAGGACA |
| | | | | CCCAACAGATCGACCAAACCCAGATCGACGAGGTCGACATCTCCGTGG |
| | | | | ACAACGAGGAAGAGGAAGAGGGCGCTGCTGAGGAGCAAATCGAGGG |
| | | | | CGAGAAGGACACCCCAACCAAGGAAGCCAAGGAAGAGCAGACCTCCG |
| | | | | GCGAGAAGATCCTGGACGACAAGGAAGCCCACAAGACCCTCGCCGAG |
| | | | | AAGTTCAAGGACAGCAACACCGCTAAGACCGGCGGCGTCGAGTTCCTC |
| | | | | GAAACCCTCATCTCCGACGTGGGCGAGGACACCCTGAAGAACCTCCAA |
| | | | | CAGGACCTCCACCAGTACTTCAAGGGCAAGCACCACCACCACCACCACT |
| | | | | GA (SEQ ID NO: 64) |
| 33 | hypothe- | PVX_ | mNNYGKLKHGKWDDGSYSERTRW | ATGAACAACTACGGCAAGCTCAAGCACGGCAAGTGGGACGACGGCTC |
| | tical | 001000 | RMLSGDDHDDLLPSCDSPGGRNDE | CTACAGCGAGAGGACCAGGTGGAGGATGCTGTCCGGCGACGACCACG |
| | protein, | | HQVNKEVSRTAPSEKVKVVDKETG | ACGACCTCCTCCCATCCTGCGACAGCCCAGGCGGCAGGAACGACGAGC |
| | conserved | | ESMLVDVGESGGKSSPGVAEESGP | ACCAAGTCAACAAGGAAGTGTCCAGGACCGCCCCAAGCGAGAAGGTG |
| | | | SLRGRDVRDVRVDQETRETLOGGA | AAGGTGGTCGACAAGGAGACCGGCGAGTCCATGCTGGTGGACGTGGG |
| | | | TNRRDLTQHGEEETGDDSKRAKQD | CGAGAGCGGCGGCAAGTCCTCCCCAGGCGTGGCTGAGGAGTCCGGCC |
| | | | DEAGVRSMLNDTVTAIKDNGSNLLR | CAAGCCTGCGCGGCAGGGACGTGCGCGACGTCAGGGTGGACCAAGAG |
| | | | SVIGQINFVQGSAELLKVANEEERQ | ACCCGCGAGACCCTGCAGGGCGGCGCCACCAACAGGCGCGACCTCAC |
| | | | PSGGSVLSKEGEEATPGDFLGGNN | CCAACACGGCGAGGAAGAGACCGGCGACGACAGCAAGCGCGCTAAGC |
| | | | PNGGEKGELPNGTKNDVMIKGYAN | AGGACGACGAGGCTGGCGTCAGGTCCATGCTCAACGACACCGTGACC |
| | | | VLLNEGKHVLVGNVRNFLSRVFNLIV | GCCATCAAGGACAACGGCTCCAACCTCCTGCGCAGCGTCATCGGCCAA |
| | | | REKIMTRMCHRGGEASIERSGEPVG | ATCAACTTCGTGCAAGGCAGCGCTGAGCTCCTGAAGGTCGCCAACGAG |
| | | | ERSGEPTGERSGDPTGERSGDPTG | GAAGAGCGCCAGCCATCCGGCGGCAGCGTGCTGTCCAAGGAAGGCGA |
| | | | ERSGEPTGERSGEPTGERSGEPTA | GGAAGCCACCCCAGGCGACTTCCTCGGCGGCAACAACCCGAACGGCG |
| | | | ERSGEPTAERSDEPTAERSDEPTAD | GCGAGAAGGGCGAGCTGCCAAACGGCACCAAGAACGACGTCATGATC |
| | | | PKGDPTNCRLPKRSATKFYQSEDLY | AAGGGCTACGCCAACGTGCTCCTGAACGAGGGCAAGCACGTCCTCGTG |
| | | | NYYSSLEEMLKGRGIRWKTDRVSR | GGCAACGTCCGCAACTTCCTGTCCAGGGTGTTCAACCTCATCGTCAGGG |
| | | | YFTFSPSKKIKDNFEEVMNNKVFIES | AGAAGATCATGACCAGGATGTGCCACAGGGGCGGCGAGGCTAGCATC |
| | | | VRSILFDSHKKNKKAVFSSFAVVVET | GAGAGGTCCGGCGAGCCAGTGGGGGAGCGCTCCGGCGAGCCAACCG |
| | | | LFSLIKEEKVIADMYSYVKLFFQDLDI | GCGAGAGGAGCGGCGACCCAACCGGCGAGAGGTCTGGCGACCCTACG |
| | | | LNLKVLHFLSSSSTENTQFVGPPDL | GGGGAGAGGAGCGGGGAGCCTACCGGCGAGCGCAGCGGGGAGCCTA |
| | | | SLTNFEYILAKIYSRSVLANILSPKMN | CGGGCGAGAGGTCCGGGGAGCCTACCGCTGAGAGAAGCGGCGAGCC |
| | | | HSDSKKLSKLLTRRENNLKFSFLEG | AACCGCTGAGAGGAGCGATGAGCCTACCGCTGAGAGGTCCGACGAGC |
| | | | VKMVHSAIPSEGVSAVVLGNAGGQ | CAACCGCTGACCCAAAGGGCGACCCAACCAACTGCCGCCTCCCAAAGA |
| | | | VNVPIPGADDTLCKFIPIRKKLLYERL | GGTCCGCCACCAAGTTCTACCAAAGCGAGGACCTGTACAACTACTACTC |
| | | | SVTRKVAEEVILDYLFRLLLRKVHEY | CAGCCTGGAGGAGATGCTCAAGGGCAGGGGCATCAGGTGGAAGACC |
| | | | VLEhhhhhh (SEQ ID NO: 65) | GACCGCGTCAGCAGGTACTTCACCTTCTCCCCAAGCAAGAAGATCAAG |
| | | | | GACAACTTCGAGGAAGTGATGAACAACAAGGTCTTCATCGAGAGCGTG |
| | | | | CGCTCCATCCTCTTCGACTCCCACAAGAAGAACAAGAAGGCCGTGTTCT |
| | | | | CCAGCTTCGCCGTGGTCGTGGAGACCCTGTTCAGCCTCATCAAGGAAG |
| | | | | AGAAGGTCATCGCCGACATGTACTCCTACGTGAAGCTGTTCTTCCAAGA |
| | | | | CCTCGACATCCTGAACCTCAAGGTCCTGCACTTCCTCTCCAGCTCCAGCA |
| | | | | CCGAGAACACCCAGTTCGTGGGCCCACCAGACCTGAGCCTCACCAACT |
| | | | | TCGAGTACATCCTCGCCAAGATCTACTCCCGCAGCGTCCTGGCCAACAT |
| | | | | CCTCAGCCCAAAGATGAACCACTCCGACAGCAAGAAGCTGTCCAAGCT |
| | | | | CCTGACCAGGCGCGAGAACAACCTGAAGTTCTCCTTCCTGGAGGGCGT |
| | | | | CAAGATGGTGCACAGCGCTATCCCATCCGAGGGCGTGAGCGCTGTGGT |
| | | | | GCTGGGCAACGCTGGCGGCCAGGTCAACGTGCCAATCCCAGGCGCCG |
| | | | | ACGACACCCTCTGCAAGTTCATCCCAATCAGGAAGAAGCTCCTGTACGA |
| | | | | GCGCCTGTCCGTCACCAGGAAGGTGGCCGAGGAAGTGATCCTGGACT |
| | | | | ACCTCTTCCGCCTCCTGCTCAGGAAGGTGCACGAGTATGTGCTGGAGC |
| | | | | ACCATCACCACCATCACTGA (SEQ ID NO: 66) |
| 34 | merozoite | PVX_ | mGNVSPPNFNDNRVNGNNGNKGN | ATGGGCAACGTGTCCCCACCAAACTTCAACGACAACAGGGTCAACGGC |
| | surface | 097625 | GNDNDVPSFIGGNNNNVNGNNDDN | AACAACGGCAACAAGGGCAACGGCAACGACAACGACGTGCCAAGCTT |
| | protein 8 | | IFNKNGKDVTRNDGDAKDGENRNN | CATCGGCGGCAACAACAACAACGTCAACGGCAACAACGACGACAACAT |
| | (GPI- | | KKNENGSGSNENNSIANADNGSGK | CTTCAACAAGAACGGCAAGGACGTGACCCGCAACGACGGCGACGCTA |
| | anchored, | | SDANANQIDEDGNKMDEASLKKILKI | AGGACGGCGAGAACCGCAACAACAAGAAGAACGAGAACGGCTCCGGC |
| | C24) | | VDEMENIQGLLDGDYSILDKYSVKLV | AGCAACGAGAACAACTCCATCGCCAACGCTGACAACGGCTCCGGCAAG |
| | | | DEDDGETNKRKIIGEYDLKMLKNILL | AGCGACGCCAACGCCAACCAAATCGACGAGGACGGCAACAAGATGGA |
| | | | FREKISRVCENKYNKNLPVLLKKCS | CGAGGCCAGCCTCAAGAAGATCCTGAAGATCGTGGACGAGATGGAGA |
| | | | NVDDPKLSKSREKIKKGLAKNNMSIE | ACATCCAGGGCCTCCTGGACGGCGACTACTCCATCCTCGACAAGTACA |
| | | | DFVVGLLEDLFEKINEHFIKDDSFDL | GCGTGAAGCTGGTCGACGAGGACGACGGCGAGACGAACAAGAGGAA |
| | | | SDYLADFELINYIIMHETSELIDELLNII | GATCATCGGCGAGTACGACCTCAAGATGCTGAAGAACATCCTCCTGTTC |
| | | | ESMNFRLESGSLEKMVKSAESGMN | AGGGAGAAGATCTCCCGCGTCTGCGAGAACAAGTACAACAAGAACCTC |
| | | | LNCKMKEDIIHLLKKSSAKFFKIEIDR | CCAGTGCTCCTGAAGAAGTGCAGCAACGTCGACGACCCAAAGCTCTCC |
| | | | KTKMIYPVQATHKGANMKQLALSFL | AAGAGCCGCGAGAAGATCAAGAAGGGCCTGGCTAAGAACAACATGTC |
| | | | QKNNVCEHKKCPLNSNCYVINGEEV | CATCGAGGACTTCGTGGTCGGCCTCCTGGAGGACCTGTTCGAGAAGAT |
| | | | CRCLPGFSDVKIDNVMNCVRDDTLD | CAACGAGCACTTCATCAAGGACGACTCCTTCGACCTCAGCGACTACCTG |
| | | | CSNNNGGCDVNATCTLIDKKIVCEC | GCCGACTTCGAGCTCATCAACTACATCATCATGCACGAGACGTCCGAGC |
| | | | KDNFEGDGIYChhhhhh | TGATCGACGAGCTCCTGAACATCATCGAGAGCATGAACTTCAGGCTGG |
| | | | (SEQ ID NO: 67) | AGTCCGGCAGCCTGGAGAAGATGGTGAAGTCCGCCGAGAGCGGCATG |
| | | | | AACCTCAACTGCAAGATGAAGGAAGACATCATCCACCTCCTGAAGAAG |
| | | | | TCCAGCGCCAAGTTCTTCAAGATCGAGATCGACCGCAAGACCAAGATG |
| | | | | ATCTACCCAGTGCAAGCCACCCACAAGGGCGCCAACATGAAGCAACTC |
| | | | | GCCCTGTCCTTCCTCCAGAAGAACAACGTCTGCGAGCACAAGAAGTGC |
| | | | | CCACTGAACAGCAACTGCTACGTGATCAACGGCGAGGAAGTGTGCAG |
| | | | | GTGCCTCCCAGGCTTCTCCGACGTCAAGATCGACAACGTGATGAACTG |
| | | | | CGTCCGCGACGACACCCTCGACTGCAGCAACAACAACGGCGGCTGCGA |
| | | | | CGTGAACGCTACCTGCACCCTGATCGACAAGAAGATCGTCTGCGAGTG |
| | | | | CAAGGACAACTTCGAGGGCGACGGCATCTACTGCCACCACCACCACCA |
| | | | | CCACTGA (SEQ ID NO: 68) |
| 35 | adenylate | PVX_ | METLLDSETLKNYEKETNEYIRKKKV | ATGGAGACGCTCCTGGACTCCGAGACGCTCAAGAACTACGAGAAGGA |
| | kinase- | 087110 | EKLFDVILKNVLVNKPENVYLYIYKNI | GACGAACGAGTACATCAGGAAGAAGAAGGTGGAGAAGCTCTTCGACG |
| | like | | YSFLLNKIFVIGPPLLKITPTLCSAIAS | TCATCCTCAAGAACGTGCTGGTCAACAAGCCAGAGAACGTGTACCTGT |
| | protein 2, | | CFSYYHLSASHMIESYTTGEVDDAA | ACATCTACAAGAACATCTACAGCTTCCTCCTGAACAAGATCTTCGTCATC |
| | putative | | ESSTSKKLVSDDLICSIVKSNINQLNA | GGCCCACCACTCCTGAAGATCACCCCAACCCTCTGCTCCGCCATCGCCT |
| | (AKLP2) | | KQKRGYVVEGFPGTNLQADSCLRH | CCTGCTTCAGCTACTACCACCTGTCCGCCAGCCACATGATCGAGAGCTA |
| | | | LPSYVFVLYADEEYIYDKYEQENNV | CACCACCGGCGAGGTGGACGACGCTGCTGAGTCCAGCACCTCCAAGAA |
| | | | KIRSDMNSQTFDENTQLFEVAEFNT | GCTCGTGAGCGACGACCTGATCTGCTCCATCGTCAAGAGCAACATCAA |
| | | | NPLKDEVKVYLRNhhhhhh | CCAACTCAACGCCAAGCAGAAGAGGGGCTACGTGGTCGAGGGCTTCC |
| | | | (SEQ ID NO: 69) | CAGGCACCAACCTCCAGGCTGACTCCTGCCTCAGGCACCTGCCAAGCTA |
| | | | | CGTGTTCGTCCTGTACGCCGACGAGGAGTACATCTACGACAAGTACGA |
| | | | | GCAGGAGAACAACGTGAAGATCAGGTCCGACATGAACAGCCAAACCT |
| | | | | TCGACGAGAACACCCAGCTGTTCGAGGTCGCCGAGTTCAACACCAACC |
| | | | | CACTCAAGGACGAGGTGAAGGTCTACCTGCGCAACCACCACCACCACC |
| | | | | ACCACTGA (SEQ ID NO: 70) |
| 36 | MSP7-like | PVX_ | mKPGVEKKKKLEEDVIGILRRKLESL | ATGAAGCCAGGCGTGGAGAAGAAGAAGAAGCTCGAAGAGGACGTCA |
| | protein | 082670 | QKRSLTNSDGKLKKEIELVKKQIQEL | TCGGCATCCTGCGCAGGAAGCTGGAGTCCCTGCAAAAGAGGTCCCTCA |
| | | | QKYEKGEAGKKVDATLGEEPGVES | CCAACAGCGACGGCAAGCTCAAGAAGGAGATCGAGCTGGTCAAGAAG |
| | | | AEEQPLSVEEAGDTQDEDRLDELE | CAAATCCAGGAGCTGCAGAAGTACGAGAAGGGCGAGGCTGGCAAGA |
| | | | GVEDFEEENLEQSEQVEEAEVVEEA | AGGTGGACGCTACCCTGGGCGAGGAGCCGGGCGTGGAGTCCGCTGAG |
| | | | EEEAGDAEEEQPAEAEEDGSLLEEA | GAGCAACCACTGAGCGTGGAGGAAGCCGGCGACACCCAGGACGAGG |
| | | | PNSVERKAEGAIAEFEEADVEEGAE | ACAGGCTCGACGAGCTGGAGGGCGTCGAGGACTTCGAGGAAGAGAAC |
| | | | ADEGVETDEGADADEASLGSFDLE | CTGGAGCAAAGCGAGCAGGTGGAGGAAGCCGAGGTGGTGGAGGAAG |
| | | | GELIEEDLQESFDLEGEQEEEDLQE | CCGAGGAAGAGGCCGGCGACGCTGAGGAAGAGCAACCGGCTGAGGC |
| | | | GFKSEEEANQGGQLPREIPPHGEEA | TGAGGAAGACGGCTCCCTCCTCGAAGAGGCCCCAAACAGCGTGGAGA |
| | | | VEPPLRGNKPSMEYVGNLHSDVGP | GGAAGGCTGAGGGCGCTATCGCTGAGTTCGAGGAAGCCGACGTCGAG |
| | | | TEGSANQISPPSVDEKGKEDGDKYK | GAAGGCGCCGAGGCCGACGAGGGCGTGGAGACGGACGAGGGCGCTG |
| | | | SASQDGGNSVGINNFGGCFQGGNS | ACGCTGACGAGGCTTCCCTGGGCAGCTTCGACCTGGAGGGCGAGCTG |
| | | | NGICPLDIFKKVLEDENFLQEFDSFIH | ATCGAGGAAGACCTCCAGGAGTCTTTCGACCTGGAGGGGGAGCAAGA |
| | | | NLYGSSKNNTPWGGDKMGNENLY | GGAAGAGGACCTCCAAGAGGGCTTCAAGAGCGAGGAAGAGGCCAAC |
| | | | MDLFTNALSFLNTIEVIhhhhhh | CAAGGCGGCCAGCTGCCAAGGGAGATCCCACCACACGGCGAGGAAGC |
| | | | (SEQ ID NO: 71) | CGTGGAGCCACCACTCCGCGGCAACAAGCCATCCATGGAGTATGTGGG |
| | | | | CAACCTGCACAGCGACGTGGGCCCAACCGAGGGCAGCGCCAACCAAA |
| | | | | TCTCCCCACCAAGCGTCGACGAGAAGGGCAAGGAAGACGGCGACAAG |
| | | | | TACAAGTCCGCCAGCCAAGACGGCGGAAACTCCGTGGGCATCAACAAC |
| | | | | TTCGGCGGATGCTTCCAGGGCGGCAACAGCAACGGCATCTGCCCACTC |
| | | | | GACATCTTCAAGAAGGTCCTGGAGGACGAGAACTTCCTGCAGGAGTTC |
| | | | | GACTCCTTCATCCACAACCTGTACGGCTCCAGCAAGAACAACACCCCAT |
| | | | | GGGGCGGCGACAAGATGGGCAACGAGAACCTCTACATGGACCTGTTC |
| | | | | ACCAACGCCCTCAGCTTCCTGAACACCATCGAGGTCATCCACCACCACC |
| | | | | ACCACCACTGA (SEQ ID NO: 72) |
| 37 | high | PVX_ | mELSHSLSVKNAPDASALNIEVEKD | ATGGAGCTCTCCCACAGCCTGTCCGTGAAGAACGCTCCAGACGCTAGC |
| | molecular | 099930 | KKKICKNAFQYINVAELLSPREEETY | GCTCTCAACATCGAGGTCGAGAAGGACAAGAAGAAGATCTGCAAGAA |
| | weight | | VQKCEEVLDTIKNDSPDESAEAEINE | CGCCTTCCAATACATCAACGTCGCCGAGCTCCTGTCCCCAAGGGAGGA |
| | rhoptry | | FILSLLHARSKYTIINDSDEEVLSKLL | AGAGACTTACGTGCAGAAGTGCGAGGAAGTGCTGGACACCATCAAGA |
| | protein-2, | | RSINGSISEEAALKRAKQLITFNRFIK | ACGACAGCCCAGACGAGTCCGCTGAGGCTGAGATCAACGAGTTCATCC |
| | putative | | DKAKVKNVQEMLVISSKADDFMNEP | TCAGCCTCCTGCACGCCCGCTCCAAGTACACCATCATCAACGACAGCGA |
| | | | KQKMLQKIIDSFELYNDYLVILGSNIN | CGAGGAAGTGCTGAGCAAGCTCCTGAGGTCCATCAACGGCAGCATCTC |
| | | | IAKRYSSETFLSIKNEKFCSDHIHLCQ | CGAGGAAGCCGCTCTCAAGAGGGCTAAGCAACTGATCACCTTCAACAG |
| | | | KFYEQSIIYYRLKVIFDNLVTYVDQNS | GTTCATCAAGGACAAGGCCAAGGTGAAGAACGTCCAGGAGATGCTCG |
| | | | KHFKKEKLLELLNMDYRVNRESKVH | TCATCTCCAGCAAGGCCGACGACTTCATGAACGAGCCAAAGCAAAAGA |
| | | | ENYVLEDETVIPTMRITDIYDQDRLIV | TGCTCCAGAAGATCATCGACAGCTTCGAGCTGTACAACGACTACCTCGT |
| | | | EVVQDGNSKLMHGRDIEKREISERYI | GATCCTGGGCTCCAACATCAACATCGCCAAGCGCTACTCCAGCGAGAC |
| | | | VTVKNLRKDLNDEGLYADLMKTVKN | GTTCCTCAGCATCAAGAACGAGAAGTTCTGCTCCGACCACATCCACCTG |
| | | | YVLSITQIDNDISNLVRELDHEDVEKh | TGCCAAAAGTTCTACGAGCAGAGCATCATCTACTACAGGCTCAAGGTC |
| | | | hhhhh (SEQ ID NO: 73) | ATCTTCGACAACCTGGTGACCTACGTCGACCAAAACTCCAAGCACTTCA |
| | | | | AGAAGGAGAAGCTCCTGGAGCTCCTGAACATGGACTACAGGGTGAAC |
| | | | | CGCGAGTCCAAGGTGCACGAGAACTACGTCCTGGAGGACGAGACTGT |
| | | | | GATCCCAACCATGCGCATCACCGACATCTACGACCAAGACAGGCTCATC |
| | | | | GTGGAGGTGGTCCAGGACGGCAACAGCAAGCTGATGCACGGCAGGG |
| | | | | ACATCGAGAAGCGCGAGATCTCCGAGAGGTACATCGTGACCGTCAAG |
| | | | | AACCTCCGCAAGGACCTGAACGACGAGGGCCTCTACGCCGACCTGATG |
| | | | | AAGACCGTGAAGAACTACGTCCTCAGCATCACCCAGATCGACAACGAC |
| | | | | ATCTCCAACCTCGTGAGGGAGCTGGACCACGAGGACGTCGAGAAGCA |
| | | | | CCACCACCACCACCACTGA (SEQ ID NO: 74) |
| 38 | IMP- | PVX_ | MEKLDIPPHEMYEDMQQAFREQDK | ATGGAGAAGCTCGACATCCCACCACACGAGATGTACGAGGACATGCAA |
| | specific | 084340 | YDFLAISDGSVINSYMKKNVVDWNN | CAGGCCTTCAGGGAGCAAGACAAGTACGACTTCCTGGCCATCTCCGAC |
| | 5'- | | RYSYNQLKNKDSLIMFLVDIFRSLFL | GGCAGCGTGATCAACTCCTACATGAAGAAGAACGTGGTCGACTGGAAC |
| | nucleoti- | | SNCIDKNIDNVLSSIEEMFTDHYYNP | AACAGGTACTCCTACAACCAGCTCAAGAACAAGGACAGCCTCATCATG |
| | dase | | MHSRLKYLIDDVGIFFTKLPITKAFHT | TTCCTGGTGGACATCTTCCGCTCCCTCTTCCTGAGCAACTGCATCGACA |
| | | | YNKKYRITKRLYAPPTFNEVRHILNL | AGAACATCGACAACGTCCTGTCCAGCATCGAGGAGATGTTCACCGACC |
| | | | AQILSLEDGLDLLTFDADETLYPDGY | ACTACTACAACCCAATGCACAGCAGGCTCAAGTACCTGATCGACGACG |
| | | | DFHDEVLASYISSLLKKMNIAIVTAAS | TGGGCATCTTCTTCACCAAGCTCCCAATCACCAAGGCCTTCCACACCTAC |
| | | | YSNDAEKYQKRLENLLRYFSKHNIE | AACAAGAAGTACAGGATCACCAAGCGCCTGTACGCCCCACCAACCTTC |
| | | | DGSYENFYVMGGESNYLFKCNEDA | AACGAGGTCCGCCACATCCTCAACCTGGCCCAAATCCTCTCCCTGGAGG |
| | | | NLYSVPEEEWYHYKKYVNKETVEQI | ACGGCCTCGACCTCCTGACCTTCGACGCCGACGAGACGCTGTACCCAG |
| | | | LDISQKCLQQVITDFKLCAQIQRKEK | ACGGCTACGACTTCCACGACGAGGTGCTCGCCAGCTACATCTCCAGCCT |
| | | | SIGLVPNKIPSANNQKEQKNYMIKYE | CCTGAAGAAGATGAACATCGCCATCGTCACCGCCGCCTCCTACAGCAA |
| | | | VLEEAVIRVKKEIVKNKITAPYCAFNG | CGACGCCGAGAAGTACCAGAAGAGGCTGGAGAACCTCCTGCGCTACTT |
| | | | GQDLWVDIGNKAEGLIILQKLLKIEKK | CTCCAAGCACAACATCGAGGACGGCAGCTACGAGAACTTCTACGTGAT |
| | | | KCCHIGDQFLHSGNDFPTRFCSLTL | GGGCGGCGAGTCCAACTACCTCTTCAAGTGCAACGAGGACGCCAACCT |
| | | | WISNPQETKACLKSIMNLNMKSFIPE | GTACAGCGTCCCAGAGGAAGAGTGGTACCACTACAAGAAGTATGTGA |
| | | | VLYENEhhhhhh | ACAAGGAGACGGTCGAGCAAATCCTCGACATCTCCCAGAAGTGCCTGC |
| | | | (SEQ ID NO: 75) | AACAAGTGATCACCGACTTCAAGCTCTGCGCCCAAATCCAGAGGAAGG |
| | | | | AGAAGTCCATCGGCCTGGTCCCAAACAAGATCCCAAGCGCCAACAACC |
| | | | | AAAAGGAGCAGAAGAACTACATGATCAAGTACGAGGTGCTCGAAGAG |
| | | | | GCCGTGATCCGCGTCAAGAAGGAGATCGTCAAGAACAAGATCACCGCT |
| | | | | CCATACTGCGCCTTCAACGGCGGCCAAGACCTGTGGGTGGACATCGGC |
| | | | | AACAAGGCCGAGGGCCTCATCATCCTGCAAAAGCTCCTGAAGATCGAG |
| | | | | AAGAAGAAGTGCTGCCACATCGGCGACCAGTTCCTCCACAGCGGCAAC |
| | | | | GACTTCCCAACCCGCTTCTGCTCCCTCACCCTGTGGATCAGCAACCCAC |
| | | | | AGGAGACGAAGGCCTGCCTCAAGTCCATCATGAACCTGAACATGAAGA |
| | | | | GCTTCATCCCAGAGGTCCTCTACGAGAACGAGCACCACCACCACCACCA |
| | | | | CTGA (SEQ ID NO: 76) |
| 39 | subpelli- | PVX_ | MEIIAEKPKVKFNFASEEYKNCDSSD | ATGGAGATCATCGCCGAGAAGCCAAAGGTCAAGTTCAACTTCGCCTCC |
| | cular | 098915 | YSECAEDYGRPNGKDYFYANRILSL | GAGGAGTACAAGAACTGCGACTCCAGCGACTACTCCGAGTGCGCTGA |
| | microtu- | | DRNSEQRRKESPSKRPGLCVDEICT | GGACTACGGCAGGCCAAACGGCAAGGACTACTTCTACGCCAACAGGAT |
| | bule | | CGFHRCPKIVKSLPFDGESNYRSEF | CCTCTCCCTGGACCGCAACAGCGAGCAGAGGCGCAAGGAGTCCCCAA |
| | protein 1, | | GPKPLPELPPRQEAKLTRSLPFEGE | GCAAGAGGCCAGGCCTCTGCGTGGACGAGATCTGCACCTGCGGCTTCC |
| | putative | | SNYRSEFGPKPLPELPPRVEQKPPK | ACCGCTGCCCAAAGATCGTCAAGTCCCTGCCATTCGACGGCGAGTCCA |
| | (SPM1) | | SLPFDGESNYRSEFGPKPLPELPPR | ACTACCGCAGCGAGTTCGGCCCAAAGCCACTCCCAGAGCTGCCACCAA |
| | | | VEQKPPKSLPFDGESNYRSEFGPKP | GGCAAGAGGCCAAGCTCACCCGCAGCCTGCCATTCGAGGGCGAGTCC |
| | | | LPELPPRVEQKPPKSLPFEGESNYR | AACTACAGGTCCGAGTTCGGGCCTAAGCCTCTGCCTGAGCTGCCACCA |
| | | | SEFGPKPLPELPPRVEQKPPKSLPF | CGCGTGGAGCAAAAGCCACCAAAGTCCCTCCCTTTCGATGGGGAGAGC |
| | | | EGESNYRSEFGPKALPELPPRVEQK | AACTACAGGAGTGAATTCGGGCCTAAGCCGCTGCCCGAGCTGCCACCA |
| | | | PPKSLPFEGESNYRSEFGPKPLPAL | CGCGTCGAGCAGAAGCCACCAAAGAGCCTCCCTTTCGATGGCGAGAGC |
| | | | PPRVETKLVKSLPFEGESNYRSEFG | AACTACAGGAGCGAATTTGGGCCTAAGCCGCTGCCGGAACTGCCACCA |
| | | | PKPLPELPPRVEQKPPKSLPFEGES | CGCGTGGAACAAAAGCCACCAAAGAGCCTGCCTTTCGAGGGGGAGTC |
| | | | NYRSEFGPKPLPALPPRVVTKLVKS | CAACTACAGGAGTGAGTTTGGGCCTAAGCCGTTGCCTGAACTGCCACC |
| | | | LPFEGESNYRSEFGPKPLPEIPPRV | ACGCGTCGAACAGAAACCACCAAAAAGCCTCCCTTTCGAGGGCGAGAG |
| | | | EQKPPKSLPFEGESNYRSEFGPKPL | CAACTACCGCTCCGAGTTCGGCCCAAAGGCTCTGCCGGAGCTGCCACC |
| | | | PELPPRVEQKPPKSLPFEGESNYRS | ACGCGTGGAACAGAAACCACCAAAGAGCCTCCCCTTCGAGGGGGAGA |
| | | | EFGPKQLPELPPRQEAKLTRSLPFE | GCAATTATCGCTCTGAGTTCGGGCCAAAGCCGCTGCCGGCTCTGCCACC |
| | | | GESSYRSEYVRKAIPICPVNLLPKYP | ACGCGTGGAGACGAAGCTCGTCAAGAGCCTCCCGTTCGAGGGGGAGA |
| | | | APTYPSEHVFWDSACKRWYhhhhhh | GCAACTATCGCTCCGAATTTGGGCCTAAACCACTGCCTGAACTGCCACC |
| | | | (SEQ ID NO: 77) | ACGCGTGGAACAGAAGCCACCAAAAAGCCTCCCCTTTGAAGGGGAGA |
| | | | | GCAATTACCGCTCCGAGTTCGGGCCCAAGCCGCTGCCGGCCCTGCCAC |
| | | | | CACGCGTGGTCACCAAGCTCGTGAAGTCCCTCCCCTTTGAAGGCGAGA |
| | | | | GCAACTACAGATCTGAGTTCGGGCCTAAGCCACTCCCAGAGATCCCAC |
| | | | | CACGCGTCGAGCAAAAACCACCAAAATCTCTCCCCTTTGAGGGTGAGA |
| | | | | GCAATTATCGCTCAGAGTTCGGGCCCAAGCCTCTGCCGGAGCTGCCAC |
| | | | | CACGCGTCGAACAGAAGCCACCAAAGAGCTTACCTTTTGAAGGGGAGA |
| | | | | GCAACTACCGCAGTGAATTCGGCCCAAAGCAGCTGCCAGAACTGCCAC |
| | | | | CAAGGCAAGAGGCCAAACTCACCCGCTCCCTGCCTTTCGAGGGCGAGT |
| | | | | CCAGCTACAGGAGCGAGTATGTGAGGAAGGCCATCCCAATCTGCCCAG |
| | | | | TCAACCTCCTGCCAAAGTACCCAGCCCCAACCTACCCATCCGAGCACGT |
| | | | | GTTCTGGGACAGCGCCTGCAAGCGCTGGTACCACCACCACCACCACCA |
| | | | | CTGA (SEQ ID NO: 78) |
| 40 | trypto- | PVX_ | mAAANRPNANGFVSPTLIGFGELSI | ATGGCTGCCGCCAACAGGCCAAACGCCAACGGCTTCGTCTCCCCAACC |
| | phan-rich | 088820 | QESEEFKRMAWNNWMLRLESDWK | CTCATCGGCTTCGGCGAGCTGTCCATCCAAGAGAGCGAGGAGTTCAAG |
| | antigen | | HFNDSVEEAKTKWLHERDSAWSD | AGGATGGCCTGGAACAACTGGATGCTCCGCCTGGAGTCCGACTGGAA |
| | (Pv-fam-a) | | WLRSLQSKWSHYSEKMLKEHKSNV | GCACTTCAACGACAGCGTGGAGGAAGCCAAGACCAAGTGGCTGCACG |
| | | | MEKSANWNDTQWGNWIKTEGRKIL | AGAGGGACTCCGCTTGGAGCGACTGGCTCCGCTCCCTGCAGAGCAAGT |
| | | | EAQWEKWIKKGDDQLQKLILDKWV | GGTCCCACTACAGCGAGAAGATGCTGAAGGAGCACAAGTCCAACGTC |
| | | | QWKNDKIRSWLSSEWKTEEDYYWA | ATGGAGAAGAGCGCCAACTGGAACGACACCCAATGGGGCAACTGGAT |
| | | | NVERATTAKWLQEAEKMHWLKWKE | CAAGACCGAGGGCCGCAAGATCCTGGAGGCCCAGTGGGAGAAGTGG |
| | | | RINRESEQWVNWVOMKESVYINVE | ATCAAGAAGGGCGACGACCAACTGCAGAAGCTCATCCTGGACAAGTG |
| | | | WKKWPKWKNDKKILFNKWSTNLVY | GGTCCAGTGGAAGAACGACAAGATCAGGTCCTGGCTCTCCAGCGAGT |
| | | | KWTLKKQWNVWIKEANTAPQVhhhh | GGAAGACCGAGGAAGACTACTACTGGGCTAACGTGGAGAGGGCTACC |
| | | | hh (SEQ ID NO: 79) | ACCGCTAAGTGGCTCCAAGAGGCCGAGAAGATGCACTGGCTGAAGTG |
| | | | | GAAGGAGAGGATCAACCGCGAGTCCGAGCAATGGGTGAACTGGGTCC |
| | | | | AGATGAAGGAGAGCGTGTACATCAACGTCGAGTGGAAGAAGTGGCCA |
| | | | | AAGTGGAAGAACGATAAGAAGATCCTGTTCAACAAGTGGAGCACCAA |
| | | | | CCTCGTGTACAAGTGGACCCTGAAGAAGCAGTGGAACGTCTGGATCAA |
| | | | | GGAAGCCAACACCGCCCCACAGGTGCACCACCACCACCACCACTGA |
| | | | | (SEQ ID NO: 80) |
| 41 | PvTRAP/ | PVX_ | mEKVVDEVKYSEEVCNESVDLYLLV | ATGGAGAAGGTGGTCGACGAGGTGAAGTACAGCGAGGAAGTGTGCA |
| | SSP2 | 082735 | DGSGSIGYPNWITKVIPMLNGLINSL | ACGAGTCCGTCGACCTCTACCTCCTGGTGGACGGCTCCGGCAGCATCG |
| | | | SLSRDTINLYMNLFGNYTTELIRLGS | GCTACCCAAACTGGATCACCAAGGTCATCCCAATGCTCAACGGCCTGAT |
| | | | GQSIDKRQALSKVTELRKTYTPYGT | CAACTCCCTCAGCCTGTCCCGCGACACCATCAACCTCTACATGAACCTG |
| | | | TNMTAALDEVQKHLNDRVNREKAIQ | TTCGGCAACTACACCACCGAGCTCATCAGGCTGGGCAGCGGCCAATCC |
| | | | LVILMTDGVPNSKYRALEVANKLKQ | ATCGACAAGCGCCAGGCCCTCAGCAAGGTGACCGAGCTGAGGAAGAC |
| | | | RNVSLAVIGVGQGINHQFNRLIAGC | CTACACCCCATACGGCACCACCAACATGACCGCCGCCCTCGACGAGGT |
| | | | RPREPNCKFYSYADWNEAVALIKPFI | GCAAAAGCACCTGAACGACAGGGTCAACCGCGAGAAGGCCATCCAGC |
| | | | AKVCTEVERVANCGPWDPWTACSV | TCGTGATCCTGATGACCGACGGCGTCCCAAACAGCAAGTACCGCGCCC |
| | | | TCGRGTHSRSRPSLHEKCTTHMVS | TGGAGGTGGCCAACAAGCTGAAGCAAAGGAACGTCTCCCTGGCCGTG |
| | | | ECEEGECPVEPEPLPVPAPLPTVPE | ATCGGCGTGGGCCAAGGCATCAACCACCAGTTCAACAGGCTGATCGCT |
| | | | DVNPRDTDDENENPNFNKGLDVPD | GGCTGCAGGCCACGCGAGCCAAACTGCAAGTTCTACAGCTACGCTGAC |
| | | | EDDDEVPPANEGADGNPVEENVFP | TGGAACGAGGCTGTGGCTCTCATCAAGCCATTCATCGCCAAGGTCTGC |
| | | | PADDSVPDESNVLPLPPAVPGGSSE | ACCGAGGTGGAGAGGGTGGCTAACTGCGGCCCATGGGACCCGTGGAC |
| | | | EFPADVQNNPDSPEELPMEQEVPQ | CGCTTGCTCCGTGACCTGCGGCAGGGGCACCCACAGCAGGTCCCGCCC |
| | | | DNNVNEPERSDSNGYGVNEKVIPN | AAGCCTGCACGAGAAGTGCACCACCCACATGGTGTCCGAGTGCGAGG |
| | | | PLDNERDMANKNKTVHPGRKDSAR | AAGGCGAGTGCCCAGTGGAGCCAGAGCCACTGCCGGTCCCAGCCCCA |
| | | | DRYARPHGSTHVNNNRANENSDIP | CTGCCAACCGTGCCAGAGGACGTCAACCCAAGGGACACCGACGACGA |
| | | | NNPVPSDYEQPEDKAKKSSNNGYK | GAACGAGAACCCAAACTTCAACAAGGGCCTCGACGTGCCAGACGAGG |
| | | | hhhhhh (SEQ ID NO: 81) | ACGACGACGAGGTCCCACCAGCTAACGAGGGCGCTGACGGCAACCCA |
| | | | | GTGGAGGAGAACGTCTTCCCACCAGCCGACGACAGCGTGCCAGACGA |
| | | | | GTCCAACGTGCTGCCACTGCCACCAGCTGTGCCAGGCGGCTCCAGCGA |
| | | | | GGAGTTCCCAGCTGACGTCCAAAACAACCCAGACTCCCCAGAGGAGCT |
| | | | | CCCGATGGAGCAAGAGGTGCCACAGGACAACAACGTCAACGAGCCAG |
| | | | | AGCGCAGCGACTCCAACGGCTACGGCGTGAACGAGAAGGTCATCCCA |
| | | | | AACCCACTGGACAACGAGAGGGACATGGCCAACAAGAACAAGACCGT |
| | | | | GCACCCGGGCAGGAAGGACAGCGCCAGGGACCGCTACGCCAGGCCAC |
| | | | | ACGGCTCCACCCACGTGAACAACAACAGGGCCAACGAGAACAGCGAC |
| | | | | ATCCCAAACAACCCAGTCCCATCCGACTACGAGCAGCCAGAGGACAAG |
| | | | | GCCAAGAAGTCCAGCAACAACGGCTACAAGCACCACCACCACCACCAC |
| | | | | TGA (SEQ ID NO: 82) |
| 42 | MSP7-like | PVX_ | mDDKKDKENEHKEDADKKNNDELK | ATGGACGACAAGAAGGACAAGGAGAACGAGCACAAGGAAGACGCCG |
| | protein | 082645 | TLKGKLQKIRVQIKDDKLPQKISEEQI | ATAAGAAGAACAACGACGAGCTCAAGACCCTGAAGGGCAAGCTCCAA |
| | | | SVLKKKLEDFKNLKSEHEAKLASEK | AAGATCAGGGTGCAGATCAAGGACGACAAGCTGCCACAAAAGATCTC |
| | | | GDTSAGGEGELGLSDKEFVGQNVK | CGAGGAGCAGATCAGCGTCCTCAAGAAGAAGCTGGAGGACTTCAAGA |
| | | | ANGDAAGVSGEQGASGGSGQGEA | ACCTCAAGTCCGAGCACGAGGCCAAGCTGGCCTCCGAGAAGGGCGAC |
| | | | GPSSPADEQDDDNEAVQWGPATEE | ACCTCCGCCGGCGGCGAGGGCGAGCTGGGCCTGTCCGACAAGGAGTT |
| | | | VVAEAMSDEGPQEQGAEGGPSNPT | CGTGGGCCAAAACGTCAAGGCCAACGGCGACGCCGCCGGCGTGAGCG |
| | | | DDQAEEATPGPSKPASGASGSQGA | GCGAGCAAGGCGCCTCCGGCGGCAGCGGCCAGGGCGAGGCTGGCCC |
| | | | SDSSNDSAEPTSAAAAAAPAGPTAA | ATCCAGCCCAGCCGACGAGCAAGACGACGACAACGAGGCTGTCCAGT |
| | | | AASPQVKHVDTLCDELLAGENKKNV | GGGGCCCAGCTACCGAGGAAGTGGTGGCTGAGGCTATGTCCGACGAG |
| | | | LDEGEDHSQYNIFRKQYDKMVLNKT | GGCCCACAAGAGCAGGGCGCTGAGGGCGGCCCAAGCAACCCAACCGA |
| | | | EYNISLKLLDTMLTNGQVEREKKNTL | CGACCAAGCTGAGGAAGCCACCCCAGGCCCATCCAAGCCAGCTTCCGG |
| | | | IKTFKKALYDKQYSEKLRNLISGVYA | CGCTTCCGGCAGCCAGGGCGCTTCCGACTCCAGCAACGACTCCGCCGA |
| | | | FAKRNNFIDGDKVKEGDYSKLFEYIG | GCCAACCAGCGCTGCCGCCGCCGCCGCCCCAGCTGGCCCAACCGCTGC |
| | | | CMMNTLELhhhhhh | CGCCGCCAGCCCACAGGTGAAGCACGTGGACACCCTCTGCGACGAGCT |
| | | | (SEQ ID NO: 83) | CCTGGCTGGCGAGAACAAGAAGAACGTGCTGGACGAGGGCGAGGAC |
| | | | | CACTCCCAATACAACATCTTCAGGAAGCAGTACGACAAGATGGTCCTCA |
| | | | | ACAAGACCGAGTACAACATCAGCCTCAAGCTCCTGGACACCATGCTGA |
| | | | | CCAACGGCCAAGTGGAGCGCGAGAAGAAGAACACCCTCATCAAGACC |
| | | | | TTCAAGAAGGCCCTGTACGACAAGCAGTACTCCGAGAAGCTCAGGAAC |
| | | | | CTGATCAGCGGCGTGTACGCCTTCGCCAAGCGCAACAACTTCATCGAC |
| | | | | GGCGACAAGGTGAAGGAAGGCGACTACAGCAAGCTCTTCGAGTACAT |
| | | | | CGGCTGCATGATGAACACCCTGGAGCTGCACCACCACCACCACCACTG |
| | | | | A (SEQ ID NO: 84) |
| 43 | early | PVX_ | mKRHATRGALHSLKSIEHEVQRKKN | ATGAAGAGGCACGCTACCCGCGGCGCCCTCCACTCCCTGAAGAGCATC |
| | trans- | 111065 | KKKKIILYSIGSILALAAVIATGVGIGM | GAGCACGAGGTGCAAAGGAAGAAGAACAAGAAGAAGAAGATCATCCT |
| | cribed | | YIKKKKKNSLEKLQQIEPQKLESKTD | CTACTCCATCGGCAGCATCCTGGCTCTGGCTGCCGTGATCGCTACCGGC |
| | membrane | | ESDPLLGKSEAAKVEVKGDSEEVPQ | GTCGGCATCGGCATGTACATCAAGAAGAAGAAGAAGAACAGCCTGGA |
| | protein | | EVSSPSEALDVEPPVSEALNMEPAV | GAAGCTGCAACAGATCGAGCCACAAAAGCTGGAGTCCAAGACCGACG |
| | (etramp | | GESANFEDSAKGEVDIEPVSEVESIE | AGAGCGACCCACTCCTGGGCAAGAGCGAGGCTGCTAAGGTGGAGGTC |
| | 10.2) | | PVSEVESIEPVSEVESIEPSVDEVMD | AAGGGCGACTCCGAGGAAGTGCCACAAGAGGTGTCCTCCCCGAGCGA |
| | | | AAEPISTEPVNVEPAGNETENIVPTS | GGCTCTGGACGTGGAGCCACCAGTCTCCGAGGCCCTGAACATGGAGCC |
| | | | FEQVNIEPAVSEAFSQERSGEETAD | AGCCGTGGGCGAGTCCGCCAACTTCGAGGACAGCGCCAAGGGCGAGG |
| | | | FEDSVKEDVIPESPPVESVTIEAENI | TCGACATCGAGCCAGTGTCCGAGGTCGAGTCTATTGAACCAGTGTCCG |
| | | | QPMNVEQMNVDPTVSDAESIEPTPV | AGGTGGAGTCTATTGAGCCAGTGTCCGAAGTCGAGAGCATCGAGCCAT |
| | | | EAVDIEPVNVEPVNVEPAVSETMSQ | CCGTGGACGAGGTCATGGACGCTGCTGAGCCAATCAGCACCGAGCCA |
| | | | EPSLDEVENVESAVNEMMSQEPSA | GTGAACGTCGAGCCAGCCGGCAACGAGACGGAGAACATCGTGCCAAC |
| | | | EETANFAHSIKEDVSPESTSVESLDV | CTCCTTCGAGCAAGTGAACATCGAGCCAGCCGTCAGCGAGGCCTTCTC |
| | | | ESSVSEPMSTDPSPVESVSMESVD | CCAAGAGAGGAGCGGCGAGGAGACGGCTGACTTCGAGGACTCCGTGA |
| | | | SETVNVESIDSETVNVEPSDETSKV | AGGAAGACGTCATCCCAGAGTCCCCACCAGTGGAGAGCGTCACCATCG |
| | | | EADVQQFTDEELSTIGNVADKASDG | AGGCCGAGAACATCCAACCGATGAACGTGGAGCAGATGAACGTGGAC |
| | | | PAPEASDFPDSIFEENLDNANPPLKL | CCAACCGTCTCCGACGCCGAGAGCATCGAGCCAACCCCAGTGGAGGCC |
| | | | EDALVDPPASDEAQPEPSHPNEAV | GTGGATATCGAGCCTGTCAACGTGGAGCCTGTCAACGTTGAGCCAGCC |
| | | | GAAKSAESAEADQISHSGSGDASPS | GTGTCCGAGACGATGAGCCAAGAGCCATCCCTCGACGAGGTGGAGAA |
| | | | APSSSDDTSGSKNSGTSGKDRLFKT | CGTCGAGAGCGCCGTCAACGAGATGATGTCCCAGGAGCCATCCGCTGA |
| | | | YDSDVEPPIVPEKYPTVGVKEAPKM | GGAGACGGCCAACTTCGCCCACTCCATCAAGGAAGACGTGAGCCCAGA |
| | | | GFAEMAFKNIFDTFSKVADASKVLTP | GAGCACCTCCGTCGAGTCCCTGGACGTGGAGTCCAGCGTCAGCGAGCC |
| | | | EKQSAPEKQSAPEKQSAPEKQSAP | AATGTCCACCGACCCAAGCCCAGTGGAGAGCGTCTCCATGGAGTCCGT |
| | | | EKHSTPPKQSTSPKESTSPKQPAPP | GGACAGCGAGACGGTGAACGTCGAGTCCATCGATTCCGAGACGGTCA |
| | | | KPSTSPKQSAPAKQSAPPKQSAPAK | ACGTGGAGCCATCCGACGAGACGAGCAAGGTGGAGGCCGACGTCCAA |
| | | | QSAPAKNAAPPQSASSSRFFSSSSN | CAGTTCACCGACGAGGAGCTCAGCACCATCGGCAACGTGGCTGACAAG |
| | | | GNKGFGLRLFSDASSSNNKKGRAG | GCTTCCGACGGCCCAGCTCCAGAGGCCTCCGACTTCCCAGACAGCATCT |
| | | | NPIIRFKRRANhhhhhh | TCGAGGAGAACCTCGACAACGCCAACCCACCACTCAAGCTGGAGGACG |
| | | | (SEQ ID NO: 85) | CTCTGGTGGACCCACCAGCTAGCGACGAGGCTCAACCAGAGCCATCCC |
| | | | | ACCCAAACGAGGCTGTGGGCGCTGCTAAGTCCGCTGAGAGCGCTGAG |
| | | | | GCTGACCAAATCAGCCACTCCGGCAGCGGCGACGCTTCCCCAAGCGCT |
| | | | | CCATCCAGCTCCGACGACACCTCCGGCAGCAAGAACTCCGGCACCAGC |
| | | | | GGCAAGGACAGGCTCTTCAAGACCTACGACTCCGACGTGGAGCCACCA |
| | | | | ATCGTCCCAGAGAAGTACCCAACCGTGGGCGTGAAGGAAGCCCCAAA |
| | | | | GATGGGCTTCGCCGAGATGGCCTTCAAGAACATCTTCGACACCTTCTCC |
| | | | | AAGGTGGCTGACGCTAGCAAGGTCCTGACCCCAGAGAAGCAATCCGCC |
| | | | | CCAGAGAAGCAGAGCGCTCCTGAGAAGCAGAGCGCTCCCGAGAAGCA |
| | | | | GAGCGCCCCAGAGAAGCACTCCACCCCACCAAAGCAATCCACCAGCCC |
| | | | | AAAGGAGTCCACCAGCCCAAAGCAGCCAGCCCCACCAAAGCCATCCAC |
| | | | | CAGCCCTAAGCAGTCCGCTCCAGCTAAGCAGTCCGCCCCACCAAAGCA |
| | | | | GAGCGCTCCAGCTAAGCAATCCGCTCCAGCTAAGAACGCTGCCCCACC |
| | | | | ACAGAGCGCCAGCTCCAGCAGGTTCTTCTCCAGCTCCAGCAACGGCAA |
| | | | | CAAGGGCTTCGGCCTCAGGCTGTTCTCCGACGCCTCCAGCTCCAACAAC |
| | | | | AAGAAGGGCAGGGCCGGCAACCCAATCATCCGCTTCAAGAGGCGCGC |
| | | | | CAACCACCACCACCACCACCACTGA (SEQ ID NO: 86) |
| 44 | hypothe- | PVX_ | MNNPAEVVAAHLRRTGNSNEIRQAS | ATGAACAACCCAGCTGAGGTGGTGGCTGCTCACCTGAGGCGCACCGGC |
| | tical | 091500 | HVESVGGSANSSLDDDDGGGYDSA | AACTCCAACGAGATCAGGCAGGCTAGCCACGTGGAGAGCGTCGGCGG |
| | protein, | | APPGELHTTGDAPPGEFRTTGVVPP | CTCCGCTAACTCCAGCCTCGACGACGACGACGGCGGCGGATACGACAG |
| | conserved | | GRQKGGKKRMFKIKKKKSLTPLHID | CGCTGCCCCACCAGGCGAGCTCCACACCACCGGCGACGCCCCACCAGG |
| | | | DGGFTQGGEAKGPDVALESFAITRK | CGAGTTCCGCACCACCGGCGTGGTCCCACCAGGCAGGCAAAAGGGCG |
| | | | RRRPPLLGRGVVESSNIELTSKLGG | GCAAGAAGCGCATGTTCAAGATCAAGAAGAAGAAGTCCCTCACCCCAC |
| | | | KLGSKLGGKLNPTLSLVASRAVDGL | TGCACATCGACGACGGCGGCTTCACCCAGGGCGGCGAGGCTAAGGGC |
| | | | LGGVHKHMQGPFSLDLDGTNNSPL | CCAGACGTGGCTCTGGAGTCCTTCGCCATCACCAGGAAGAGGCGCAGG |
| | | | ATPIVTPNLYSNISTPFNMHNGIPPS | CCACCACTCCTGGGCCGCGGCGTGGTCGAGTCCAGCAACATCGAGCTC |
| | | | APAPMALPPQGVQVPLPNAQPQPP | ACCAGCAAGCTGGGCGGCAAGCTCGGCTCCAAGCTGGGCGGCAAGCT |
| | | | PSVATTATAAPAATSPMASPTTPTP | CAACCCGACCCTCAGCCTGGTGGCCTCCAGGGCCGTGGACGGCCTCCT |
| | | | AASTGVPPPPGIQLATNAMTYPQMN | GGGCGGCGTGCACAAGCACATGCAAGGCCCATTCAGCCTCGACCTGGA |
| | | | MQNVMTANQMAQNPAFNIHPTATN | CGGCACCAACAACTCCCCACTGGCCACCCCAATCGTCACCCCAAACCTC |
| | | | LRDDPGNVNYNEVVTITIGIVICLFLF | TACTCCAACATCAGCACCCCATTCAACATGCACAACGGCATCCCACCAA |
| | | | CFVFGCIVKMCKPAKRRRhhhhhh | GCGCTCCAGCTCCAATGGCTCTGCCACCACAAGGCGTGCAGGTCCCAC |
| | | | (SEQ ID NO: 87) | TCCCAAACGCCCAACCACAACCACCACCATCCGTGGCTACCACCGCTAC |
| | | | | CGCTGCTCCAGCTGCTACCAGCCCAATGGCTTCCCCAACCACCCCAACC |
| | | | | CCAGCTGCTAGCACCGGCGTGCCACCACCACCAGGCATCCAGCTGGCC |
| | | | | ACCAACGCCATGACCTACCCACAGATGAACATGCAGAACGTCATGACC |
| | | | | GCCAACCAAATGGCCCAGAACCCAGCCTTCAACATCCACCCGACCGCTA |
| | | | | CCAACCTCAGGGACGACCCAGGCAACGTGAACTACAACGAGGTGGTC |
| | | | | ACCATCACCATCGGCATCGTCATCTGCCTCTTCCTGTTCTGCTTCGTGTT |
| | | | | CGGCTGCATCGTCAAGATGTGCAAGCCGGCTAAGCGCAGGCGCCATCA |
| | | | | CCACCACCACCACTGA (SEQ ID NO: 88) |
| 45 | hypothe- | PVX_ | mSKTGNNNRNAKNAKGGGGGGKR | ATGTCCAAGACCGGCAACAACAACAGGAACGCCAAGAACGCTAAGGG |
| | tical | 090145 | GNNEANKNDGMSGKGSQKGKKKD | CGGCGGCGGCGGCGGCAAGAGGGGCAACAACGAGGCCAACAAGAAC |
| | protein, | | PGGGGTPKGQGKGPEQGKQKNKK | GACGGCATGTCCGGCAAGGGCAGCCAAAAGGGCAAGAAGAAGGACC |
| | conserved | | GEDSHFDEYIKDMKNSQDEDNFMD | CAGGCGGCGGCGGCACCCCGAAGGGCCAGGGCAAGGGCCCAGAGCA |
| | | | ELNRFEKNFHDEDFESDENLFNYGK | AGGCAAGCAGAAGAACAAGAAGGGCGAGGACTCCCACTTCGACGAGT |
| | | | GGTHSGEFNKIGELNSGNYNEMKP | ACATCAAGGACATGAAGAACAGCCAAGACGAGGACAACTTCATGGAC |
| | | | DANDYQYFDNEDILEGDEDLTNIWN | GAGCTCAACAGGTTCGAGAAGAACTTCCACGACGAGGACTTCGAGTCC |
| | | | KNMQNFEPSTLLTFEIQGNSEEYLF | GACGAGAACCTGTTCAACTACGGCAAGGGCGGCACCCACTCCGGCGA |
| | | | EEVTSLNTYFRGVFYSNNESDDNKI | GTTCAACAAGATCGGCGAGCTCAACAGCGGCAACTACAACGAGATGA |
| | | | LFFITDPDGEVIYKKEASEGIFYFYTQ | AGCCAGACGCCAACGACTACCAGTACTTCGACAACGAGGACATCCTGG |
| | | | KIGVYTITLKNSKWMGKKLTTVALGL | AGGGCGACGAGGACCTGACCAACATCTGGAACAAGAACATGCAAAAC |
| | | | GESPSLKSEHIKDFTNYIDKIVAETKR | TTCGAGCCAAGCACCCTCCTGACCTTCGAGATCCAGGGCAACTCCGAG |
| | | | LKNELKYLSSKHMTHIEKMKKITNKA | GAGTACCTCTTCGAGGAAGTGACCAGCCTGAACACCTACTTCCGCGGC |
| | | | FLYCFIKLFVLVFLSLFTIYYIKNLVSN | GTCTTCTACTCCAACAACGAGAGCGACGACAACAAGATCCTGTTCTTCA |
| | | | KRVLhhhhhh (SEQ ID NO: 89) | TCACCGACCCAGACGGCGAGGTCATCTACAAGAAGGAAGCCTCCGAG |
| | | | | GGCATCTTCTACTTCTACACCCAAAAGATCGGCGTGTACACCATCACCC |
| | | | | TCAAGAACAGCAAGTGGATGGGCAAGAAGCTGACCACCGTGGCTCTG |
| | | | | GGCCTGGGCGAGTCCCCAAGCCTCAAGAGCGAGCACATCAAGGACTTC |
| | | | | ACCAACTACATCGACAAGATCGTCGCCGAGACGAAGAGGCTGAAGAA |
| | | | | CGAGCTCAAGTACCTGTCCAGCAAGCACATGACCCACATCGAGAAGAT |
| | | | | GAAGAAGATCACCAACAAGGCCTTCCTCTACTGCTTCATCAAGCTCTTC |
| | | | | GTGCTGGTCTTCCTCTCCCTGTTCACCATCTACTACATCAAGAACCTCGT |
| | | | | GAGCAACAAGCGCGTCCTGCACCACCACCACCACCACTGA |
| | | | | (SEQ ID NO: 90) |
| 46 | hypothe- | PVX_ | MNNHQAVKQQMNPKGSKEQNRMV | ATGAACAACCACCAAGCCGTCAAGCAACAGATGAACCCAAAGGGCTCC |
| | tical | 119265 | APNSNMPGGMRDLAYHRNNGNNE | AAGGAGCAGAACAGGATGGTGGCCCCAAACAGCAACATGCCAGGCGG |
| | protein, | | MGKMNMNANGQQHNAGSSNTYNS | CATGAGGGACCTCGCTTACCACAGGAACAACGGCAACAACGAGATGG |
| | conserved | | NSINNNNYSLGLYIDNPQNAFVFDE | GCAAGATGAACATGAACGCCAACGGCCAACAGCACAACGCCGGCTCCA |
| | | | NDLKTLFSHYKGAKNIRILNDKAAAQ | GCAACACCTACAACTCCAACTCCATCAACAACAACAACTACTCCCTCGG |
| | | | ITFNDKNMIQQVRKDINGLTITDIGTI | CCTGTACATCGACAACCCACAAAACGCCTTCGTCTTCGACGAGAACGAC |
| | | | RCIILNEGKIVEQFLPFSANDPASAQ | CTCAAGACCCTGTTCAGCCACTACAAGGGCGCCAAGAACATCAGGATC |
| | | | QKGGSNQSGDSTVDMLKKLANLLQ | CTCAACGACAAGGCTGCCGCCCAGATCACCTTCAACGACAAGAACATG |
| | | | PERAMDSSMAPKMGDNGGLSATG | ATCCAACAGGTCAGGAAGGACATCAACGGCCTGACCATCACCGACATC |
| | | | SVNMGASIATNVGMGGNMPTNANM | GGCACCATCCGCTGCATCATCCTCAACGAGGGCAAGATCGTGGAGCAA |
| | | | GGVITTNANVSANVSANVSANPMPG | TTCCTGCCATTCTCCGCCAACGACCCGGCTAGCGCTCAACAGAAGGGC |
| | | | KNQVKNKMGNHAIYNNGGSHFNQA | GGCTCCAACCAAAGCGGCGACTCCACCGTGGACATGCTCAAGAAGCTC |
| | | | HMNKGEPGENNPYATKRLSRIELIDI | GCTAACCTCCTGCAGCCAGAGAGGGCCATGGACTCCAGCATGGCCCCA |
| | | | FGFPVEFDVMKKILGKNNSNISYIKE | AAGATGGGCGACAACGGCGGCCTCTCCGCTACCGGCTCCGTCAACATG |
| | | | QTNNSVSIEIKGKPFNEAPIVERMHV | GGCGCCTCCATCGCCACCAACGTGGGCATGGGCGGCAACATGCCAACC |
| | | | SVSSDDLIGYKKATELIVKLLNSIFEE | AACGCCAACATGGGCGGCGTCATCACCACCAACGCCAACGTGAGCGCC |
| | | | FYDFCYEKNYPVPENLSFKRHEYMY | AACGTCTCCGCTAACGTGAGCGCTAACCCAATGCCAGGCAAGAACCAA |
| | | | NPDGSTKYVGFKDKWHVMKDSYRT | GTGAAGAACAAGATGGGCAACCACGCCATCTACAACAACGGCGGCTCC |
| | | | DYSFRKNKGLQKNDKDKRMHGGAF | CACTTCAACCAGGCCCACATGAACAAGGGCGAGCCAGGCGAGAACAA |
| | | | GGHPNLSIGYANQNAPQGDFKEMN | CCCATACGCCACCAAGAGGCTCAGCCGCATCGAGCTGATCGACATCTTC |
| | | | hhhhhh (SEQ ID NO: 91) | GGCTTCCCAGTCGAGTTCGACGTGATGAAGAAGATCCTCGGCAAGAAC |
| | | | | AACAGCAACATCTCCTACATCAAGGAGCAAACCAACAACTCCGTCAGC |
| | | | | ATCGAGATCAAGGGCAAGCCATTCAACGAGGCCCCAATCGTGGAGCG |
| | | | | CATGCACGTGTCCGTCTCCAGCGACGACCTCATCGGCTACAAGAAGGC |
| | | | | CACCGAGCTGATCGTCAAGCTCCTGAACAGCATCTTCGAGGAGTTCTAC |
| | | | | GACTTCTGCTACGAGAAGAACTACCCAGTGCCAGAGAACCTGTCCTTCA |
| | | | | AGAGGCACGAGTACATGTACAACCCAGACGGCAGCACCAAGTATGTG |
| | | | | GGCTTCAAGGACAAGTGGCACGTGATGAAGGACTCCTACAGGACCGA |
| | | | | CTACAGCTTCCGCAAGAACAAGGGCCTCCAGAAGAACGACAAGGACA |
| | | | | AGAGGATGCACGGCGGCGCTTTCGGCGGACACCCAAACCTGAGCATC |
| | | | | GGCTACGCCAACCAAAACGCCCCACAGGGCGACTTCAAGGAGATGAA |
| | | | | CCACCACCACCACCACCACTGA (SEQ ID NO: 92) |
| 47 | rhoptry | PVX_ | mREAKGSVRDGKQYVKTKSPTYTP | ATGCGCGAGGCTAAGGGCTCCGTGCGCGACGGCAAGCAATACGTCAA |
| | neck | 117880 | QKKTKVIFYMPGQEQEEEEDDNDP | GACCAAGAGCCCAACCTACACCCCACAGAAGAAGACCAAGGTCATCTT |
| | protein | | NGSKKNGKSDTGANKGTHMGSKTD | CTACATGCCAGGCCAAGAGCAAGAGGAAGAGGAAGACGACAACGACC |
| | 2, puta- | | AGNSPSGLNKGSGVGSGSRPASNN | CAAACGGCTCCAAGAAGAACGGCAAGAGCGACACCGGCGCCAACAAG |
| | tive | | YKGNAGGGINIDMSPHGDNSNKGQ | GGCACCCACATGGGCTCCAAGACCGACGCTGGCAACTCCCCGAGCGGC |
| | (RON2) | | QGNAGLNKNQEDTLRDEYEKIRKQE | CTCAACAAGGGCTCCGGCGTGGGCTCCGGCAGCAGGCCAGCCAGCAA |
| | | | EEEEERINNORRADMKRAQRGKNK | CAACTACAAGGGCAACGCCGGCGGCGGCATCAACATCGACATGTCCCC |
| | | | FGDDKGVQDShhhhhh | ACACGGCGACAACAGCAACAAGGGCCAACAGGGCAACGCCGGCCTCA |
| | | | (SEQ ID NO: 93) | ACAAGAACCAAGAGGACACCCTGAGGGACGAGTACGAGAAGATCCGC |
| | | | | AAACAAGAGGAAGAGGAAGAGGAGCGCATCAACAACCAAAGGCGCG |
| | | | | CTGACATGAAGAGGGCTCAGAGGGGCAAGAACAAGTTCGGCGACGAC |
| | | | | AAGGGCGTGCAAGACAGCCACCACCACCACCACCACTGA |
| | | | | (SEQ ID NO: 94) |
| 48 | trypto- | PVX_ | mSSQSAVDYIEQEPLDILNLEEGDLE | ATGTCCAGCCAAAGCGCCGTGGACTACATCGAGCAGGAGCCACTCGAC |
| | phan-rich | 121897 | VTEQWKDNEWHNWKLKLEEDWDS | ATCCTCAACCTCGAAGAGGGCGACCTGGAGGTCACCGAGCAGTGGAA |
| | antigen | | FSTSLIRDKKDFMKIKTDELNGWLNL | GGACAACGAGTGGCACAACTGGAAGCTCAAGCTCGAAGAGGACTGGG |
| | (Pv-fam-a) | | EENKWNNFSGYLSDGYKNYLLKKS | ACTCCTTCAGCACCTCCCTCATCAGGGACAAGAAGGACTTCATGAAGAT |
| | | | EKWNDADWENWANTEMVAHLDKD | CAAGACCGACGAGCTGAACGGCTGGCTCAACCTGGAGGAGAACAAGT |
| | | | YHLWSLNTERSVNALVRGEWNQW | GGAACAACTTCAGCGGCTACCTCTCCGACGGCTACAAGAACTACCTCCT |
| | | | QHDKMSSWLSSDWKKVGAMYWDL | GAAGAAGTCCGAGAAGTGGAACGACGCCGACTGGGAGAACTGGGCC |
| | | | QESRNWASYSHTDDMKEHWIKWN | AACACCGAGATGGTGGCCCACCTCGACAAGGACTACCACCTCTGGAGC |
| | | | DRNARENIEWSKWVQNKEYFIMYA | CTGAACACCGAGAGGTCCGTGAACGCTCTGGTCCGCGGCGAGTGGAA |
| | | | RHSDIEQWKYDNYALYSTWRNDFIN | CCAATGGCAGCACGACAAGATGTCCAGCTGGCTCTCCAGCGACTGGAA |
| | | | RWVSEKKWNSILNhhhhhh | GAAGGTCGGCGCCATGTACTGGGACCTGCAGGAGAGCAGGAACTGGG |
| | | | (SEQ ID NO: 95) | CCAGCTACTCCCACACCGACGACATGAAGGAGCACTGGATCAAGTGGA |
| | | | | ACGACAGGAACGCCCGCGAGAACATCGAGTGGTCCAAGTGGGTGCAA |
| | | | | AACAAGGAGTACTTCATCATGTACGCCCGCCACAGCGACATCGAGCAG |
| | | | | TGGAAGTACGACAACTACGCCCTCTACTCCACCTGGAGGAACGACTTC |
| | | | | ATCAACCGCTGGGTCAGCGAGAAGAAGTGGAACTCCATCCTGAACCAC |
| | | | | CACCACCACCACCACTGA (SEQ ID NO: 96) |
| 49 | trypto- | PVX_ | mKSSNEIERLTHVKLKDTSEWTENV | ATGAAGTCCAGCAACGAGATCGAGAGGCTCACCCACGTGAAGCTGAA |
| | phan-rich | 125728 | EEWVKDEWHEWMDEVQMDWKEF | GGACACCTCCGAGTGGACCGAGAACGTGGAGGAGTGGGTCAAGGAC |
| | antigen | | NSSLESEKNKWFGKKEKEMMELIKS | GAGTGGCACGAGTGGATGGACGAGGTCCAGATGGACTGGAAGGAGTT |
| | (Pv-fam-a) | | IEDKWLDFNENMHEVLNYAILKISLM | CAACTCCAGCCTGGAGTCCGAGAAGAACAAGTGGTTCGGCAAGAAGG |
| | | | WSFSEWQKWINKDGKRIIENQWER | AGAAGGAGATGATGGAGCTGATCAAGAGCATCGAGGACAAGTGGCTC |
| | | | WTISNKNLYYKIIMKEWFKWKNKKIK | GACTTCAACGAGAACATGCACGAGGTGCTCAACTACGCCATCCTCAAG |
| | | | QWLKRNWLHHEGRILENWERLPYT | ATCTCCCTGATGTGGTCCTTCAGCGAGTGGCAAAAGTGGATCAACAAG |
| | | | KILAMSEKKPWFNSNAQVINERDYF | GACGGCAAGAGGATCATCGAGAACCAGTGGGAGCGCTGGACCATCAG |
| | | | LIWIKKKEDFLVNEERDKWENWEYY | CAACAAGAACCTGTACTACAAGATCATCATGAAGGAGTGGTTCAAGTG |
| | | | KNDFFQTWMDSFLSHWLNIKKRDIL | GAAGAACAAGAAGATCAAGCAATGGCTCAAGAGGAACTGGCTGCACC |
| | | | HSQShhhhhh | ACGAGGGCAGGATCCTGGAGAACTGGGAGCGCCTGCCATACACCAAG |
| | | | (SEQ ID NO: 97) | ATCCTCGCCATGTCCGAGAAGAAGCCATGGTTCAACAGCAACGCCCAA |
| | | | | GTGATCAACGAGAGGGACTACTTCCTGATCTGGATCAAGAAGAAGGA |
| | | | | AGACTTCCTCGTCAACGAGGAGCGCGACAAGTGGGAGAACTGGGAGT |
| | | | | ACTACAAGAACGACTTCTTCCAAACCTGGATGGACTCCTTCCTCAGCCA |
| | | | | CTGGCTGAACATCAAGAAGCGCGACATCCTCCACTCCCAGAGCCACCA |
| | | | | CCACCACCACCACTGA (SEQ ID NO: 98) |
| 50 | reticu- | PVX_ | mRLKHDHNLLPNYANLMRDDQNGQ | ATGAGGCTCAAGCACGACCACAACCTCCTGCCAAACTACGCCAACCTG |
| | locyte | 090330 | NSENRGDNINNHNKNHNDQNNHNG | ATGAGGGACGACCAAAACGGCCAGAACTCCGAGAACCGCGGCGACAA |
| | binding | | NNDNSINSEYLKTSHLQNSSAMVHL | CATCAACAACCACAACAAGAACCACAACGACCAAAACAACCACAACGG |
| | protein 2 | | NDHKITTKPARYSYIQRSKIYAFNPN | CAACAACGACAACTCCATCAACAGCGAGTACCTCAAGACCAGCCACCT |
| | precursor | | NKKIENINNELHShhhhhh | GCAGAACTCCAGCGCCATGGTGCACCTCAACGACCACAAGATCACCAC |
| | (PvRBP-2), | | (SEQ ID NO: 99) | CAAGCCAGCCAGGTACTCCTACATCCAACGCAGCAAGATCTACGCCTTC |
| | putative | | | AACCCAAACAACAAGAAGATCGAGAACATCAACAACGAGCTGCACTCC |
| | | | | CACCACCACCACCACCACTGA (SEQ ID NO: 100) |
| 51 | histone- | PVX_ | mSMEQGTPIVFPHKEGTILTKGTNN | ATGTCCATGGAGCAAGGCACCCCAATCGTGTTCCCACACAAGGAAGGC |
| | lysine N- | 123685 | LAVAHKEEVHRSEEETTLKGLKEEL | ACCATCCTCACCAAGGGCACCAACAACCTGGCCGTGGCCCACAAGGAA |
| | methyltra | | PHEHTLAIQKYDPSFGRGGSPGSGS | GAGGTGCACAGGAGCGAGGAAGAGACGACCCTCAAGGGCCTGAAGG |
| | nsferase, | | TEHTNGSFSNSYETILYNKSNDVVK | AAGAGCTCCCACACGAGCACACCCTGGCCATCCAGAAGTACGACCCAA |
| | H3 lysine- | | NLKEIKKGAPFGGVISDAVSCPASSS | GCTTCGGCCGCGGCGGCTCCCCAGGCAGCGGCAGCACCGAGCACACC |
| | 4 specific, | | SNTGGNKNLCFSNMMKLSKKILGFP | AACGGCTCCTTCAGCAACTCCTACGAGACGATCCTCTACAACAAGTCCA |
| | putative | | LLTDFERGMSTNQPCLPLSDHLKRL | ACGACGTGGTCAAGAACCTGAAGGAGATCAAGAAGGGCGCTCCATTC |
| | (SET10) | | SVCTVCYSKHNDLAKAIICRVTKMHF | GGCGGCGTGATCTCCGACGCCGTCTCCTGCCCGGCCTCCAGCTCCAGC |
| | | | EANYNDGLGDEDMFKTSSECIQSVI | AACACCGGCGGCAACAAGAACCTCTGCTTCAGCAACATGATGAAGCTC |
| | | | RELANTIKEYRKRELSGAYVQELAR | TCCAAGAAGATCCTGGGCTTCCCACTCCTGACCGACTTCGAGAGGGGC |
| | | | SGSSSYRSCSSSSYSSRGGSCAGS | ATGAGCACCAACCAACCATGCCTCCCACTGAGCGACCACCTCAAGCGC |
| | | | RGDGLAGSHGEIHAVIAGPPLTDDH | CTGTCCGTGTGCACCGTCTGCTACAGCAAGCACAACGACCTGGCCAAG |
| | | | NDIGAEAHSPSSSLKLPPQKPFYGM | GCCATCATCTGCAGGGTGACCAAGATGCACTTCGAGGCCAACTACAAC |
| | | | MSDPPCSDRRPGDTNNPFENNTPP | GACGGCCTCGGCGACGAGGACATGTTCAAGACCTCCAGCGAGTGCATC |
| | | | LLWDNKVNYTDDYTCKRGEVNSTL | CAATCCGTGATCCGCGAGCTGGCCAACACCATCAAGGAGTACAGGAAG |
| | | | GKRPHEEDNKGSSQKKSKLRTKPS | CGCGAGCTGTCCGGCGCCTACGTCCAAGAGCTCGCTAGGTCCGGCTCC |
| | | | NDTIGGENGDSLKGGTDEGKTHEG | AGCTCCTACAGGAGCTGCAGCTCCAGCTCCTACAGCTCCAGGGGCGGC |
| | | | GGNVGSCTAQGGADQLPRSDLCRD | AGCTGCGCTGGCTCCCGCGGCGACGGCCTCGCCGGCTCCCACGGCGAG |
| | | | PRGDPCVDPLPEQHAHRSKDENQK | ATCCACGCCGTCATCGCTGGCCCACCACTGACCGACGACCACAACGAC |
| | | | GDKNDIHFAGEKLDEIEAPGDQKGN | ATCGGCGCTGAGGCTCACAGCCCAAGCTCCAGCCTCAAGCTGCCACCA |
| | | | YVTLENISKASNFIPLLGVELGSTKIQ | CAAAAGCCATTCTACGGCATGATGTCCGACCCACCATGCTCCGACAGG |
| | | | REFTNGTYVGTVTEQIKDEHGNPFF | CGCCCAGGCGACACCAACAACCCATTCGAGAACAACACCCCACCACTCC |
| | | | VVTYEDGDAEWMTPCFLFQELLKQ | TGTGGGACAACAAGGTGAACTACACCGACGACTACACCTGCAAGAGG |
| | | | STNSVDYPLATTFKEVFNPEFKKDL | GGCGAGGTCAACTCCACCCTCGGCAAGCGCCCACACGAGGAAGACAA |
| | | | KLSNCSLELKIERRKRKSNCESASN | CAAGGGCTCCAGCCAGAAGAAGTCCAAGCTCAGGACCAAGCCAAGCA |
| | | | NNSVSKRQKHAQEENSSRKKKQRF | ACGACACCATCGGCGGCGAGAACGGCGACAGCCTGAAGGGCGGCACC |
| | | | hhhhhh (SEQ ID NO: 101) | GACGAGGGCAAGACCCACGAGGGCGGCGGCAACGTGGGCTCCTGCAC |
| | | | | CGCCCAAGGCGGCGCCGACCAGCTCCCAAGGTCCGACCTGTGCAGGG |
| | | | | ACCCACGCGGCGACCCATGCGTCGACCCACTCCCAGAGCAACACGCCC |
| | | | | ACCGCTCCAAGGACGAGAACCAGAAGGGCGACAAGAACGACATCCAC |
| | | | | TTCGCCGGCGAGAAGCTCGACGAGATCGAGGCCCCAGGCGACCAAAA |
| | | | | GGGCAACTACGTGACCCTGGAGAACATCAGCAAGGCCTCCAACTTCAT |
| | | | | CCCGCTCCTGGGCGTGGAGCTGGGCAGCACCAAGATCCAACGCGAGTT |
| | | | | CACCAACGGCACCTACGTGGGCACCGTCACCGAGCAGATCAAGGACG |
| | | | | AGCACGGCAACCCATTCTTCGTGGTCACCTACGAGGACGGCGACGCTG |
| | | | | AGTGGATGACCCCATGCTTCCTCTTCCAAGAGCTCCTGAAGCAGAGCAC |
| | | | | CAACTCCGTGGACTACCCACTGGCCACCACCTTCAAGGAAGTGTTCAAC |
| | | | | CCAGAGTTCAAGAAGGACCTCAAGCTGAGCAACTGCTCCCTGGAGCTG |
| | | | | AAGATCGAGAGGCGCAAGAGGAAGTCCAACTGCGAGAGCGCCTCCAA |
| | | | | CAACAACAGCGTGTCCAAGCGCCAAAAGCACGCCCAAGAGGAGAACT |
| | | | | CCTCCAGGAAGAAGAAGCAGCGCTTCCACCACCACCACCACCACTGA |
| | | | | (SEQ ID NO: 102) |
| 52 | reticu- | PVX_ | mTFNDGSDEISTAQKYKTDVEGIIDK | ATGACCTTCAACGACGGCAGCGACGAGATCTCCACCGCCCAAAAGTAC |
| | locyte | 125738 | LNVIDETINGINSTLDELLELGNNCQL | AAGACCGACGTGGAGGGCATCATCGACAAGCTGAACGTCATCGACGA |
| | binding | | HRTFLISSSLNNKIAKFLVEIREQKEN | GACGATCAACGGCATCAACAGCACCCTGGACGAGCTCCTGGAGCTCGG |
| | protein 1 | | TKKCFQYVKRNHQHLANFVSELHKT | CAACAACTGCCAACTCCACAGGACCTTCCTGATCTCCAGCTCCCTCAAC |
| | precursor, | | QGGIFENVNLVDNTPDADKYYHEFM | AACAAGATCGCCAAGTTCCTCGTGGAGATCAGGGAGCAGAAGGAGAA |
| | putative | | EIEQEATKIVKDIKKEIYHLNDDVDEP | CACCAAGAAGTGCTTCCAATACGTGAAGCGCAACCACCAGCACCTGGC |
| | | | VLEKRIKDVINTYNKLKTKKVQMDQS | CAACTTCGTCTCCGAGCTCCACAAGACCCAAGGCGGCATCTTCGAGAA |
| | | | YKNMYITKLREVEGSHDLFNQVAQLI | CGTCAACCTGGTGGACAACACCCCAGACGCCGACAAGTACTACCACGA |
| | | | RGETDKKGKALSERENNLHSIYNFV | GTTCATGGAGATCGAGCAAGAGGCCACCAAGATCGTCAAGGACATCA |
| | | | KLHETELHNLYAKYTPEYMEKINKIF | AGAAGGAGATCTACCACCTGAACGACGACGTGGACGAGCCAGTCCTG |
| | | | DDINARMIAVDLNDDHSSEYSDVKR | GAGAAGAGGATCAAGGACGTGATCAACACCTACAACAAGCTGAAGAC |
| | | | HEHEAMLLMDATNNLSKEVEMMQN | CAAGAAGGTCCAGATGGACCAGTCCTACAAGAACATGTACATCACCAA |
| | | | ESGGKNDGINGGKSQLVEDYTNTM | GCTGAGGGAGGTGGAGGGCAGCCACGACCTGTTCAACCAAGTCGCCC |
| | | | SEFTEQAKTVAKKIHDSKGDYANMF | AGCTCATCAGGGGCGAGACGGACAAGAAGGGCAAGGCCCTGTCCGAG |
| | | | DHIRENEAMLERIDLKKKDIKEILAHL | CGCGAGAACAACCTCCACAGCATCTACAACTTCGTGAAGCTGCACGAG |
| | | | NRMKEYLLKKLSEEEKLHHMREKLE | ACGGAGCTCCACAACCTGTACGCCAAGTACACCCCAGAGTACATGGAG |
| | | | EVNTSTDEIVKKFRTYDQMVDISQNI | AAGATCAACAAGATCTTCGACGACATCAACGCCAGGATGATCGCCGTG |
| | | | DIKNVQSKRYDSVDEIDKEMSYIKTH | GACCTCAACGACGACCACAGCTCCGAGTACAGCGACGTCAAGCGCCAC |
| | | | NKDLIDSKFIVERALENDKRKKSEMA | GAGCACGAGGCCATGCTCCTGATGGACGCCACCAACAACCTGTCCAAG |
| | | | QIFSTISRDNSSMYEYAKSFFDSVLK | GAAGTGGAGATGATGCAGAACGAGAGCGGCGGCAAGAACGACGGCA |
| | | | EIEKLTQMIRNMDKLINENEAVMEKL | TCAACGGCGGCAAGTCCCAACTCGTGGAGGACTACACCAACACCATGA |
| | | | KDQRRELQNVENASTDLGKLEEVD | GCGAGTTCACCGAGCAGGCCAAGACCGTCGCCAAGAAGATCCACGACT |
| | | | KMAQTKSETELSERNDSRNAKDGA | CCAAGGGCGACTACGCCAACATGTTCGACCACATCAGGGAGAACGAG |
| | | | TYSTLMDDKETDSVNGEETKQENV | GCCATGCTGGAGCGCATCGACCTCAAGAAGAAGGACATCAAGGAGAT |
| | | | VVKKGLPPQTDIYTSVVLKNDRNDQ | CCTCGCCCACCTGAACAGGATGAAGGAGTACCTCCTGAAGAAGCTGTC |
| | | | KSEKIGEKKSNKPVGTEENIQHSSYL | CGAGGAAGAGAAGCTCCACCACATGCGCGAGAAGCTCGAAGAGGTGA |
| | | | NNDNSNNDIDVGTLYTLGGYNAPND | ACACGAGCACCGACGAGATCGTCAAGAAGTTCCGCACCTACGACCAAA |
| | | | NYNTNESGDDINEEAKKKRNAVLFV | TGGTGGACATCTCCCAGAACATCGACATCAAGAACGTGCAAAGCAAGC |
| | | | YVGGLFSALFICIGAVFYLLHRKIGIE | GCTACGACTCCGTCGACGAGATCGACAAGGAGATGTCCTACATCAAGA |
| | | | GVGKSDHEKKPTIEDTKIEVFEETNG | CCCACAACAAGGACCTGATCGACAGCAAGTTCATCGTCGAGAGGGCCC |
| | | | SKRNVKDEVIDVPFVDMEDNLhhhhh | TGGAGAACGACAAGCGCAAGAAGAGCGAGATGGCCCAAATCTTCAGC |
| | | | h (SEQ ID NO: 103) | ACCATCTCCAGGGACAACAGCTCCATGTACGAGTACGCCAAGAGCTTC |
| | | | | TTCGACTCCGTGCTGAAGGAGATCGAGAAGCTCACCCAGATGATCCGC |
| | | | | AACATGGACAAGCTCATCAACGAGAACGAGGCCGTCATGGAGAAGCT |
| | | | | GAAGGACCAAAGGCGCGAGCTCCAGAACGTGGAGAACGCCTCCACCG |
| | | | | ACCTCGGCAAGCTCGAAGAGGTGGACAAGATGGCCCAGACCAAGAGC |
| | | | | GAGACGGAGCTGTCCGAGAGGAACGACAGCCGCAACGCTAAGGACG |
| | | | | GCGCTACCTACTCCACCCTCATGGACGACAAGGAGACGGACAGCGTGA |
| | | | | ACGGCGAGGAGACGAAGCAAGAGAACGTGGTCGTGAAGAAGGGCCT |
| | | | | GCCACCACAGACCGACATCTACACCAGCGTCGTGCTCAAGAACGACAG |
| | | | | GAACGACCAAAAGTCCGAGAAGATCGGCGAGAAGAAGAGCAACAAGC |
| | | | | CAGTGGGCACCGAGGAGAACATCCAGCACAGCTCCTACCTCAACAACG |
| | | | | ACAACTCCAACAACGACATCGACGTGGGCACCCTCTACACCCTGGGCG |
| | | | | GCTACAACGCCCCAAACGACAACTACAACACCAACGAGAGCGGCGACG |
| | | | | ACATCAACGAGGAAGCCAAGAAGAAGAGGAACGCCGTGCTCTTCGTCT |
| | | | | ACGTGGGCGGCCTCTTCTCCGCCCTGTTCATCTGCATCGGCGCCGTGTT |
| | | | | CTACCTCCTGCACCGCAAGATCGGCATCGAGGGCGTCGGCAAGAGCGA |
| | | | | CCACGAGAAGAAGCCAACCATCGAGGACACCAAGATCGAGGTGTTCG |
| | | | | AGGAGACGAACGGCTCCAAGCGCAACGTCAAGGACGAGGTCATCGAC |
| | | | | GTGCCATTCGTCGACATGGAGGACAACCTCCACCACCACCACCACCACT |
| | | | | GA (SEQ ID NO: 104) |
| 53 | PvDBP | PVX_ | mGEHKTDSKTDNGKGANNLVMLDY | ATGGGCGAGCACAAGACCGACTCCAAGACCGACAACGGCAAGGGCGC |
| | (reqion | 110810 | ETSSNGQPAGTLDNVLEFVTGHEG | CAACAACCTGGTCATGCTCGACTACGAGACGTCCTCCAACGGCCAGCC |
| | II); | | NSRKNSSNGGNPYDIDHKKTISSAII | AGCTGGCACCCTGGACAACGTGCTGGAGTTCGTCACCGGCCACGAGG |
| | Duffy | | NHAFLQNTVMKNCNYKRKRRERDW | GCAACAGCAGGAAGAACTCCAGCAACGGCGGCAACCCATACGACATC |
| | receptor | | DCNTKKDVCIPDRRYQLCMKELTNL | GACCACAAGAAGACCATCTCCAGCGCCATCATCAACCACGCCTTCCTGC |
| | precursor | | VNNTDTNFHRDITFRKLYLKRKLIYD | AGAACACCGTGATGAAGAACTGCAACTACAAGAGGAAGAGGCGCGAG |
| | (DBP) | | AAVEGDLLLKLNNYRYNKDFCKDIR | CGCGACTGGGACTGCAACACCAAGAAGGACGTCTGCATCCCAGACAG |
| | | | WSLGDFGDIIMGTDMEGIGYSKVVE | GCGCTACCAACTCTGCATGAAGGAGCTGACCAACCTCGTGAACAACAC |
| | | | NNLRSIFGTDEKAQQRRKQWWNES | CGACACCAACTTCCACAGGGACATCACCTTCCGCAAGCTGTACCTCAAG |
| | | | KAQIWTAMMYSVKKRLKGNFIWICK | AGGAAGCTGATCTACGACGCTGCTGTGGAGGGCGACCTCCTGCTCAAG |
| | | | LNVAVNIEPQIYRWIREWGRDYVSE | CTCAACAACTACAGGTACAACAAGGACTTCTGCAAGGACATCCGCTGG |
| | | | LPTEVQKLKEKCDGKINYTDKKVCK | TCCCTGGGCGACTTCGGCGACATCATCATGGGCACCGACATGGAGGGC |
| | | | VPPCQNACKSYDQWITRKKNQWDV | ATCGGCTACTCCAAGGTGGTCGAGAACAACCTCCGCAGCATCTTCGGC |
| | | | LSNKFISVKNAEKVQTAGIVTPYDILK | ACCGACGAGAAGGCCCAACAGAGGCGCAAGCAATGGTGGAACGAGTC |
| | | | QELDEFNEVAFENEINKRDGAYIELC | CAAGGCCCAGATCTGGACCGCCATGATGTACAGCGTGAAGAAGAGGC |
| | | | VCSVEEAKKNTQEVVhhhhhh | TGAAGGGCAACTTCATCTGGATCTGCAAGCTCAACGTGGCCGTCAACA |
| | | | (SEQ ID NO: 105) | TCGAGCCACAGATCTACAGGTGGATCAGGGAGTGGGGCAGGGACTAC |
| | | | | GTCTCCGAGCTGCCAACCGAGGTGCAAAAGCTCAAGGAGAAGTGCGA |
| | | | | CGGCAAGATCAACTACACCGACAAGAAGGTGTGCAAGGTCCCACCATG |
| | | | | CCAAAACGCCTGCAAGAGCTACGACCAGTGGATCACCAGGAAGAAGA |
| | | | | ACCAATGGGACGTCCTGTCCAACAAGTTCATCAGCGTGAAGAACGCCG |
| | | | | AGAAGGTCCAGACCGCCGGCATCGTGACCCCATACGACATCCTGAAGC |
| | | | | AAGAGCTCGACGAGTTCAACGAGGTGGCCTTCGAGAACGAGATCAAC |
| | | | | AAGCGCGACGGCGCCTACATCGAGCTCTGCGTGTGCAGCGTCGAGGA |
| | | | | AGCCAAGAAGAACACCCAAGAGGTGGTCCACCACCACCACCACCACTG |
| | | | | A (SEQ ID NO: 106) |
| 54 | MSP3.10 | PVX_ | mVIGGSPNNEAPNSSRHHLRNGFP | ATGGTCATCGGCGGCTCCCCAAACAACGAGGCCCCAAACTCCAGCAGG |
| | [merozo- | 097720 | GKNDSLPHEEPNNLEGKNESSDQC | CACCACCTCCGCAACGGCTTCCCAGGCAAGAACGACTCCCTCCCACACG |
| | ite | | DTINLGOVTEKEKKTIEQASVQAQD | AGGAGCCAAACAACCTGGAGGGCAAGAACGAGTCCAGCGACCAATGC |
| | surface | | ATKPEANNAEQIQAELQKVKTAKDE | GACACCATCAACCTGGGCCAGGTGACCGAGAAGGAGAAGAAGACCAT |
| | protein | | SATAAKDAETAKKNAVDAGKGLDAA | CGAGCAAGCTAGCGTCCAAGCTCAGGACGCTACCAAGCCAGAGGCCA |
| | 3 alpha | | KGAIKKAEEAAAEAKKQAGIAEKAEK | ACAACGCCGAGCAAATCCAGGCCGAGCTCCAAAAGGTGAAGACCGCT |
| | (MSP3a)] | | DAEAAGKKDKLEDVNSQVQIAVEAS | AAGGACGAGTCCGCTACCGCTGCTAAGGACGCTGAGACGGCCAAGAA |
| | | | TKAKDKKTEAEIAVEIVKAVVAKEEA | GAACGCTGTGGACGCTGGCAAGGGCCTGGACGCCGCCAAGGGCGCCA |
| | | | QKASDEAQKACEKAQKAHAKAQKA | TCAAGAAGGCTGAGGAAGCCGCCGCCGAGGCCAAGAAGCAGGCTGGC |
| | | | SDTTKTVETFKTNAEAAAKNAKEKA | ATCGCCGAGAAGGCTGAGAAGGACGCTGAGGCTGCTGGCAAGAAGG |
| | | | GNANKAATEAESANELSVAKQKAKD | ACAAGCTGGAGGACGTGAACAGCCAAGTCCAGATCGCCGTGGAGGCC |
| | | | AEEAAKEAKKEQVKAEIAAEVAKAK | TCCACCAAGGCCAAGGACAAGAAGACCGAGGCCGAGATCGCCGTGGA |
| | | | VAKEEADAAQKKAEAAKKIVDKIAQD | GATCGTCAAGGCCGTGGTCGCCAAGGAAGAGGCCCAAAAGGCTAGCG |
| | | | TKVPEAQREAKLATQTASKATEAAT | ACGAGGCTCAGAAGGCTTGCGAGAAGGCCCAAAAGGCTCACGCTAAG |
| | | | EAGKKAQEAEESSKEAEEKAETSDA | GCTCAGAAGGCTTCCGACACCACCAAGACCGTGGAGACGTTCAAGACC |
| | | | VKGKADAAEKAAGEAKKASIETEIAI | AACGCCGAGGCTGCCGCCAAGAACGCCAAGGAGAAGGCTGGCAACGC |
| | | | EVAKAEVLNAEVKKTAQEAEKDATE | TAACAAGGCTGCTACCGAGGCTGAGAGCGCTAACGAGCTCTCCGTGGC |
| | | | AKEQAEKAKAAAEEAKTHGEKAEKV | CAAGCAGAAGGCCAAGGACGCCGAGGAAGCCGCCAAGGAAGCCAAG |
| | | | GESTKAHSDEAQQENKNAKDASEE | AAGGAGCAAGTCAAGGCTGAGATCGCTGCTGAGGTGGCTAAGGCTAA |
| | | | AENRAVDALEEAYAVEAHLARTKNA | GGTGGCTAAGGAAGAGGCCGACGCTGCTCAGAAGAAGGCTGAGGCC |
| | | | AESAKSATDMSELEKAKEEAIDAANI | GCCAAGAAGATCGTGGACAAGATCGCCCAAGACACCAAGGTGCCGGA |
| | | | AHQKWLKATQAATIAKEKKEAAKVA | GGCTCAGAGGGAGGCTAAGCTGGCTACCCAGACCGCTAGCAAGGCTA |
| | | | AEKAQTAANVVKDKAAKAEAKKAET | CCGAGGCCGCCACCGAGGCTGGCAAGAAGGCTCAAGAGGCCGAGGA |
| | | | EAVKAAVEARAAAEEAKQEAAKVGA | GTCCAGCAAGGAAGCCGAGGAGAAGGCTGAGACGAGCGACGCTGTG |
| | | | SKEPQETKNKANVEAEATGNEAKKA | AAGGGCAAGGCTGACGCTGCTGAGAAGGCTGCTGGCGAGGCCAAGAA |
| | | | EDAAEEAKEAAKKANEATDANVARS | GGCTTCCATCGAGACGGAGATCGCCATCGAGGTCGCCAAGGCCGAGG |
| | | | EADKAIAAAKKAKKAREKAAYGLLKT | TGCTCAACGCCGAGGTCAAGAAGACCGCTCAAGAGGCCGAGAAGGAC |
| | | | KNQYVLEPLDISPESADNITSKEEQV | GCTACCGAGGCCAAGGAGCAAGCCGAGAAGGCCAAGGCTGCCGCCGA |
| | | | KEEMEDQGDEDSNEAEVEEALPNG | GGAAGCCAAGACCCACGGCGAGAAGGCTGAGAAGGTGGGCGAGAGC |
| | | | SGAQEEDVNLEMDDEEEVEEVEEN | ACCAAGGCCCACTCCGACGAGGCCCAACAGGAGAACAAGAACGCCAA |
| | | | VATNQQTGGKREKRNTNDTVDDTN | GGACGCCAGCGAGGAAGCCGAGAACAGGGCTGTGGACGCTCTCGAAG |
| | | | ADKQFGDEFDTYNDIKKVTEALVKS | AGGCCTACGCTGTGGAGGCTCACCTGGCTAGGACCAAGAACGCTGCTG |
| | | | MTSLVSDDPSVGDTINEFLSDMNHL | AGTCCGCTAAGAGCGCTACCGACATGTCCGAGCTGGAGAAGGCCAAG |
| | | | FLSWhhhhhh | GAAGAGGCCATCGACGCCGCCAACATCGCCCACCAAAAGTGGCTCAAG |
| | | | (SEQ ID NO: 107) | GCTACCCAGGCTGCTACCATCGCTAAGGAGAAGAAGGAAGCCGCCAA |
| | | | | GGTGGCTGCTGAGAAGGCTCAGACCGCTGCCAACGTGGTCAAGGACA |
| | | | | AGGCTGCTAAGGCTGAGGCCAAGAAGGCTGAGACGGAGGCCGTCAAG |
| | | | | GCTGCTGTGGAGGCCAGGGCCGCCGCCGAGGAAGCCAAACAAGAGGC |
| | | | | CGCTAAGGTCGGCGCTAGCAAGGAGCCACAAGAGACGAAGAACAAGG |
| | | | | CTAACGTGGAGGCTGAGGCTACCGGCAACGAGGCCAAGAAGGCCGAG |
| | | | | GACGCTGCTGAGGAAGCCAAGGAAGCCGCCAAGAAGGCTAACGAGGC |
| | | | | TACCGACGCTAACGTGGCTAGGTCCGAGGCTGACAAGGCTATCGCCGC |
| | | | | CGCCAAGAAGGCCAAGAAGGCCCGCGAGAAGGCTGCTTACGGCCTCC |
| | | | | TGAAGACCAAGAACCAATACGTGCTGGAGCCACTGGACATCTCCCCAG |
| | | | | AGAGCGCCGACAACATCACCTCCAAGGAAGAGCAGGTGAAGGAAGAG |
| | | | | ATGGAGGACCAAGGCGACGAGGACAGCAACGAGGCCGAGGTGGAGG |
| | | | | AAGCCCTGCCAAACGGCTCCGGCGCTCAAGAGGAAGACGTCAACCTG |
| | | | | GAGATGGACGACGAGGAAGAGGTGGAGGAAGTGGAGGAGAACGTG |
| | | | | GCCACCAACCAACAGACCGGCGGCAAGAGGGAGAAGCGCAACACCAA |
| | | | | CGACACCGTCGACGACACCAACGCCGACAAGCAATTCGGCGACGAGTT |
| | | | | CGACACCTACAACGACATCAAGAAGGTGACCGAGGCCCTCGTCAAGTC |
| | | | | CATGACCAGCCTGGTGTCCGACGACCCATCCGTGGGCGACACCATCAA |
| | | | | CGAGTTCCTCAGCGACATGAACCACCTCTTCCTGTCCTGGCACCACCA |
| | | | | CCACCACCACTGA (SEQ ID NO: 108) |
| 55 | sexual | PVX_ | mENNKIKGGKVPPPSVPTGNNSDN | ATGGAGAACAACAAGATCAAGGGCGGCAAGGTGCCACCACCATCCGT |
| | stage | 000930 | NVPKKDGGENNPPPDAENALQELK | CCCAACCGGCAACAACTCCGACAACAACGTGCCAAAGAAGGACGGCG |
| | antigen | | NFTKNLEKKTTTNRNIIISTTVINMVLL | GCGAGAACAACCCACCACCAGACGCCGAGAACGCCCTCCAAGAGCTGA |
| | s16, | | VLLSGLIGYNTKKGFKKGQMGSVKE | AGAACTTCACCAAGAACCTGGAGAAGAAGACCACCACCAACAGGAAC |
| | putative | | VTPEAQKGKLhhhhhh | ATCATCATCTCCACCACCGTCATCAACATGGTGCTCCTGGTCCTCCTGA |
| | | | (SEQ ID NO: 109) | GCGGCCTGATCGGCTACAACACCAAGAAGGGCTTCAAGAAGGGCCAAA |
| | | | | TGGGCTCCGTGAAGGAAGTGACCCCAGAGGCCCAGAAGGGCAAGCTC |
| | | | | CACCACCACCACCACCACTGA (SEQ ID NO: 110) |
| 56 | | Posi- | | |
| | | tive | | |
| | | Cont- | | |
| | | rol? | | |
| 57 | | Nega- | | |
| | | tive | | |
| | | Cont- | | |
| | | rol? | | |

TABLE 5
list of protein references for additional 25 proteins

| Protein | | Protein | |
|---|---|---|---|
| Code | Protein Name | Reference | Source |
| X1 | PVX_094350 | PVX_094350 | Ehime University |
| X2 | PVX_099930 | PVX_099930 | Ehime University |
| X3 | PVX_114330 | PVX_114330 | Ehime University |
| X4 | PVX_088820 | PVX_088820 | Ehime University |
| X5 | PVX_080665 | PVX_080665 | Ehime University |
| X6 | PVX_092995 | PVX_092995 | Ehime University |
| X7 | PVX_087885 | PVX_087885 | Ehime University |
| X8 | PVX_003795 | PVX_003795 | Ehime University |
| X9 | PVX_087110 | PVX_087110 | Ehime University |
| X10 | PVX_087670 | PVX_087670 | Ehime University |
| X11 | PVX_081330 | PVX_081330 | Ehime University |
| X12 | PVX_122805 | PVX_122805 | Ehime University |
| V1 | RBP1b (P7) | PVX_098582 | WEHI |
| V2 | RBP2a (P9) | PVX_121920 | WEHI |
| V3 | RBP2b (P25) | PVX_094255 | WEHI |
| V4 | RBP2cNB (M5) | PVX_090325 | WEHI |
| V12 | RBP1a (P5) | PVX_098585 | WEHI |
| V5 | RBP2-P2 (P55) | PVX_101590 | WEHI |
| V11 | PvEBP | KMZ83376.1 | IPP |
| V10 | Pv DBPII (AH) | AAY34130.1 | IPP |
| V13 | Pv DBPII (SaII) | PVX_110810 | IPP |
| V6 | PvDBP R3-5 | PVX_110810 | WEHI |
| V7 | PvGAMA | PVX_088910 | WEHI |
| V8 | PvRipr | PVX_095055 | WEHI |
| V9 | PvCYRPA | PVX_090240 | WEHI |

List of protein sequences (insert aa sequence)
X1:
(SEQ ID NO: 111)
ENPVRHSVDIKSEDFVVLISLQNLQTFIMIGYTAVNKDHLNFDFSYLWALCIGTGLFIYSL
ISFVLIRSLALSKIDIGKYVLELLFSLSIIATCSLSIIIDSFKIANMQLLFFSFALTGYAYYNL
MSLFFFCTLVGMTIQYNLSFTGFRAHSTSFFFLDMLSYLVQMIGGNILYFRMYELCTLIVI
SKRNPCKYVVASKEVKQVEKQIFSSLFNSYMCIKSKTYSDLTCTNDLLNKDSQSVVGRD
TNPKWNSPIGTSYQDKVNHTKKLLLRRGKRDKRYPKGGGGARLTCAKHSAYHNSRSL
ANCASKNTPICTTNFRISNTLSLKNHFNPNLTLEASPPVCKKCVSEKNSHKDNEYKNGEE
RKKAKRGIKSGTANKSNQLGNHGGDATQVANPTYRTTSHGGDATQVAYPTYRTTSHG
GDATQVDSPTHPTTSHGGNNSSSGHPQDDEVLIPIRGTNATNDAAATYNSNASWIKTAA
VIDVSVEGKQKKGGHQTFAGNPVNSSANFPSDKKPSYNSHRNGGTPPPNEQLRYYACPC
YQTHSSGSSLSEVPSGQTTKRKNSAHNSVEGGNPKMDNQQSRRVSNKRVDGATGEEHD
HPSDPPADNPNGNSNTYHC
X2
(SEQ ID NO: 112)
ELSHSLSVKNAPDASALNIEVEKDKKKICKNAFQYINVAELLSPREEETYVQKCEEVLDT
IKNDSPDESAEAEINEFILSLLHARSKYTIINDSDEEVLSKLLRSINGSISEEAALKRAKQLI
TFNRFIKDKAKVKNVQEMLVISSKADDFMNEPKQKMLQKIIDSFELYNDYLVILGSNINI
AKRYSSETFLSIKNEKFCSDHIHLCQKFYEQSIIYYRLKVIFDNLVTYVDQNSKHFKKEKL
LELLNMDYRVNRESKVHENYVLEDETVIPTMRITDIYDQDRLIVEVVQDGNSKLMHGR
DIEKREISERYIVTVKNLRKDLNDEGLYADLMKTVKNYVLSITQIDNDISNLVRELDHED
VEK
X3
(SEQ ID NO: 113)
LPWTKKRKAVNQMGIIKDMSQELRTKAEQLPTPEDISAKIHRVDKEVIDKLNKDIIEEEN
LDKHKPHVCQEPAYERDYSYLCPEDWVKNSNDQCWGIDYDGHCEALKYFQDYSVEEK
KEFEMNCCVLWPKLKNEGMKGAHKKDLLRGSISSNNGLIIKPKYL
X4
(SEQ ID NO: 114)
ELKKNNAALTSQRSSSRTTSTRSYKNAPKNSTSFLSRLSILIFALSCAIFVNTASGAAANR
PNANGFVSPTLIGFGELSIQESEEFKRMAWNNWMLRLESDWKHFNDSVEEAKTKWLHE
RDSAWSDWLRSLQSKWSHYSEKMLKEHKSNVMEKSANWNDTQWGNWIKTEGRKILE
AQWEKWIKKGDDQLQKLILDKWVQWKNDKIRSWLSSEWKTEEDYYWANVERATTAK
WLQEAEKMHWLKWKERINRESEQWVNWVQMKESVYINVEWKKWPKWKNDKKILFN
KWSTNLVYKWTLKKQWNVWIKEANTAPQV
X5
(SEQ ID NO: 115)
KGVTLSCVFSHASEEREGGTGTFALSNEPIYYAPSGGLAPCALISRGLSGDEEGSGEDGG
EDGDGDGGEDSAEDNAEDGDDDGGEDGGLPGGRFPYEEGKKSSLVSDAPSDLLDGDA
DEHAAEDGGAKRKMSKKEEEAEDNKIDKLVNAEMKKLEAGEEANKDPDAEPEKEDQG
SGQGQRAKLRCSNKLNYIQVTANGQREGDLFGENDGESAPAFVEIPHEVEEESGGVPTK
HDEAGEAAAAEEPHNRVDRAEKENNAKDLKFVEGERERQRSSPPSNGYSQNSFVELKG
VPDKLPPNFTNSLGSSPTHSNLEKPVYKHLPWSILASDSGSNTGSWADVNSSTYNVSPFS
FTSIRSGNSLHLLPMNFQIQNSIVKVTDEEYDKLKLKNSVKVYDKNALVDYKYEIFEVKE
GEEYNDGNDPYEERNGEEGDAGGEGGSDGEGDADSKSYQNNKSDGRGFFDGTLVTYTI
IILAGVIILLLSFVIYYYDIINKVKRRMSAKRKNNKSMAIANDTSAGMYMGDTYMENPH
V
X6
(SEQ ID NO: 116)
SQGCSGYRLPPPKRWFTFTSRPYCKTAAYYELKHMPYYVDAVSASENVKHEKWNNWL
KEMKISLTEKLEKESQEYMEKLEQQWDEFMKNSEDKWRHYNPQMEEEYQCSVYPLGL
KWDDEKWTAWFYEKGLWCLKKSFKTWLTDSKKGYNTYMKNLLQEFGKQFYEDWCR
RPEKRREDKICKRWGQKGLRNDNYYSLKWMQWRNWKNRNHDQKHVWVTLMKDAL
KEYTGPEFKLWTEFRKEKIDFYKQWMQAFAEQWTQDKQWNTWTEERNEYMKKKKEE
EAKKKAASKKKAASKKGGAAKKAPAKKAPTKKAAPGTKAPAKKAAPKKVAAPNAA
X7
(SEQ ID NO: 117)
KEAVKKGSKKAMKQPMHKPNLLEEEDFEEKESFSDDEMNGFMEESMDASKLDAKKAK
TTLRSSEKKKTPTSGMSGMSGSGATSAATEAATNMNATAMNAAAKGNSEASKKQTDL
SNEDLFNDELTEEVIADSYEEGGNVGSEEAESLTNAFDDKLLDQGVNENTLLNDNMIYN
VNMVPHKKRELYISPHKHTSAASSKNGKHHAADADALDKKLRAHELLELENGEGSNSV
IVETEEVDVDLNGGKSSGSVSFLSSVVFLLIGLLCFTN
X8
(SEQ ID NO: 118)
NLSNDCKKGANNSFKLIVHTSDDILTLKWKVTGEGAAPGNKADVKKYKLPTLERPFTSV
QVHSANAKSKIIESKFYDIGSGMPAQCSAIATNCFLSGSLEIEHCYHCTLLEKKLAQDSEC
FKYVSSEAKELIEKDTPIKAQEEDANSADHKLIESIDVILKAVYKSDKDEEKKELITPEEV
DENLKKELANYCTLLKEVDTSGTLNNHQMANEEETFRNLTRLLRMHSEENVVTLQDKL
RNAAICIKHIDKWILNKRGLTLPEEGYPSEGYPPEEYPPEELLKEIEKEKSALNDEAFAKD
TNGVIHLDKPPNEMKFKSPYFKKSKYCNNEYCDRWKDKTSCMSNIEVEEQGDCGLCWI
FASKLHLETIRCMRGYGHFRSSALFVANCSKRKPEDRCNVGSNPTEFLQIVKDTGFLPLE
SDLPYSYSDAGNSCPNKRNKWTNLWGDTKLLYHKRPNQFAQTLGYVSYESSRFEHSID
LFIDILKREIQNKGSVIIYIKTNNVIDYDENGRVVHSLCGHKDADHAANLIGYGNYISAGG
EKRSYWIVRNSWGYYWGDEGNFKVDMYGPEGCKRNFIHTAVVFKIDLGIVEVPKKDEG
SIYSYFVQYVPNFLHSLFYVSYGKGADKGAAVVTGQAGGAVVTGQTETPTPEAAKNGD
QPGAQGSEAEVAEGGQAGNEAPGGLQESAVSSQTSEVTPQSSITAPQIGAVAPQIGAAAP
QIDVAAPQIDVVAPQTRSVDAPQTSSVAAHPPNVTPQNVTLGEGQHAGGVGSLIPADN
X9
(SEQ ID NO: 119)
ETLLDSETLKNYEKETNEYIRKKKVEKLFDVILKNVLVNKPENVYLYIYKNIYSFLLNKIF
VIGPPLLKITPTLCSAIASCFSYYHLSASHMIESYTTGEVDDAAESSTSKKLVSDDLICSIV
KSNINQLNAKQKRGYVVEGFPGTNLQADSCLRHLPSYVFVLYADEEYIYDKYEQENNV
KIRSDMNSQTFDENTQLFEVAEFNTNPLKDEVKVYLRN
X10
(SEQ ID NO: 120)
YPKKNFDKPDPTSPYQGQYGESEEQRQGYGIPPNPIMINLIGNQDQRPNVLQQFGINNK
NVMQFLINMFVYVAAILVSLKIWDYMSYSKCDYYKDLLLRIVRYQSHMNDGKMA
X11
(SEQ ID NO: 121)
SRIDKQPIQSSYLFQDNAVPPVRFSAVDADLFSIGVVHTEEQIFMDDANWVISSVPSKYL
NLHLLKTGSRPHFSHFSVSMNTGCNLFIASPVGETFPLSPSKDGATWKAFETDDSVEVIH
RETKEKRIYKLKFIPLKSGALLKVDVLKGIPFWVISQGRKILPTICSGDEEVLSNPQNEVF
KECTSSSSLSPEFDCLAGLSTYHRDKKNHTWKTSSGSIGQFIKIFFNKPVQITKFRFKPRD
DLLSWPSEVALQFDTDEEVIIPILHTHNMGQNTTRLEHPIITTSVKVEVRDMYERASENT
GGSFEVIGSTCOMMEDDYMTHHAVIDITECDRRLESLPDVMPLTKGSKFLAICPRPCLSS
SNGGVIYGSDVYSTDSAVCGAAVHAGVCSREGEGSCHFLVVVRGGRANFVGALQNNV
LSLSRGGGGSGSGSSTSSDGDGDSDSSTSRANFSFSLSSASGFGGGPRGAHAEAAPSSYSI
VFKPRDHLAPTNGFLVDSGREFTSYGSVAYGWKREVSPSSSFSSPSPSYTSPPLEEPTLLR
GDSSSFNGIYSGGIEFPPASASQNCISQLDCQTNFWKFQMQENGTYFVQVLVGNKTSPEK
QKAFVELNGVPIIKGVDLGPDEVFVATDRVQVTNRALVLTSTCLGGESACSRARVSIMA
VQIVKT
X12
(SEQ ID NO: 122)
NGMNKDKDAEITPPPFIVLPGGKKIHMLQSEYEYDVLRDMYRTDEANGGSGEKESHPSG
DGAIRRNEFFKLFHHREGHYKFVIKNVPTKLSDLLQKGGNEQETDLFPLLYRSLQFACSA
DGTWPYARREVAFFKNGSVHCEAEFQNELSVRRTPRSGKKSFGRFPRGTLIKSSDLRSKI
VEGNSYDKRAAPLKSEKKKKALFLHPESVLYKMEEIFFYENPSVKSEIVGFVLFHDVCT
VTSLGHGAHPVNSPFLGSDLLEMIFGYCILHGFKKIRVKSESLNYETGIRTSFIEILLNGKT
ALEHLGLRLTNVAKFSKELYYVITGYTWKSDLVLSPIVRFEHDLYVHHDIEERFFLYVNK
MYRNMLHDLSFSCDENYYPYKNCYDIYPSVRRSQNNLCLFELNPIYEELKELFPDSCNIG
QRVRKCYEEIKKNVVCTHNGEGGEDGCKYYQFIVNTFIKPRRKTSFFIYHNMYVQEYLS
KKSYPYYLLLSEVIKNEENNFLEKGNYDLVADAQTHLFLNYVLQNSTFFIFWNFSTEFW
KRFRYIQAGPTGATSTPQKGQAVFCPMAYAYEFVEHLDTFYVRG
V6
(SEQ ID NO: 123)
SVEEAKKNTQEVVTNVDNAAKSQATNSNPISQPVDSSKAEKVPGDSTHGNVNSG
QDSSTTGKAVTGDGQNGNQTPAESDVQRSDIAESVSAKNVDPQKSVSKRSDDTASVTGI
AEAGKENLGASNSRPSESTVEANSPGDDTVNSASIPVVSGENPLVTPYNGLRHSKDNSDS
DGPAESMANPDSNSKGETGKGQDNDMAKATKDSSNSSDGTSSATGDTTDAVDREINKG
VPEDRDKTVGSKDGGGEDNSANKDAATVVGEDRIRENSAGGSTNDRSKNDTEKNGAS
TPDSKQSEDATALSKTESLESTESGDRTTNDTTNSLENKNGGKEKDLQKHDFKSNDTPN
EEPNSDQTTDAEGHDRDSIKNDKAERRKHMNKDTFTKNTNSHHLN
V7
(SEQ ID NO: 124)
IRNGNNPQALVPEKGADPSGGQNNRSGENQDTCEIQKMAEEMMEKMMKEKDV
FSSIMEPLQSKLTDDHLCSKMKYTNICLHEKDKTPLTFPCTSPQYEQLIHRFTYKKLCNS
KVAFSNVLLKSFIDKKNEENTFNTIIQNYKVLSTCIDDDLKDIYNASIELFSDIRTSVTEITE
KLWSKNMIEVLKTREQTIAGILCELRNGNNSPLVSNSFSYENFGILKVNYEGLLNQAYAA
FSDYYSYFPAFAISMLEKGGLVDRLVAIHESLTNYRTRNILKKINEKSKNEVLNNEEIMH
SLSSYKHHAGGTRGAFLQSRDVREVTQGDVSVDEKGDRATTAGGNQSASVAAAAPKD
AGPTVAAPNTAATLKTAASPNAAATNTAAPPNMGATSPLSNPLYGTSSLQPKDVAVLV
RDLLKNTNIIKFENNEPTSQMDDEEIKKLIESSFFDLSDNTMLMRLLIKPQAAILLIIESFIM
MTPSPTRDAKTYCKKALVNGQLIETSDLNAATEEDDLINEFSSRYNLFYERLKLEEL
V8
(SEQ ID NO: 125)
KEYCDQLSFCDVGLTHHFDTYCKNDQYLFVHYTCEDLCKTCGPNSSCYGNKYK
HKCLCNSPFESKKNHSICEARGSCDAQVCGKNQICKMVDAKATCTCADKYQNVNGVC
LPEDKCDLLCPSNKSCLLENGKKICKCINGLTLQNGECVCSDSSQIEEGHLCVPKNKCKR
KEYQQLCTNEKEHCVYDEQTDIVRCDCVDHFKRNERGICIPVDYCKNVTCKENEICKVV
NNTPTCECKENLKRNSNNECVFNNMCLVNKGNCPIDSECIYHEKKRHQCLCHKKGLVA
INGKCVMQDMCRSDQNKCSENSICVNQVNKEPLCICLFNYVKSRSGDSPEGGQTCVVD
NPCLAHNGGCSPNEVCTFKNGKVSCACGENYRPRGKDSPTGQAVKRGEATKRGDAGQ
PGQAHSANENACLPKTSEADQTFTFQYNDDAAIILGSCGIIQFVQKSDQVIWKINSNNHF
YIFNYDYPSEGQLSAQVVNKQESSILYLKKTHAGKVFYADFELGHQGCSYGNMFLYAH
REEA
V9
(SEQ ID NO: 126)
SKNIIILNDEITTIKSPIHCITDIYFLFRNELYKTCIQHVIKGRTEIHVLVQKKINSAW
ETQTTLFKDHMWFELPSVFNFIHNDEIIIVICRYKQRSKREGTICKRWNSVTGTIYQKEDV
QIDKEAFANKNLESYQSVPLTVKNKKFLLICGILSYEYKTANKDNFISCVASEDKGRTWG
TKILINYEELQKGVPYFYLRPIIFGDEFGFYFYSRISTNNTARGGNYMTCTLDVTNEGKKE
YKFKCKHVSLIKPDKSLQNVAKLNGYYITSYVKKDNFNECYLYYTEQNAIVVKPKVQN
DDLNGCYGGSFVKLDESKALFIYSTGYGVQNIHTLYYTRYD
List of polynucleotide sequences (insert bp sequence)
X1
(SEQ ID NO: 127)
GAGAACCCCGTGAGGCACTCGGTGGACATAAAGTCGGAAGACTTCGTCGTCC
TGATTTCGCTCCAAAACCTGCAGACCTTCATCATGATAGGGTACACAGCCGTGAACA
AAGACCACCTGAATTTCGACTTCTCCTACTTATGGGCCCTCTGCATCGGGACGGGCC
TCTTCATATACTCCCTCATCAGCTTTGTACTCATAAGATCCCTAGCACTGTCAAAAAT
AGACATAGGCAAATACGTCCTGGAGCTGCTATTCAGTTTGAGTATAATCGCCACATG
TTCACTCTCCATAATAATTGACTCTTTCAAAATAGCCAACATGCAGTTGCTTTTTTTT
TCGTTCGCTTTAACGGGCTATGCCTACTACAATTTGATGAGCCTCTTCTTTTTCTGCA
CACTGGTAGGAATGACCATTCAGTACAATTTAAGTTTCACTGGGTTCAGAGCGCATT
CGACTTCTTTCTTCTTTTTAGATATGCTATCTTACCTAGTGCAAATGATAGGAGGGAA
CATCCTCTACTTTCGCATGTACGAGCTGTGTACCCTAATCGTCATTTCGAAGAGGAA
CCCCTGCAAGTATGTTGTCGCATCGAAGGAAGTGAAACAAGTGGAGAAGCAAATTT
TCTCTTCTTTATTTAATTCTTACATGTGCATCAAGTCCAAAACTTATTCAGATTTAAC
CTGCACTAATGATCTGTTAAATAAAGACAGTCAATCTGTTGTCGGTAGGGATACGAA
CCCTAAGTGGAACTCCCCCATTGGTACTTCCTACCAGGATAAGGTCAATCATACGAA
GAAGTTACTCCTTCGGAGGGGAAAACGGGACAAACGCTACCCCAAAGGGGGAGGG
GGAGCTCGACTAACATGTGCAAAACATAGTGCCTACCATAATAGCCGAAGTCTTGCC
AACTGTGCCAGTAAGAATACCCCCATTTGCACAACTAACTTTAGGATATCTAACACC
CTTTCACTTAAAAATCATTTCAACCCTAACCTAACCTTAGAAGCGTCTCCCCCCGTTT
GTAAAAAATGCGTTTCGGAAAAGAATAGCCATAAGGATAATGAGTACAAAAACGGG
GAAGAGAGAAAAAAAGCAAAACGTGGTATCAAGTCGGGCACTGCAAACAAGTCTA
ACCAGTTGGGCAACCACGGGGGGGACGCTACGCAGGTGGCTAATCCTACCTACAGA
ACTACTTCCCACGGGGGGGACGCAACCCAGGTGGCTTATCCTACCTACAGAACTACT
TCCCACGGGGGGGACGCAACGCAGGTGGATAGTCCTACCCACCCAACTACCTCCCA
TGGGGGGAACAACTCGTCGAGCGGGCACCCCCAAGACGACGAAGTGCTCATCCCCA
TTAGGGGAACCAACGCCACTAACGATGCAGCCGCCACCTACAACTCGAACGCTAGT
TGGATCAAAACCGCTGCGGTTATTGACGTGTCTGTGGAGGGGAAGCAGAAAAAGGG
GGGACATCAAACGTTCGCGGGCAATCCCGTAAATTCATCCGCTAATTTCCCATCGGA
CAAGAAACCTTCCTACAACTCGCACCGCAACGGAGGTACTCCCCCCCCAAATGAAC
AACTCAGGTACTACGCCTGCCCCTGCTACCAGACCCACTCCAGCGGATCGTCCCTCA
GTGAGGTGCCCTCGGGACAAACGACGAAGCGGAAAAATAGTGCGCACAACTCGGTT
GAAGGGGGAAACCCCAAAATGGATAATCAGCAAAGTCGCCGCGTGAGTAACAAGC
GGGTAGATGGCGCAACGGGTGAGGAACATGACCACCCAAGTGACCCCCCCGCAGAT
AACCCAAATGGAAACTCCAACACCTACCACTGC
X2
(SEQ ID NO: 128)
GAGCTGAGCCACAGCTTGTCCGTGAAGAACGCGCCGGACGCGAGCGCGCTG
AACATCGAGGTGGAGAAGGACAAAAAGAAGATCTGCAAAAACGCATTCCAATACAT
AAACGTAGCTGAGCTGTTGTCCCCAAGGGAGGAAGAAACCTACGTGCAGAAATGTG
AAGAGGTCCTAGACACAATAAAGAATGACAGTCCAGATGAATCGGCAGAAGCAGA
GATAAACGAATTTATACTGAGCTTACTGCACGCTCGTTCTAAGTATACCATAATAAA
TGACTCAGATGAGGAGGTACTGAGCAAGCTCCTGAGGAGTATCAACGGATCGATAA
GTGAAGAGGCAGCGTTGAAGAGAGCCAAACAGCTAATCACATTCAATCGGTTTATA
AAAGACAAAGCGAAGGTAAAAAATGTGCAAGAGATGCTAGTAATAAGTAGCAAAG
CAGATGACTTCATGAATGAGCCGAAGCAAAAAATGCTCCAAAAAATTATAGATTCG
TTTGAACTGTATAATGATTACCTAGTCATTTTAGGGTCAAATATTAACATCGCCAAG
AGGTACTCCTCAGAAACGTTTCTTTCTATTAAAAATGAAAAGTTCTGCTCAGACCAC
ATCCACTTATGCCAGAAGTTCTACGAGCAGTCTATCATTTACTACAGATTGAAGGTT
ATTTTTGATAACCTGGTGACTTATGTAGATCAAAATTCCAAGCATTTTAAAAAGGAA
AAGTTGCTGGAGCTTCTAAATATGGATTATAGGGTCAATCGAGAGTCGAAGGTGCAT
GAAAATTACGTGCTGGAGGATGAGACGGTCATCCCCACGATGCGCATTACAGACAT
TTACGATCAAGATAGGCTAATTGTTGAGGTCGTTCAGGATGGAAATAGCAAGCTGAT
GCACGGCAGGGATATTGAGAAGAGGGAAATCAGCGAGAGGTACATCGTCACCGTGA
AGAACCTGCGCAAGGACCTCAACGACGAGGGGCTCTACGCCGACTTGATGAAGACC
GTCAAGAACTACGTGCTCTCCATCACGCAGATCGACAACGACATTTCCAACCTCGTG
CGCGAGCTCGACCACGAGGATGTGGAGAAG
X3
(SEQ ID NO: 129)
CTACCATGGACGAAGAAAAGAAAGGCGGTGAACCAAATGGGCATCATAAAA
GATATGTCGCAGGAGCTTAGGACTAAGGCCGAACAGCTTCCAACCCCCGAGGATAT
ATCAGCCAAAATTCACAGAGTAGATAAAGAGGTCATCGATAAGTTAAACAAAGACA
TCATAGAGGAAGAAAATTTAGACAAGCACAAACCGCACGTCTGCCAGGAGCCAGCA
TACGAGAGGGACTATTCGTACCTATGTCCCGAAGACTGGGTGAAGAACTCCAACGA
TCAGTGCTGGGGCATAGACTACGATGGTCACTGTGAAGCGCTAAAATATTTCCAAGA
TTATTCTGTAGAGGAGAAAAAAGAATTTGAAATGAACTGCTGCGTCTTGTGGCCTAA
GCTAAAAAATGAAGGCATGAAAGGAGCGCACAAGAAGGACCTCCTAAGGGGATCG
ATAAGTTCAAACAATGGGTTAATAATAAAGCCGAAATATTTG
X4
(SEQ ID NO: 130)
GAATTGAAGAAGAACAATGCCGCGTTGACCTCACAAAGGTCATCTTCTAGAA
CCACATCCACAAGGAGCTACAAAAATGCCCCAAAAAATTCCACTTCATTCCTTTCTC
GTTTATCTATTCTGATATTTGCCTTATCATGTGCTATTTTTGTAAATACTGCATCAGG
GGCGGCAGCTAATAGACCAAACGCGAATGGCTTTGTGTCACCTACTTTAATAGGATT
TGGCGAATTAAGCATCCAAGAATCAGAAGAATTCAAAAGAATGGCTTGGAATAATT
GGATGTTGCGATTGGAGTCCGACTGGAAACATTTTAACGATTCTGTTGAAGAAGCCA
AAACCAAATGGCTTCATGAAAGAGACTCAGCTTGGTCTGATTGGCTTCGTTCCTTGC
AAAGTAAATGGTCTCACTATAGTGAAAAAATGCTTAAAGAACACAAAAGTAATGTT
ATGGAAAAATCAGCCAACTGGAATGACACGCAATGGGGAAATTGGATAAAAACTGA
AGGAAGAAAAATTCTAGAAGCGCAATGGGAAAAATGGATTAAAAAAGGTGATGAC
CAATTACAAAAGTTAATTTTAGATAAATGGGTTCAATGGAAAAATGATAAGATCCG
ATCCTGGTTATCCAGTGAATGGAAAACCGAAGAAGATTACTACTGGGCAAATGTAG
AGCGCGCTACAACAGCAAAATGGTTGCAAGAAGCAGAGAAAATGCATTGGCTTAAA
TGGAAAGAAAGAATTAACAGAGAGTCTGAACAATGGGTGAACTGGGTCCAAATGAA
AGAAAGCGTTTACATCAATGTAGAATGGAAAAAATGGCCCAAATGGAAAAATGATA
AAAAAATTCTATTTAACAAATGGTCAACTAACCTTGTCTACAAATGGACACTGAAAA
AGCAGTGGAACGTTTGGATTAAGGAAGCAAATACTGCACCCCAAGTT
X5
(SEQ ID NO: 131)
AAGGGTGTCACCTTGAGTTGCGTTTTTTCCCATGCGAGTGAGGAACGTGAGG
GTGGCACAGGGACATTTGCTTTGAGCAATGAGCCGATTTATTACGCCCCTAGTGGGG
GGCTGGCGCCGTGCGCGCTCATCAGCAGAGGGTTAAGCGGGGATGAGGAGGGTAGC
GGCGAGGACGGCGGTGAAGATGGCGACGGAGATGGTGGTGAAGACAGCGCTGAGG
ACAACGCTGAGGATGGAGACGATGATGGTGGCGAAGATGGCGGCTTGCCCGGGGGA
CGCTTCCCATACGAAGAAGGAAAAAAGAGTAGCCTTGTGAGCGACGCACCCAGCGA
CCTCCTGGATGGAGATGCGGATGAACATGCCGCCGAAGATGGGGGAGCGAAGCGAA
AGATGAGTAAGAAGGAGGAAGAGGCGGAGGATAACAAAATTGACAAGTTGGTAAA
TGCGGAAATGAAAAAGCTCGAGGCAGGGGAAGAGGCGAACAAGGATCCCGACGCA
GAACCAGAAAAAGAGGACCAGGGAAGTGGCCAAGGACAAAGGGCGAAGCTGAGGT
GCTCAAACAAGCTAAATTACATACAGGTGACGGCGAATGGCCAAAGGGAGGGCGAC
CTCTTTGGCGAGAACGACGGGGAGAGCGCCCCAGCTTTCGTGGAGATACCCCACGA
GGTTGAGGAGGAAAGCGGCGGTGTGCCCACAAAGCATGACGAAGCGGGGGAAGCA
GCTGCGGCGGAGGAACCACATAACCGCGTCGACCGAGCGGAAAAAGAAAACAACG
CGAAGGACTTAAAATTTGTGGAGGGGGAGCGAGAAAGACAAAGGAGCAGCCCCCC
CTCGAATGGATATTCCCAAAACAGCTTTGTCGAACTGAAAGGTGTGCCCGATAAATT
GCCCCCTAATTTTACCAACTCGCTTGGTAGCTCCCCAACGCACAGTAATTTGGAGAA
ACCAGTTTATAAGCACTTACCCTGGTCTATCCTGGCATCCGACTCTGGTTCGAACAC
CGGGTCCTGGGCAGACGTCAACAGTAGTACCTACAATGTGAGTCCATTCAGTTTCAC
CTCAATACGTAGTGGTAACTCTCTGCATCTACTGCCGATGAATTTCCAAATCCAAAA
CTCCATCGTGAAAGTAACTGATGAGGAGTATGACAAATTGAAGCTTAAAAACAGCG
TCAAAGTGTATGACAAAAATGCCCTGGTAGATTATAAGTATGAAATTTTTGAGGTGA
AGGAAGGGGAGGAATATAATGATGGGAATGACCCTTATGAGGAAAGGAATGGGGA
AGAAGGGGATGCAGGTGGAGAGGGGGGTTCCGATGGGGAGGGAGATGCAGATTCT
AAATCATATCAAAATAACAAATCGGATGGACGTGGGTTCTTCGATGGGACCTTAGTA
ACCTACACCATTATCATTTTAGCTGGTGTTATAATTCTGCTGCTAAGTTTTGTCATTT
ATTACTACGATATAATAAATAAGGTGAAGAGGCGAATGAGTGCCAAGCGGAAGAAC
AACAAATCTATGGCCATCGCGAATGATACATCCGCGGGGATGTACATGGGCGACAC
CTACATGGAGAATCCCCACGTT
X6
(SEQ ID NO: 132)
TCACAAGGATGTTCAGGATACCGTTTACCACCACCAAAAAGATGGTTTACCTT
CACTTCTCGACCATACTGTAAAACAGCTGCATATTATGAACTTAAACATATGCCATA
TTATGTAGATGCAGTTAGTGCATCAGAAAACGTAAAACATGAGAAATGGAATAACT
GGTTAAAAGAAATGAAAATATCATTAACTGAAAAATTAGAAAAAGAATCACAAGAA
TATATGGAAAAATTGGAACAGCAATGGGATGAATTTATGAAAAATTCAGAAGATAA
ATGGAGGCATTATAATCCCCAAATGGAAGAAGAATATCAATGTAGTGTTTATCCACT
TGGATTAAAATGGGATGATGAAAAGTGGACTGCATGGTTTTATGAAAAAGGATTAT
GGTGTTTGAAGAAAAGCTTTAAAACATGGCTCACTGATTCTAAAAAAGGTTACAAC
ACCTACATGAAAAATCTTTTACAGGAATTTGGTAAACAATTTTATGAAGATTGGTGT
CGTAGACCTGAAAAACGTCGTGAAGATAAAATTTGCAAGAGATGGGGACAAAAAG
GATTACGTAATGACAATTACTATTCGTTAAAGTGGATGCAGTGGAGAAATTGGAAA
AACAGAAACCACGATCAAAAACATGTGTGGGTAACTCTTATGAAGGATGCGCTAAA
GGAATATACGGGGCCCGAATTCAAATTATGGACTGAGTTTAGAAAAGAAAAGATAG
ACTTTTACAAGCAATGGATGCAAGCTTTCGCCGAACAGTGGACACAAGACAAACAA
TGGAATACGTGGACTGAAGAAAGAAATGAATATATGAAAAAGAAAAAAGAAGAAG
AAGCAAAAAAAAAAGCAGCATCAAAAAAAAAAGCAGCATCAAAAAAAGGAGGAG
CAGCAAAAAAGGCACCAGCAAAAAAGGCACCAACAAAAAAAGCCGCACCAGGAAC
AAAGGCACCAGCAAAAAAAGCAGCACCTAAAAAAGTTGCAGCACCAAATGCAGCA
X7
(SEQ ID NO: 133)
AAGGAGGCAGTGAAGAAGGGGTCCAAGAAGGCAATGAAGCAGCCCATGCAC
AAGCCGAACCTTCTTGAAGAGGAAGACTTTGAGGAGAAAGAATCCTTTTCGGATGA
CGAGATGAATGGGTTCATGGAGGAGAGCATGGATGCTTCTAAGTTGGATGCGAAGA
AGGCCAAGACGACCCTCAGGAGCTCGGAGAAGAAGAAGACTCCAACGAGCGGAAT
GAGTGGAATGAGTGGAAGCGGCGCCACCAGCGCAGCCACCGAGGCAGCCACGAAC
ATGAACGCCACCGCCATGAACGCCGCTGCTAAGGGCAACAGCGAGGCGAGCAAAA
AGCAAACCGACTTGTCCAACGAAGACCTGTTCAACGACGAGCTCACAGAAGAGGTC
ATTGCAGATTCGTACGAAGAGGGAGGAAACGTGGGAAGCGAGGAAGCCGAAAGCC
TCACAAATGCATTTGACGACAAGCTACTAGACCAAGGAGTGAATGAAAATACTCTG
CTGAACGACAACATGATTTACAACGTCAATATGGTTCCACATAAGAAGCGAGAATT
ATACATCTCCCCACACAAGCATACCTCTGCAGCAAGCAGTAAAAATGGCAAACATC
ATGCGGCGGACGCGGACGCTTTGGACAAAAAACTGAGGGCTCACGAGCTGCTCGAG
CTGGAAAACGGAGAAGGCAGCAACTCAGTCATTGTCGAAACGGAAGAAGTGGATGT
TGACCTAAACGGAGGAAAGTCAAGCGGCTCCGTGTCCTTCCTCAGCTCCGTAGTCTT
CTTGCTCATCGGATTGTTATGTTTCACCAAT
X8
(SEQ ID NO: 134)
AACCTGAGCAACGATTGCAAAAAAGGAGCCAACAACAGCTTTAAGTTAATCG
TGCACACCAGCGATGATATTTTGACACTCAAGTGGAAGGTCACTGGGGAAGGGGCA
GCTCCAGGCAACAAAGCAGATGTAAAGAAGTACAAACTCCCTACCCTAGAGAGGCC
TTTCACTTCCGTGCAAGTGCATTCAGCCAACGCCAAGTCGAAGATAATCGAAAGCAA
ATTTTACGACATTGGCAGCGGCATGCCAGCCCAGTGCAGCGCGATCGCCACGAACT
GCTTCCTCAGCGGCAGCCTCGAAATCGAGCACTGCTACCACTGCACCCTGTTGGAGA
AGAAGCTGGCCCAAGACAGCGAGTGCTTCAAGTACGTCTCGAGTGAAGCGAAGGAG
TTGATCGAGAAAGACACGCCGATTAAAGCTCAAGAAGAAGACGCCAACTCTGCAGA
CCACAAACTGATCGAGTCCATAGACGTGATACTAAAGGCAGTGTACAAATCAGATA
AAGATGAGGAAAAGAAGGAGCTCATCACCCCGGAGGAAGTGGACGAAAATTTGAA
GAAAGAGCTAGCCAATTATTGTACCCTACTGAAGGAGGTAGACACAAGTGGCACTC
TTAACAACCACCAGATGGCAAACGAAGAGGAAACGTTCAGAAATTTGACTCGACTG
TTGCGAATGCATAGCGAAGAAAACGTGGTGACCCTTCAGGACAAACTGAGAAACGC
AGCCATATGCATCAAGCACATCGACAAGTGGATTCTTAACAAGAGGGGGTTGACCC
TACCGGAAGAAGGGTACCCATCGGAAGGGTACCCCCCAGAAGAGTACCCCCCGGAG
GAACTCCTCAAAGAAATCGAGAAGGAAAAAAGCGCTCTGAATGATGAAGCGTTCGC
TAAAGATACCAACGGAGTCATCCACCTGGATAAGCCTCCCAACGAAATGAAATTTA
AATCCCCCTATTTTAAAAAGAGCAAATACTGTAACAATGAGTACTGTGATAGGTGGA
AAGATAAAACGAGTTGCATGTCAAATATAGAAGTGGAAGAGCAAGGGGATTGCGG
GCTCTGTTGGATTTTCGCCTCTAAGTTACACTTAGAAACGATCAGGTGCATGAGAGG
GTATGGCCACTTCCGCAGCTCCGCTCTGTTTGTGGCCAACTGCTCGAAGAGGAAGCC
AGAAGATAGATGCAACGTGGGTTCTAACCCTACAGAGTTTCTTCAAATTGTTAAGGA
CACGGGATTTTTACCTCTAGAGTCCGATCTCCCCTACAGCTATAGCGACGCGGGGAA
CTCCTGCCCCAATAAAAGAAACAAGTGGACCAACCTGTGGGGGGATACCAAACTGC
TGTATCATAAGAGACCCAATCAGTTTGCACAAACACTCGGGTACGTTTCCTACGAAA
GCAGTCGCTTTGAGCACAGCATCGACCTCTTCATAGACATCCTCAAAAGGGAAATTC
AAAACAAAGGCTCCGTTATCATTTACATAAAAACCAACAATGTCATCGATTATGACT
TTAATGGAAGAGTCGTCCACAGCCTATGTGGCCATAAGGATGCAGATCATGCCGCTA
ACCTGATCGGTTATGGTAACTACATCAGTGCTGGTGGGGAGAAGAGGTCCTATTGGA
TTGTGCGAAACAGCTGGGGGTACTACTGGGGAGATGAAGGCAACTTTAAGGTTGAC
ATGTACGGCCCGGAGGGATGCAAACGGAACTTCATCCACACGGCTGTTGTGTTTAAG
ATAGACCTGGGCATCGTCGAAGTCCCGAAGAAGGACGAGGGGTCCATTTATAGCTA
CTTCGTTCAGTACGTCCCCAACTTTTTGCACAGCCTTTTCTACGTGAGTTACGGTAAG
GGTGCTGATAAGGGAGCGGCGGTGGTGACAGGGCAGGCGGGAGGAGCGGTAGTCA
CAGGACAGACTGAAACGCCCACTCCGGAGGCCGCTAAAAATGGGGATCAGCCAGG
AGCACAGGGTAGCGAGGCAGAAGTCGCGGAGGGTGGCCAGGCAGGAAATGAAGCC
CCGGGAGGGTTGCAAGAGAGTGCTGTTTCGTCGCAAACGAGTGAGGTTACGCCGCA
ATCTAGTATAACTGCTCCGCAAATCGGTGCAGTTGCCCCACAAATCGGTGCAGCTGC
CCCACAAATCGATGTAGCCGCCCCACAAATCGATGTAGTCGCCCCACAAACGAGGT
CCGTTGACGCCCCCCAAACGAGCTCGGTTGCCGCCCACCCCCCAAACGTGACGCCGC
AGAACGTGACGCTTGGGGAGGGCCAGCACGCGGGGGGTGTAGGCTCCCTCATCCCC
GCGGACAAC
X9
(SEQ ID NO: 135)
GAAACCCTGCTAGACAGCGAAACGTTAAAGAACTACGAAAAGGAAACGAAC
GAATACATTCGCAAAAAAAAAGTGGAGAAACTGTTCGATGTTATTTTAAAAAATGTT
CTGGTAAACAAACCGGAAAATGTATACCTGTACATATACAAGAACATTTATTCCTTC
CTTTTGAACAAAATTTTTGTGATCGGCCCTCCTTTGCTGAAAATTACTCCCACCTTAT
GTTCTGCGATTGCCAGCTGCTTTAGCTACTACCACCTCAGCGCCTCGCACATGATCG
AGTCTTACACTACTGGTGAAGTAGATGACGCTGCAGAGAGTTCCACAAGCAAAAAG
TTAGTCAGTGACGACTTAATCTGCTCCATCGTTAAAAGCAACATAAACCAGCTGAAC
GCGAAGCAAAAGCGGGGGTATGTAGTCGAAGGGTTCCCCGGCACCAATCTTCAGGC
AGACAGTTGCCTACGGCATTTGCCATCTTACGTTTTTGTCCTGTACGCCGACGAAGA
GTACATTTATGACAAGTACGAACAAGAGAACAACGTAAAAATTCGTTCAGACATGA
ACAGCCAAACTTTTGATGAAAACACACAGTTGTTCGAAGTGGCCGAGTTCAACACG
AATCCGCTGAAGGATGAGGTAAAGGTCTACTTAAGGAAC
X10
(SEQ ID NO: 136)
TATCCAAAAAAGAACTTCGACAAACCCGACCCAACTTCCCCATACCAAGGAC
AATATGGAGAGTCTGAGGAACAAAGACAAGGTTATGGAATCCCCCCCAACCCAACC
ATGATTAACCTTACTGGTAACCAAGACCAACGACCAAATGTATTGCAACAATTTGGA
ATAAACAACAAAAATGTAATGCAGTTTTTAATAAACATGTTTGTGTACGTTGCTGCT
ATATTAGTTAGTTTAAAAATATGGGACTACATGTCTTACAGCAAATGTGATTATTAC
AAAGATTTATTATTAAGAATTGTAAGATACCAATCACACATGAATGATGGTAAGATG
GCC
X11
(SEQ ID NO: 137)
AGCCGCATCGACAAGCAGCCCATCCAGAGCAGCTACCTCTTCCAGGATAACG
CAGTCCCGCCTGTTCGATTCTCCGCAGTAGATGCAGACCTGTTTTCCATTGGAGTAGT
TCACACAGAGGAGCAAATATTTATGGACGACGCCAACTGGGTGATTAGCAGCGTGC
CCAGTAAGTACCTGAACTTGCATCTACTCAAAACGGGTTCTAGACCCCATTTTTCGC
ACTTCTCCGTATCTATGAACACGGGTTGCAACCTATTCATCGCTTCACCGGTGGGGG
AAACCTTCCCCTTGAGTCCCTCCAAAGATGGAGCGACGTGGAAAGCATTTGAAACG
GACGACAGTGTAGAGGTGATTCACAGAGAGACGAAGGAAAAGAGAATCTATAAGC
TCAAGTTCATTCCTCTGAAGAGTGGGGCTCTCCTAAAGGTTGACGTTTTGAAGGGAA
TTCCCTTTTGGGTTATCTCACAAGGGAGGAAAATCCTACCAACGATTTGTTCTGGAG
ATGAGGAGGTGCTATCAAACCCACAGAATGAGGTCTTCAAAGAGTGCACATCGTCG
AGTAGTCTCTCTCCCGAATTTGATTGTCTAGCCGGGCTGAGCACCTACCATAGGGAT
AAGAAGAACCACACGTGGAAAACGTCTAGCGGATCTATAGGTCAGTTTATAAAGAT
CTTCTTCAATAAGCCCGTACAAATTACCAAGTTTAGGTTTAAGCCCAGAGACGACCT
GCTGTCTTGGCCCTCCGAAGTAGCTCTCCAATTCGATACCGATGAGGAGGTGATCAT
ACCAATTCTGCATACGCACAATATGGGGCAGAACACGACTAGGCTAGAACACCCAA
TCATCACCACCTCTGTTAAGGTAGAAGTGAGAGACATGTACGAACGGGCAAGTGAA
AATACAGGAGGTTCTTTCGAGGTAATTGGAAGCACATGCCAGATGATGGAAGACGA
CTACATGACGCACCATGCTGTTATAGACATCACCGAGTGTGATCGTAGGTTGGAGTC
CCTCCCAGATGTTATGCCCTTAACGAAGGGGAGCAAATTTCTGGCCATTTGTCCCCG
CCCCTGCTTGAGCAGCTCCAATGGGGGAGTCATTTACGGGTCAGATGTTTATTCCAC
AGATTCTGCCGTATGTGGGGCGGCCGTACACGCGGGGGTGTGCAGCCGTGAGGGGG
AGGGCAGCTGCCACTTCCTCGTTGTGGTGCGCGGCGGGCGGGCCAACTTCGTGGGG
GCTCTCCAGAACAACGTCCTGTCTCTCAGTCGGGGTGGTGGCGGTAGCGGTAGCGGT
AGCTCCACCAGTAGCGATGGCGATGGCGATAGCGATAGCTCCACCAGTAGGGCCAA
CTTCTCATTTTCCCTCTCCAGTGCGTCAGGGTTCGGGGGGGGTCCGCGCGGGGCCCA
CGCAGAAGCCGCGCCAAGCAGCTACTCCATTGTGTTCAAGCCGAGGGACCATTTGG
CTCCAACGAACGGCTTTCTAGTAGACTCAGGGAGAGAGTTCACCAGCTACGGAAGC
GTTGCCTACGGATGGAAGAGGGAGGTTTCTCCTTCGTCCTCTTTTTCCTCTCCTTCTC
CTAGCTACACTTCCCCCCCGTTGGAAGAACCGACGCTGCTTAGGGGGGACTCCTCCT
CATTCAATGGGATTTACTCCGGGGGGATAGAATTCCCCCCCGCCTCGGCTAGCCAAA
ATTGCATTTCCCAACTGGATTGCCAGACCAACTTCTGGAAGTTTCAGATGCAAGAAA
ATGGCACCTACTTTGTGCAGGTGCTAGTGGGGAATAAAACTTCCCCTGAGAAGCAG
AAGGCCTTCGTCGAGCTGAATGGCGTTCCCATCATAAAGGGGGTGGACCTTGGCCCA
GACGAGGTCTTCGTCGCCACTGACCGCGTGCAGGTGACGAACCGGGCCCTCGTCCTC
ACGTCCACTTGCCTGGGCGGCGAGAGTGCCTGCTCGCGGGCGCGCGTCAGCATCAT
GGCGGTCCAGATTGTGAAGACG
X12
(SEQ ID NO: 138)
AACGGTATGAATAAAGACAAAGACGCAGAGATTACTCCCCCTCCGTTCATCG
TCTTGCCGGGTGGAAAAAAAATCCACATGCTGCAAAGCGAATACGAGTATGACGTT
CTGCGGGATATGTACCGAACGGATGAGGCGAATGGGGGAAGTGGTGAGAAGGAGA
GTCACCCCTCTGGGGATGGTGCAATCAGAAGAAACGAATTTTTTAAACTTTTTCACC
ACAGGGAGGGTCATTATAAGTTTGTTATCAAAAATGTTCCCACCAAATTGAGCGACC
TTTTGCAGAAAGGTGGCAACGAACAGGAGACAGACCTATTTCCTCTTTTATACAGGA
GTCTGCAATTCGCATGCAGCGCAGACGGGACGTGGCCATATGCCAGAAGAGAGGTG
GCCTTTTTTAAAAACGGGAGCGTCCACTGCGAAGCGGAATTTCAAAACGAGTTATCA
GTGAGGAGAACCCCCCGAAGTGGGAAGAAATCATTTGGACGTTTTCCAAGGGGGAC
ACTAATAAAAAGTAGCGACCTGAGGAGCAAAATTGTGGAGGGGAATTCTTATGATA
AAAGGGCCGCACCCCTGAAGAGTGAAAAAAAAAAGAAGGCTCTCTTTTTACACCCA
GAAAGTGTGCTATACAAAATGGAAGAAATATTTTTTTATGAAAATCCAAGTGTCAAA
AGTGAAATTGTCGGGTTTGTTCTTTTTCATGATGTGTGCACAGTAACGTCCTTAGGAC
ATGGAGCACATCCCGTTAACTCCCCCTTTTTGGGAAGCGACCTGCTGGAGATGATAT
TTGGCTACTGCATTTTACACGGGTTTAAAAAAATCAGAGTGAAAAGCGAATCCTTAA
ATTACGAAACTGGGATAAGGACCTCATTCATTGAGATTTTACTCAACGGAAAAACA
GCACTTGAACATTTAGGGTTAAGACTTACAAACGTAGCGAAGTTTTCTAAAGAACTG
TATTATGTAATCACTGGGTATACGTGGAAAAGTGATTTGGTGCTATCACCCATAGTA
AGGTTTGAACATGATTTATACGTGCATCACGACATAGAGGAGCGATTTTTCCTTTAC
GTGAATAAAATGTATAGGAATATGCTCCACGATTTGTCCTTCTCTTGTGATGAAAAT
TATTATCCTTATAAAAATTGTTATGACATCTACCCCTCCGTGAGAAGGAGTCAAAAT
AATCTTTGTCTCTTCGAACTGAATCCCATATATGAAGAATTGAAGGAGCTCTTTCCA
GACTCTTGTAATATTGGCCAACGCGTTAGAAAATGCTATGAGGAGATAAAAAAAAA
CGTTGTCTGCACACATAACGGTGAAGGAGGAGAAGACGGATGTAAGTACTACCAAT
TTATTGTAAATACATTCATAAAGCCGAGGAGGAAAACGTCCTTTTTTATTTATCACA
ATATGTATGTACAGGAATATCTTTCAAAGAAATCCTACCCCTATTACTTGCTACTCA
GTGAGGTTATAAAAAATGAAGAAAATAACTTTCTCGAAAAAGGCAACTACGACTTA
GTGGCCGATGCACAGACGCACCTCTTCTTAAATTACGTTTTGCAAAATTCTACCTTTT
TTATCTTTTGGAATTTCTCTACCGAATTTTGGAAAAGGTTTCGGTACATCCAGGCTGG
CCCAACCGGGGCCACTTCCACACCGCAGAAGGGGCAAGCTGTGTTTTGCCCCATGG
CCTATGCGTACGAATTTGTGGAGCACCTCGACACGTTTTATGTGAGGGGG
V6
(SEQ ID NO: 139)
TCCGTTGAAGAGGCTAAAAAAAATACTCAGGAAGTTGTGACAAATGTGGACA
ATGCTGCTAAATCTCAGGCCACCAATTCAAATCCGATAAGTCAGCCTGTAGATAGTA
GTAAAGCGGAGAAGGTTCCAGGAGATTCTACGCATGGAAATGTTAACAGTGGCCAA
GATAGTTCTACCACAGGTAAAGCTGTTACGGGGGATGGTCAAAATGGAAATCAGAC
ACCTGCAGAAAGCGATGTACAGCGAAGTGATATTGCCGAAAGTGTAAGTGCTAAAA
ATGTTGATCCGCAGAAATCTGTAAGTAAAAGAAGTGACGACACTGCAAGCGTTACA
GGTATTGCCGAAGCTGGAAAGGAAAACTTAGGCGCATCAAATAGTCGACCTTCTGA
GTCCACCGTTGAAGCAAATAGCCCAGGTGATGATACTGTGAACAGTGCATCTATACC
TGTAGTGAGTGGTGAAAACCCATTGGTAACCCCCTATAATGGTTTGAGGCATTCGAA
AGACAATAGTGATAGCGATGGACCTGCGGAATCAATGGCGAATCCTGATTCAAATA
GTAAAGGTGAGACGGGAAAGGGGCAAGATAATGATATGGCGAAGGCTACTAAAGA
TAGTAGTAATAGTTCAGATGGTACCAGCTCTGCTACGGGTGATACTACTGATGCAGT
TGATAGGGAAATTAATAAAGGTGTTCCTGAGGATAGGGATAAAACTGTAGGAAGTA
AAGATGGAGGGGGGGAAGATAACTCTGCAAATAAGGATGCAGCGACTGTAGTTGGT
GAGGATAGAATTCGTGAGAACAGCGCTGGTGGTAGCACTAATGATAGATCAAAAAA
TGACACGGAAAAGAACGGGGCCTCTACCCCTGACAGTAAACAAAGTGAGGATGCAA
CTGCGCTAAGTAAAACCGAAAGTTTAGAATCAACAGAAAGTGGAGATAGAACTACT
AATGATACAACTAACAGTTTAGAAAATAAAAATGGAGGAAAAGAAAAGGATTTACA
AAAGCATGATTTTAAAAGTAATGATACGCCGAATGAAGAACCAAATTCTGATCAAA
CTACAGATGCAGAAGGACATGACAGGGATAGCATCAAAAATGATAAAGCAGAAAG
GAGAAAGCATATGAATAAAGATACTTTTACGAAAAATACAAATAGTCACCATTTAA
AT
V7
(SEQ ID NO: 140)
ATACGGAATGGAAACAACCCGCAGGCATTAGTTCCTGAAAAGGGCGCTGACC
CGAGTGGGGGCCAGAACAACCGCTCCGGAGAAAACCAAGACACGTGCGAAATTCA
AAAGATGGCCGAAGAAATGATGGAAAAAATGATGAAGGAAAAAGACGTGTTTAGC
TCCATCATGGAACCTCTCCAGAGCAAATTAACTGACGATCATCTGTGTTCAAAAATG
AAATATACGAACATTTGTCTTCACGAAAAGGACAAAACTCCCTTGACCTTCCCCTGC
ACAAGTCCGCAGTACGAACAGCTAATTCATCGCTTCACTTATAAAAAGTTGTGCAAC
TCCAAGGTGGCCTTTAGCAACGTCTTGCTCAAATCCTTCATCGATAAAAAAAATGAA
GAAAACACATTTAACACGATCATACAGAATTACAAAGTTCTGTCCACTTGCATTGAC
GATGATTTGAAGGACATTTATAATGCATCCATAGAGTTATTCTCCGACATAAGAACC
TCCGTCACAGAAATTACCGAAAAGTTGTGGTCCAAAAATATGATCGAAGTTTTAAAG
ACAAGAGAGCAAACCATTGCAGGCATTTTATGTGAGTTAAGAAATGGAAATAATTC
TCCCCTAGTATCGAACAGTTTTTCCTATGAAAATTTTGGAATTCTCAAGGTTAATTAT
GAGGGATTACTAAACCAGGCGTATGCGGCCTTTTCAGACTACTATTCATACTTTCCC
GCTTTTGCCATTAGCATGTTAGAAAAGGGAGGGTTGGTCGACCGCTTGGTCGCCATC
CATGAGAGCTTGACCAACTACAGGACGAGAAATATTCTCAAGAAGATCAATGAGAA
GTCCAAAAATGAGGTCCTCAATAATGAAGAAATTATGCACAGCTTGAGCAGTTACA
AGCACCATGCCGGGGGCACGCGTGGCGCCTTCCTGCAGTCCAGAGATGTGCGCGAA
GTTACGCAAGGAGATGTGAGCGTTGATGAGAAGGGCGACCGGGCCACCACCGCGGG
GGGCAACCAAAGCGCAAGCGTGGCTGCGGCGGCCCCGAAGGATGCGGGCCCAACC
GTGGCTGCTCCTAACACTGCTGCTACGCTCAAAACGGCTGCTTCCCCCAACGCGGCT
GCTACTAACACTGCTGCTCCCCCCAACATGGGTGCCACCTCCCCGCTGAGCAACCCC
CTGTACGGCACCAGCTCCCTGCAGCCAAAGGACGTCGCGGTGCTGGTCAGAGATCT
GCTCAAGAACACGAACATCATCAAGTTCGAGAATAACGAACCGACTAGCCAAATGG
ACGATGAAGAAATTAAGAAGCTCATTGAGAGCTCCTTTTTCGACTTGAGCGACAACA
CCATGTTAATGCGGTTGCTCATAAAGCCGCAGGCGGCCATCTTACTAATCATTGAGT
CCTTCATTATGATGACGCCCTCCCCCACGAGGGACGCCAAGACCTATTGCAAGAAA
GCCCTAGTTAATGGCCAGCTAATCGAAACCTCAGATTTAAACGCGGCGACGGAGGA
AGACGACCTCATAAACGAGTTTTCCAGCAGGTACAATTTATTCTACGAGAGGCTCAA
GCTGGAGGAGTTG
V8
(SEQ ID NO: 141)
AAGGAGTACTGCGACCAGCTTAGCTTTTGCGATGTGGGATTGACACACCACT
TTGATACGTATTGTAAGAATGACCAGTACCTGTTCGTTCACTACACTTGTGAGGACC
TCTGCAAAACGTGTGGCCCTAATTCGTCCTGCTACGGAAACAAGTACAAACATAAGT
GCCTGTGCAATAGCCCCTTCGAGAGTAAAAAGAACCATTCCATTTGCGAAGCACGA
GGTAGCTGCGATGCACAGGTATGCGGCAAGAATCAAATTTGCAAAATGGTAGACGC
TAAAGCAACATGCACATGTGCAGATAAATACCAAAATGTGAATGGGGTGTGTCTAC
CGGAAGATAAGTGCGACCTTCTGTGCCCCTCAAACAAATCGTGCCTGCTGGAAAATG
GGAAAAAAATATGCAAGTGCATTAATGGGTTGACTCTACAGAACGGCGAGTGCGTC
TGCTCGGATAGCAGCCAAATTGAAGAAGGACACCTCTGTGTGCCCAAGAATAAATG
TAAACGGAAGGAGTACCAACAGCTCTGCACCAATGAGAAGGAACACTGTGTGTATG
ATGAGCAGACGGACATTGTGCGGTGCGACTGCGTGGACCACTTCAAGCGGAACGAA
CGGGGAATTTGCATCCCAGTCGACTACTGCAAAAATGTCACCTGCAAGGAAAATGA
GATTTGCAAAGTTGTTAATAATACACCCACATGTGAGTGTAAAGAAAATTTAAAAA
GAAATAGTAACAATGAATGTGTATTCAATAACATGTGTCTTGTTAATAAAGGGAACT
GCCCCATTGATTCGGAGTGCATTTATCACGAGAAAAAAAGGCATCAGTGTTTGTGCC
ATAAGAAGGGCCTCGTCGCCATTAATGGCAAGTGCGTCATGCAGGACATGTGCAGG
AGCGATCAGAACAAATGCTCCGAAAATTCCATTTGTGTAAATCAAGTGAATAAAGA
ACCGCTGTGCATATGTTTGTTTAATTATGTGAAGAGTCGGTCGGGCGACTCGCCCGA
GGGTGGACAGACGTGCGTGGTGGACAATCCCTGCCTCGCGCACAACGGGGGCTGCT
CGCCAAACGAGGTTTGCACGTTCAAAAATGGAAAGGTAAGTTGCGCCTGCGGGGAG
AACTACCGCCCCAGGGGGAAGGACAGCCCAACGGGACAAGCGGTCAAACGGGGGG
AAGCGACCAAACGGGGTGACGCGGGTCAGCCCGGGCAGGCGCACTCAGCAAATGA
GAACGCGTGCCTGCCCAAGACGTCCGAGGCGGACCAAACCTTCACCTTCCAGTACA
ACGACGACGCGGCCATCATTCTCGGGTCCTGCGGAATTATACAGTTTGTGCAAAAGA
GCGATCAGGTCATTTGGAAAATTAACAGCAACAATCACTTTTACATTTTTAATTATG
ACTATCCATCTGAGGGTCAGCTGTCGGCACAAGTCGTGAACAAGCAGGAGAGCAGC
ATTTTGTACTTAAAGAAAACCCACGCGGGGAAAGTCTTTTACGCCGACTTTGAGTTG
GGTCATCAGGGATGCTCCTACGGAAACATGTTTCTCTACGCCCACCGGGAGGAGGCT
V9
(SEQ ID NO: 142)
AGCAAAAACATTATTATTCTGAACGATGAAATTACCACCATTAAAAGCCCGA
TTCATTGCATTACCGATATTTATTTTCTGTTTCGCAACGAACTGTATAAAACCTGCAT
TCAGCATGTGATTAAAGGCCGCACCGAAATTCATGTGCTGGTGCAGAAAAAAATTA
ACAGCGCGTGGGAAACCCAGACCACCCTGTTTAAAGATCATATGTGGTTTGAACTGC
CGAGCGTGTTTAACTTTATTCATAACGATGAAATTATTATTGTGATTTGCCGCTATAA
ACAGCGCAGCAAACGCGAAGGCACCATTTGCAAACGCTGGAACAGCGTGACCGGCA
CCATTTATCAGAAAGAAGATGTGCAGATTGATAAAGAAGCGTTTGCGAACAAAAAC
CTGGAAAGCTATCAGAGCGTGCCGCTGACCGTGAAAAACAAAAAATTTCTGCTGAT
TTGCGGCATTCTGAGCTATGAATATAAAACCGCGAACAAAGATAACTTTATTAGCTG
CGTGGCGAGCGAAGATAAAGGCCGCACCTGGGGCACCAAAATTCTGATTAACTATG
AAGAACTGCAGAAAGGCGTGCCGTATTTTTATCTGCGCCCGATTATTTTTGGCGATG
AATTTGGCTTTTATTTTTATAGCCGCATTAGCACCAACAACACCGCGCGCGGCGGCA
ACTATATGACCTGCACCCTGGATGTGACCAACGAAGGCAAAAAAGAATATAAATTT
AAATGCAAACATGTGAGCCTGATTAAACCGGATAAAAGCCTGCAGAACGTGGCGAA
ACTGAACGGCTATTATATTACCAGCTATGTGAAAAAAGATAACTTTAACGAATGCTA
TCTGTATTATACCGAACAGAACGCGATTGTGGTGAAACCGAAAGTGCAGAACGATG
ATCTGAACGGCTGCTATGGCGGCAGCTTTGTGAAACTGGATGAAAGCAAAGCGCTG
TTTATTTATAGCACCGGCTATGGCGTGCAGAACATTCATACCCTGTATTATACCCGCT
ATGAT

TABLE 6
references associated with proteins

| Protein | 5′ position | amino acid | |
|---|---|---|---|
| Code | to 3′ (bp) | position | reference |
| X1 | (4-1845) | | Lu J Proteomics 2014 |
| X2 | (67-1161) | | Lu J Proteomics 2014 |
| X3 | (70-555) | | Lu J Proteomics 2014 |
| X4 | (4-948) | | Lu J Proteomics 2014 |
| X5 | (73-1659) | | Lu J Proteomics 2014 |
| X6 | (73-1074) | | Lu J Proteomics 2014 |
| X7 | (1384-2190) | | Lu J Proteomics 2014 |
| X8 | (559-2871) | | Lu J Proteomics 2014 |
| X9 | (4-660) | | Lu J Proteomics 2014 |
| X10 | (4-342) | | Lu J Proteomics 2014 |
| X11 | (1264-3261) | | Lu J Proteomics 2014 |
| X12 | (1957-3702) | | Lu J Proteomics 2014 |
| V1 | | 140 to 1275 | Hietanen 2015 Infection and |
| | | | Immunity PMID: 26712206 |
| V2 | | 160 to 1135 | Hietanen 2015 Infection and |
| | | | Immunity PMID: 26712206 |
| V3 | | 161 to 1454 | Hietanen 2015 Infection and |
| | | | Immunity PMID: 26712206 |
| V4 | | 501 to 1300 | Hietanen 2015 Infection and |
| | | | Immunity PMID: 26712206 |
| V12 | | 160 to 1170 | Hietanen 2015 Infection and |
| | | | Immunity PMID: 26712206 |
| V5 | | 161-641 | Franca 2017 Elife PMID: |
| | | | 28949293 |
| V11 | | Region II | Franca 2017 Elife PMID: |
| | | | 28949293 |
| V10 | | Region II | |
| V13 | | Region II | |
| V6 | (1522-2697) | | |
| V7 | (29-551) | | |
| V8 | (552-1075) | | |
| V9 | (30-366) | | |

APPENDIX IIIA

| | Area Under Curve (1 antigen) | | | Top 1% of 2 antigen combis | | | Top 1% of 3 antigen combis | | | Top 1% of 4 antigen combis | | | (<9 m GMT)/(12 m GMT) | | | (<9 m GMT)/(-ve control GMT) | | |
|---|---|---|---|---|---|---|---|---|---|---|---|---|---|---|---|---|---|---|
| | Thailand | Brazil | Solomons | Thailand | Brazil | Solomons | Thailand | Brazil | Solomons | Thailand | Brazil | Solomons | Thailand | Brazil | Solomons | Thailand | Brazil | Solomons |
| RBP2a | 0.849 | 0.818 | 0.868 | 100 | 100 | 100 | 95.3 | 96.2 | 100 | 89.7 | 98.5 | 100 | 10.85 | 8.53 | 11.84 | 31.33 | 26.31 | 13.91 |
| L01 | 0.812 | 0.787 | 0.697 | 5.9 | 5.9 | 0 | 21.1 | 13.5 | 2.3 | 43.5 | 23.9 | 4.3 | 7.41 | 4.49 | 4.09 | 10.73 | 17.26 | 2.1 |
| L31 | 0.805 | 0.762 | 0.766 | 0 | 0 | 0 | 2.6 | 2.6 | 2.3 | 5 | 2.7 | 3.7 | 3.9 | 3.05 | 2.56 | 8.62 | 12.32 | 5.1 |
| X087885 | 0.807 | 0.748 | 0.697 | 5.9 | 0 | 0 | 16.7 | 4.7 | 7 | 20.3 | 9.2 | 14.6 | 4.28 | 1.79 | 1.2 | 9.82 | 34.44 | 15.93 |
| PvEBP | 0.747 | 0.739 | 0.707 | 0 | 0 | 0 | 1.8 | 1.8 | 1.8 | 5 | 2.4 | 3.1 | 6.53 | 5.18 | 2.01 | 21.12 | 8.91 | 2.61 |
| L55 | 0.79 | 0.781 | 0.643 | 5.9 | 5.9 | 0 | 14.6 | 12.3 | 1.5 | 17.2 | 20.9 | 2.6 | 4.94 | 4.42 | 1.95 | 7.9 | 7.91 | 1.19 |
| PvRipr | 0.754 | 0.772 | 0.646 | 0 | 0 | 5.9 | 1.8 | 5.6 | 2 | 3 | 9.1 | 3.1 | 5.01 | 4.32 | 2.57 | 7.02 | 7.89 | 1.07 |
| L54 | 0.79 | 0.727 | 0.654 | 5.9 | 0 | 0 | 3.5 | 2.6 | 1.8 | 5.6 | 4.4 | 3.1 | 4.4 | 2.98 | 1.88 | 5.39 | 3.82 | 1.3 |
| L07 | 0.747 | 0.765 | 0.599 | 0 | 0 | 0 | 2.3 | 4.7 | 1.8 | 3.1 | 5.3 | 2.5 | 2.56 | 3.11 | 1.45 | 4.3 | 6.29 | 1.35 |
| L30 | 0.732 | 0.61 | 0.609 | 0 | 0 | 0 | 1.2 | 2.3 | 2.9 | 2.3 | 3.8 | 5.4 | 4.14 | 1.53 | 1.55 | 13.36 | 2.24 | 1.79 |
| PVDBPII | 0.74 | 0.773 | 0.639 | 0 | 0 | 5.9 | 0.6 | 3.2 | 3.2 | 1.7 | 2.6 | 4 | 2.76 | 4.89 | 1.79 | 5.14 | 15.42 | 1.34 |
| L34 | 0.767 | 0.746 | 0.67 | 0 | 0 | 0 | 3.8 | 7.3 | 0.6 | 4.5 | 16.6 | 2.2 | 3.22 | 2.99 | 1.84 | 3.87 | 4.78 | 1.46 |
| X092995 | 0.792 | 0.703 | 0.642 | 5.9 | 0 | 0 | 13.7 | 1.5 | 2 | 11.5 | 1.9 | 5.6 | 2.88 | 1.41 | 1.03 | 4.64 | 8.55 | 4.19 |
| L12 | 0.755 | 0.731 | 0.637 | 5.9 | 0 | 0 | 3.2 | 3.8 | 1.8 | 3.5 | 6.1 | 2.9 | 3.19 | 2.73 | 1.46 | 3.81 | 3.47 | 1.8 |
| rBP1b | 0.533 | 0.578 | 0.525 | 5.9 | 5.9 | 0 | 17.5 | 4.1 | 1.2 | 24.1 | 4.7 | 2.5 | 1.23 | 1.44 | 1.11 | 0.67 | 0.79 | 0.84 |
| L23 | 0.759 | 0.753 | 0.658 | 0 | 0 | 0 | 1.5 | 7 | 1.2 | 4 | 14.8 | 2.9 | 2.95 | 2.67 | 1.86 | 4.3 | 5.09 | 1.59 |
| L02 | 0.746 | 0.724 | 0.677 | 0 | 0 | 0 | 1.5 | 2.3 | 2.3 | 2.7 | 3.7 | 3.9 | 3.7 | 3 | 1.76 | 3.89 | 4.07 | 1.82 |
| L32 | 0.705 | 0.651 | 0.493 | 0 | 0 | 5.9 | 1.8 | 1.2 | 17 | 3.7 | 1.9 | 30.2 | 2.79 | 3.17 | 1.61 | 2.24 | 0.81 | 0.31 |
| L28 | 0.759 | 0.755 | 0.667 | 5.9 | 0 | 0 | 2.6 | 1.2 | 1.2 | 3.8 | 2.5 | 2.6 | 2.92 | 2.44 | 1.43 | 5.74 | 5.24 | 2.14 |
| L19 | 0.758 | 0.67 | 0.654 | 0 | 0 | 0 | 1.5 | 0.9 | 3.2 | 2.6 | 2.3 | 6.5 | 3.66 | 2.18 | 1.09 | 6.58 | 3.11 | 4.89 |
| L36 | 0.727 | 0.698 | 0.682 | 0 | 0 | 0 | 1.5 | 0.9 | 2 | 3.2 | 1.8 | 2.8 | 2.95 | 2.44 | 1.99 | 3.28 | 3.2 | 1.8 |
| L41 | 0.702 | 0.66 | 0.686 | 0 | 0 | 0 | 1.5 | 0.6 | 2 | 2.3 | 1.7 | 3.8 | 2.12 | 1.91 | 1.72 | 4.99 | 3.03 | 1.9 |
| X088820 | 0.723 | 0.666 | 0.633 | 5.9 | 0 | 0 | 4.4 | 0.6 | 3.8 | 4 | 1.8 | 6.7 | 1.9 | 1.28 | 0.99 | 4.04 | 8.58 | 5.87 |
| PvDBP.Sa | 0.716 | 0.751 | 0.616 | 0 | 0 | 5.9 | 0.3 | 2.6 | 8.8 | 1.7 | 2.6 | 7.2 | 3.01 | 4.78 | 1.85 | 3.96 | 12.35 | 0.83 |
| RBP2a | 0.692 | 0.731 | 0.662 | 0 | 0 | 0 | 3.5 | 1.2 | 0.9 | 5.4 | 1.8 | 1.6 | 2.42 | 2.49 | 1.47 | 2.46 | 4.6 | 1.5 |
| L18 | 0.736 | 0.663 | 0.622 | 0 | 0 | 0 | 2.3 | 2 | 2.3 | 3.1 | 4.5 | 3.8 | 2.22 | 1.41 | 0.93 | 2.53 | 2.33 | 4.31 |
| RBP2cNB | 0.744 | 0.7 | 0.551 | 0 | 0 | 5.9 | 1.5 | 1.2 | 11.1 | 3.6 | 1.9 | 6.6 | 3.02 | 2.3 | 1.57 | 3.87 | 3.23 | 0.64 |
| L27 | 0.735 | 0.663 | 0.585 | 0 | 0 | 5.9 | 2.9 | 1.5 | 2 | 4.5 | 2.4 | 2.7 | 2.34 | 2.24 | 1.66 | 1.67 | 1.2 | 0.63 |
| L42 | 0.697 | 0.632 | 0.593 | 0 | 0 | 0 | 1.5 | 0.9 | 2 | 2.9 | 1.8 | 3 | 2.81 | 1.91 | 1.85 | 4.44 | 2.89 | 1.19 |
| L14 | 0.701 | 0.637 | 0.581 | 0 | 0 | 0 | 3.5 | 1.2 | 1.5 | 4.1 | 2 | 3.1 | 1.94 | 1.51 | 1.33 | 2.85 | 2.23 | 1.07 |
| X099930 | 0.71 | 0.63 | 0.573 | 5.9 | 0 | 0 | 3.8 | 0.9 | 1.5 | 4.1 | 1.7 | 2.5 | 1.75 | 1.27 | 0.94 | 2.85 | 3.15 | 2.07 |
| PvDBP.R3 | 0.685 | 0.67 | 0.554 | 0 | 0 | 5.9 | 2 | 1.2 | 2.6 | 4.1 | 3 | 2.7 | 2.51 | 2.19 | 1.73 | 2.57 | 3.11 | 0.51 |
| L22 | 0.725 | 0.622 | 0.562 | 0 | 5.9 | 0 | 2.3 | 4.1 | 1.5 | 3 | 5.6 | 2.4 | 1.98 | 1.25 | 0.99 | 2.28 | 2.13 | 1.3 |
| RBP1a | 0.668 | 0.669 | 0.565 | 5.9 | 0 | 0 | 0 | 1.5 | 0.9 | 1.2 | 2.7 | 1.9 | 2.4 | 2.32 | 2.49 | 1.45 | 2.06 | 2.59 |
| PvCYRPA | 0.779 | 0.563 | 0.532 | 0 | 0 | 5.9 | 0.6 | 0.9 | 14 | 2 | 1.9 | 10.3 | 2.37 | 1.25 | 1.46 | 4.55 | 1.59 | 0.31 |
| L10 | 0.719 | 0.588 | 0.553 | 0 | 5.9 | 0 | 1.2 | 6.1 | 1.2 | 2.4 | 9.3 | 2.3 | 2.14 | 1.31 | 1.04 | 3.61 | 1.39 | 1.43 |
| L24 | 0.656 | 0.595 | 0.605 | 0 | 5.9 | 0 | 5.3 | 2.9 | 1.2 | 5.5 | 5.6 | 2.8 | 2.01 | 1.33 | 0.88 | 1.75 | 1.71 | 5.03 |
| L21 | 0.653 | 0.597 | 0.602 | 0 | 0 | 0 | 1.5 | 1.8 | 1.8 | 3 | 2.6 | 4.1 | 2 | 1.55 | 0.93 | 1.47 | 1.35 | 3.08 |
| L51 | 0.679 | 0.625 | 0.547 | 5.9 | 0 | 5.9 | 4.1 | 1.8 | 3.5 | 6.2 | 3.7 | 5.4 | 1.85 | 1.48 | 1.31 | 2.04 | 1.74 | 0.89 |
| L25 | 0.67 | 0.593 | 0.58 | 0 | 5.9 | 0 | 0.9 | 2.5 | 0.9 | 2.1 | 6 | 2.8 | 1.61 | 1.14 | 0.96 | 2.04 | 1.76 | 2.05 |
| L33 | 0.65 | 0.608 | 0.584 | 0 | 0 | 0 | 1.8 | 1.2 | 0.9 | 3.7 | 3.1 | 1.6 | 1.83 | 1.43 | 1.37 | 1.63 | 1.82 | 1.05 |
| L20 | 0.674 | 0.619 | 0.544 | 0 | 0 | 0 | 1.5 | 1.2 | 1.5 | 2.7 | 2.1 | 2.9 | 1.71 | 1.31 | 1.23 | 2.2 | 2.08 | 0.82 |
| X114330 | 0.666 | 0.594 | 0.577 | 0 | 0 | 0 | 1.5 | 1.2 | 1.5 | 2.2 | 2.6 | 3 | 1.44 | 1.15 | 1.03 | 2.35 | 2.2 | 1.78 |
| L50 | 0.713 | 0.604 | 0.494 | 0 | 5.9 | 5.9 | 1.2 | 6.4 | 11.1 | 2.9 | 8.6 | 7.3 | 2.15 | 1.55 | 1.4 | 2.53 | 1.34 | 0.45 |
| L06 | 0.686 | 0.583 | 0.54 | 0 | 0 | 0 | 1.5 | 1.8 | 1.2 | 2.5 | 3.1 | 2.3 | 1.91 | 1.33 | 0.92 | 2.23 | 1.41 | 1.57 |
| L05 | 0.686 | 0.607 | 0.499 | 0 | 0 | 0 | 2 | 2.3 | 2 | 3.9 | 4.7 | 3.4 | 2.23 | 1.44 | 1.03 | 2.1 | 1.9 | 0.72 |
| X080665 | 0.678 | 0.595 | 0.522 | 0 | 5.9 | 0 | 1.5 | 3.8 | 1.2 | 2.1 | 6.2 | 3.6 | 1.8 | 1.25 | 0.9 | 2.64 | 1.8 | 1.21 |
| L39 | 0.673 | 0.56 | 0.537 | 5.9 | 0 | 0 | 4.1 | 1.2 | 1.5 | 4 | 2.4 | 2.8 | 1.64 | 1.12 | 0.96 | 2.96 | 1.57 | 1.5 |
| X094350 | 0.641 | 0.602 | 0.516 | 0 | 0 | 0 | 1.5 | 2 | 1.8 | 2.7 | 3.2 | 4.2 | 1.47 | 1.3 | 0.96 | 1.79 | 1.7 | 1.15 |
| L11 | 0.652 | 0.594 | 0.49 | 0 | 5.9 | 5.9 | 3.8 | 4.4 | 5 | 5.3 | 7.7 | 10.7 | 1.58 | 1.29 | 0.96 | 1.67 | 1.29 | 0.92 |
| L38 | 0.64 | 0.543 | 0.552 | 0 | 5.9 | 0 | 1.2 | 5.3 | 1.5 | 3 | 6.3 | 2.6 | 1.59 | 1.2 | 1.19 | 1.18 | 1 | 0.89 |
| L37 | 0.628 | 0.608 | 0.487 | 0 | 5.9 | 5.9 | 2.6 | 2 | 3.2 | 5.1 | 3.7 | 4.9 | 1.54 | 1.6 | 1.15 | 1.17 | 0.92 | 0.73 |
| PvGAMA | 0.646 | 0.57 | 0.495 | 0 | 0 | 5.9 | 2.3 | 1.2 | 6.7 | 5.3 | 2.5 | 6.5 | 1.64 | 1.49 | 1.32 | 1.45 | 0.74 | 0.53 |
| L49 | 0.577 | 0.532 | 0.6 | 0 | 5.9 | 5.9 | 1.8 | 19.6 | 8.2 | 2.5 | 11.9 | 13.6 | 1.26 | 1.08 | 0.89 | 1.24 | 0.4 | 0.34 |
| L47 | 0.641 | 0.513 | 0.539 | 0 | 5.9 | 5.9 | 0.9 | 5.8 | 4.7 | 1.9 | 6.8 | 4.8 | 1.52 | 1.29 | 1.21 | 1.73 | 0.51 | 0.38 |
| L48 | 0.552 | 0.586 | 0.523 | 5.9 | 0 | 0 | 2.9 | 1.2 | 1.2 | 4.8 | 2.4 | 2.7 | 1.16 | 1.23 | 0.98 | 1.3 | 1.56 | 1.23 |
| RBP2.P2 | 0.596 | 0.544 | 0.515 | 5.9 | 5.9 | 5.9 | 5 | 14.6 | 17 | 6.5 | 8.9 | 24.9 | 1.48 | 1.34 | 1.16 | 0.94 | 0.66 | 0.46 |
| L03 | 0.579 | 0.503 | 0.566 | 5.9 | 5.9 | 0 | 2.6 | 2.3 | 2 | 3.8 | 4.1 | 4.4 | 1.59 | 1.14 | 0.93 | 0.82 | 0.8 | 0.51 |
| L52 | 0.526 | 0.562 | 0.524 | 5.9 | 5.9 | 5.9 | 4.4 | 4.7 | 4.1 | 4.9 | 4.8 | 6.3 | 1.29 | 1.4 | 1.07 | 0.56 | 0.6 | 0.58 |
| L40 | 0.564 | 0.55 | 0.495 | 0 | 0 | 0 | 1.8 | 1.5 | 1.2 | 3.3 | 2.7 | 3.2 | 1.23 | 1.01 | 0.91 | 1.08 | 1.79 | 1.09 |

APPENDIX IIIB

| | (<9 m) > (>12 m GMT + | | | (<9 m) > (-ve cont GMT + | | | | | | | | |
|---|---|---|---|---|---|---|---|---|---|---|---|---|
| | 2*ds(>12 m)) | | | 2*sd(-ve cont)) | | | age trend | | | age trend (P value) | | |
| | Thailand | Brazil | Solomons | Thailand | Brazil | Solomons | Thailand | Brazil | Solomons | Thailand | Brazil | Solomons |
| RBP2a | 34.7 | 19 | 47 | 70.8 | 64.4 | 45.7 | 1.02 | 0.63 | 1.06 | 0 | 0 | 0 |
| L01 | 36.1 | 0 | 24.3 | 51.4 | 56.6 | 14.3 | 0.39 | 0.52 | 0.24 | 0 | 0 | 0.0043 |
| L31 | 22.2 | 0 | 7.8 | 25 | 38 | 7.4 | 0.41 | 0.34 | 0.23 | 0 | 0 | 3.00E−04 |
| X087885 | 15.3 | 7.8 | 5.7 | 41.7 | 81 | 50.9 | 0.53 | 0.13 | −0.1 | 0 | 2.00E−04 | 0.0466 |
| PvEBP | 26.4 | 22.9 | 20 | 55.3 | 41 | 7.8 | 1.08 | 0.59 | 0.21 | 0 | 0 | 0 |
| L55 | 27.8 | 17.1 | 13.9 | 38.9 | 29.8 | 3.5 | 0.48 | 0.46 | 0.44 | 0 | 0 | 0 |
| PvRipr | 25 | 15.1 | 23.5 | 31.9 | 29.3 | 4.8 | 0.55 | 0.42 | 0.2 | 0 | 0 | 0.0013 |
| L54 | 23.6 | 16.1 | 14.3 | 26.4 | 19 | 2.2 | 0.48 | 0.33 | 0.24 | 0 | 0 | 0 |
| L07 | 22.2 | 0 | 8.3 | 27.8 | 41.5 | 3.9 | 0.22 | 0.34 | 0.19 | 0 | 0 | 4.00E−04 |
| L30 | 23.6 | 9.8 | 10.9 | 47.2 | 11.7 | 9.6 | 0.85 | 0.16 | 0.05 | 0 | 2.00E−04 | 0.4217 |
| PVDBPII | 15.3 | 19 | 10.4 | 20.8 | 47.3 | 3.5 | 0.4 | 0.63 | 0.1 | 0 | 0 | 0.076 |
| L34 | 15.3 | 12.2 | 10.9 | 12.5 | 19 | 3.9 | 0.35 | 0.35 | 0.18 | 0 | 0 | 2.00E−04 |
| X092995 | 12.5 | 3.4 | 1.7 | 15.3 | 34.1 | 10 | 0.33 | 0.09 | −0.03 | 0 | 0.0034 | 0.4924 |
| L12 | 23.6 | 12.7 | 5.2 | 16.7 | 15.1 | 3 | 0.36 | 0.22 | −0.07 | 0 | 0 | 0.1928 |
| rBP1b | 2.8 | 4.4 | 4.3 | 0 | 0 | 0 | −0.12 | 0.12 | −0.06 | 0.001 | 1.00E−04 | 0.1077 |
| L23 | 9.7 | 13.7 | 11.7 | 12.5 | 19.5 | 5.7 | 0.29 | 0.22 | 0.1 | 0 | 0 | 0.0824 |
| L02 | 15.3 | 10.7 | 7.4 | 15.3 | 13.7 | 2.6 | 0.31 | 0.4 | 0.02 | 0 | 0 | 0.6554 |
| L32 | 13.9 | 20.5 | 10 | 4.2 | 3.9 | 0.4 | 0.15 | 0.31 | 0.25 | 0.0016 | 0 | 1.00E−04 |
| L28 | 18.1 | 12.7 | 8.3 | 45.8 | 33.2 | 9.1 | 0.46 | 0.32 | 0.26 | 0 | 0 | 0 |
| L19 | 20.8 | 9.8 | 3.9 | 33.3 | 19.5 | 10.9 | 0.62 | 0.31 | −0.14 | 0 | 0 | 0.0036 |
| L36 | 18.1 | 14.6 | 11.3 | 36.1 | 22 | 10.4 | 0.63 | 0.36 | 0.3 | 0 | 0 | 0 |
| L41 | 9.7 | 9.3 | 7.8 | 29.2 | 17.6 | 8.3 | 0.39 | 0.41 | 0.32 | 0 | 0 | 0 |
| X088820 | 12.5 | 0 | 0 | 15.3 | 35.6 | 14.8 | 0.17 | 0.07 | −0.02 | 0 | 0.0032 | 0.5905 |
| PvDBP.Sa | 18.1 | 16.6 | 11.3 | 16.7 | 36.6 | 1.3 | 0.39 | 0.61 | 0.18 | 0 | 0 | 0.0016 |
| RBP2a | 18.1 | 13.2 | 9.1 | 18.1 | 22.4 | 3.5 | 0.3 | 0.34 | 0.1 | 0 | 0 | 0.0144 |
| L18 | 15.3 | 3.4 | 4.3 | 11.1 | 6.3 | 10.4 | 0.11 | 0.08 | −0.17 | 0.0022 | 0.0106 | 1.00E−04 |
| RBP2cNB | 23.6 | 16.6 | 10 | 18.1 | 17.6 | 1.7 | 0.43 | 0.35 | 0.44 | 0 | 0 | 0 |
| L27 | 15.3 | 13.2 | 10 | 0 | 0 | 0 | 0.1 | 0.3 | 0.15 | 0.0021 | 0 | 3.00E−04 |
| L42 | 16.7 | 12.7 | 16.1 | 29.2 | 20 | 7 | 0.5 | 0.3 | 0.27 | 0 | 0 | 0 |
| L14 | 12.5 | 3.9 | 5.2 | 9.7 | 5.9 | 1.3 | 0.05 | 0.18 | 0.02 | 0.1401 | 0 | 0.6094 |
| X099930 | 5.6 | 6.8 | 1.7 | 8.3 | 17.6 | 6.1 | 0.06 | 0.02 | −0.06 | 0.0734 | 0.4923 | 0.1513 |
| PvDBP.R3 | 13.9 | 9.8 | 8.7 | 13.9 | 11.2 | 0.9 | 0.36 | 0.33 | 0.16 | 0 | 0 | 0.0047 |
| L22 | 9.7 | 3.4 | 3 | 4.2 | 5.9 | 2.6 | 0.11 | 0.16 | −0.08 | 0.0012 | 0 | 0.0611 |
| RBP1a | 18.1 | 16.1 | 10.4 | 8.3 | 18 | 1.3 | 0.36 | 0.44 | 0.12 | 0 | 0 | 0.0239 |
| PvCYRPA | 16.7 | 0 | 4.8 | 29.2 | 11.7 | 0 | 0.43 | −0.02 | 0.15 | 0 | 0.6208 | 0.0046 |
| L10 | 8.3 | 4.4 | 3 | 12.5 | 4.4 | 1.3 | 0.47 | 0.16 | −0.17 | 0 | 0 | 3.00E−04 |
| L24 | 9.7 | 6.8 | 3.9 | 4.2 | 7.3 | 7 | 0.12 | 0.14 | −0.21 | 0.0069 | 3.00E−04 | 0 |
| L21 | 8.3 | 6.3 | 3.5 | 2.8 | 6.3 | 6.1 | 0.04 | 0.13 | −0.19 | 0.3593 | 4.00E−04 | 0 |
| L51 | 4.2 | 3.9 | 4.8 | 2.8 | 3.9 | 2.6 | 0.25 | 0.22 | 0.31 | 0 | 0 | 0 |
| L25 | 11.1 | 2.4 | 0.9 | 6.9 | 4.9 | 3.9 | 0.04 | 0.04 | −0.15 | 0.3008 | 0.232 | 0.0025 |
| L33 | 11.1 | 4.9 | 5.2 | 6.9 | 5.9 | 0.9 | 0.21 | 0.22 | 0.24 | 0 | 0 | 0 |
| L20 | 9.7 | 0 | 4.3 | 0 | 0 | 0 | 0.01 | 0.11 | 0.02 | 0.7715 | 1.00E−04 | 0.7011 |
| X114330 | 5.6 | 5.9 | 3 | 8.3 | 10.7 | 4.3 | 0.11 | 0.05 | −0.09 | 4.00E−04 | 0.103 | 0.054 |
| L50 | 11.1 | 5.4 | 6.5 | 5.6 | 4.4 | 0.9 | 0.13 | 0.27 | 0.2 | 6.00E−04 | 0 | 0 |
| L06 | 6.9 | 4.4 | 1.7 | 2.8 | 3.4 | 0.4 | −0.03 | 0.01 | −0.35 | 0.4684 | 0.6901 | 0 |
| L05 | 12.5 | 8.8 | 3.5 | 5.6 | 9.8 | 0.4 | 0.13 | 0.15 | −0.11 | 0.0018 | 1.00E−04 | 0.0232 |
| X080665 | 4.2 | 4.4 | 1.3 | 2.8 | 4.4 | 0.4 | 0.14 | 0.08 | −0.09 | 7.00E−04 | 0.0263 | 0.0757 |
| L39 | 6.9 | 3.9 | 3.5 | 6.9 | 4.4 | 3.5 | 0.04 | 0.07 | −0.15 | 0.2562 | 0.053 | 0.0064 |
| X094350 | 2.8 | 0 | 1.3 | 0 | 0 | 0 | 0.01 | 0.12 | 0.11 | 0.7336 | 0 | 0.0116 |
| L11 | 6.9 | 3.4 | 2.6 | 1.4 | 2.4 | 0 | 0.16 | 0.1 | −0.1 | 0 | 0.0027 | 0.0126 |
| L38 | 6.9 | 3.4 | 3.9 | 0 | 0 | 0 | −0.03 | 0.1 | 0.06 | 0.465 | 0.0011 | 0.0898 |
| L37 | 2.8 | 4.9 | 3.9 | 0 | 2.4 | 1.3 | −0.03 | 0.16 | 0.05 | 0.3436 | 0 | 0.2103 |
| PvGAMA | 9.7 | 6.8 | 9.1 | 6.9 | 2.9 | 0.9 | 0.19 | 0.14 | 0.05 | 0 | 0 | 0.1987 |
| L49 | 9.7 | 3.9 | 3 | 0 | 0 | 0 | −0.09 | 0 | −0.21 | 0.0088 | 0.9079 | 2.00E−04 |
| L47 | 12.5 | 4.4 | 5.2 | 5.6 | 1 | 0 | 0.02 | 0.15 | −0.06 | 0.5816 | 0 | 0.3004 |
| L48 | 0 | 0 | 3.5 | 0 | 0 | 0 | −0.08 | 0 | −0.14 | 0.0173 | 0.9939 | 0.0011 |
| RBP2.P2 | 5.6 | 4.9 | 4.3 | 0 | 0 | 0 | −0.01 | 0.13 | −0.02 | 0.7196 | 0 | 0.5467 |
| L03 | 2.8 | 0 | 3 | 1.4 | 4.4 | 0.4 | −0.03 | 0.03 | −0.16 | 0.4053 | 0.3609 | 2.00E−04 |
| L52 | 1.4 | 5.9 | 3 | 0 | 0.5 | 0 | −0.15 | 0.15 | 0.01 | 2.00E−04 | 0 | 0.8287 |
| L40 | 9.7 | 0 | 0 | 0 | 0 | 0 | −0.09 | 0.04 | −0.15 | 0.0058 | 0.1846 | 0.0018 |

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and apparatuses which may further include any and all elements from any other disclosed methods, systems, and apparatuses, including any and all elements corresponding to target particle separation, focusing/concentration. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

1.-46. (canceled)

47. A diagnostic test for Plasmodium vivax, to determine a likelihood of a specific timing of infection by P. vivax in a subject by determining a level of antibodies to a plurality of antigens in a sample from the subject, wherein the level is measured of at least one antibody to protein selected from at least one of PVX_084720 (SEQ ID NO:35) or PVX_000930 (SEQ ID NO:109) wherein the level of antibody is correlated with the time since infection.

48. The test of claim 47, wherein the level is further measured of at least one antibody to protein selected from at least one protein selected from the group consisting of PVX_099980 (L01) (SEQ ID NO:1), PVX_112670 (SEQ ID NO:23), PVX_087885 (SEQ ID NO:45), PVX_082650 (SEQ ID NO:59), PVX_088860 (SEQ ID NO:5), PVX_112680 SEQ ID NO:11), PVX_112675 SEQ ID NO:21), PVX_092990 (SEQ ID NO:39), PVX_091710 (SEQ ID NO:43), PVX_117385 SEQ ID NO:49), PVX_098915 (SEQ ID NO:77), PVX_088820 (SEQ ID NO:79), PVX_117880 (SEQ ID NO:7), PVX_121897 (SEQ ID NO:95), PVX_125728 (SEQ ID NO:97), PVX_001000 (SEQ ID NO:65), PVX_084340 SEQ ID NO:75), PVX_090330 (SEQ ID NO:99), PVX 125738 (SEQ ID NO: 103), PVX_096995 (SEQ ID NO:3), PVX_097715 (SEQ ID NO:13), PVX_094830 (SEQ ID NO: 19), PVX_101530 (SEQ ID NO:9), PVX_090970 (SEQ ID NO:27), PVX_003770 (SEQ ID NO:37), PVX_112690 (SEQ ID NO:41), PVX_003555 (SEQ ID NO:47), PVX_094255 (SEQ ID NO:61), PVX_090265 (SEQ ID NO:53), PVX_099930 (SEQ ID NO:73), PVX 123685 (SEQ ID NO: 101), PVX_002550 (SEQ ID NO:25), PVX_082700 (SEQ ID NO:55), PVX_097680 (SEQ ID NO:63), PVX_097625 (SEQ ID NO:67), PVX_082670 (SEQ ID NO:41), PVX 082735 (SEQ ID NO:81), PVX_082645 (SEQ ID NO:83), PVX_097720 (SEQ ID NO:107), PVX_000930 (SEQ ID NO:109), PVX_094350, PVX_114330, PVX_088820 (SEQ ID NO:79), PVX_080665, PVX_092995, PVX_087885 (SEQ ID NO:45), PVX_003795, PVX 087110 (SEQ ID NO:69), PVX_087670, PVX_081330, PVX 122805, RBP1b (P7), RBP2a (P9), RBP2b (P25) (PVX_094255) (SEQ ID NO:61), RBP2cNB (M5), RBP2-P2 (P55), PvDBP R3-5, PvGAMA, PyRipr, PvCYRPA, Pv DBPII (AH), PvEBP, RBP1a (P5) and Pv DBP (SacI).

49. The test of claim 47, wherein the level is measured of antibody to protein PVX_084720 (SEQ ID NO:35) and PVX_000930 (SEQ ID NO:109) and of antibody to at least one protein selected from the group consisting of PVX_099980 (L01) (SEQ ID NO:1), PVX_112670 (SEQ ID NO:23), PVX_087885 (SEQ ID NO:45), PVX_082650 (SEQ ID NO:59), PVX_088860 (SEQ ID NO:5), PVX_112680 SEQ ID NO: II), PVX_112675 SEQ ID NO:21), PVX_092990 (SEQ ID NO:39), PVX_091710 (SEQ ID NO:43), PVX_117385 SEQ ID NO:49), PVX_098915 (SEQ ID NO:77), PVX_088820 (SEQ ID NO:79), PVX_117880 (SEQ ID NO:7), PVX_121897 (SEQ ID NO:95), PVX_125728 (SEQ ID NO:97), PVX_001000 (SEQ ID NO:65), PVX_084340 SEQ ID NO:75), PVX_090330 (SEQ ID NO:99), PVX_125738 (SEQ ID NO:103), PVX 096995 (SEQ ID NO:3), PVX_097715 (SEQ ID NO:13), PVX_094830 (SEQ ID NO:19), PVX_101530 (SEQ ID NO:9), PVX_090970 (SEQ ID NO:27), PVX_003770 (SEQ ID NO:37), PVX_112690 (SEQ ID NO:41), PVX_003555 (SEQ ID NO:47), PVX_090265 (SEQ ID NO:53), PVX_099930 (SEQ ID NO:73), PVX_123685 (SEQ ID NO:101), PVX_002550 (SEQ ID NO:25), PVX_082700 (SEQ ID NO:55), PVX_097680 (SEQ ID NO:63), PVX_097625 (SEQ ID NO:67), PVX_082670 (SEQ ID NO:41), PVX_082735 (SEQ ID NO:81), PVX_082645 (SEQ ID NO:83), PVX_097720 (SEQ ID NO:107), PVX_000930 (SEQ ID NO: 109), PVX_094350, PVX_114330, PVX_088820 (SEQ ID NO:79), PVX_080665, PVX_092995, PVX_087885 (SEQ ID NO:45), PVX 003795, PVX_087110 (SEQ ID NO:69), PVX_087670, PVX_081330, PVX_122805, RBP1b (P7), RBP2a (P9), RBP2b (P25) (PVX_094255) (SEQ ID NO:61), RBP2cNB (M5), RBP2-P2 (P55), PvDBP R3-5, PvGAMA, PvRipr, PvCYRPA, Pv DBPII (AH), PvEBP, RBP1a (P5) and Pv DBP (SacI).

50. The test of claim 47, wherein the level is measured of antibody to protein PVX_084720 (SEQ ID NO:35) and PVX_000930 (SEQ ID NO:109) and of antibody to at least two proteins selected from the group consisting of PVX_099980 (L01) (SEQ ID NO:1), PVX_112670 (SEQ ID NO:23), PVX_087885 (SEQ ID NO:45), PVX_082650 (SEQ ID NO:59), PVX_088860 (SEQ ID NO:5), PVX_112680 SEQ ID NO: 11), PVX_112675 SEQ ID NO:21), PVX_092990 (SEQ ID NO:39), PVX_091710 (SEQ ID NO:43), PVX_117385 SEQ ID NO:49), PVX_098915 (SEQ ID NO:77), PVX_088820 (SEQ ID NO:79), PVX_117880 (SEQ ID NO:7), PVX 121897 (SEQ ID NO:95), PVX_125728 (SEQ ID NO:97), PVX_001000 (SEQ ID NO:65), PVX_084340 SEQ ID NO:75), PVX_090330 (SEQ ID NO:99), PVX_125738 (SEQ ID NO: 103), PVX_096995 (SEQ ID NO:3), PVX_097715 (SEQ ID NO:13), PVX_094830 (SEQ ID NO:19), PVX_101530 (SEQ ID NO:9), PVX_090970 (SEQ ID NO:27), PVX_003770 (SEQ ID NO:37), PVX_112690 (SEQ ID NO:41), PVX_003555 (SEQ ID NO:47), PVX_090265 (SEQ ID NO:53), PVX_099930 (SEQ ID NO:73), PVX 123685 (SEQ ID NO:101), PVX_002550 (SEQ ID NO:25), PVX_082700 (SEQ ID NO:55), PVX_097680 (SEQ ID NO:63), PVX_097625 (SEQ ID NO:67), PVX_082670 (SEQ ID NO:41), PVX_082735 (SEQ ID NO:81), PVX_082645 (SEQ ID NO:83), PVX 097720 (SEQ ID NO: 107), PVX_000930 (SEQ ID NO:109), PVX_094350, PVX_114330, PVX_088820 (SEQ ID NO:79), PVX_080665, PVX_092995, PVX_087885 (SEQ ID NO:45), PVX_003795, PVX_087110 (SEQ ID NO:69), PVX_087670, PVX_081330, PVX_122805, RBP1b (P7), RBP2a (P9), RBP2b (P25) (PVX_094255) (SEQ ID NO:61), RBP2cNB (M5), RBP2-P2 (P55), PvDBP R3-5, PvGAMA, PvRipr, PvCYRPA, Pv DBPII (AH), PvEBP, RBP1a (P5) and Pv DBP (SacI).

51. The test of claim 47, wherein a model of the decay of antibody titers over time is used to determine the time since last infection.

52. The test of claim 51, comprising determining a level of 2 to 8 antibodies.

53. The test of claim 47, wherein the level of antibodies is measured at a plurality of time points.

54. The test of claim 47, wherein antibody levels are measured in the subject and time since infection is estimated continuously, wherein antibody level is compared with a titration curve to provide an estimate of antibody.

55. The test of claim 54, wherein antibody levels are measured according to a method selected from the group consisting of bead-based assays, the enzyme linked immunosorbent assay (ELISA), protein microarrays and the luminescence immunoprecipitation system (LIPS).

56. A method for diagnosis of P. vivax, comprising performing the diagnostic test of claim 47, wherein the level of antibody and the timing of infection identifies individuals with a high probability of being infected with liver-stage hypnozoites.

57. The test of claim 47, wherein said specific timing identifies whether and when an infection occurred within an elapsed time period of 0 to 12 months.

58. The test of claim 57, wherein said time period is differentiated by month, by week, or by day.

59. The test of claim 57, wherein a particular time period is determined as a binary decision of a more recent or an older infection, with each time point as a cut-off.

60. The test of claim 59, wherein said cut off determines whether an infection in a subject was within the past 9 months or later than the past 9 months.

61. The test of claim 47, comprising further determining an estimate of the time since last P. vivax blood-stage infection according to the time since last PCR-detectable blood-stage parasitemia, or as the time since last infective mosquito bite.

62. The test of claim 61 comprising determining a frequency of infections during a particular time period and/or time since last infection.

63. The test of claim 47 for detecting an asymptomatic infection by P. vivax.

64. The test of claim 47 for detecting a dormant infection, wherein the level of antibody indicates P. vivax is present in the liver but is not present at significant levels in the blood.

65. The test of claim 47 for detecting antibodies to malarial proteins that are present in the blood wherein the level of antibody and the timing of infection indicate a high degree of probability of liver-stage infection.

66. The test of claim 47 wherein the level of antibody and the timing of infection provides for determining progression of infection by P. vivax in a population of a plurality of subjects.

67. The test of claim 47 wherein the level of antibody and the timing of infection provides for determining whether the infection is starting or whether the infection has reached a peak in terms of exposure of individuals who are naïve to the particular strain of P. vivax causing the infection.

68. The test of claim 47 for measuring antibodies in the blood of the subject at a plurality of time points to determine decay in the level of each antibody in the blood; and fitting such decay to a suitable model to determine at least one infection parameter selected from probability of liver-stage infection, determination of the progression of infection, and rate of propagation of the Plasmodium species in a population.

69. The test of claim 68, wherein decay in the level of a plurality of different antibodies is determined and the different antibodies are selected to have a range of different half-lives.

70. The test of claim 68, wherein from two up to twenty different antibodies are measured.

71. The test of claim 47, wherein a model for determining at least one parameter about the infection in the subject is selected from the group consisting of linear discriminant analysis (LDA), quadratic discriminant analysis (QDA), combined antibody dynamics (CAD), decision trees, random forests, boosted trees and modified decision trees.


SEQUENCE LISTING (142 sequences)

SEQ ID NO: 1 - protein, 115 aa, Plasmodium vivax
MNESKEILSQLLNVQTQLLTMSSEHTCIDTNVPDNAACYRYLDGTEEWRCLLTFKEEGGKCVPASNVTCKDNNGGCAPEAECKMTDSNKIVCKCTKEGSEPLFEGVFCSHHHHHH

SEQ ID NO: 2 - DNA, 348 bp, Plasmodium vivax
  source 1..348: mol_type = genomic DNA
atgaacgagtccaaggagatcctcagccaactcctgaacgtgcaaacccagctcctgaccatgtccagcgagcacacctgcatcgacaccaacgtcccagacaacgccgcctgctacaggtacctggacggcaccgaggagtggcgctgcctcctgaccttcaaggaagagggcggcaagtgcgtgccagcctccaacgtcacctgcaaggacaacaacggcggctgcgctccagaggctgagtgcaagatgaccgacagcaacaagatcgtgtgcaagtgcaccaaggaaggctccgagccactcttcgagggcgtcttctgcagccaccaccaccaccaccactga

SEQ ID NO: 3 - protein, 427 aa, Plasmodium vivax
MKTETVTSRSNPHQAIEYANQGPSRDKVEEWKRNAWTDWMVQLDDDWKDFNAQIEEEKKAWIEEKEGDWVILLKHLQNKWLHFNPNLDAEYQTDMLAKSETWDERQWKMWISTEGKQLLEMDLKKWFTNNEMIYCKWTMDEWNEWKNEKIKEWVTSEWKESEDQYWSKYDDATIQTLTVAERNQWFKWKERIYREGIEWKNWIAIKESKFVNANWNSWSEWKNEKRLEFNDWIEAFVEKWIRQKQWLIWTDERKNFANRQKAAPGGVAAAPGVFAPRPAFGAPSGFAPRPGFAAPSQPPRYSFAAASGYVAPSATSEAAPATSEAPASAEATTALSSETTTPVNPEETAASPEAATPVNPEETAASSETTTVNPEATPVNPEAPVAEPEKKEEEPAAEPLLAIEPAQTEPAALEAAPSTSAHHHHHH

SEQ ID NO: 4 - DNA, 1284 bp, Plasmodium vivax
  source 1..1284: mol_type = genomic DNA
atgaagaccgagacggtgacctccaggagcaacccacaccaagccatcgagtacgccaaccagggcccatccagggacaaggtggaggagtggaagcgcaacgcctggaccgactggatggtccaactcgacgacgactggaaggacttcaacgcccagatcgaggaagagaagaaggcctggattgaggagaaggaaggcgactgggtcatcctcctgaagcacctccaaaacaagtggctgcacttcaacccaaacctcgacgccgagtaccagaccgacatgctggccaagtccgagacgtgggacgagaggcagtggaagatgtggatcagcaccgagggcaagcagctcctggagatggacctcaagaagtggttcaccaacaacgagatgatctactgcaagtggaccatggacgagtggaacgagtggaagaacgagaagatcaaggagtgggtgacctccgagtggaaggagagcgaggaccaatactggtccaagtacgacgacgccaccatccaaaccctgaccgtcgccgagcgcaaccagtggttcaagtggaaggagaggatctaccgcgagggcatcgagtggaagaactggatcgccatcaaggagagcaagttcgtgaacgccaactggaactcctggtctgagtggaagaacgagaaaaggctggagttcaacgactggatcgaggccttcgtcgagaagtggatccgccaaaagcagtggctgatctggaccgacgagaggaagaacttcgccaaccgccaaaaggctgctccaggcggcgtggctgccgccccaggcgtcttcgccccacgcccagccttcggcgccccatccggcttcgccccaaggccaggcttcgctgctccaagccagccaccacgctactccttcgctgccgccagcggctacgtggctccatccgctaccagcgaggctgctccagccacctccgaggccccagccagcgccgaggctaccaccgctctctccagcgagacgaccaccccagtcaacccagaggagacggctgctagcccggaggctgctaccccagtgaacccggaggagacggctgcctccagcgagacgacgacggtcaacccagaggccaccccggtgaacccagaggctccagtggctgagccagagaagaaggaagaggagccagctgctgagccactgctcgctatcgagccagctcaaaccgagccagctgctctggaggctgctccatccaccagcgcccaccaccaccaccaccactga

SEQ ID NO: 5 - protein, 387 aa, Plasmodium vivax
MQLELEPAPDYESTSPTVPVRLLLHDDYAPNAEDMFGPEASQVMTNLYETIDEDGTTTDGYQNGSDDDQSNQSDSNDDAVMLNYLSNETDSFDELIDEIDNHKKKKKIYSPLRKPVLKRSDSSDSLSDYELDEVLRQTENEPEEDEDLDLSLEDSFEVINYPWKDILESSPYSTDHTNEEDFSSLEELELEDPVQEMNFGKLKFFEIGDPDLLIRKTPITPNTKTKSGLEKNGNNTEASNINQHEKEKMDKRKRRTHKQFKNPIENFSVTTTYDDFLKQNGLRDHPSKHQKDSSEPFVLDQYNYRNAKFKNVRFYILRMLYDNIKDIGLKEFQYLKSHKYEVEEFIKNILRNNLICLTFSQEDHLFNDAHLLIEKASIKSEHHHHHH

SEQ ID NO: 6 - DNA, 1164 bp, Plasmodium vivax
  source 1..1164: mol_type = genomic DNA
atgcagctggagctggagccagccccagactacgagtccaccagcccaaccgtgccagtcaggctcctgctccacgacgactacgccccaaacgccgaggacatgttcggcccagaggcctcccaagtgatgaccaacctctacgagacgatcgacgaggacggcaccaccaccgacggctaccaaaacggctccgacgacgaccaaagcaaccagtccgacagcaacgacgacgccgtcatgctcaactacctgtccaacgagacggacagcttcgacgagctcatcgacgagatcgacaaccacaagaagaagaagaagatctactccccactcaggaagccagtgctgaagcgcagcgactccagcgactccctgagcgactacgagctcgacgaggtcctgcgccagaccgagaacgagccagaggaagacgaggacctggacctctccctggaggacagcttcgaggtcatcaactacccatggaaggacatcctggagtccagcccatacagcaccgaccacaccaacgaggaagacttctccagcctggaggagctggagctggaggacccagtccaagagatgaacttcggcaagctgaagttcttcgagatcggcgacccagacctgctcatcaggaagaccccaatcaccccaaacaccaagaccaagtccggcctggagaagaacggcaacaacaccgaggccagcaacatcaaccagcacgagaaggagaagatggacaagcgcaagaggcgcacccacaagcaattcaagaacccaatcgagaacttctccgtgaccaccacctacgacgacttcctcaagcaaaacggcctgagggaccacccaagcaagcaccagaaggactccagcgagccattcgtgctcgaccaatacaactaccgcaacgccaagttcaagaacgtcaggttctacatcctccgcatgctgtacgacaacatcaaggacatcggcctcaaggagttccagtacctgaagtcccacaagtacgaggtcgaggagttcatcaagaacatcctcaggaacaacctcatctgcctgaccttcagccaagaggaccacctgttcaacgacgcccacctgctcatcgagaaggcctccatcaagagcgagcaccaccaccaccaccactga

SEQ ID NO: 7 - protein, 822 aa, Plasmodium vivax
MNAGDGQGVYGGNGINNPLVYHVQHGVNIPNSNSDKKASDHTPDEDEDTYGRTRNKRYMHRNPGEKYKGSNSPHDSNDDSGDTEYELNEGDVKRLTPKNKKGATTEEVDTYPYGKKTNGSEFPRMNGSETGHYGYNNTGSGGHNDENGYTPIIVKYDNTHAKNRANEIEENLNKGEYSRIKMAKGKKGQKSGGYESDGEDSDVDSSNVFYVDNGQDMLIKEKMSRSEGPDEMSEEGLNVKYKAQRGPVNYHFSNYMNLDKRNTLSSNEIELQKMIGPKFSEEVNKYCRLNEPSSKKGEFLNVSFEYSRALEELRSEMINELQKRKAVGSNYYNNILNAIYTSMNRKNANFGRDAYEDKSFISEANSFRNEEMQPLSAKYNKILRQYLCHVFVGNPGVNQLERLYFHNLALGELIEPIRRKYNKLASSSVGLNYEIYIASSSNIYLMGHLLMLSLAYLSYNSYFVQGLKPFYSLETMLMANSDYSFFMYNEVCNVYYHPKGTFNKDITFIPIESRPGRHSTYVGERKVTCDLLELILNAYTLINVHEIQKVFNTSEAYGYENSISFGHNAVRIFSQVCPRDDAKNTFGCDFEKSTLYNSKVLKMDEGDKENQRSLKRAFDMLRTFAEIESTSHLGDPSPNYISLIFEQNLYTDFYKYLFWYDNRELINVQIRNAGRRKKGKKVKFVYDEFVKRGKQLKDKLIKIDAKYNARSKALLVFYALVDKYANIFRKSENVRKFFLNDVSSIRHHLYLNSVLTKSPKSNLDSMKKTLEELQSLTNAPLKFIVRGNNLKFLNNVAKFENLFYVNLFIMSSLSRKHHHHHH

SEQ ID NO: 8 - DNA, 2469 bp, Plasmodium vivax
  source 1..2469: mol_type = genomic DNA
atgaacgctggcgacggccaaggcgtgtacggcggaaacggcatcaacaacccactcgtgtaccacgtccagcacggcgtcaacatcccaaactccaacagcgacaagaaggccagcgaccacaccccagacgaggacgaggacacctacggcaggacccgcaacaagaggtacatgcaccgcaacccaggcgagaagtacaagggctccaacagcccacacgactccaacgacgacagcggcgacaccgagtacgagctgaacgagggcgacgtgaagaggctcaccccaaagaacaagaagggcgccaccaccgaggaagtggacacctacccatacggcaagaagaccaacggcagcgagttcccacgcatgaacggctccgagacgggccactacggctacaacaacaccggcagcggcggccacaacgacgagaacggctacaccccaatcatcgtgaagtacgacaacacccacgccaagaacagggccaacgagatcgaggagaacctcaacaagggcgagtactcccgcatcaagatggccaagggcaagaagggccaaaagtccggcggctacgagagcgacggcgaggactccgacgtcgactccagcaacgtgttctacgtcgacaacggccaggacatgctgatcaaggagaagatgtccaggagcgagggcccagacgagatgagcgaggaaggcctcaacgtgaagtacaaggcccaaaggggcccagtcaactaccacttctccaactacatgaacctggacaagcgcaacaccctctccagcaacgagatcgagctccagaagatgatcggcccaaagttcagcgaggaagtgaacaagtactgcaggctgaacgagccatccagcaagaagggcgagttcctcaacgtctccttcgagtacagcagggccctggaggagctgaggtccgagatgatcaacgagctgcaaaagcgcaaggccgtgggcagcaactactacaacaacatcctcaacgccatctacacctccatgaacaggaagaacgccaacttcggccgcgacgcctacgaggacaagtccttcatcagcgaggccaacagcttcaggaacgaggagatgcaaccactctccgccaagtacaacaagatcctgcgccagtacctctgccacgtgttcgtcggcaacccaggcgtgaaccaactggagcgcctgtacttccacaacctcgccctgggcgagctgatcgagccaatcaggcgcaagtacaacaagctggcctccagctccgtcggcctcaactacgagatctacatcgccagctccagcaacatctacctcatgggccacctcctgatgctcagcctggcctacctgtcctacaacagctacttcgtgcagggcctcaagccattctactccctcgaaaccatgctcatggccaactccgactacagcttcttcatgtacaacgaggtgtgcaacgtctactaccacccaaagggcaccttcaacaaggacatcaccttcatcccaatcgagagcaggccaggcaggcactccacctacgtgggcgagaggaaggtcacctgcgacctcctggagctcatcctgaacgcctacaccctgatcaacgtgcacgagatccaaaaggtcttcaacaccagcgaggcctacggctacgagaactccatcagcttcggccacaacgccgtgaggatcttctcccaggtctgcccacgcgacgacgccaagaacaccttcggctgcgacttcgagaagagcaccctgtacaactccaaggtgctcaagatggacgagggcgacaaggagaaccagaggtccctgaagcgcgccttcgacatgctccgcaccttcgccgagatcgagtccaccagccacctcggcgacccaagcccaaactacatctccctgatcttcgagcaaaacctctacaccgacttctacaagtacctgttctggtacgacaacagggagctcatcaacgtgcagatccgcaacgccggcaggcgcaagaagggcaagaaggtgaagttcgtctacgacgagttcgtcaagaggggcaagcaactgaaggacaagctcatcaagatcgacgccaagtacaacgcccgcagcaaggccctcctggtgttctacgccctggtcgacaagtacgccaacatcttcaggaagtccgagaacgtgcgcaagttcttcctcaacgacgtctccagcatcaggcaccacctctacctgaacagcgtgctgaccaagtccccaaagagcaacctcgacagcatgaagaagaccctggaggagctgcagtccctcaccaacgccccactgaagttcatcgtcaggggcaacaacctgaagttcctcaacaacgtggccaagttcgagaacctgttctacgtgaacctcttcatcatgtccagcctctcccgcaagcaccaccaccaccaccactga

SEQ ID NO: 9 - protein, 337 aa, Plasmodium vivax
MNVNKKSSGEENNTKQALGLRVSRTLAKDGANENAEEGLSEEEEEAVEEGEEEAVEEGEEEVVEEEGEEVVEGEEEEVVEGEEEVVEDEEVVEGEEYAEGEEPVEGEEYAEGEEPVEGEEPVEVEEYAEGEEPVEGEEYAEGEEPVEGEEVVEGEEVVEGEEVAEGEEVAEGEEVAEGEEAVEGEEVAEGEEVAEGEEVAEGEEAAEEGAAEEGATEEGATEEGATKEEATEKAAEGEETAESEKPAEEQPTTFVETVEKKVEPVSKPPFKPLFPVDEKYLETLEDIAQSFLKEFQEAEGKRKQKKVKKRAKKITKKLAKEYAKKFKSKKKHHHHHH

SEQ ID NO: 10 - DNA, 1014 bp, Plasmodium vivax
  source 1..1014: mol_type = genomic DNA
atgaacgtcaacaagaagtccagcggcgaggagaacaacaccaagcaagctctgggcctgagggtgtcccgcaccctcgctaaggacggcgccaacgagaacgccgaggaaggcctcagcgaggaagaggaagaggccgtcgaggaaggcgaggaagaggccgtggaggaaggcgaggaagaggtggtcgaggaagagggcgaggaagtggtcgagggcgaggaagaggaagtggtggagggggaggaagaggtggtggaggatgaggaagtggtggagggcgaggagtacgctgagggcgaggagccggtggagggggaggagtacgccgagggggaggagccagtggagggcgaggagccagtggaggtggaggagtacgcggagggggaggagccggtggaaggtgaggagtacgccgagggcgaggagcctgtcgagggggaggaagtggtggaaggcgaggaagtggtggaaggtgaggaagtggctgagggcgaggaagtggccgagggggaggaagtggccgagggcgaggaagccgtggagggcgaggaagtggcggagggggaggaagtggcggaaggcgaggaagtggccgaaggcgaggaagccgctgaggaaggcgctgccgaggaaggcgccacggaggaaggcgctaccgaggaaggcgccaccaaggaagaggccaccgagaaggctgctgagggcgaggagacggctgagtccgagaagccagctgaggagcaaccaaccaccttcgtggagacggtcgagaagaaggtggagccagtcagcaagccaccattcaagccactcttcccagtcgacgagaagtacctcgaaaccctggaggacatcgcccaatccttcctgaaggagttccaagaggccgagggcaagaggaagcagaagaaggtgaagaagcgcgccaagaagatcaccaagaagctcgccaaggagtacgccaagaagttcaagtccaagaagaagcaccaccaccaccaccactga

SEQ ID NO: 11 - protein, 288 aa, Plasmodium vivax
MPKPDQKNLKGGVKNAPLQQRKGSVPINPPKPVNDKLKDGSNKTETKNAKNTLSKPPMQVTDKSKDEAKKTPLQSTPKLTPKTKEVPKESNMEMWLKDTKDEYENLKCQYRTCLYDWFRKINDEYNELLNKLEEKWAKFPNDPKNKDVFDNLKTSSLKNDEKKAQWMRKNLKDLMREQVDEWLEGKKKIYEGMSPTYWDAWEKKIAKGLMGAAWYKMNSSGRTKEWDKLRNELETRYNKKIKSLWGGFHRDVYFRFKEWIEEVFNKWIENKQIDTWMNSGKKHHHHHH

SEQ ID NO: 12 - DNA, 867 bp, Plasmodium vivax
  source 1..867: mol_type = genomic DNA
atgccaaagccagaccaaaagaacctcaagggcggcgtgaagaacgccccactgcaacagaggaagggctccgtgccaatcaacccaccaaagccagtcaacgacaagctcaaggacggcagcaacaagaccgagacgaagaacgccaagaacaccctgtccaagccaccaatgcaagtgaccgacaagagcaaggacgaggccaagaagaccccactccagtccaccccaaagctgaccccaaagaccaaggaagtgccaaaggagagcaacatggagatgtggctcaaggacaccaaggacgagtacgagaacctcaagtgccagtacaggacctgcctgtacgactggttccgcaagatcaacgacgagtacaacgagctcctgaacaagctggaggagaagtgggccaagttcccaaacgacccaaagaacaaggacgtgttcgacaacctcaagacctccagcctgaagaacgacgagaagaaggcccagtggatgaggaagaacctcaaggacctgatgagggagcaggtggacgagtggctggagggcaagaagaagatctacgagggcatgtccccaacctactgggacgcctgggagaagaagatcgctaagggcctgatgggcgctgcttggtacaagatgaactcctccggcaggaccaaggagtgggacaagctcaggaacgagctcgaaacccgctacaacaagaagatcaagtccctctggggcggcttccacagggacgtgtacttccgcttcaaggagtggatcgaggaagtgttcaacaagtggatcgagaacaagcaaatcgacacctggatgaacagcggcaagaagcaccaccaccaccaccactga

SEQ ID NO: 13 - protein, 438 aa, Plasmodium vivax
MQYSIVKNEITKRRKPKIRNESPPDGNSPGGGKNNAAGNNGGGDNNAKNKAANKAANNAANKAANNAANNAANNAANNAANNAANNAANNAANNAANNAANNAANNANEQNGNKKKKGKPKKEEADLPVQAQNENDRNKIEDIADEAELFAEEAKMLADLASKRSKEVEQILSSIPENKFGSEPKEDAIFAAKDAVRASEDAMKAAQKARAAETVTQANEEKDKAKTAKELAERSAQIVKKNAVEALKEFGKIAEAAEMEAIKIPIPENLKPKKKVKQPRAAAQKVEPTQATAHKVVPPPAEPPRAPSPPPPPAKPEAAPPAKEVAPAVTTPEAPKEEAPKADAAPAAPQPAAESKVAKEPTDQSAENQSDSLYKETNIKEGTEEAGTGQEQKQEPELQNLLEQQMNIFYILVQFFKSKIKALIKFLLILVSHHHHHH

SEQ ID NO: 14 - DNA, 1317 bp, Plasmodium vivax
  source 1..1317: mol_type = genomic DNA
atgcaatactccatcgtgaagaacgagatcaccaagaggcgcaagccaaagatcaggaacgagtccccaccagacggcaacagcccaggcggcggcaagaacaacgctgctggcaacaacggcggcggcgacaacaacgccaagaacaaggctgctaacaaggctgctaacaacgccgccaacaaggccgccaacaacgctgctaacaacgccgcgaacaacgccgccaacaacgccgccaacaacgcagctaacaacgccgctaacaacgcggccaacaacgccgcgaacaacgcggcgaacaacgctgccaacaacgccaacgagcaaaacggcaacaagaagaagaagggcaagccaaagaaggaagaggccgacctcccagtgcaagcccagaacgagaacgacaggaacaagatcgaggacatcgctgacgaggctgagctgttcgctgaggaagccaagatgctcgccgacctggcctccaagcgcagcaaggaagtggagcagatcctctccagcatcccagagaacaagttcggctccgagccaaaggaagacgccatcttcgctgctaaggacgccgtgagggctagcgaggacgccatgaaggctgctcaaaaggccagggccgctgagacggtcacccaggccaacgaggagaaggacaaggctaagaccgctaaggagctggctgagaggtccgctcaaatcgtgaagaagaacgccgtcgaggccctgaaggagttcggcaagatcgccgaggccgccgagatggaggccatcaagatcccaatcccagagaacctgaagccaaagaagaaggtgaagcaaccaagggccgccgcccaaaaggtggagccaacccaagctaccgctcacaaggtggtgccaccaccagctgagccaccacgcgccccatccccaccaccaccaccagctaagccagaggctgccccaccagctaaggaagtggctccagctgtcaccaccccagaggctccaaaggaagaggccccaaaggctgacgctgctccagctgccccacagccagccgccgagtccaaggtcgccaaggagccaaccgaccagagcgccgagaaccaatccgacagcctctacaaggagacgaacatcaaggaaggcaccgaggaagccggcaccggccaagagcagaagcaagagccagagctccaaaacctcctggagcaacagatgaacatcttctacatcctggtgcagttcttcaagtccaagatcaaggccctcatcaagttcctcctgatcctggtcagccatcaccaccaccaccactga

SEQ ID NO: 15 - protein, 355 aa, Plasmodium vivax
MDENTGWPIDYEFNSKTLPSIEVKLSPPENPLPQVAAEIKLLESARLKLEEGMMQKLEDEYNKSLSSAKIKIQDTVEKSLSIFNDPNMLGSVISNSVKMLRSENVKKRTENVQAKHNLKKMQTVNQAKSGPLPPPELRKHTSFLEQNYVNRVLPSVKISLSELTEPSVEIKEKIEEMEQYRTDEEVAMFEMAISEFSILTDITILELEKQIQLQLNPFLVDKKVVHRALTKELKELEQREEKQKIKENFQRQSSFIEAGEDEDTGNILNVKISQTDYGYPTVDELVMQMQKRRDISEKLERQKILDLQMKLLKAQSEMIKDALHFALSKVIAQYSPLVETMKLESMRMLHHHHHH

SEQ ID NO: 16 - DNA, 1068 bp, Plasmodium vivax
  source 1..1068: mol_type = genomic DNA
atggacgagaacaccggctggccaatcgactacgagttcaactccaagaccctgccaagcatcgaggtgaagctctccccaccagagaacccactgccacaagtcgccgccgagatcaagctcctggagagcgcccgcctcaagctcgaagagggcatgatgcagaagctggaggacgagtacaacaagtccctgtccagcgccaagatcaagatccaagacaccgtggagaagtccctcagcatcttcaacgacccaaacatgctgggctccgtgatctccaacagcgtcaagatgctcaggagcgagaacgtgaagaagcgcaccgagaacgtccaggccaagcacaacctcaagaagatgcagaccgtcaaccaagccaagagcggcccactcccaccaccagagctgcgcaagcacacctccttcctggagcaaaactacgtgaacagggtcctgccatccgtgaagatctccctcagcgagctgaccgagccaagcgtcgagatcaaggagaagatcgaggagatggagcagtacaggaccgacgaggaagtggccatgttcgagatggccatctccgagttcagcatcctcaccgacatcaccatcctggagctggagaagcaaatccagctccaactgaacccattcctcgtcgacaagaaggtggtccacagggccctgaccaaggagctcaaggagctggagcagcgcgaggagaagcaaaagatcaaggagaacttccagaggcaatccagcttcatcgaggctggcgaggacgaggacaccggcaacatcctcaacgtgaagatctcccagaccgactacggctacccaaccgtggacgagctcgtcatgcagatgcaaaagaggcgcgacatctccgagaagctggagcgccagaagatcctcgacctgcagatgaagctcctgaaggcccagagcgagatgatcaaggacgccctccacttcgccctgtccaaggtcatcgcccaatacagcccactcgtcgagacgatgaagctggagagcatgaggatgctccaccaccaccaccaccactga

SEQ ID NO: 17 - protein, 624 aa, Plasmodium vivax
MSSDGKSSASAKSGSKSGSKYGGSSYSDYSAYDSGSASSVGSREFENEMYEFALQHPMEKLTKEMDILKNDYTKVKEEEGKILDEEHKEIEEKRKEERLKMLAEGDVEKNKGDEEINFIKHDYTDTRIRGGFTEFLSNLNPFKKEIKPMKKEISLITYIPDKIVNKEKIMRDLGISHKYEPYQQSILYTCPNSVFFFDSMENLRKELDKNHEKEAITNKILDHNKECLKNFGLFDFELPDNKTKLGNVIGSIGEYHVRLYEIENDLLKYQPSLDYMTLADDYKLVKNDVNTLENVNFCLLNPKTLEDFLKKKEIMELMGEDPIAYEEKFTKYMEESINCHLESLIYEDLDSSQDTKIVLKNVKSKLYLLQNGLTYKSKKLINKLFNEIQKNPEPIFEKLTWIYENMYHLKRDYTFLAFKTVCDKYVSHNSIYTSLQGMTSYIIEYTRLYGACFKNITIYNAVISGIHEQMKNLMKLMPRSGLLSDVHFEALLHKENKKITRTDYVLNDYDPSVKAYALTQVERLPMVSVINSFFEAKKKALSKMLAQMKLDLFTLTNEDLKIPNDKGANSKLTAKLISIYKAEIKKYFKEMRDDYVFLIKARYKGHYKKNYLLYKRLEHHHHHH

SEQ ID NO: 18 - DNA, 1875 bp, Plasmodium vivax
  source 1..1875: mol_type = genomic DNA
atgagcagcgacggcaagtccagcgcttccgctaagtccggcagcaagtccggcagcaagtacggcggctccagctactccgactacagcgcctacgactccggcagcgcctccagcgtgggcagccgcgagttcgagaacgagatgtacgagttcgccctgcaacacccgatggagaagctcaccaaggagatggacatcctgaagaacgactacaccaaggtgaaggaagaggaaggcaagatcctcgacgaggagcacaaggagatcgaggagaagaggaaggaagagcgcctcaagatgctggccgagggcgacgtggagaagaacaagggcgacgaggagatcaacttcatcaagcacgactacaccgacaccaggatccgcggcggcttcaccgagttcctctccaacctgaacccattcaagaaggagatcaagccgatgaagaaggagatctccctcatcacctacatcccagacaagatcgtcaacaaggagaagatcatgcgcgacctgggcatctcccacaagtacgagccataccaacagagcatcctctacacctgcccaaactccgtgttcttcttcgacagcatggagaacctcaggaaggagctggacaagaaccacgagaaggaagccatcaccaacaagatcctcgaccacaacaaggagtgcctcaagaacttcggcctgttcgacttcgagctcccagacaacaagaccaagctgggcaacgtcatcggctccatcggcgagtaccacgtgaggctctacgagatcgagaacgacctcctgaagtaccaaccaagcctggactacatgaccctcgccgacgactacaagctggtgaagaacgacgtcaacaccctggagaacgtgaacttctgcctcctgaacccaaagaccctggaggacttcctcaagaagaaggagatcatggagctgatgggcgaggacccaatcgcctacgaggagaagttcaccaagtacatggaggagtccatcaactgccacctggagagcctgatctacgaggacctcgactccagccaagacaccaagatcgtgctcaagaacgtcaagtccaagctgtacctcctgcagaacggcctcacctacaagagcaagaagctcatcaacaagctgttcaacgagatccagaagaacccagagccaatcttcgagaagctcacctggatctacgagaacatgtaccacctgaagcgcgactacaccttcctcgccttcaagaccgtgtgcgacaagtatgtgtcccacaacagcatctacacctccctgcaaggcatgaccagctacatcatcgagtacaccaggctctacggcgcctgcttcaagaacatcaccatctacaacgccgtcatctccggcatccacgagcagatgaagaacctcatgaagctgatgccaaggtccggcctcctgagcgacgtgcacttcgaggccctcctgcacaaggagaacaagaagatcacccgcaccgactacgtgctcaacgactacgacccatccgtcaaggcctacgccctgacccaagtggagaggctcccaatggtgtccgtcatcaacagcttcttcgaggccaagaagaaggccctcagcaagatgctggcccagatgaagctcgacctgttcaccctgaccaacgaggacctcaagatcccaaacgacaagggcgccaactccaagctcaccgccaagctgatcagcatctacaaggccgagatcaagaagtacttcaaggagatgagggacgactacgtcttcctgatcaaggcccgctacaaggggcactacaagaagaactacctcctgtacaagcgcctggagcaccaccaccaccaccactga

SEQ ID NO: 19 - protein, 239 aa, Plasmodium vivax
MNTRASKFANSKRKRNGNAMRENKLNNDDVDHYSFLSLRTANEEKAATENDSNNAKKEGEENTNGNEKKNEENGSGNEKRNEENNANEKKNEQTNDQSNGQSNSQTNIPKKNEAVPPEKKINKENLLEYGTHDKDGHFIPSYKTLTDEILSTNNSLERASSFLKIACSHIMKIVEFIPESKLSSQYIKVESKNVYIKDITSECQNIFFSLEKLTMTMIVLNSKMNKLVYVQDKHHHHHH

SEQ ID NO: 20 - DNA, 720 bp, Plasmodium vivax
  source 1..720: mol_type = genomic DNA
atgaacaccagggcctccaagttcgccaacagcaagaggaagcgcaacggcaacgccatgcgcgagaacaagctcaacaacgacgacgtggaccactactccttcctcagcctgaggaccgctaacgaggagaaggctgctaccgagaacgactccaacaacgccaagaaggaaggcgaggagaacaccaacggcaacgagaagaagaacgaggagaacggcagcggcaacgagaagcgcaacgaggagaacaacgctaacgagaagaagaacgagcaaaccaacgaccagtccaacggccaatccaacagccagaccaacatcccaaagaagaacgaggccgtcccaccagagaagaagatcaacaaggagaacctcctggagtacggcacccacgacaaggacggccacttcatcccaagctacaagaccctcaccgacgagatcctgtccaccaacaacagcctggagagggcctccagcttcctgaagatcgcctgctcccacatcatgaagatcgtggagttcatcccagagtccaagctgtccagccaatacatcaaggtggagagcaagaacgtctacatcaaggacatcacctccgagtgccagaacatcttcttcagcctggagaagctgaccatgaccatgatcgtcctcaacagcaagatgaacaagctggtctacgtgcaagacaagcaccaccaccaccaccactga

SEQ ID NO: 21 - protein, 287 aa, Plasmodium vivax
MPKPAQNLKGGVKKPSLQQTKSPLPSKPPKPVNDKLKDDSNKTETKDAKNGLNKPPKNINDKVKDGENKTPSQDLNEPSFKLPMRQKASSWDAWLKGTKKDYENLKCFAKGNLYDWLCSVRDSFELYLQSLESKWTSCSDNTTTVFLCECLAESSGWGDPQWESWVKKELKEQLKTEAQAWISTKKKDFDGLTSKYFSLWKDHRRKELEEEAWKTKASSGGLSEWEELTDKMNTRYTNNLDNMWSNYSGDLLFRFDEWSPEVLEKWIESKQWNQWVKKVRKHHHHHH

SEQ ID NO: 22 - DNA, 864 bp, Plasmodium vivax
  source 1..864: mol_type = genomic DNA
atgccaaagccagcccaaaacctcaagggcggcgtgaagaagccatccctccaacagaccaagtccccactgccaagcaagccaccaaagccagtcaacgacaagctcaaggacgacagcaacaagaccgagacgaaggacgccaagaacggcctgaacaagccaccaaagaacatcaacgacaaggtgaaggacggcgagaacaagaccccatcccaagacctcaacgagccaagcttcaagctgccaatgaggcaaaaggcctccagctgggacgcttggctcaagggcaccaagaaggactacgagaacctgaagtgcttcgccaagggcaacctctacgactggctgtgctccgtccgcgacagcttcgagctctacctgcaatccctggagagcaagtggacctcctgcagcgacaacaccaccaccgtgttcctctgcgagtgcctcgctgagtccagcggctggggcgacccacagtgggagtcctgggtcaagaaggagctcaaggagcaactgaagaccgaggcccaggcctggatcagcaccaagaagaaggacttcgacggcctcacctccaagtacttcagcctgtggaaggaccacaggcgcaaggagctggaggaagaggcctggaagaccaaggcctccagcggcggcctctccgagtgggaggagctgaccgacaagatgaacaccaggtacaccaacaacctcgacaacatgtggtccaactacagcggcgacctcctgttccgcttcgacgagtggtccccagaggtgctggagaagtggatcgagagcaagcagtggaaccagtgggtgaagaaggtcaggaagcaccaccaccaccaccactga

SEQ ID NO: 23 - protein, 309 aa, Plasmodium vivax
MVTEGGDNLDDDLGGDLEGLLGDDAEGGAAGGEGAAAAASAEGLSGEVENELLYVKEDDDDAPAATPDEKPSTSGEETPAAFVDLVNETVPPPAKAPLPLQTKAPQGPKIKDWNQWMKQAKKDFSGYKGTMHTQRHEWTKEKEDELQKFCKYLEKRWMNYTGNIDRECRSDFLKSTQNWNESQWNKWVKSEGKHHMNKQFQKWLDYNKYKLQDWTNTEWNKWKTTVKEQLDDEEWKKKEAAGKTKEWIKCTDKMEKKCLKKTKKHCKNWEKKANSSFKKWEGDFTKKWTSNKQWNSWCKELEKHHHHHH

SEQ ID NO: 24 - DNA, 930 bp, Plasmodium vivax
  source 1..930: mol_type = genomic DNA
atggtgaccgagggcggcgacaacctcgacgacgacctcggcggcgacctggagggcctcctgggcgacgacgctgagggcggcgccgccggcggcgagggcgctgccgccgccgcctccgccgagggcctgagcggcgaggtggagaacgagctcctctacgtgaaggaagacgacgacgacgctccagctgctaccccagacgagaagccatccaccagcggcgaggagacgccagctgctttcgtggacctcgtcaacgagacggtgccaccaccagctaaggccccactcccactgcaaaccaaggccccacagggcccaaagatcaaggactggaaccagtggatgaagcaggccaagaaggacttctccggctacaagggcaccatgcacacccaaaggcacgagtggaccaaggagaaggaagacgagctgcagaagttctgcaagtacctggagaagcgctggatgaactacaccggcaacatcgacagggagtgccgctccgacttcctgaagagcacccaaaactggaacgagtcccagtggaacaagtgggtgaagagcgagggcaagcaccacatgaacaagcaattccagaagtggctggactacaacaagtacaagctccaagactggaccaacaccgagtggaacaagtggaagaccaccgtcaaggagcagctggacgacgaggagtggaagaagaaggaagccgccggcaagaccaaggagtggatcaagtgcaccgacaagatggagaagaagtgcctcaagaagaccaagaagcactgcaagaactgggagaagaaggccaactccagcttcaagaagtgggagggcgacttcaccaagaagtggacctccaacaagcagtggaacagctggtgcaaggagctggagaagcaccaccaccaccaccactga

SEQ ID NO: 25 - protein, 933 aa, Plasmodium vivax
MAVEVVQEAADEVLEEEKIEEPLEIVEEEPVQVAAEEPVEEVLEEVVQEAADEVMEEEKIEEPLEIVAEEPLEIVAEEPVQVAAEEVLVEKEEVNENILNIVEEIKESIVDKLEANEEASEEGNEDLLESAEEAAEEVAEEAVDTTTEADVVETVEEEAANATTEVSAEESLEVSTEAPEETTESESHETFEEDILKNLEENKEANENALEDIKEMKEEFLDYVEQRVEDNENVLVDLLQHLERNAHVNESVLEDLEEIKEDLLANIQMAEETRKEVTDASAESAEEVEEPVEVSAEVAAEEPVEVAAEEPVEVTAEEPVEVTAEEPVEIPTEENIFDVIEEIKEKVLENLEETTAESVAESVGEGADENALDVLKEMQESLLENFGQKIEANENILASVLENIQEKVELNKSVLVDVLAELKEEAVSQRETAQEVAAELVEEAAEVPAVEPVEEEVVEPAVEVVEEPVEEEVVEPVVDVIEEPAVEVVEVPVEETVEEPVEVTAEEPVEVTAEEPVEETVEEPVVEVVEEPVEEPVVEAIEEPVVEPVVEPAVEVIEDATEEPVEEAAEEPDVEVAEGSAIESVEEAFEQIIEDAAQVIAEESVEETAEQILEQATQAVTEEAADAADVADAEEAVGTAQVVTEESVAEAIEDTVEEISAEPIQATIEGIVGEVVESVEENIEAVEEAIKDIVEGAVEGAPELSLEEMIEDVMVGTVAEEDSAKEAAEETVEEVVQEDAAEEEAAKEAAEETVEEAEREATQEAVEETVEDVVEEVSAEAVEEIVLETPEGTSDESVETVVEHAVEDSLGETIATIVDDVAEETTEKSEESVVDNLGVKVEEVLDVDVEEVAQEAADDVIMRVSENESEGESGAESGEEVEELESALFEVEKDIKKKVLDMFSGNVEFDEKESEKLALDLQKNLLSHHHHHH

SEQ ID NO: 26 - DNA, 2802 bp, Plasmodium vivax
  source 1..2802: mol_type = genomic DNA
atggctgtggaggtggtccaagaggccgctgacgaggtgctcgaagaggagaagatcgaggagccactggagatcgtggaggaagagccagtgcaagtcgccgccgaggagccagtcgaggaagtgctcgaagaggtggtgcaagaggccgccgacgaggtcatggaggaagagaagatcgaggagcctctggagatcgtcgctgaagaacctctggagatcgtggctgaggagcctgtgcaggtggctgccgaggaagtgctggtcgagaaggaagaggtgaacgagaacatcctcaacatcgtggaggagatcaaggagagcatcgtcgacaagctggaggccaacgaggaagccagcgaggaaggcaacgaggacctcctggagtccgctgaggaagccgctgaggaagtggctgaggaagccgtggacaccaccaccgaggctgacgtggtggagacggtggaggaagaggccgctaacgctaccaccgaggtgtccgctgaggagagcctggaggtgtccaccgaggctccagaggagacgaccgagtccgagagccacgagacgttcgaggaagacatcctgaagaacctggaggagaacaaggaagccaacgagaacgccctggaggacatcaaggagatgaaggaagagttcctcgactacgtggagcaaagggtcgaggacaacgagaacgtgctggtcgacctcctgcagcacctggagcgcaacgcccacgtgaacgagagcgtcctggaggacctggaggagatcaaggaagacctcctggccaacatccaaatggccgaggagacgaggaaggaagtgaccgacgcttccgctgagagcgctgaggaagtggaggagcccgtcgaggtgtccgctgaggtggctgctgaggagcctgtcgaggtggccgccgaggagccagtggaggtcaccgctgaggagcctgttgaggtgacggctgaggagccagtggagatcccaaccgaggagaacatcttcgacgtgatcgaggagatcaaggagaaggtcctggagaacctggaggagacgaccgctgagagcgtggctgagtccgtgggcgagggcgctgacgagaacgccctggacgtgctcaaggagatgcaagagagcctcctggagaacttcggccagaagatcgaggccaacgagaacatcctggccagcgtgctggagaacatccaggagaaggtcgagctgaacaagtccgtgctcgtcgacgtgctggccgagctcaaggaagaggccgtgtcccaaagggagacggctcaagaggtggctgctgagctggtggaggaagccgctgaggtcccagctgtggagccagtcgaggaagaggtggtggagccagctgtggaggtggtggaggagcctgtggaggaagaggtggtcgagccagtggtcgacgtgatcgaggagcctgccgtggaggtcgtggaggtcccagtggaggagacggtcgaggagcctgtggaggttaccgcggaggagcctgtggaggtcacggccgaggagcctgtcgaggagacggtggaggagccagtggtcgaggtggtcgaggagccagttgaggagcctgtggtcgaggccatcgaggagcccgtcgtcgagccagtggtcgagccagccgtcgaggtcatcgaggacgctacggaggagcccgtggaggaagccgccgaggagccggacgtggaggtggctgagggcagcgctatcgagtccgtggaggaagccttcgagcaaatcatcgaggacgccgcccaagtgatcgctgaggagagcgtggaggagacggctgagcaaatcctggagcaagccacccaggccgtgaccgaggaagccgctgacgctgctgacgtggctgacgctgaggaagccgtgggcaccgctcaagtcgtcaccgaggagagcgtggctgaggctatcgaggacaccgtcgaggagatctccgccgagccaatccaggccaccatcgagggcatcgtgggcgaggtcgtcgagtccgtcgaggagaacatcgaggccgtggaggaagccatcaaggacatcgtggagggcgctgtggagggcgctccagagctcagcctggaggagatgatcgaggacgtcatggtgggcaccgtggctgaggaagactccgctaaggaagccgctgaggagacggtggaggaagtggtgcaagaggacgctgctgaggaagaggccgccaaggaagccgccgaagagacggtggaggaagccgagagggaggctacccaagaggccgtcgaggagacggttgaggacgtggtcgaggaagtgtccgctgaggctgtggaggagatcgtcctcgaaaccccggagggcacctccgacgagagcgtggagacggtggtggagcacgctgtggaggactccctgggcgagacgatcgccaccatcgtggacgacgtcgccgaggagacgaccgagaagtccgaggagagcgtggtcgacaacctgggcgtcaaggtggaggaagtgctcgacgtcgacgtggaggaagtggcccaagaggccgccgacgacgtgatcatgcgcgtcagcgagaacgagtccgagggcgagagcggcgctgagtccggcgaggaagtggaggagctggagagcgccctcttcgaggtggagaaggacatcaagaagaaggtcctcgacatgttcagcggcaacgtggagttcgacgagaaggagtccgagaagctcgccctggacctccagaagaacctcctgtcccaccaccaccaccaccactga

SEQ ID NO: 27 - protein, 242 aa, Plasmodium vivax
MTYMLMKDDDSHDDKDDENEEKKKKEGKTNKDTNKIIKGESMTREDLLQLLNEMLKLQTDMKNIVKDLIVVAKKNSYDFMSVYNVAKTYNTVDPLGKYQIEMPEFDKVVENYHFDPEVKETVSKLMSSQENYYANMSETATLNVDKIIEIHHFMLNELYKIDPEFKKIPNKHELDPKLIALVIQSIVSAKVEEEFNLTSEDVEASIANQQYALTSNMEFARVNIQMQTIMNKFMGDHHHHHH

SEQ ID NO: 28 - DNA, 729 bp, Plasmodium vivax
  source 1..729: mol_type = genomic DNA
atgacctacatgctcatgaaggacgacgactcccacgacgacaaggacgacgagaacgaggagaagaagaagaaggaaggcaagaccaacaaggacaccaacaagatcatcaagggcgagagcatgaccagggaggacctcctgcaactcctgaacgagatgctcaagctgcagaccgacatgaagaacatcgtcaaggacctcatcgtggtcgccaagaagaactcctacgacttcatgagcgtgtacaacgtcgccaagacctacaacaccgtggacccactgggcaagtaccaaatcgagatgccagagttcgacaaggtggtcgagaactaccacttcgacccagaggtgaaggagacggtgtccaagctcatgtccagccaggagaactactacgccaacatgagcgagacggccaccctgaacgtcgacaagatcatcgagatccaccacttcatgctcaacgagctgtacaagatcgacccagagttcaagaagatcccaaacaagcacgagctggacccaaagctcatcgccctcgtgatccaatccatcgtgagcgccaaggtcgaggaagagttcaacctcacctccgaggacgtcgaggccagcatcgccaaccaacagtacgccctgacctccaacatggagttcgcccgcgtgaacatccaaatgcagaccatcatgaacaagttcatgggcgaccaccaccaccaccaccactga

SEQ ID NO: 29 - protein, 245 aa, Plasmodium vivax
MAGGVSEEAIKKLKEIKKLELDILKDFMKQDAGHADLYKKYHCIASDYISGNPKGSSAEGPNLAKKGEKSKKGEKHQNGEKPQNGEKPKKSFIEKIASFVSIFSYNNVSKIYSEHVQRIFPKARDHAGDGSAGDAIYPDDKIETGKKQNQSSYVQLSALNLMKRNMFLGGKDKSSEHFEVGNLGSFYMIFGARNTDYPWACSCDPLQLIDYKEKKRNYVLCSNQVDMSIQNADLFCNPKHHHHHH

SEQ ID NO: 30 - DNA, 738 bp, Plasmodium vivax
  source 1..738: mol_type = genomic DNA
atggccggcggcgtcagcgaggaagccatcaagaagctcaaggagatcaagaagctggagctggacatcctgaaggacttcatgaagcaagacgccggccacgccgacctctacaagaagtaccactgcatcgccagcgactacatctccggcaacccaaagggctccagcgctgagggcccaaacctggccaagaagggcgagaagagcaagaagggcgagaagcaccaaaacggcgagaagccacagaacggcgagaagccaaagaagtccttcatcgagaagatcgcctccttcgtgagcatcttctcctacaacaacgtcagcaagatctactccgagcacgtgcaaaggatcttcccaaaggcccgcgaccacgctggcgacggcagcgccggcgacgccatctacccagacgacaagatcgagacgggcaagaagcaaaaccagtccagctacgtccagctctccgccctcaacctgatgaagcgcaacatgttcctgggcggcaaggacaagtccagcgagcacttcgaagtgggcaacctcggcagcttctacatgatcttcggcgccaggaacaccgactacccatgggcctgctcctgcgacccactccagctgatcgactacaaggagaagaagcgcaactacgtgctctgcagcaaccaagtcgacatgtccatccagaacgccgacctgttctgcaacccaaagcaccaccaccaccaccactga

SEQ ID NO: 31 - protein, 363 aa, Plasmodium vivax
MVSCTSLCLYIIYSLFLLNNVSLSIQVKTNEIKNGQNGSVQLKEKGGGVNLAPKVGTNITQKRDTKMAKKTVTKVAKKKVTKVAEKTGTKVADKTGTKVADKTGTKVADKTGTKVAEKTGTKVADKTGTKVAEKTGTNISQKEDEKGPPKEDTQGTQKADAKAIQQADAQVSEKWKKKEWKEWIKKAESDLDIFNALMDNEKEKKWYSEKEKEWNKWIKGVEKKWMHYNKNIYVEYRSLVFWVGLKWVESQWEKWILSDGLEFLVMDWKKWIKENKSNFDEWLKSEWDTWTNSQMEEWKSSNWKLNEDKRWEMWENDKKWIKWLYLKDWINCSKWKKRIQKESKEWLRWTKLKEEMYHHHHHH

SEQ ID NO: 32 - DNA, 1092 bp, Plasmodium vivax
  source 1..1092: mol_type = genomic DNA
atggtgtcctgcaccagcctctgcctgtacatcatctacagcctcttcctcctgaacaacgtgtccctgagcatccaagtcaagaccaacgagatcaagaacggccaaaacggctccgtccagctcaaggagaagggcggcggcgtgaacctggctccaaaggtcggcaccaacatcacccagaagagggacaccaagatggccaagaagaccgtgaccaaggtcgccaagaagaaggtcacgaaggtcgccgagaagaccggcaccaaggtggccgacaagaccggcaccaaggtcgctgataagacggggacgaaggtcgctgataagaccgggacgaaggtggctgagaagacggggacgaaggttgctgataagacggggaccaaggtggctgagaagaccggcaccaacatcagccaaaaggaagacgagaagggcccaccaaaggaagacacccaaggcacccagaaggccgacgccaaggccatccaacaggccgacgcccaggtgagcgagaagtggaagaagaaggagtggaaggagtggatcaagaaggccgagtccgacctcgacatcttcaacgccctgatggacaacgagaaggagaagaagtggtacagcgagaaggagaaggagtggaacaagtggatcaagggcgtggagaagaagtggatgcactacaacaagaacatctacgtcgagtacaggtccctcgtgttctgggtcggcctgaagtgggtggagtcccaatgggagaagtggatcctcagcgacggcctggagttcctggtcatggactggaagaagtggatcaaggagaacaagtccaacttcgacgagtggctcaagagcgagtgggacacctggaccaactcccagatggaggagtggaagtccagcaactggaagctgaacgaggacaagcgctgggagatgtgggagaacgacaagaagtggatcaagtggctctacctgaaggactggatcaactgcagcaagtggaagaagaggatccaaaaggagtccaaggagtggctccgctggaccaagctgaaggaagagatgtaccaccaccaccaccaccactga

SEQ ID NO: 33 - protein, 415 aa, Plasmodium vivax
MGEDAEVENAKYRIPAGRCPVFGKGIVIENSDVSFLRPVATGDQKLKDGGFAFPNANDHISPMTLANLKERYKDNVEMMKLNDIALCRTHAASFVMAGDQNSSYRHPAVYDEKEKTCHMLYLSAQENMGPRYCSPDAQNRDAVFCFKPDKNESFENLVYLSKNVRNDWDKKCPRKNLGNAKFGLWVDGNCEEIPYVKEVEAEDLRECNRIVFGASASDQPTQYEEEMTDYQKIQQGFRQNNREMIKSAFLPVGAFNSDNFKSKGRGFNWANFDSVKKKCYIFNTKPTCLINDKNFIATTALSHPQEVDLEFPCSIYKDEIEREIKKQSRNMNLYSVDGERIVLPRIFISNDKESIKCPCEPERISNSTCNFYVCNCVEKRAEIKENNQVVIKEEFRDYYENGEEKSNKQHHHHHH

SEQ ID NO: 34 - DNA, 1248 bp, Plasmodium vivax
  source 1..1248: mol_type = genomic DNA
atgggcgaggacgccgaggtggagaacgccaagtacaggatcccagctggcaggtgcccagtgttcggcaagggcatcgtcatcgagaactccgacgtgagcttcctccgcccagtggctaccggcgaccaaaagctgaaggacggcggattcgccttcccaaacgccaacgaccacatctccccaatgaccctcgccaacctgaaggagaggtacaaggacaacgtggagatgatgaagctcaacgacatcgctctgtgcaggacccacgctgctagcttcgtgatggctggcgaccagaactccagctacaggcacccagccgtctacgacgagaaggagaagacctgccacatgctctacctgtccgcccaagagaacatgggcccaaggtactgctccccagacgctcagaacagggacgctgtcttctgcttcaagccagacaagaacgagtccttcgagaacctcgtgtacctgagcaagaacgtcaggaacgactgggacaagaagtgcccacgcaagaacctcggcaacgccaagttcggcctgtgggtggacggcaactgcgaggagatcccatacgtgaaggaagtggaggccgaggacctcagggagtgcaacaggatcgtcttcggcgcttccgctagcgaccaaccaacccagtacgaggaagagatgaccgactaccaaaagatccaacagggcttcaggcagaacaaccgcgagatgatcaagtccgccttcctcccagtgggcgccttcaactccgacaacttcaagagcaagggccgcggcttcaactgggccaacttcgacagcgtgaagaagaagtgctacatcttcaacaccaagccaacctgcctgatcaacgacaagaacttcatcgccaccaccgccctctcccacccacaagaggtcgacctggagttcccatgcagcatctacaaggacgagatcgagagggagatcaagaagcagtcccgcaacatgaacctctacagcgtggacggcgagaggatcgtcctgccacgcatcttcatctccaacgacaaggagagcatcaagtgcccatgcgagccagagaggatctccaacagcacctgcaacttctacgtgtgcaactgcgtcgagaagagggccgagatcaaggagaacaaccaagtggtcatcaaggaagagttcagggactactacgagaacggcgaggagaagtccaacaagcagcaccaccaccaccaccactga

SEQ ID NO: 35 - protein, 892 aa, Plasmodium vivax
MNGNRNLNIKPTCHKSGKNDKANGSDNIANKGGAQHAANGATGTPSGSSNGKKGATTTSASAGQAGASGGMAAPGMNPNFEQMMKPLNDMFKGNGEGLNIENIMNSDMFQNFFNSLMGGNPHDGAGGGQEILFKDMLNAMNAQGGGAPGAAATSGGANKDPNISVSPEQLNKINQLKDKLENVLKNVGVDVEQLKENMQNENIMQNKDALRDLLANLPMNPGMMQNMMAGKDGNMFNMDPNQMMNMFNQLSQGKMNMKDFGMGDFMPPPVHANDQDAEDDSRGKAFVTNSSNNDINFAHKLNAFEYSNGPSEGMFQLYGMNNDDGVIDDGMSDSVGKNSALDVSGGSINRNLSDGDSAKEDSDESNANATSNSNATVPNKGGHEGGSANEVYSNEEELITSSGSKGDANKLAGTGGYKNNNAFLDLNNLKKDASAAKYGKDNSGDKSNGGNSNGGNNKVMNKRIGGKKKKTFKKKKNPGQIPFKMETLQKLVKEYTNTSNQKIMEKIIKKYVSMSNQSARGNSEEEDDEEEAEDEKSAKDKNSEKEAELNMNEFSVKDIKKLISEGILTYEDLTEEELKKLAKPDDMFYELSPYANEEKDLSLNETSGVSNEQLNAFLRKNGSYHMSYDSKAIDYLKQKKAEKKEEEQEDDNFYDAYKQIKNSYEGIPSNYYHDAPQLIGENYVFTSVYDKKKELIDFLKRSNGATDSSNSSAGKDKGNSAESGTYKSKYYDKYMKKLSEYRRREAFKILKKRRAQEKKMQKKQEMQNNSSNEVDYSEYFKKNGFINSSNGTVKTFSKDQLDNMVKQFNSDGDDIPSSSGAGADLGDNYSGVSGGGQFSPSGGSGNNPSGYVTFDGQNIVGPNENEEEEPTEDVLNEDDDNADDDDHHHHHH

SEQ ID NO: 36 - DNA, 2679 bp, Plasmodium vivax
  source 1..2679: mol_type = genomic DNA
atgaacggcaacaggaacctgaacatcaagccaacctgccacaagagcggcaagaacgacaaggccaacggctccgacaacatcgctaacaagggcggcgcccaacacgctgctaacggcgccaccggcaccccaagcggctccagcaacggcaagaagggcgctacgaccaccagcgcttccgctggccaagctggcgcttccggcggcatggccgccccaggcatgaacccaaacttcgagcagatgatgaagccactgaacgacatgttcaagggcaacggcgagggcctcaacatcgagaacatcatgaacagcgacatgttccagaacttcttcaactccctgatgggcggcaacccacacgacggcgctggcggcggccaagagatcctgttcaaggacatgctcaacgccatgaacgcccaaggcggcggcgccccaggcgctgccgccacctccggcggcgccaacaaggacccaaacatcagcgtctccccagagcagctgaacaagatcaaccaactcaaggacaagctggagaacgtgctcaagaacgtgggcgtcgacgtggagcagctcaaggagaacatgcaaaacgagaacatcatgcagaacaaggacgctctgagggacctcctggctaacctcccgatgaacccaggcatgatgcaaaacatgatggccggcaaggacggcaacatgttcaacatggacccaaaccagatgatgaacatgttcaaccaactcagccagggcaagatgaacatgaaggacttcggcatgggcgacttcatgccaccaccagtccacgccaacgaccaagacgctgaggacgactcccgcggcaaggctttcgtgaccaactccagcaacaacgacatcaacttcgcccacaagctgaacgccttcgagtacagcaacggcccatccgagggcatgttccagctctacggcatgaacaacgacgacggcgtcatcgacgacggcatgagcgactccgtcggcaagaacagcgctctggacgtgagcggcggctccatcaacaggaacctcagcgacggcgactccgccaaggaagacagcgacgagtccaacgccaacgccaccagcaactccaacgccaccgtcccaaacaagggcggccacgagggcggcagcgctaacgaggtgtactccaacgaggaagagctgatcacctccagcggctccaagggcgacgctaacaagctggctggcaccggcggctacaagaacaacaacgccttcctcgacctgaacaacctgaagaaggacgccagcgccgccaagtacggcaaggacaacagcggcgacaagtccaacggcggcaactccaacggcggcaacaacaaggtcatgaacaagcgcatcggcggcaagaagaagaagaccttcaagaagaagaagaacccaggccaaatcccattcaagatggagacgctccagaagctggtcaaggagtacaccaacaccagcaaccaaaagatcatggagaagatcatcaagaagtatgtgtccatgtccaaccagagcgccaggggcaactccgaggaagaggacgacgaggaagaggccgaggacgagaagagcgccaaggacaagaactccgagaaggaagccgagctgaacatgaacgagttcagcgtcaaggacatcaagaagctcatctccgagggcatcctgacctacgaggacctcaccgaggaagagctcaagaagctggccaagccagacgacatgttctacgagctcagcccatacgccaacgaggagaaggacctctccctgaacgagacgagcggcgtgtccaacgagcaactgaacgccttcctccgcaagaacggctcctaccacatgagctacgactccaaggccatcgactacctgaagcaaaagaaggccgagaagaaggaagaggagcaagaggacgacaacttctacgacgcctacaagcaaatcaagaacagctacgagggcatcccatccaactactaccacgacgccccacagctcatcggcgagaactacgtcttcaccagcgtgtacgacaagaagaaggagctgatcgacttcctcaagaggtccaacggcgctaccgactccagcaactccagcgctggcaaggacaagggcaacagcgctgagtccggcacctacaagagcaagtactacgacaagtacatgaagaagctgtccgagtacaggcgcagggaggccttcaagatcctcaagaagcgcagggcccaggagaagaagatgcaaaagaagcaggagatgcaaaacaactccagcaacgaggtggactactccgagtacttcaagaagaacggcttcatcaactccagcaacggcaccgtcaagaccttcagcaaggaccaactggacaacatggtgaagcagttcaactccgacggcgacgacatcccatccagctccggcgctggcgctgacctcggcgacaactacagcggcgtgtccggcggcggccaattcagcccatccggcggcagcggcaacaacccatccggctacgtcaccttcgacggccagaacatcgtgggcccaaacgagaacgaggaagaggagccaaccgaggacgtgctcaacgaggacgacgacaacgccgacgacgacgaccaccaccaccaccaccactga

SEQ ID NO: 37 - protein, 350 aa, Plasmodium vivax
MPLEVSLWGQGNAHLGTQTSRLLRESGRNGQANRVNQADQADQVASPPISGKERRRGIGMTSNLQLLSGEDEKDSTSEEAPNLEGKDNADAGKDGEKEPSEKQSGDVDPTVTDAERAKDENASVSEEEQMKTLDSGEDHTDDGNADGGQGGGDGNDENQKGDGKEKEGGEEKKEDGKDDHEKGEKGSEGESGEKDEAAPKGDAAEKDKKLESKTADAKVSEHKADDANPGGNKDSPEGESPKEGNPDDPSQKNPEAAGDDDSRLHLDNLDDKVPHYSALRNNRVEKGVTDTMVLNDIIGENAKSCSVDNGGCADDQICIRIDNIGIKCICKEGHLFGDKCILTKHHHHHH

SEQ ID NO: 38 - DNA, 1053 bp, Plasmodium vivax
  source 1..1053: mol_type = genomic DNA
atgccgctggaggtgtccctgtggggccagggcaacgctcacctcggcacccaaacctcccgcctgctcagggagtccggcaggaacggccaggccaacagggtgaaccaggctgaccaggctgaccaagtggcttccccaccaatctccggcaaggagaggcgcaggggcatcggcatgacctccaacctccaactcctgagcggcgaggacgagaaggactccaccagcgaggaagccccaaacctggagggcaaggacaacgctgacgctggcaaggatggcgagaaggagccatccgagaagcagagcggcgacgtggacccaaccgtcaccgacgctgagagggctaaggacgagaacgcttccgtcagcgaggaagagcagatgaagaccctggacagcggcgaggaccacaccgacgacggcaacgctgacggcggacaaggcggcggcgacggcaacgacgagaaccaaaagggcgacggcaaggagaaggaaggcggcgaggagaagaaggaagacggcaaggacgaccacgagaagggcgagaagggctccgagggcgagagcggcgagaaggacgaggctgctccaaagggcgacgctgccgagaaggacaagaagctggagtccaagaccgccgacgccaaggtgagcgagcacaaggctgacgacgctaacccaggcggcaacaaggactccccagagggcgagagcccaaaggaaggcaacccagacgacccatcccagaagaacccggaggctgctggcgacgacgacagccgcctccacctggacaacctcgacgacaaggtcccacactactccgccctgcgcaacaacagggtggagaagggcgtcaccgacaccatggtgctgaacgacatcatcggcgagaacgccaagtcctgcagcgtggacaacggcggctgcgctgacgaccaaatctgcatcaggatcgacaacatcggcatcaagtgcatctgcaaggaaggccacctcttcggcgacaagtgcatcctgaccaagcaccaccaccaccaccactga

SEQ ID NO: 39 - protein, 296 aa, Plasmodium vivax
MDVLQLVIPSEEDIQLDKPKKDELGSGILSILDVHYQDVPKEFMEEEEETAVYPLKPEDFAKEDSQSTEWLTFIQGLEGDWERLEVSLNKARERWMEQRNKEWAGWLRLIENKWSEYSQISTKGKDPAGLRKREWSDEKWKKWFKAEVKSQIDSHLKKWMNDTHSNLFKILVKDMSQFENKKTKEWLMNHWKKNERGYGSESFEVMTTSKLLNVAKSREWYRANPNINRERRELMKWFLLKENEYLGQEWKKWTHWKKVKFFVFNSMCTTFSGKRLTKEEWNQFVNEIKVHHHHHH

SEQ ID NO: 40 - DNA, 891 bp, Plasmodium vivax
  source 1..891: mol_type = genomic DNA
atggacgtgctccaactggtcatcccaagcgaggaagacatccagctcgacaagccaaagaaggacgagctgggcagcggcatcctctccatcctggacgtgcactaccaagacgtcccaaaggagttcatggaggaagaggaagagacggccgtgtacccactcaagccagaggacttcgccaaggaagactcccaaagcaccgagtggctcaccttcatccaaggcctggagggcgactgggagaggctggaggtgtccctgaacaaggccagggagcgctggatggagcaaaggaacaaggagtgggctggctggctcaggctgatcgagaacaagtggtccgagtacagccagatctccaccaagggcaaggacccggctggcctcaggaagcgcgagtggtccgacgaaaagtggaagaagtggttcaaggccgaggtgaagagccaaatcgactcccacctgaagaagtggatgaacgacacccacagcaacctcttcaagatcctggtcaaggacatgtcccagttcgagaacaagaagaccaaggagtggctcatgaaccactggaagaagaacgagaggggctacggctccgagagcttcgaggtcatgaccaccagcaagctcctgaacgtcgccaagtccagggagtggtaccgcgccaacccaaacatcaaccgcgagaggcgcgagctcatgaagtggttcctcctgaaggagaacgagtacctgggccaagagtggaagaagtggacccactggaagaaggtgaagttcttcgtcttcaacagcatgtgcaccaccttctccggcaagcgcctgaccaaggaagagtggaaccagttcgtgaacgagatcaaggtccaccaccaccaccaccactga

SEQ ID NO: 41 - protein, 291 aa, Plasmodium vivax
MEAMPKFPQNNLKGGLKDSPLKQPKSPLINGPPKPVNDKLKDDSNKTETKDAKNGLNKPPKNINDKVKDGENKTPSQDLNEPSFKLPMRQKESSWYTWLKGTKKDYETLKCFAKGNLYDWLCNVRESFDLYLQSLEKKWTTCSDSATTLFLCECFAESSGWNDSQWGNWMNNQLKEQLKTEAEAWISTKKKDFDGLTSKYFSLWKDHRRKELDADEWKNKVSSGGLSEWEELTNKMNTRYRNNLDNMWSHFSRDLFFNFDEWAPQVLEKWIENKQWNRWVKKVRKHHHHHH

SEQ ID NO: 42 - DNA, 876 bp, Plasmodium vivax
  source 1..876: mol_type = genomic DNA
atggaggccatgccaaagttcccacaaaacaacctcaagggcggcctgaaggactccccactcaagcagccaaagagcccactgatcaacggcccaccaaagccagtgaacgacaagctcaaggacgactccaacaagaccgagacgaaggacgccaagaacggcctgaacaagccaccaaagaacatcaacgacaaggtcaaggacggcgagaacaagaccccatcccaagacctcaacgagccaagcttcaagctgccaatgaggcagaaggagtccagctggtacacctggctcaagggcaccaagaaggactacgagacgctgaagtgcttcgccaagggcaacctctacgactggctgtgcaacgtgcgcgagtccttcgacctctacctgcaaagcctggagaagaagtggaccacctgctccgacagcgctaccaccctcttcctgtgcgagtgcttcgccgagtccagcggctggaacgactcccagtggggcaactggatgaacaaccaactcaaggagcagctgaagaccgaggccgaggcctggatcagcaccaagaagaaggacttcgacggcctcacctccaagtacttcagcctgtggaaggaccacaggcgcaaggagctcgacgccgacgagtggaagaacaaggtgtccagcggcggcctcagcgagtgggaggagctgaccaacaagatgaacaccaggtaccgcaacaacctcgacaacatgtggtcccacttcagcagggacctgttcttcaacttcgacgagtgggccccacaagtcctggagaagtggatcgagaacaagcagtggaaccgctgggtgaagaaggtccgcaagcaccaccaccaccaccactga

SEQ ID NO: 43 - protein, 866 aa, Plasmodium vivax
MQKAPNNGKNNYGLNDDELGAILFGLNYDSIAKNKDNLEKRKNVENESIFLRNFANEDTSKNTQSEKAQKEIKIETETESVNSNEKEVATSQKSDTSNKNSSVENEKIELKNDELLGKNFEKDKVNKKGDNTNTTNNHDLTNSSEKQGVDIRGSKNMNNYLQKTGDTNIEKSESLQKDVNIKNHNEEANDAKRLDSAQTNNEKSKISKDTIDKDVQSNELTNLASNRSNKKSQGLAKKENELKSANLEENHNAKKDLLKKDQKREDGKKITHPENSNSDQYGVQVSLNDEEKNTNTKSVSHSEDHSASYSGEKFGTHVSNSQKDMLKNIRPVQFDESAYGKLNGGSPENDENEILNKINKNNENNFSEKVALRKGTKDRNEYEYFKLKSNDFKVLGIINKYSSRGGFSISVDCGGYDDFDEVPGVSNLLQHAIFYKSEKRNTTLLSELGKYSSEYNSCTSESSTSYYATAHSEDIYHLLNLFAENLFYPVFSEEHIQNEVKEINNKYISIENNLESCLKIASQYITNFKYSKFFVNGNYTTLCENVLKNRLSIKNILTEFHKKCYQPRNMSLTILLGNKVNTADHYNMKDVENMVVHIFGKIKNESYPIDGDVIGKRINRMESERVNLYGKKDSYNDANFIHIEGRNEKEAAFLQSMNELHYALDLNQKSRYVEIIKKEEWGDQLYLYWSSKTNAELCKKIEEFGSMTFLREIFSDFRRNGLYYKISVENKYVYDLEVTSICNKYYLNFGILVKLTQRGRTNLAHLIHICNVFVNEIGKLFDRDSLDKGISKYILDYYREKALVTDLKFNSDNVNVSLDDLVIYSKRLLVHADDPSSLLTIHSLIEDKHKNDFRNHIKITHHHHHH

SEQ ID NO: 44 - DNA, 2601 bp, Plasmodium vivax
  source 1..2601: mol_type = genomic DNA
atgcaaaaggccccaaacaacggcaagaacaactacggcctcaacgacgacgagctgggcgccatcctcttcggcctgaactacgacagcatcgccaagaacaaggacaacctggagaagaggaagaacgtcgagaacgagtccatcttcctgcgcaacttcgccaacgaggacaccagcaagaacacccaatccgagaaggcccagaaggagatcaagatcgagacggagacggagtccgtcaacagcaacgagaaggaagtggccacctcccagaagagcgacacctccaacaagaactccagcgtcgagaacgagaagatcgagctgaagaacgacgagctcctgggcaagaacttcgagaaggacaaggtgaacaagaagggcgacaacaccaacaccaccaacaaccacgacctcaccaactccagcgagaagcaaggcgtcgacatcaggggcagcaagaacatgaacaactacctccaaaagaccggcgacaccaacatcgagaagtccgagagcctgcagaaggacgtgaacatcaagaaccacaacgaggaagccaacgacgccaagaggctggacagcgcccagaccaacaacgagaagagcaagatctccaaggacaccatcgacaaggacgtgcaatccaacgagctcaccaacctggccagcaaccgctccaacaagaagagccagggcctcgccaagaaggagaacgagctcaagtccgccaacctggaggagaaccacaacgccaagaaggacctcctgaagaaggaccaaaagagggaggacggcaagaagatcacccacccagagaactccaacagcgaccaatacggcgtgcaagtgtccctgaacgacgaggagaagaacaccaacaccaagtccgtcagccactccgaggaccacagcgcttcctacagcggcgagaagttcggcacccacgtctccaacagccaaaaggacatgctcaagaacatccgcccagtgcagttcgacgagagcgcttacggcaagctcaacggcggctccccagagaacgacgagaacgagatcctgaacaagatcaacaagaacaacgagaacaacttcagcgagaaggtggccctcaggaagggcaccaaggaccgcaacgagtacgagtacttcaagctcaagtccaacgacttcaaggtcctgggcatcatcaacaagtactccagcaggggcggcttctccatcagcgtggactgcggcggatacgacgacttcgacgaggtgccaggcgtctccaacctcctgcaacacgccatcttctacaagagcgagaagcgcaacaccaccctcctgtccgagctcggcaagtactccagcgagtacaacagctgcacctccgagtccagcaccagctactacgccaccgcccactccgaggacatctaccacctcctgaacctcttcgccgagaacctgttctacccagtcttcagcgaggagcacatccaaaacgaggtgaaggagatcaacaacaagtacatctccatcgagaacaacctggagagctgcctgaagatcgcctcccagtacatcaccaacttcaagtacagcaagttcttcgtcaacggcaactacaccaccctctgcgagaacgtgctcaagaacaggctgagcatcaagaacatcctgaccgagttccacaagaagtgctaccagccacgcaacatgtccctcaccatcctcctgggcaacaaggtcaacaccgccgaccactacaacatgaaggacgtggagaacatggtggtccacatcttcggcaagatcaagaacgagtcctacccaatcgacggcgacgtcatcggcaagaggatcaaccgcatggagagcgagagggtcaacctctacggcaagaaggactcctacaacgacgccaacttcatccacatcgagggccgcaacgagaaggaagccgccttcctccaaagcatgaacgagctgcactacgccctcgacctgaaccagaagtcccgctacgtggagatcatcaagaaggaagagtggggcgaccaactctacctgtactggtccagcaagaccaacgccgagctctgcaagaagatcgaggagttcggcagcatgaccttcctccgcgagatcttctccgacttcaggcgcaacggcctgtactacaagatcagcgtggagaacaagtatgtgtacgacctggaggtgacctccatctgcaacaagtactacctgaacttcggcatcctcgtcaagctgacccaaaggggccgcaccaacctcgctcacctgatccacatctgcaacgtgttcgtcaacgagatcggcaagctcttcgacagggacagcctggacaagggcatctccaagtacatcctcgactactaccgcgagaaggccctcgtgaccgacctgaagttcaacagcgacaacgtgaacgtctccctcgatgacctggtcatctacagcaagaggctcctggtgcacgccgacgacccatccagcctcctgaccatccactccctcatcgaggacaagcataagaacgacttccgcaaccacatcaagatcacccaccaccaccaccaccactga

SEQ ID NO: 45 - protein, 276 aa, Plasmodium vivax
MKEAVKKGSKKAMKQPMHKPNLLEEEDFEEKESFSDDEMNGFMEESMDASKLDAKKAKTTLRSSEKKKTPTSGMSGMSGSGATSAATEAATNMNATAMNAAAKGNSEASKKQTDLSNEDLFNDELTEEVIADSYEEGGNVGSEEAESLTNAFDDKLLDQGVNENTLLNDNMIYNVNMVPHKKRELYISPHKHTSAASSKNGKHHAADADALDKKLRAHELLELENGEGSNSVIVETEEVDVDLNGGKSSGSVSFLSSVVFLLIGLLCFTNHHHHHH

SEQ ID NO: 46 - DNA, 831 bp, Plasmodium vivax
  source 1..831: mol_type = genomic DNA
atgaaggaagccgtgaagaagggctccaagaaggccatgaagcaaccaatgcacaagccaaacctcctggaggaagaggacttcgaggagaaggagtccttcagcgacgacgagatgaacggcttcatggaggagtccatggacgccagcaagctggacgccaagaaggccaagaccaccctcaggtccagcgagaagaagaagaccccaacctccggcatgagcggcatgtccggcagcggcgctaccagcgctgctaccgaggccgccaccaacatgaacgctaccgccatgaacgctgccgccaagggcaactccgaggctagcaagaagcaaaccgacctctccaacgaggacctgttcaacgacgagctcaccgaggaagtgatcgccgacagctacgaggaaggcggcaacgtgggctccgaggaagccgagagcctgaccaacgccttcgacgacaagctcctggaccagggcgtgaacgagaacaccctcctgaacgacaacatgatctacaacgtgaacatggtcccacacaagaagagggagctctacatctccccacacaagcacaccagcgccgcctccagcaagaacggcaagcaccacgctgctgacgctgacgctctggacaagaagctcagggctcacgagctcctggagctggagaacggcgagggctccaacagcgtgatcgtcgagacggaggaagtggacgtggacctgaacggcggcaagtcctccggctccgtcagcttcctctccagcgtggtcttcctcctgatcggcctcctgtgcttcaccaaccaccaccaccaccaccactga

SEQ ID NO: 47 - protein, 649 aa, Plasmodium vivax
MDDNGRRLPRKAAPPVDKAKQDVMKDIVNYLSKNMLAFVRQKRNVSGKEGEAPTGPSGAQGGDSSQYASKFTFTDHSVDFSKYNKLDKEKFAAKDDLKSRLKNEVVASMLDTEGDILTEEFGYLLRNYFDKVKLEEKKSQEAESAKPAEQEEEAEEAPEQKEEATAEKATEETTEAATEETTEAATEETTEAATEETTEAATEETTEAATEETTEAATEETTEAATEETTEAATEEATEGATEEGAEETTEEATEEGAEEATEEGAEEATEEGAEETTEEATEEGAEETTEETTEEGAEEEATEEGAEETTEEGAEEAAEEGAEEGAEAATEEATEEATEEATEEATEEATEEATEEATAEVAEAATPEKVTEEATEEATEEGDNEPAEQAAEKEEDVKGGLMDNETYYNTLQELYEEIENDDKKEKEKIQKAKEQEELEKKLFKESKKGKKKEKKRRKKLCKMAKIVEKYAEEIPKDSERSLRYDKEEHIDDPDEMDDLLFGEFKTLEKYGTHKTSTFYYEMTCFDERLRDFEINTKLKEMEEVPEKWELLSLYWQSYRNERHKYLAVKKYLLEKFLELKTNQSTEALPKYNKKWKQCEEIVDNNFTKQHEHVNDVFYTFVAKENLSRDEFKEILNDVRASWHHHHHH

SEQ ID NO: 48 - DNA, 1950 bp, Plasmodium vivax
  source 1..1950: mol_type = genomic DNA
atggacgacaacggcaggcgcctcccaaggaaggctgccccaccagtggacaaggccaagcaggacgtgatgaaggacatcgtcaactacctctccaagaacatgctggccttcgtgaggcaaaagcgcaacgtctccggcaaggaaggcgaggctccaaccggcccaagcggcgctcaaggcggcgactccagccagtacgccagcaagttcaccttcaccgaccactccgtggacttcagcaagtacaacaagctcgacaaggagaagttcgccgccaaggacgacctcaagtccaggctgaagaacgaggtggtcgccagcatgctcgacaccgagggcgacatcctgaccgaggagttcggctacctcctgcgcaactacttcgacaaggtcaagctggaggagaagaagtcccaagaggccgagagcgctaagccagctgagcaagaggaagaggccgaggaagccccagagcaaaaggaagaggccaccgctgagaaggctaccgaggagacgaccgaggctgccacggaggagacgacggaggccgccacggaggagacgaccgaggccgccaccgaggagacgacggaggctgccactgaagagacgaccgaggctgcgacggaagagacgaccgaggccgcgacggaagagacgactgaggctgccactgaggagacgacggaagctgctaccgaggaagccaccgagggcgctaccgaggaaggcgctgaggagacgacggaggaagccacggaggaaggcgctgaggaagccaccgaggaaggcgccgaggaagccacggaggaaggcgcagaggagacgacagaggaagccacggaggaaggcgccgaagagacgaccgaagagacgaccgaggaaggcgcggaggaagaggccactgaggaaggcgccgaggagacgactgaggaaggcgcagaggaagccgctgaggaaggcgctgaggaaggcgctgaggccgccacggaggaagccaccgaggaagccacggaggaagccacggaggaagccacagaggaagccactgaggaagccacagaggaagccacagctgaggtggctgaggctgctaccccagagaaggtcacagaggaagccacagaggaagccaccgaggaaggcgacaacgagccagctgagcaggctgctgagaaggaagaggacgtgaagggcggcctcatggacaacgagacgtactacaacaccctccaagagctgtacgaggagatcgagaacgacgacaagaaggagaaggagaagatccaaaaggccaaggagcaagaggagctggagaagaagctgttcaaggagtccaagaagggcaagaagaaggagaagaagaggcgcaagaagctctgcaagatggccaagatcgtcgagaagtacgccgaggagatcccaaaggactccgagaggagcctgcgctacgacaaggaagagcacatcgacgacccagacgagatggacgacctcctgttcggcgagttcaagaccctggagaagtacggcacccacaagacctccaccttctactacgagatgacctgcttcgacgagaggctccgcgacttcgagatcaacaccaagctgaaggagatggaggaagtgccagagaagtgggagctcctgtccctctactggcagagctacaggaacgagcgccacaagtacctggccgtcaagaagtacctcctggagaagttcctggagctgaagaccaaccaaagcaccgaggccctgccaaagtacaacaagaagtggaagcagtgcgaggagatcgtcgacaacaacttcaccaagcaacacgagcacgtgaacgacgtcttctacaccttcgtggccaaggagaacctctccagggacgagttcaaggagatcctgaacgacgtccgcgccagctggcaccaccaccaccaccactga

SEQ ID NO: 49 - protein, 332 aa, Plasmodium vivax
MRCCTKDAVNVESPKKVVVGETEEDTREEENPYEDLPTVTVTLSDGSVYTGTTKDNRVHGRGVLKYVNGDQYEGEFVDGKKEGKGKWTDKENNTYEGDWVKDKRHGHGVYKTAEGFIFEGEFANNKREGKGTIITPEKTKYVCSFQDDEEVGEVEFFFANGDHALGYIKDGYLCQNGRYEFKNGDIYVGNFEKGLFHGEGYYKWNNDANYTIYEGNYSEGKKHGKGQLINKDGRILCGMFRDNNMDGEFLEISPQGNQTKVLYDKGFFVKVLDKIEENLDVQEFLKDSIIHTTIFSDPTTYKKLYEITEKKKPQFRLNLKRTQPTSHHHHHH

SEQ ID NO: 50 - DNA, 999 bp, Plasmodium vivax
  source 1..999: mol_type = genomic DNA
atgaggtgctgcaccaaggacgccgtcaacgtggagtccccaaagaaggtggtcgtgggcgagacggaggaagacaccagggaggaagagaacccatacgaggacctcccaaccgtcaccgtgaccctgtccgacggcagcgtctacaccggcaccaccaaggacaacagggtgcacggccgcggcgtcctcaagtatgtgaacggcgaccaatacgagggcgagttcgtcgacggcaagaaggaaggcaagggcaagtggaccgacaaggagaacaacacctacgagggcgactgggtcaaggacaagaggcacggccacggcgtgtacaagaccgctgagggcttcatcttcgagggcgagttcgccaacaacaagcgcgagggcaagggcaccatcatcaccccagagaagaccaagtatgtgtgcagcttccaagacgacgaggaagtgggcgaggtggagttcttcttcgccaacggcgaccacgccctcggctacatcaaggacggctacctgtgccagaacggccgctacgagttcaagaacggcgacatctacgtgggcaacttcgagaagggcctgttccacggcgagggctactacaagtggaacaacgacgccaactacaccatctacgagggcaactactccgagggcaagaagcacggcaagggccaactcatcaacaaggacggcaggatcctgtgcggcatgttccgcgacaacaacatggacggcgagttcctggagatcagcccacaaggcaaccagaccaaggtcctctacgacaagggcttcttcgtcaaggtgctggacaagatcgaggagaacctcgacgtgcaggagttcctgaaggactccatcatccacaccaccatcttcagcgacccaaccacctacaagaagctgtacgagatcaccgagaagaagaagccacaattcaggctcaacctgaagcgcacccagccaacctcccaccaccaccaccaccactga

SEQ ID NO: 51 - protein, 309 aa, Plasmodium vivax
MNKLGTSLVEDATANGEFGLRVQRLLGGSRSSRDSIFADSFYDDDDDDDDNNDKLFDYDSDHKSRREVKDRHHRHRHSHSHRHKRRHSHKHRTSSRSRREKEESSTTNDDDDEVLSLSRFDVDDDKDDRSHSRYSVDYDDENDDEPSSSRPASTDYDDIIDLTNARRSGSKYRISSMDIELYPEHEDEYLFEGKRRSGGVLKKADNYCENKIFDALSALDKYKEYYGEERRVMKQAAYRKATKVFAIPGAAALSPLIITLFLTTSNVVALPLAASAVILGGILYKKSKDKSDYGRPHLKSITYHHHHHH

SEQ ID NO: 52 - DNA, 930 bp, Plasmodium vivax
  source 1..930: mol_type = genomic DNA
atgaacaagctgggcaccagcctcgtggaggacgctaccgctaacggcgagttcggcctccgcgtccaaaggctgctgggcggctccaggtccagccgcgacagcatcttcgccgactccttctacgatgatgacgacgacgacgacgacaacaacgacaagctgttcgactacgacagcgaccacaagtccaggcgcgaggtgaaggacaggcaccacaggcacaggcacagccactcccaccgccacaagaggcgccacagccacaagcacaggacctccagccgctccaggcgcgagaaggaagagtccagcaccaccaacgacgacgacgacgaggtgctcagcctgtccaggttcgacgtcgacgacgacaaggacgacaggagccactcccgctacagcgtggactacgacgacgagaacgacgacgagccatccagctccaggccagcctccaccgactacgacgacatcatcgacctcaccaacgctaggcgcagcggctccaagtaccgcatcagctccatggacatcgagctctacccagagcacgaggacgagtacctgttcgagggcaagaggcgcagcggcggcgtcctgaagaaggctgacaactactgcgagaacaagatcttcgacgccctctccgccctggacaagtacaaggagtactacggcgaggagaggcgcgtgatgaagcaggccgcctacaggaaggccaccaaggtcttcgctatcccaggcgctgccgccctcagcccactgatcatcaccctcttcctgaccaccagcaacgtggtggctctcccactggctgcttccgccgtcatcctcggcggcatcctgtacaagaagagcaaggacaagtccgactacggccgcccacacctcaagtccatcacctaccaccaccaccaccaccactga

SEQ ID NO: 53 - protein, 332 aa, Plasmodium vivax
MEAARGVSGLVPSSNSLQEITLRYKDKLLNMDKEQMILTLGVTMIAITSAVAFGVLATHGDINDFLGVESDEESEKKKEIVEKSEEWKRKEWSNWLKKLEQDWKVFNEKLQNEKKTFLEEKEEDWNTWIKSVEKKWTHFNPNMDKEFHTNMMRRSINWTESQWREWIQTEGRLYLDIEWKKWFFENQSRLDELIVKKWIQWKKDKIINWLMSDWKRAEQEHWEEFEEKSWSSKFFQIFEKRNYEDFKDRVSDEWEDWFEWVKRKDNIFITNVLDQWIKWKEEKNLLYNNWADAFVTNWINKKQWVVWVNERRNLAAKAKAALNKKKHHHHHH

SEQ ID NO: 54 - DNA, 999 bp, Plasmodium vivax
  source 1..999: mol_type = genomic DNA
atggaggctgccaggggcgtgtccggcctcgtcccatccagcaacagcctccaagagatcaccctgcgctacaaggacaagctcctgaacatggacaaggagcagatgatcctcaccctgggcgtcaccatgatcgctatcacctccgctgtggctttcggcgtcctggctacccacggcgacatcaacgacttcctgggcgtcgagtccgacgaggagagcgagaagaagaaggagatcgtggagaagtccgaggagtggaagaggaaggagtggagcaactggctcaagaagctggagcaagactggaaggtcttcaacgagaagctccagaacgagaagaagaccttcctggaggagaaggaagaggactggaacacctggatcaagtccgtggagaagaagtggacccacttcaacccaaacatggacaaggagttccacaccaacatgatgaggcgctccatcaactggaccgagagccaatggcgcgagtggatccagaccgagggcaggctctacctggacatcgagtggaagaagtggttcttcgagaaccaaagcaggctcgacgagctgatcgtgaagaagtggatccagtggaagaaggacaagatcatcaactggctcatgtccgactggaagcgcgccgagcaagagcactgggaggagttcgaggagaagagctggtccagcaagttcttccagatcttcgagaagcgcaactacgaggacttcaaggaccgcgtgagcgacgagtgggaggactggttcgagtgggtcaagcgcaaggacaacatcttcatcaccaacgtgctggaccagtggatcaagtggaaggaagagaagaacctcctgtacaacaactgggccgacgccttcgtcaccaactggatcaacaagaagcagtgggtggtctgggtgaacgagaggcgcaacctcgctgctaaggctaaggctgccctgaacaagaagaagcaccaccaccaccaccactga

SEQ ID NO: 55 - protein, 405 aa, Plasmodium vivax
MTKGPSGPPPNKKLNANALHFLRGKLELLNKISEEQVVSPDFKKNVELLKKKIEELQGKAEKDKSKTDGEDTTPKEQQEDQNVSQNGLEEQAPSDSNEGEAQEENTQVKNVIFTEKEEAVDEEAEKEDTAVISEKANFPNEESQGNDETQTQESIEGEASPGVVVDETDDSPEGEPLSGLETEGNSSAESAPNEPDVNTTHTAVDTHMPADANIGVDTNMPFDTPPHPSGENPGAPQETHLPSIDENANRRASRMKHMSSFLNGLLTNQSNNKKEIFFHPYYGPYFNHGGYYNYDPYYNYAPAYNPFVSQARDYEVIKKLLDACFNKGEGADPNVPCIIDIFKKVLDDERFRNELKTFMYDLYEFLKKNDVLSDDEKKNELMRFFFDNAFQLVNPMFYYHHHHHH

SEQ ID NO: 56 - DNA, 1218 bp, Plasmodium vivax
  source 1..1218: mol_type = genomic DNA
atgaccaagggcccatccggcccaccaccaaacaagaagctcaacgccaacgccctccacttcctgaggggcaagctggagctcctgaacaagatctccgaggagcaagtggtcagcccagacttcaagaagaacgtcgagctcctcaagaagaagatcgaggagctccagggcaaggccgagaaggacaagtccaagaccgacggcgaggacaccaccccaaaggagcaacaagaggaccaaaacgtgagccagaacggcctggaggagcaagctccgtccgacagcaacgagggcgaggctcaagaggagaacacccaggtcaagaacgtgatcttcaccgagaaggaagaggccgtcgacgaggaagccgagaaggaagacaccgccgtgatctccgagaaggccaacttcccaaacgaggagagccagggcaacgacgagacgcaaacccaagagtccatcgagggcgaggctagcccgggcgtggtggtggacgagacggacgactccccggagggcgagccactcagcggcctcgaaaccgagggcaactccagcgctgagtccgctccaaacgagccagacgtcaacaccacccacaccgctgtggacacccacatgccagctgacgccaacatcggcgtcgacaccaacatgccattcgacaccccaccacacccaagcggcgagaacccgggcgccccacaagagacgcacctcccatccatcgacgagaacgccaacaggcgcgccagcaggatgaagcacatgtccagcttcctgaacggcctcctgaccaaccagtccaacaacaagaaggagatcttcttccacccatactacggcccatacttcaaccacggcggatactacaactacgacccatactacaactacgccccagcctacaacccattcgtcagccaagcccgcgactacgaggtcatcaagaagctcctggacgcctgcttcaacaagggcgagggcgctgacccaaacgtcccatgcatcatcgacatcttcaagaaggtgctcgacgacgagaggttccgcaacgagctgaagaccttcatgtacgacctctacgagttcctgaagaagaacgacgtcctcagcgacgacgagaagaagaacgagctgatgaggttcttcttcgacaacgccttccagctcgtgaacccaatgttctactaccaccaccaccaccaccactga

SEQ ID NO: 57 - protein, 875 aa, Plasmodium vivax
MFSGGVGDDEEEEEEEEGEEGESERDDSERDYAGRDDAGRDDAERNDAERDDAERNDAERDDAERDHAERDHADKAESDRESSLEANENRLVKLSEGGESEPALLEVEEDIKQTVLGMFSLKGEFDEAESEKLALDLQKNLLSMLSGNMEDNDDEYEDIDEEYEEVEEDYEEEKLGKPVEVVVEDATEEAVDEVVGVVQEPEEEGAEESDKDTGEVSEEEVAKEAADEVMEEEKKEEAGEPSVVVEEPSVVVKEPSVVVKEPSVVVEEPSVVVEEPSVVVEEPSVVVEEPAFTVEEPAFTVEEPAITVEEPAITVEEPVFTVEEPVFTVEEPAFTVEEPAFTVEEPAFTVEEPATTVEELVEEVLKVAEEEVATEAVEKDGEEAEEQVTEESVEEDEEESGEEEGEESEEEETEESAEEEVAKESVEEEVAKEAEESEESGEESAEEEKEKAEEPVAPVDEVLKEGMQKIEESVKEALGVVQEAVDKVAEEEQTEQAQGPAEAGPVGVVKEPEEEEESEEEGEEGEEGEEGEEEEEEESEEEESEEGESEAGESEAGKSDAAESEVAESEAGEPAEDQAGMDAKMKDELLGMLSEKMKAEGKDLDKLPPEVKKNLLDMLAGNMEMDDEEEEGEEEGEDLGNEELDLQKNLLEMLSGKGGFNPNMLGNLKELEALQKSVPGLMGKAQGISPAEIESLKSMFSGAFDSRGFKGMPQMKLPAELQSIMMPKKEEKGKPQGAQAKAKVPAKAGQVQKPKAQDIMPSRRIRDLFVLPKEIFGSLKNFKESALKFANHIGLNLETIKKHLTTVKNFLLRVDAVVDKEIGNIIEAGKSPQNVVQANEGFLDKMKRLVNKYKIFSIPFFAGMGSFGFHHHHHH

SEQ ID NO: 58 - DNA, 2628 bp, Plasmodium vivax
  source 1..2628: mol_type = genomic DNA
atgttcagcggcggcgtgggcgacgacgaggaagaggaagaggaagaggaaggcgaggaaggcgagagcgagagggacgactccgagagggactacgctggcagggacgatgccggcagggacgacgccgagaggaacgacgccgagcgcgatgatgctgagcgcaacgacgccgagcgcgacgacgccgagagggaccacgccgagcgcgaccacgccgacaaggccgagtccgacagggagtccagcctggaggccaacgagaacaggctggtgaagctcagcgagggcggcgagtccgagccagctctcctggaggtggaggaagacatcaagcaaaccgtcctgggcatgttcagcctcaagggcgagttcgacgaggccgagtccgagaagctcgccctggacctccagaagaacctcctgtccatgctcagcggcaacatggaggacaacgacgacgagtacgaggacatcgacgaggagtacgaggaagtggaggaagactacgaggaagagaagctcggcaagccagtggaggtggtcgtggaggacgccaccgaggaagccgtggacgaggtggtgggcgtcgtgcaagagccagaggaagagggcgctgaggagagcgacaaggacaccggcgaggtgtccgaggaagaggtggccaaggaagccgccgacgaggtcatggaggaagagaagaaggaagaggccggcgagccatccgtggtggtggaggagccaagcgtggtcgtgaaggagccatccgtcgtggtcaaggagccttccgtggtcgtggaggagcctagcgtcgtcgtcgaggagccttccgtcgtggtggaggagcccagcgtggtcgtcgaggagccagccttcaccgtggaggagcctgccttcaccgtcgaggagccagccatcaccgtggaggagcccgctatcacggtggaggagccagtgttcaccgtggaagaacccgtgttcaccgtggaagagcccgccttcaccgttgaggagcccgccttcaccgtagaagagcctgccttcaccgttgaagaaccagctaccaccgtggaggagctggtggaggaagtgctcaaggtggctgaggaagaggtggctaccgaggctgtggagaaggacggcgaggaagccgaggagcaagtcaccgaggagagcgtcgaggaagacgaggaagagtccggcgaggaagagggcgaggagagcgaggaagaggagaccgaggagtccgctgaggaagaggtggcgaaggagagcgtggaggaagaggtggctaaggaagccgaggagtccgaggagagcggggaggagagcgctgaggaagagaaggagaaggccgaggagccagtggctccagtggacgaggtcctgaaggaaggcatgcagaagatcgaggagagcgtgaaggaagccctgggcgtggtccaagaggccgtggacaaggtcgccgaggaagagcagaccgagcaggctcagggcccagctgaggctggcccagtcggcgtggtcaaggagcctgaggaagaggaagagtctgaggaagagggcgaggaaggcgaggaaggcgaggaaggcgaggaagaggaagaggaagagagtgaggaagaggagtctgaggaaggcgagtccgaggctggggagagcgaggctggcaagagcgacgccgccgagtccgaggtggccgagagcgaggccggcgagccggctgaggaccaagctggcatggacgccaagatgaaggacgagctcctgggcatgctgagcgagaagatgaaggccgagggcaaggacctggacaagctcccaccagaggtcaagaagaacctcctggacatgctcgccggcaacatggagatggacgatgaggaagaggaaggcgaggaagagggcgaagacctgggcaacgaggagctcgacctccagaagaacctcctggagatgctctccggcaagggcggcttcaacccaaacatgctgggcaacctcaaggagctggaggccctccaaaagagcgtgccaggcctgatgggcaaggctcagggcatctccccagctgagatcgagtccctcaagagcatgttctccggcgccttcgacagcaggggcttcaagggcatgccacagatgaagctgccagccgagctccagtccatcatgatgccaaagaaggaagagaagggcaagccacaaggcgctcaagctaaggctaaggtgccagctaaggctggccaagtccagaagccaaaggcccaggacatcatgccaagcaggcgcatccgcgacctgttcgtgctcccaaaggagatcttcggcagcctgaagaacttcaaggagtccgccctcaagttcgccaaccacatcggcctgaacctggagaccatcaagaagcacctcaccaccgtgaagaacttcctcctgagggtcgacgccgtggtcgacaaggagatcggcaacatcatcgaggccggcaagtccccacaaaacgtggtccaggccaacgagggcttcctggacaagatgaagcgcctcgtgaacaagtacaagatcttcagcatcccattcttcgccggcatgggctccttcggcttccaccatcaccaccatcactga

SEQ ID NO: 59 - protein, 437 aa, Plasmodium vivax
MQLGIQKKKKNLEQDAMHALMKKLESLYKLSATDNGEIFNKEIDALKKQIDQLHQHGGGNEGESLGHLLESEAADDSGKKTIFGVDEDDLDNYDADFIGQSKGKIKGQADTTAVAKPPTGSGAGAHGSHSPPKPSVLVVPGKSGKEDSVATLENGYESIHGEDEPREDSTSHDSPPALPVGRSEGDSSASGGGTEGQQPDPASARGSQASGGRGGGDQTNTTQPAGGQQSSSAARSLQAPHAGDSQLPNAGGDPQSPAAAGHQQPPTSPPANNEGTTVTQESALAATPPKGTADSNDAKIKYLDKLYDEVLTTSDNTSGIHVPDYHSKYNTIRQKYEYSMNPVEYEIVKNLFNVGFKNDGAASSDATPLVDVFKKALADEKFQAEFDNFVHGLYGFAKRHSYLSEARMKDNKLYSDLLKNAISLMSTLQVSHHHHHH

SEQ ID NO: 60 - DNA, 1314 bp, Plasmodium vivax
  source 1..1314: mol_type = genomic DNA
atgcagctcggcatccaaaagaagaagaagaacctggagcaggacgccatgcacgccctcatgaagaagctggagagcctgtacaagctctccgccaccgacaacggcgagatcttcaacaaggagatcgacgccctgaagaagcaaatcgaccagctccaccaacacggcggcggaaacgagggcgagagcctgggccacctcctggagagcgaggctgctgacgactccggcaagaagaccatcttcggcgtggacgaggacgacctggacaactacgacgccgacttcatcggccagtccaagggcaagatcaagggccaggctgacaccaccgctgtggctaagccaccaaccggcagcggcgctggcgctcacggcagccactccccaccaaagccatccgtgctcgtggtcccaggcaagagcggcaaggaagactccgtcgccaccctggagaacggctacgagagcatccacggcgaggacgagccaagggaggacagcacctcccacgactccccaccagctctcccagtgggccgcagcgagggcgactccagcgcttccggcggcggcaccgagggccaacagccagacccagctagcgccaggggcagccaggcttccggcggcaggggcggcggcgaccaaaccaacaccacccaaccagctggcggccaacagtccagctccgctgctaggagcctgcaggccccacacgctggcgacagccagctcccaaacgccggcggcgacccacaatccccagctgccgccggccaccaacagccaccaacctccccaccagccaacaacgagggcaccaccgtgacccaagagtccgctctggctgctaccccaccaaagggcaccgccgactccaacgacgccaagatcaagtacctggacaagctctacgacgaggtgctgaccaccagcgacaacacctccggcatccacgtcccagactaccacagcaagtacaacaccatccgccaaaagtacgagtactccatgaacccagtggagtacgagatcgtcaagaacctcttcaacgtgggcttcaagaacgacggcgctgccagctccgacgctaccccactggtggacgtcttcaagaaggccctcgccgacgagaagttccaggccgagttcgacaacttcgtccacggcctgtacggcttcgccaagaggcacagctacctctccgaggcccgcatgaaggacaacaagctgtacagcgacctcctgaagaacgccatcagcctgatgtccaccctccaagtgtcccaccaccaccaccaccactga

SEQ ID NO: 61 - protein, 675 aa, Plasmodium vivax
MAAYNTVLQIYKYSDDIVRKQEKCEQLVKDGKDICLKFKSINEIKVMIQNSKGKESTLSAKVSHSFNKLSELNKIKCNDESYDAILETPSREELNKLRSTFKQEKDTIANQAKLSGYKTDFETHIGKLNDLAKIVDNLKASETLPKNIEEKKTSINLISTKLETIEKEIESINSSFDQLLEKGKKCEMTKYKLVRDSLSTKINDHSAIIKDNQKKATEYLTYIQNNHISIFKDIDMLNENLGEKSVSRYAIAKIEEANDLSAQLTAAVSEYEAIANSIRKEFTNISDHTEMDTLENEAKMLKEHYDNLINKKNIITELHNKINLIKLLEIRATSDKYVDIAELLGEVVKDQKKKLQEAKNKLDTLKDHIAVKEKELINHDSSFTLVSIKAFDEIYDDIKYNVGQLHTLEVTNFDELKKGKTYEENVTHLLNRRETLQNDLHNYEEKDKLKNTNIEMSNEENNQIRQTSEVIKKLESEFQNLLKIIQQSNTLCSNDNIKQFISDILKKVETIRERFVKNFPEREKYHQIEINYNEIKGIVKEVDTNPEISIFTEKINTYIRQKIRSAHHLEDAQKIKDIIEDVTSNYRKIKSKLSQVNNALDRIKIKKSEMDTLFESLSKENANNYNSAKYFLVDSDKIIKHLEDQVSKMSSLISYAEREIKELEEKVYSHHHHHH

SEQ ID NO: 62 - DNA, 2028 bp, Plasmodium vivax
  source 1..2028: mol_type = genomic DNA
atggccgcctacaacaccgtgctccaaatctacaagtactccgacgacatcgtgaggaagcaagagaagtgcgagcagctggtcaaggacggcaaggacatctgcctcaagttcaagtccatcaacgagatcaaggtcatgatccagaacagcaagggcaaggagtccaccctcagcgccaaggtgtcccacagcttcaacaagctcagcgagctgaacaagatcaagtgcaacgacgagagctacgacgccatcctcgaaaccccatccagggaggagctcaacaagctgcgcagcaccttcaagcaagagaaggacaccatcgccaaccaggccaagctctccggctacaagaccgacttcgagacgcacatcggcaagctcaacgacctggccaagatcgtggacaacctcaaggccagcgagacgctgccaaagaacatcgaggagaagaagacctccatcaacctcatcagcaccaagctcgaaaccatcgagaaggagatcgagtccatcaactccagcttcgaccaactcctggagaagggcaagaagtgcgagatgaccaagtacaagctcgtcagggactccctgagcaccaagatcaacgaccactccgccatcatcaaggacaaccaaaagaaggccaccgagtacctcacctacatccagaacaaccacatcagcatcttcaaggacatcgacatgctcaacgagaacctgggcgagaagtccgtgagcaggtacgccatcgccaagatcgaggaagccaacgacctctccgctcaactcaccgctgccgtcagcgagtacgaggctatcgccaactccatccgcaaggagttcaccaacatctccgaccacaccgagatggacaccctggagaacgaggccaagatgctgaaggagcactacgacaacctcatcaacaagaagaacatcatcaccgagctccacaacaagatcaacctgatcaagctcctggagatccgcgccaccagcgacaagtatgtggacatcgccgagctcctgggcgaggtggtcaaggaccaaaagaagaagctgcaagaggccaagaacaagctcgacaccctgaaggaccacatcgccgtgaaggagaaggagctgatcaaccacgactccagcttcaccctcgtcagcatcaaggccttcgacgagatctacgacgacatcaagtacaacgtgggccaactccacaccctggaggtcaccaacttcgacgagctcaagaagggcaagacctacgaggagaacgtgacccacctcctgaacaggcgcgagacgctccagaacgacctgcacaactacgaggagaaggacaagctcaagaacaccaacatcgagatgtccaacgaggagaacaaccaaatcaggcagaccagcgaggtcatcaagaagctggagtccgagttccaaaacctcctgaagatcatccaacagtccaacaccctctgcagcaacgataacatcaagcagttcatcagcgacatcctgaagaaggtggagacgatcagggagcgcttcgtcaagaacttcccagagcgcgagaagtaccaccaaatcgagatcaactacaacgagatcaagggcatcgtgaaggaagtggacaccaacccagagatctccatcttcaccgagaagatcaacacctacatcaggcaaaagatcaggagcgctcaccacctggaggacgctcagaagatcaaggacatcatcgaggacgtgacctccaactacaggaagatcaagtccaagctgagccaagtcaacaacgccctcgaccgcatcaagatcaagaagagcgagatggacaccctcttcgagtccctgagcaaggagaacgccaacaactacaacagcgccaagtacttcctggtggactccgacaagatcatcaagcacctggaggaccaagtgtccaagatgtccagcctgatcagctacgccgagcgcgagatcaaggagctggaggagaaggtctactcccaccaccaccaccaccactga

SEQ ID NO: 63 - protein, 1003 aa, Plasmodium vivax
MNVATRGEIVNLKNPNLRNGWSMKNLSAQNEENIVHSDGSDDVTDKEEDGEVLEGQKGSPKKSAEQKVHAQEEVNKESLKSKAQNAKAEAEKAAKAAESAKENTLDALEKVNVPTELNNEKNFAESAATEAKKQEKISTEAAEEVKEIEVDGQLEKLKNEEEKTAKKARKQEIKTEIAEQAAKAQAAKTEAETAQKDATTAKDEAIKETGKPKSQNTTKAVTMATEEEKKTKDEAQTASEKAGKTAEEAQKEVGKETADDDKEVSQLEEEIKELERILKIVKDLASEASSASDNAKKAKLKTQIAAEVVKAEKARIEAEEAEKEAGEAKTKTEATEKEVLKISDESKAAKVKKAVEKAKEAEKQAKSEAEKAKGMADDAGGKGTTNLEDVLTKLSEVLTSVKSLASNAEVASKNAKKEMTKAQIAAEVAKAEKAKIEAENAKLLADTASKAAENIAKSSKAAKIANNVSTIAAEKSKVATEAADEAAKALDETENPESKIAEVTEKATKAVNAAEEAKKEKAKAEVAVEVAHAEVAKEKAQEAKEAAKQVADKSKLEKAIQAADKASEKANEASKLAEEALSNLESLEKETGEIVEKVNAIEQKVQTAKNAAIEAHKEKTKAEIAVEVAKAEEAKKEADNAKVAAEKAKETAEKIAKTSKSTEKITEEVRKATEFAKTAGDETTLAATKAESEIPSEEKNQKELLDSIKQKAESAFQASQEAIKAKTEAENFLEIAKEVPKAEAAKEEAQKAATAAEEAKTEVLKIAEEVNKSDASESEKKKIETAANETAGEAEKAATFAKEAADAAKDTNKAVTLAVAKEKVEKALKAAKEAKKANEKASYALIRTKKQYALEPLEITSEAGYNITEKEEQVKEEIEEQDDKASEEEEEDTQQIDQTQIDEVDISVDNEEEEEGAAEEQIEGEKDTPTKEAKEEQTSGEKILDDKEAHKTLAEKFKDSNTAKTGGVEFLETLISDVGEDTLKNLQQDLHQYFKGKHHHHHH

SEQ ID NO: 64 - DNA, 3012 bp, Plasmodium vivax
  source 1..3012: mol_type = genomic DNA
atgaacgtcgccaccaggggcgagatcgtgaacctgaagaacccaaacctccgcaacggctggagcatgaagaacctgtccgcccaaaacgaggagaacatcgtccactccgacggcagcgacgacgtgaccgacaaggaagaggacggcgaggtgctggagggccagaagggcagcccaaagaagtccgccgagcaaaaggtccacgcccaagaggaagtgaacaaggagtccctcaagagcaaggcccaaaacgccaaggctgaggctgagaaggctgctaaggctgccgagtccgccaaggagaacaccctcgacgccctggagaaggtgaacgtcccaaccgagctcaacaacgagaagaacttcgctgagagcgctgctaccgaggccaagaagcaggagaagatctccaccgaggccgccgaggaagtgaaggagatcgaggtggacggccaactggagaagctgaagaacgaggaagagaagaccgccaagaaggccaggaagcaggagatcaagaccgagatcgctgagcaagctgctaaggctcaggctgctaagaccgaggccgagacggcccaaaaggacgccaccaccgccaaggacgaggccatcaaggagacgggcaagccaaagagccagaacaccaccaaggccgtcaccatggccaccgaggaagagaagaagaccaaggacgaggctcaaaccgcttccgagaaggctggcaagaccgctgaggaagcccagaaggaagtgggcaaggagacggccgacgacgacaaggaagtgtcccaactcgaagaggagatcaaggagctggagaggatcctcaagatcgtgaaggacctggctagcgaggcctccagcgcttccgacaacgccaagaaggccaagctcaagacccaaatcgctgctgaggtggtcaaggctgagaaggctaggatcgaggctgaggaagccgagaaggaagccggcgaggctaagaccaagaccgaggctaccgagaaggaagtgctgaagatctccgacgagagcaaggccgccaaggtcaagaaggccgtggagaaggccaaggaagccgagaagcaagccaagtccgaggctgagaaggctaagggcatggctgacgacgccggcggcaagggcaccaccaacctggaggacgtgctcaccaagctgagcgaggtcctgacctccgtgaagtccctggcttccaacgctgaggtggcttccaagaacgccaagaaggagatgaccaaggctcagatcgctgctgaggtggctaaggctgagaaggccaagatcgaggccgagaacgccaagctgctggctgacaccgctagcaaggctgccgagaacatcgccaagtccagcaaggccgccaagatcgccaacaacgtcagcaccatcgccgccgagaagtccaaggtggctaccgaggctgctgacgaggctgccaaggccctcgacgagacggagaacccagagtccaagatcgccgaggtgaccgagaaggctaccaaggctgtgaacgctgctgaggaagccaagaaggagaaggccaaggctgaggtggctgtggaggtggctcacgctgaggtggctaaggagaaggcccaagaggccaaggaagccgccaagcaggtggccgacaagagcaagctggagaaggccatccaagccgccgacaaggccagcgagaaggccaacgaggcctccaagctcgccgaggaagccctcagcaacctggagtccctggagaaggagacgggcgagatcgtcgagaaggtgaacgccatcgagcaaaaggtgcagaccgccaagaacgccgccatcgaggcccacaaggagaagaccaaggctgagatcgctgtggaggtcgccaaggccgaggaagccaagaaggaagccgacaacgccaaggtggctgctgagaaggctaaggagacggccgagaagatcgccaagacctccaagagcaccgagaagatcaccgaggaagtgaggaaggctaccgagttcgctaagaccgctggcgacgagacgaccctggctgctaccaaggctgagagcgagatcccatccgaggagaagaaccaaaaggagctcctggacagcatcaagcagaaggccgagagcgccttccaagcctcccaagaggccatcaaggccaagaccgaggccgagaacttcctggagatcgccaaggaagtgccaaaggccgaggccgccaaggaagaggcccaaaaggctgctacggccgctgaggaagccaagaccgaggtcctcaagatcgccgaggaagtgaacaagtccgacgcctccgagagcgagaagaagaagatcgagacggctgctaacgagacggctggcgaggccgagaaggccgctaccttcgctaaggaagccgctgacgctgctaaggacaccaacaaggccgtcaccctggccgtggccaaggagaaggtcgagaaggccctcaaggccgccaaggaagccaagaaggccaacgagaaggccagctacgccctgatccgcaccaagaagcagtacgccctggagccactggagatcacctccgaggccggctacaacatcaccgagaaggaagagcaagtgaaggaagagatcgaggagcaggacgacaaggccagcgaggaagaggaagaggacacccaacagatcgaccaaacccagatcgacgaggtcgacatctccgtggacaacgaggaagaggaagagggcgctgctgaggagcaaatcgagggcgagaaggacaccccaaccaaggaagccaaggaagagcagacctccggcgagaagatcctggacgacaaggaagcccacaagaccctcgccgagaagttcaaggacagcaacaccgctaagaccggcggcgtcgagttcctcgaaaccctcatctccgacgtgggcgaggacaccctgaagaacctccaacaggacctccaccagtacttcaagggcaagcaccaccaccaccaccactga

SEQ ID NO: 65 - protein, 656 aa, Plasmodium vivax
MNNYGKLKHGKWDDGSYSERTRWRMLSGDDHDDLLPSCDSPGGRNDEHQVNKEVSRTAPSEKVKVVDKETGESMLVDVGESGGKSSPGVAEESGPSLRGRDVRDVRVDQETRETLQGGATNRRDLTQHGEEETGDDSKRAKQDDEAGVRSMLNDTVTAIKDNGSNLLRSVIGQINFVQGSAELLKVANEEERQPSGGSVLSKEGEEATPGDFLGGNNPNGGEKGELPNGTKNDVMIKGYANVLLNEGKHVLVGNVRNFLSRVFNLIVREKIMTRMCHRGGEASIERSGEPVGERSGEPTGERSGDPTGERSGDPTGERSGEPTGERSGEPTGERSGEPTAERSGEPTAERSDEPTAERSDEPTADPKGDPTNCRLPKRSATKFYQSEDLYNYYSSLEEMLKGRGIRWKTDRVSRYFTFSPSKKIKDNFEEVMNNKVFIESVRSILFDSHKKNKKAVFSSFAVVVETLFSLIKEEKVIADMYSYVKLFFQDLDILNLKVLHFLSSSSTENTQFVGPPDLSLTNFEYILAKIYSRSVLANILSPKMNHSDSKKLSKLLTRRENNLKFSFLEGVKMVHSAIPSEGVSAVVLGNAGGQVNVPIPGADDTLCKFIPIRKKLLYERLSVTRKVAEEVILDYLFRLLLRKVHEYVLEHHHHHH

SEQ ID NO: 66 - DNA, 1971 bp, Plasmodium vivax
  source 1..1971: mol_type = genomic DNA
atgaacaactacggcaagctcaagcacggcaagtgggacgacggctcctacagcgagaggaccaggtggaggatgctgtccggcgacgaccacgacgacctcctcccatcctgcgacagcccaggcggcaggaacgacgagcaccaagtcaacaaggaagtgtccaggaccgccccaagcgagaaggtgaaggtggtcgacaaggagaccggcgagtccatgctggtggacgtgggcgagagcggcggcaagtcctccccaggcgtggctgaggagtccggcccaagcctgcgcggcagggacgtgcgcgacgtcagggtggaccaagagacccgcgagaccctgcagggcggcgccaccaacaggcgcgacctcacccaacacggcgaggaagagaccggcgacgacagcaagcgcgctaagcaggacgacgaggctggcgtcaggtccatgctcaacgacaccgtgaccgccatcaaggacaacggctccaacctcctgcgcagcgtcatcggccaaatcaacttcgtgcaaggcagcgctgagctcctgaaggtcgccaacgaggaagagcgccagccatccggcggcagcgtgctgtccaaggaaggcgaggaagccaccccaggcgacttcctcggcggcaacaacccgaacggcggcgagaagggcgagctgccaaacggcaccaagaacgacgtcatgatcaagggctacgccaacgtgctcctgaacgagggcaagcacgtcctcgtgggcaacgtccgcaacttcctgtccagggtgttcaacctcatcgtcagggagaagatcatgaccaggatgtgccacaggggcggcgaggctagcatcgagaggtccggcgagccagtgggggagcgctccggcgagccaaccggcgagaggagcggcgacccaaccggcgagaggtctggcgaccctacgggggagaggagcggggagcctaccggcgagcgcagcggggagcctacgggcgagaggtccggggagcctaccgctgagagaagcggcgagccaaccgctgagaggagcgatgagcctaccgctgagaggtccgacgagccaaccgctgacccaaagggcgacccaaccaactgccgcctcccaaagaggtccgccaccaagttctaccaaagcgaggacctgtacaactactactccagcctggaggagatgctcaagggcaggggcatcaggtggaagaccgaccgcgtcagcaggtacttcaccttctccccaagcaagaagatcaaggacaacttcgaggaagtgatgaacaacaaggtcttcatcgagagcgtgcgctccatcctcttcgactcccacaagaagaacaagaaggccgtgttctccagcttcgccgtggtcgtggagaccctgttcagcctcatcaaggaagagaaggtcatcgccgacatgtactcctacgtgaagctgttcttccaagacctcgacatcctgaacctcaaggtcctgcacttcctctccagctccagcaccgagaacacccagttcgtgggcccaccagacctgagcctcaccaacttcgagtacatcctcgccaagatctactcccgcagcgtcctggccaacatcctcagcccaaagatgaaccactccgacagcaagaagctgtccaagctcctgaccaggcgcgagaacaacctgaagttctccttcctggagggcgtcaagatggtgcacagcgctatcccatccgagggcgtgagcgctgtggtgctgggcaacgctggcggccaggtcaacgtgccaatcccaggcgccgacgacaccctctgcaagttcatcccaatcaggaagaagctcctgtacgagcgcctgtccgtcaccaggaaggtggccgaggaagtgatcctggactacctcttccgcctcctgctcaggaaggtgcacgagtatgtgctggagcaccatcaccaccatcactga

SEQ ID NO: 67 - protein, 447 aa, Plasmodium vivax
MGNVSPPNFNDNRVNGNNGNKGNGNDNDVPSFIGGNNNNVNGNNDDNIFNKNGKDVTRNDGDAKDGENRNNKKNENGSGSNENNSIANADNGSGKSDANANQIDEDGNKMDEASLKKILKIVDEMENIQGLLDGDYSILDKYSVKLVDEDDGETNKRKIIGEYDLKMLKNILLFREKISRVCENKYNKNLPVLLKKCSNVDDPKLSKSREKIKKGLAKNNMSIEDFVVGLLEDLFEKINEHFIKDDSFDLSDYLADFELINYIIMHETSELIDELLNIIESMNFRLESGSLEKMVKSAESGMNLNCKMKEDIIHLLKKSSAKFFKIEIDRKTKMIYPVQATHKGANMKQLALSFLQKNNVCEHKKCPLNSNCYVINGEEVCRCLPGFSDVKIDNVMNCVRDDTLDCSNNNGGCDVNATCTLIDKKIVCECKDNFEGDGIYCHHHHHH

SEQ ID NO: 68 - DNA, 1344 bp, Plasmodium vivax
  source 1..1344: mol_type = genomic DNA
atgggcaacgtgtccccaccaaacttcaacgacaacagggtcaacggcaacaacggcaacaagggcaacggcaacgacaacgacgtgccaagcttcatcggcggcaacaacaacaacgtcaacggcaacaacgacgacaacatcttcaacaagaacggcaaggacgtgacccgcaacgacggcgacgctaaggacggcgagaaccgcaacaacaagaagaacgagaacggctccggcagcaacgagaacaactccatcgccaacgctgacaacggctccggcaagagcgacgccaacgccaaccaaatcgacgaggacggcaacaagatggacgaggccagcctcaagaagatcctgaagatcgtggacgagatggagaacatccagggcctcctggacggcgactactccatcctcgacaagtacagcgtgaagctggtcgacgaggacgacggcgagacgaacaagaggaagatcatcggcgagtacgacctcaagatgctgaagaacatcctcctgttcagggagaagatctcccgcgtctgcgagaacaagtacaacaagaacctcccagtgctcctgaagaagtgcagcaacgtcgacgacccaaagctctccaagagccgcgagaagatcaagaagggcctggctaagaacaacatgtccatcgaggacttcgtggtcggcctcctggaggacctgttcgagaagatcaacgagcacttcatcaaggacgactccttcgacctcagcgactacctggccgacttcgagctcatcaactacatcatcatgcacgagacgtccgagctgatcgacgagctcctgaacatcatcgagagcatgaacttcaggctggagtccggcagcctggagaagatggtgaagtccgccgagagcggcatgaacctcaactgcaagatgaaggaagacatcatccacctcctgaagaagtccagcgccaagttcttcaagatcgagatcgaccgcaagaccaagatgatctacccagtgcaagccacccacaagggcgccaacatgaagcaactcgccctgtccttcctccagaagaacaacgtctgcgagcacaagaagtgcccactgaacagcaactgctacgtgatcaacggcgaggaagtgtgcaggtgcctcccaggcttctccgacgtcaagatcgacaacgtgatgaactgcgtccgcgacgacaccctcgactgcagcaacaacaacggcggctgcgacgtgaacgctacctgcaccctgatcgacaagaagatcgtctgcgagtgcaaggacaacttcgagggcgacggcatctactgccaccaccaccaccaccactga

SEQ ID NO: 69 - protein, 226 aa, Plasmodium vivax
METLLDSETLKNYEKETNEYIRKKKVEKLFDVILKNVLVNKPENVYLYIYKNIYSFLLNKIFVIGPPLLKITPTLCSAIASCFSYYHLSASHMIESYTTGEVDDAAESSTSKKLVSDDLICSIVKSNINQLNAKQKRGYVVEGFPGTNLQADSCLRHLPSYVFVLYADEEYIYDKYEQENNVKIRSDMNSQTFDENTQLFEVAEFNTNPLKDEVKVYLRNHHHHHH

SEQ ID NO: 70 - DNA, 681 bp, Plasmodium vivax
  source 1..681: mol_type = genomic DNA
atggagacgctcctggactccgagacgctcaagaactacgagaaggagacgaacgagtacatcaggaagaagaaggtggagaagctcttcgacgtcatcctcaagaacgtgctggtcaacaagccagagaacgtgtacctgtacatctacaagaacatctacagcttcctcctgaacaagatcttcgtcatcggcccaccactcctgaagatcaccccaaccctctgctccgccatcgcctcctgcttcagctactaccacctgtccgccagccacatgatcgagagctacaccaccggcgaggtggacgacgctgctgagtccagcacctccaagaagctcgtgagcgacgacctgatctgctccatcgtcaagagcaacatcaaccaactcaacgccaagcagaagaggggctacgtggtcgagggcttcccaggcaccaacctccaggctgactcctgcctcaggcacctgccaagctacgtgttcgtcctgtacgccgacgaggagtacatctacgacaagtacgagcaggagaacaacgtgaagatcaggtccgacatgaacagccaaaccttcgacgagaacacccagctgttcgaggtcgccgagttcaacaccaacccactcaaggacgaggtgaaggtctacctgcgcaaccaccaccaccaccaccactga

SEQ ID NO: 71 - protein, 395 aa, Plasmodium vivax
MKPGVEKKKKLEEDVIGILRRKLESLQKRSLTNSDGKLKKEIELVKKQIQELQKYEKGEAGKKVDATLGEEPGVESAEEQPLSVEEAGDTQDEDRLDELEGVEDFEEENLEQSEQVEEAEVVEEAEEEAGDAEEEQPAEAEEDGSLLEEAPNSVERKAEGAIAEFEEADVEEGAEADEGVETDEGADADEASLGSFDLEGELIEEDLQESFDLEGEQEEEDLQEGFKSEEEANQGGQLPREIPPHGEEAVEPPLRGNKPSMEYVGNLHSDVGPTEGSANQISPPSVDEKGKEDGDKYKSASQDGGNSVGINNFGGCFQGGNSNGICPLDIFKKVLEDENFLQEFDSFIHNLYGSSKNNTPWGGDKMGNENLYMDLFTNALSFLNTIEVIHHHHHH

SEQ ID NO: 72 - DNA, 1188 bp, Plasmodium vivax
  source 1..1188: mol_type = genomic DNA
atgaagccaggcgtggagaagaagaagaagctcgaagaggacgtcatcggcatcctgcgcaggaagctggagtccctgcaaaagaggtccctcaccaacagcgacggcaagctcaagaaggagatcgagctggtcaagaagcaaatccaggagctgcagaagtacgagaagggcgaggctggcaagaaggtggacgctaccctgggcgaggagccgggcgtggagtccgctgaggagcaaccactgagcgtggaggaagccggcgacacccaggacgaggacaggctcgacgagctggagggcgtcgaggacttcgaggaagagaacctggagcaaagcgagcaggtggaggaagccgaggtggtggaggaagccgaggaagaggccggcgacgctgaggaagagcaaccggctgaggctgaggaagacggctccctcctcgaagaggccccaaacagcgtggagaggaaggctgagggcgctatcgctgagttcgaggaagccgacgtcgaggaaggcgccgaggccgacgagggcgtggagacggacgagggcgctgacgctgacgaggcttccctgggcagcttcgacctggagggcgagctgatcgaggaagacctccaggagtctttcgacctggagggggagcaagaggaagaggacctccaagagggcttcaagagcgaggaagaggccaaccaaggcggccagctgccaagggagatcccaccacacggcgaggaagccgtggagccaccactccgcggcaacaagccatccatggagtatgtgggcaacctgcacagcgacgtgggcccaaccgagggcagcgccaaccaaatctccccaccaagcgtcgacgagaagggcaaggaagacggcgacaagtacaagtccgccagccaagacggcggaaactccgtgggcatcaacaacttcggcggatgcttccagggcggcaacagcaacggcatctgcccactcgacatcttcaagaaggtcctggaggacgagaacttcctgcaggagttcgactccttcatccacaacctgtacggctccagcaagaacaacaccccatggggcggcgacaagatgggcaacgagaacctctacatggacctgttcaccaacgccctcagcttcctgaacaccatcgaggtcatccaccaccaccaccaccactga

SEQ ID NO: 73 - protein, 372 aa, Plasmodium vivax
MELSHSLSVKNAPDASALNIEVEKDKKKICKNAFQYINVAELLSPREEETYVQKCEEVLDTIKNDSPDESAEAEINEFILSLLHARSKYTIINDSDEEVLSKLLRSINGSISEEAALKRAKQLITFNRFIKDKAKVKNVQEMLVISSKADDFMNEPKQKMLQKIIDSFELYNDYLVILGSNINIAKRYSSETFLSIKNEKFCSDHIHLCQKFYEQSIIYYRLKVIFDNLVTYVDQNSKHFKKEKLLELLNMDYRVNRESKVHENYVLEDETVIPTMRITDIYDQDRLIVEVVQDGNSKLMHGRDIEKREISERYIVTVKNLRKDLNDEGLYADLMKTVKNYVLSITQIDNDISNLVRELDHEDVEKHHHHHH

SEQ ID NO: 74 - DNA, 1119 bp, Plasmodium vivax
  source 1..1119: mol_type = genomic DNA
atggagctctcccacagcctgtccgtgaagaacgctccagacgctagcgctctcaacatcgaggtcgagaaggacaagaagaagatctgcaagaacgccttccaatacatcaacgtcgccgagctcctgtccccaagggaggaagagacttacgtgcagaagtgcgaggaagtgctggacaccatcaagaacgacagcccagacgagtccgctgaggctgagatcaacgagttcatcctcagcctcctgcacgcccgctccaagtacaccatcatcaacgacagcgacgaggaagtgctgagcaagctcctgaggtccatcaacggcagcatctccgaggaagccgctctcaagagggctaagcaactgatcaccttcaacaggttcatcaaggacaaggccaaggtgaagaacgtccaggagatgctcgtcatctccagcaaggccgacgacttcatgaacgagccaaagcaaaagatgctccagaagatcatcgacagcttcgagctgtacaacgactacctcgtgatcctgggctccaacatcaacatcgccaagcgctactccagcgagacgttcctcagcatcaagaacgagaagttctgctccgaccacatccacctgtgccaaaagttctacgagcagagcatcatctactacaggctcaaggtcatcttcgacaacctggtgacctacgtcgaccaaaactccaagcacttcaagaaggagaagctcctggagctcctgaacatggactacagggtgaaccgcgagtccaaggtgcacgagaactacgtcctggaggacgagactgtgatcccaaccatgcgcatcaccgacatctacgaccaagacaggctcatcgtggaggtggtccaggacggcaacagcaagctgatgcacggcagggacatcgagaagcgcgagatctccgagaggtacatcgtgaccgtcaagaacctccgcaaggacctgaacgacgagggcctctacgccgacctgatgaagaccgtgaagaactacgtcctcagcatcacccagatcgacaacgacatctccaacctcgtgagggagctggaccacgaggacgtcgagaagcaccaccaccaccaccactga

SEQ ID NO: 75 - protein, 450 aa, Plasmodium vivax
MEKLDIPPHEMYEDMQQAFREQDKYDFLAISDGSVINSYMKKNVVDWNNRYSYNQLKNKDSLIMFLVDIFRSLFLSNCIDKNIDNVLSSIEEMFTDHYYNPMHSRLKYLIDDVGIFFTKLPITKAFHTYNKKYRITKRLYAPPTFNEVRHILNLAQILSLEDGLDLLTFDADETLYPDGYDFHDEVLASYISSLLKKMNIAIVTAASYSNDAEKYQKRLENLLRYFSKHNIEDGSYENFYVMGGESNYLFKCNEDANLYSVPEEEWYHYKKYVNKETVEQILDISQKCLQQVITDFKLCAQIQRKEKSIGLVPNKIPSANNQKEQKNYMIKYEVLEEAVIRVKKEIVKNKITAPYCAFNGGQDLWVDIGNKAEGLIILQKLLKIEKKKCCHIGDQFLHSGNDFPTRFCSLTLWISNPQETKACLKSIMNLNMKSFIPEVLYENEHHHHHH

SEQ ID NO: 76 - DNA, 1353 bp, Plasmodium vivax
  source 1..1353: mol_type = genomic DNA
atggagaagctcgacatcccaccacacgagatgtacgaggacatgcaacaggccttcagggagcaagacaagtacgacttcctggccatctccgacggcagcgtgatcaactcctacatgaagaagaacgtggtcgactggaacaacaggtactcctacaaccagctcaagaacaaggacagcctcatcatgttcctggtggacatcttccgctccctcttcctgagcaactgcatcgacaagaacatcgacaacgtcctgtccagcatcgaggagatgttcaccgaccactactacaacccaatgcacagcaggctcaagtacctgatcgacgacgtgggcatcttcttcaccaagctcccaatcaccaaggccttccacacctacaacaagaagtacaggatcaccaagcgcctgtacgccccaccaaccttcaacgaggtccgccacatcctcaacctggcccaaatcctctccctggaggacggcctcgacctcctgaccttcgacgccgacgagacgctgtacccagacggctacgacttccacgacgaggtgctcgccagctacatctccagcctcctgaagaagatgaacatcgccatcgtcaccgccgcctcctacagcaacgacgccgagaagtaccagaagaggctggagaacctcctgcgctacttctccaagcacaacatcgaggacggcagctacgagaacttctacgtgatgggcggcgagtccaactacctcttcaagtgcaacgaggacgccaacctgtacagcgtcccagaggaagagtggtaccactacaagaagtatgtgaacaaggagacggtcgagcaaatcctcgacatctcccagaagtgcctgcaacaagtgatcaccgacttcaagctctgcgcccaaatccagaggaaggagaagtccatcggcctggtcccaaacaagatcccaagcgccaacaaccaaaaggagcagaagaactacatgatcaagtacgaggtgctcgaagaggccgtgatccgcgtcaagaaggagatcgtcaagaacaagatcaccgctccatactgcgccttcaacggcggccaagacctgtgggtggacatcggcaacaaggccgagggcctcatcatcctgcaaaagctcctgaagatcgagaagaagaagtgctgccacatcggcgaccagttcctccacagcggcaacgacttcccaacccgcttctgctccctcaccctgtggatcagcaacccacaggagacgaaggcctgcctcaagtccatcatgaacctgaacatgaagagcttcatcccagaggtcctctacgagaacgagcaccaccaccaccaccactga

SEQ ID NO: 77 - protein, 527 aa, Plasmodium vivax
MEIIAEKPKVKFNFASEEYKNCDSSDYSECAEDYGRPNGKDYFYANRILSLDRNSEQRRKESPSKRPGLCVDEICTCGFHRCPKIVKSLPFDGESNYRSEFGPKPLPELPPRQEAKLTRSLPFEGESNYRSEFGPKPLPELPPRVEQKPPKSLPFDGESNYRSEFGPKPLPELPPRVEQKPPKSLPFDGESNYRSEFGPKPLPELPPRVEQKPPKSLPFEGESNYRSEFGPKPLPELPPRVEQKPPKSLPFEGESNYRSEFGPKALPELPPRVEQKPPKSLPFEGESNYRSEFGPKPLPALPPRVETKLVKSLPFEGESNYRSEFGPKPLPELPPRVEQKPPKSLPFEGESNYRSEFGPKPLPALPPRVVTKLVKSLPFEGESNYRSEFGPKPLPEIPPRVEQKPPKSLPFEGESNYRSEFGPKPLPELPPRVEQKPPKSLPFEGESNYRSEFGPKQLPELPPRQEAKLTRSLPFEGESSYRSEYVRKAIPICPVNLLPKYPAPTYPSEHVFWDSACKRWYHHHHHH

SEQ ID NO: 78 - DNA, 1584 bp, Plasmodium vivax
  source 1..1584: mol_type = genomic DNA
atggagatcatcgccgagaagccaaaggtcaagttcaacttcgcctccgaggagtacaagaactgcgactccagcgactactccgagtgcgctgaggactacggcaggccaaacggcaaggactacttctacgccaacaggatcctctccctggaccgcaacagcgagcagaggcgcaaggagtccccaagcaagaggccaggcctctgcgtggacgagatctgcacctgcggcttccaccgctgcccaaagatcgtcaagtccctgccattcgacggcgagtccaactaccgcagcgagttcggcccaaagccactcccagagctgccaccaaggcaagaggccaagctcacccgcagcctgccattcgagggcgagtccaactacaggtccgagttcgggcctaagcctctgcctgagctgccaccacgcgtggagcaaaagccaccaaagtccctccctttcgatggggagagcaactacaggagtgaattcgggcctaagccgctgcccgagctgccaccacgcgtcgagcagaagccaccaaagagcctccctttcgatggcgagagcaactacaggagcgaatttgggcctaagccgctgccggaactgccaccacgcgtggaacaaaagccaccaaagagcctgcctttcgagggggagtccaactacaggagtgagtttgggcctaagccgttgcctgaactgccaccacgcgtcgaacagaaaccaccaaaaagcctccctttcgagggcgagagcaactaccgctccgagttcggcccaaaggctctgccggagctgccaccacgcgtggaacagaaaccaccaaagagcctccccttcgagggggagagcaattatcgctctgagttcgggccaaagccgctgccggctctgccaccacgcgtggagacgaagctcgtcaagagcctcccgttcgagggggagagcaactatcgctccgaatttgggcctaaaccactgcctgaactgccaccacgcgtggaacagaagccaccaaaaagcctcccctttgaaggggagagcaattaccgctccgagttcgggcccaagccgctgccggccctgccaccacgcgtggtcaccaagctcgtgaagtccctcccctttgaaggcgagagcaactacagatctgagttcgggcctaagccactcccagagatcccaccacgcgtcgagcaaaaaccaccaaaatctctcccctttgagggtgagagcaattatcgctcagagttcgggcccaagcctctgccggagctgccaccacgcgtcgaacagaagccaccaaagagcttaccttttgaaggggagagcaactaccgcagtgaattcggcccaaagcagctgccagaactgccaccaaggcaagaggccaaactcacccgctccctgcctttcgagggcgagtccagctacaggagcgagtatgtgaggaaggccatcccaatctgcccagtcaacctcctgccaaagtacccagccccaacctacccatccgagcacgtgttctgggacagcgcctgcaagcgctggtaccaccaccaccaccaccactga

SEQ ID NO: 79 - protein, 266 aa, Plasmodium vivax
MAAANRPNANGFVSPTLIGFGELSIQESEEFKRMAWNNWMLRLESDWKHFNDSVEEAKTKWLHERDSAWSDWLRSLQSKWSHYSEKMLKEHKSNVMEKSANWNDTQWGNWIKTEGRKILEAQWEKWIKKGDDQLQKLILDKWVQWKNDKIRSWLSSEWKTEEDYYWANVERATTAKWLQEAEKMHWLKWKERINRESEQWVNWVQMKESVYINVEWKKWPKWKNDKKILFNKWSTNLVYKWTLKKQWNVWIKEANTAPQVHHHHHH

SEQ ID NO: 80 - DNA, 801 bp, Plasmodium vivax
  source 1..801: mol_type = genomic DNA
atggctgccgccaacaggccaaacgccaacggcttcgtctccccaaccctcatcggcttcggcgagctgtccatccaagagagcgaggagttcaagaggatggcctggaacaactggatgctccgcctggagtccgactggaagcacttcaacgacagcgtggaggaagccaagaccaagtggctgcacgagagggactccgcttggagcgactggctccgctccctgcagagcaagtggtcccactacagcgagaagatgctgaaggagcacaagtccaacgtcatggagaagagcgccaactggaacgacacccaatggggcaactggatcaagaccgagggccgcaagatcctggaggcccagtgggagaagtggatcaagaagggcgacgaccaactgcagaagctcatcctggacaagtgggtccagtggaagaacgacaagatcaggtcctggctctccagcgagtggaagaccgaggaagactactactgggctaacgtggagagggctaccaccgctaagtggctccaagaggccgagaagatgcactggctgaagtggaaggagaggatcaaccgcgagtccgagcaatgggtgaactgggtccagatgaaggagagcgtgtacatcaacgtcgagtggaagaagtggccaaagtggaagaacgataagaagatcctgttcaacaagtggagcaccaacctcgtgtacaagtggaccctgaagaagcagtggaacgtctggatcaaggaagccaacaccgccccacaggtgcaccaccaccaccaccactga

SEQ ID NO: 81 - protein, 475 aa, Plasmodium vivax
MEKVVDEVKYSEEVCNESVDLYLLVDGSGSIGYPNWITKVIPMLNGLINSLSLSRDTINLYMNLFGNYTTELIRLGSGQSIDKRQALSKVTELRKTYTPYGTTNMTAALDEVQKHLNDRVNREKAIQLVILMTDGVPNSKYRALEVANKLKQRNVSLAVIGVGQGINHQFNRLIAGCRPREPNCKFYSYADWNEAVALIKPFIAKVCTEVERVANCGPWDPWTACSVTCGRGTHSRSRPSLHEKCTTHMVSECEEGECPVEPEPLPVPAPLPTVPEDVNPRDTDDENENPNFNKGLDVPDEDDDEVPPANEGADGNPVEENVFPPADDSVPDESNVLPLPPAVPGGSSEEFPADVQNNPDSPEELPMEQEVPQDNNVNEPERSDSNGYGVNEKVIPNPLDNERDMANKNKTVHPGRKDSARDRYARPHGSTHVNNNRANENSDIPNNPVPSDYEQPEDKAKKSSNNGYKHHHHHH

SEQ ID NO: 82 - DNA, 1428 bp, Plasmodium vivax
  source 1..1428: mol_type = genomic DNA
atggagaaggtggtcgacgaggtgaagtacagcgaggaagtgtgcaacgagtccgtcgacctctacctcctggtggacggctccggcagcatcggctacccaaactggatcaccaaggtcatcccaatgctcaacggcctgatcaactccctcagcctgtcccgcgacaccatcaacctctacatgaacctgttcggcaactacaccaccgagctcatcaggctgggcagcggccaatccatcgacaagcgccaggccctcagcaaggtgaccgagctgaggaagacctacaccccatacggcaccaccaacatgaccgccgccctcgacgaggtgcaaaagcacctgaacgacagggtcaaccgcgagaaggccatccagctcgtgatcctgatgaccgacggcgtcccaaacagcaagtaccgcgccctggaggtggccaacaagctgaagcaaaggaacgtctccctggccgtgatcggcgtgggccaaggcatcaaccaccagttcaacaggctgatcgctggctgcaggccacgcgagccaaactgcaagttctacagctacgctgactggaacgaggctgtggctctcatcaagccattcatcgccaaggtctgcaccgaggtggagagggtggctaactgcggcccatgggacccgtggaccgcttgctccgtgacctgcggcaggggcacccacagcaggtcccgcccaagcctgcacgagaagtgcaccacccacatggtgtccgagtgcgaggaaggcgagtgcccagtggagccagagccactgccggtcccagccccactgccaaccgtgccagaggacgtcaacccaagggacaccgacgacgagaacgagaacccaaacttcaacaagggcctcgacgtgccagacgaggacgacgacgaggtcccaccagctaacgagggcgctgacggcaacccagtggaggagaacgtcttcccaccagccgacgacagcgtgccagacgagtccaacgtgctgccactgccaccagctgtgccaggcggctccagcgaggagttcccagctgacgtccaaaacaacccagactccccagaggagctcccgatggagcaagaggtgccacaggacaacaacgtcaacgagccagagcgcagcgactccaacggctacggcgtgaacgagaaggtcatcccaaacccactggacaacgagagggacatggccaacaagaacaagaccgtgcacccgggcaggaaggacagcgccagggaccgctacgccaggccacacggctccacccacgtgaacaacaacagggccaacgagaacagcgacatcccaaacaacccagtcccatccgactacgagcagccagaggacaaggccaagaagtccagcaacaacggctacaagcaccaccaccaccaccactga

SEQ ID NO: 83 - protein, 362 aa, Plasmodium vivax
MDDKKDKENEHKEDADKKNNDELKTLKGKLQKIRVQIKDDKLPQKISEEQISVLKKKLEDFKNLKSEHEAKLASEKGDTSAGGEGELGLSDKEFVGQNVKANGDAAGVSGEQGASGGSGQGEAGPSSPADEQDDDNEAVQWGPATEEVVAEAMSDEGPQEQGAEGGPSNPTDDQAEEATPGPSKPASGASGSQGASDSSNDSAEPTSAAAAAAPAGPTAAAASPQVKHVDTLCDELLAGENKKNVLDEGEDHSQYNIFRKQYDKMVLNKTEYNISLKLLDTMLTNGQVEREKKNTLIKTFKKALYDKQYSEKLRNLISGVYAFAKRNNFIDGDKVKEGDYSKLFEYIGCMMNTLELHHHHHH

SEQ ID NO: 84 - DNA, 1089 bp, Plasmodium vivax
  source 1..1089: mol_type = genomic DNA
atggacgacaagaaggacaaggagaacgagcacaaggaagacgccgataagaagaacaacgacgagctcaagaccctgaagggcaagctccaaaagatcagggtgcagatcaaggacgacaagctgccacaaaagatctccgaggagcagatcagcgtcctcaagaagaagctggaggacttcaagaacctcaagtccgagcacgaggccaagctggcctccgagaagggcgacacctccgccggcggcgagggcgagctgggcctgtccgacaaggagttcgtgggccaaaacgtcaaggccaacggcgacgccgccggcgtgagcggcgagcaaggcgcctccggcggcagcggccagggcgaggctggcccatccagcccagccgacgagcaagacgacgacaacgaggctgtccagtggggcccagctaccgaggaagtggtggctgaggctatgtccgacgagggcccacaagagcagggcgctgagggcggcccaagcaacccaaccgacgaccaagctgaggaagccaccccaggcccatccaagccagcttccggcgcttccggcagccagggcgcttccgactccagcaacgactccgccgagccaaccagcgctgccgccgccgccgccccagctggcccaaccgctgccgccgccagcccacaggtgaagcacgtggacaccctctgcgacgagctcctggctggcgagaacaagaagaacgtgctggacgagggcgaggaccactcccaatacaacatcttcaggaagcagtacgacaagatggtcctcaacaagaccgagtacaacatcagcctcaagctcctggacaccatgctgaccaacggccaagtggagcgcgagaagaagaacaccctcatcaagaccttcaagaaggccctgtacgacaagcagtactccgagaagctcaggaacctgatcagcggcgtgtacgccttcgccaagcgcaacaacttcatcgacggcgacaaggtgaaggaaggcgactacagcaagctcttcgagtacatcggctgcatgatgaacaccctggagctgcaccaccaccaccaccactga

SEQ ID NO: 85 - protein, 723 aa, Plasmodium vivax
MKRHATRGALHSLKSIEHEVQRKKNKKKKIILYSIGSILALAAVIATGVGIGMYIKKKKKNSLEKLQQIEPQKLESKTDESDPLLGKSEAAKVEVKGDSEEVPQEVSSPSEALDVEPPVSEALNMEPAVGESANFEDSAKGEVDIEPVSEVESIEPVSEVESIEPVSEVESIEPSVDEVMDAAEPISTEPVNVEPAGNETENIVPTSFEQVNIEPAVSEAFSQERSGEETADFEDSVKEDVIPESPPVESVTIEAENIQPMNVEQMNVDPTVSDAESIEPTPVEAVDIEPVNVEPVNVEPAVSETMSQEPSLDEVENVESAVNEMMSQEPSAEETANFAHSIKEDVSPESTSVESLDVESSVSEPMSTDPSPVESVSMESVDSETVNVESIDSETVNVEPSDETSKVEADVQQFTDEELSTIGNVADKASDGPAPEASDFPDSIFEENLDNANPPLKLEDALVDPPASDEAQPEPSHPNEAVGAAKSAESAEADQISHSGSGDASPSAPSSSDDTSGSKNSGTSGKDRLFKTYDSDVEPPIVPEKYPTVGVKEAPKMGFAEMAFKNIFDTFSKVADASKVLTPEKQSAPEKQSAPEKQSAPEKQSAPEKHSTPPKQSTSPKESTSPKQPAPPKPSTSPKQSAPAKQSAPPKQSAPAKQSAPAKNAAPPQSASSSRFFSSSSNGNKGFGLRLFSDASSSNNKKGRAGNPIIRFKRRANHHHHHH

SEQ ID NO: 86 - DNA, 2172 bp, Plasmodium vivax
  source 1..2172: mol_type = genomic DNA
atgaagaggcacgctacccgcggcgccctccactccctgaagagcatcgagcacgaggtgcaaaggaagaagaacaagaagaagaagatcatcctctactccatcggcagcatcctggctctggctgccgtgatcgctaccggcgtcggcatcggcatgtacatcaagaagaagaagaagaacagcctggagaagctgcaacagatcgagccacaaaagctggagtccaagaccgacgagagcgacccactcctgggcaagagcgaggctgctaaggtggaggtcaagggcgactccgaggaagtgccacaagaggtgtcctccccgagcgaggctctggacgtggagccaccagtctccgaggccctgaacatggagccagccgtgggcgagtccgccaacttcgaggacagcgccaagggcgaggtcgacatcgagccagtgtccgaggtcgagtctattgaaccagtgtccgaggtggagtctattgagccagtgtccgaagtcgagagcatcgagccatccgtggacgaggtcatggacgctgctgagccaatcagcaccgagccagtgaacgtcgagccagccggcaacgagacggagaacatcgtgccaacctccttcgagcaagtgaacatcgagccagccgtcagcgaggccttctcccaagagaggagcggcgaggagacggctgacttcgaggactccgtgaaggaagacgtcatcccagagtccccaccagtggagagcgtcaccatcgaggccgagaacatccaaccgatgaacgtggagcagatgaacgtggacccaaccgtctccgacgccgagagcatcgagccaaccccagtggaggccgtggatatcgagcctgtcaacgtggagcctgtcaacgttgagccagccgtgtccgagacgatgagccaagagccatccctcgacgaggtggagaacgtcgagagcgccgtcaacgagatgatgtcccaggagccatccgctgaggagacggccaacttcgcccactccatcaaggaagacgtgagcccagagagcacctccgtcgagtccctggacgtggagtccagcgtcagcgagccaatgtccaccgacccaagcccagtggagagcgtctccatggagtccgtggacagcgagacggtgaacgtcgagtccatcgattccgagacggtcaacgtggagccatccgacgagacgagcaaggtggaggccgacgtccaacagttcaccgacgaggagctcagcaccatcggcaacgtggctgacaaggcttccgacggcccagctccagaggcctccgacttcccagacagcatcttcgaggagaacctcgacaacgccaacccaccactcaagctggaggacgctctggtggacccaccagctagcgacgaggctcaaccagagccatcccacccaaacgaggctgtgggcgctgctaagtccgctgagagcgctgaggctgaccaaatcagccactccggcagcggcgacgcttccccaagcgctccatccagctccgacgacacctccggcagcaagaactccggcaccagcggcaaggacaggctcttcaagacctacgactccgacgtggagccaccaatcgtcccagagaagtacccaaccgtgggcgtgaaggaagccccaaagatgggcttcgccgagatggccttcaagaacatcttcgacaccttctccaaggtggctgacgctagcaaggtcctgaccccagagaagcaatccgccccagagaagcagagcgctcctgagaagcagagcgctcccgagaagcagagcgccccagagaagcactccaccccaccaaagcaatccaccagcccaaaggagtccaccagcccaaagcagccagccccaccaaagccatccaccagccctaagcagtccgctccagctaagcagtccgccccaccaaagcagagcgctccagctaagcaatccgctccagctaagaacgctgccccaccacagagcgccagctccagcaggttcttctccagctccagcaacggcaacaagggcttcggcctcaggctgttctccgacgcctccagctccaacaacaagaagggcagggccggcaacccaatcatccgcttcaagaggcgcgccaaccaccaccaccaccaccactga

SEQ ID NO: 87 - protein, 373 aa, Plasmodium vivax
MNNPAEVVAAHLRRTGNSNEIRQASHVESVGGSANSSLDDDDGGGYDSAAPPGELHTTGDAPPGEFRTTGVVPPGRQKGGKKRMFKIKKKKSLTPLHIDDGGFTQGGEAKGPDVALESFAITRKRRRPPLLGRGVVESSNIELTSKLGGKLGSKLGGKLNPTLSLVASRAVDGLLGGVHKHMQGPFSLDLDGTNNSPLATPIVTPNLYSNISTPFNMHNGIPPSAPAPMALPPQGVQVPLPNAQPQPPPSVATTATAAPAATSPMASPTTPTPAASTGVPPPPGIQLATNAMTYPQMNMQNVMTANQMAQNPAFNIHPTATNLRDDPGNVNYNEVVTITIGIVICLFLFCFVFGCIVKMCKPAKRRRHHHHHH

SEQ ID NO: 88 - DNA, 1122 bp, Plasmodium vivax
  source 1..1122: mol_type = genomic DNA
atgaacaacccagctgaggtggtggctgctcacctgaggcgcaccggcaactccaacgagatcaggcaggctagccacgtggagagcgtcggcggctccgctaactccagcctcgacgacgacgacggcggcggatacgacagcgctgccccaccaggcgagctccacaccaccggcgacgccccaccaggcgagttccgcaccaccggcgtggtcccaccaggcaggcaaaagggcggcaagaagcgcatgttcaagatcaagaagaagaagtccctcaccccactgcacatcgacgacggcggcttcacccagggcggcgaggctaagggcccagacgtggctctggagtccttcgccatcaccaggaagaggcgcaggccaccactcctgggccgcggcgtggtcgagtccagcaacatcgagctcaccagcaagctgggcggcaagctcggctccaagctgggcggcaagctcaacccgaccctcagcctggtggcctccagggccgtggacggcctcctgggcggcgtgcacaagcacatgcaaggcccattcagcctcgacctggacggcaccaacaactccccactggccaccccaatcgtcaccccaaacctctactccaacatcagcaccccattcaacatgcacaacggcatcccaccaagcgctccagctccaatggctctgccaccacaaggcgtgcaggtcccactcccaaacgcccaaccacaaccaccaccatccgtggctaccaccgctaccgctgctccagctgctaccagcccaatggcttccccaaccaccccaaccccagctgctagcaccggcgtgccaccaccaccaggcatccagctggccaccaacgccatgacctacccacagatgaacatgcagaacgtcatgaccgccaaccaaatggcccagaacccagccttcaacatccacccgaccgctaccaacctcagggacgacccaggcaacgtgaactacaacgaggtggtcaccatcaccatcggcatcgtcatctgcctcttcctgttctgcttcgtgttcggctgcatcgtcaagatgtgcaagccggctaagcgcaggcgccatcaccaccaccaccactga

SEQ ID NO: 89 - protein, 361 aa, Plasmodium vivax
MSKTGNNNRNAKNAKGGGGGGKRGNNEANKNDGMSGKGSQKGKKKDPGGGGTPKGQGKGPEQGKQKNKKGEDSHFDEYIKDMKNSQDEDNFMDELNRFEKNFHDEDFESDENLFNYGKGGTHSGEFNKIGELNSGNYNEMKPDANDYQYFDNEDILEGDEDLTNIWNKNMQNFEPSTLLTFEIQGNSEEYLFEEVTSLNTYFRGVFYSNNESDDNKILFFITDPDGEVIYKKEASEGIFYFYTQKIGVYTITLKNSKWMGKKLTTVALGLGESPSLKSEHIKDFTNYIDKIVAETKRLKNELKYLSSKHMTHIEKMKKITNKAFLYCFIKLFVLVFLSLFTIYYIKNLVSNKRVLHHHHHH

SEQ ID NO: 90 - DNA, 1086 bp, Plasmodium vivax
  source 1..1086: mol_type = genomic DNA
atgtccaagaccggcaacaacaacaggaacgccaagaacgctaagggcggcggcggcggcggcaagaggggcaacaacgaggccaacaagaacgacggcatgtccggcaagggcagccaaaagggcaagaagaaggacccaggcggcggcggcaccccgaagggccagggcaagggcccagagcaaggcaagcagaagaacaagaagggcgaggactcccacttcgacgagtacatcaaggacatgaagaacagccaagacgaggacaacttcatggacgagctcaacaggttcgagaagaacttccacgacgaggacttcgagtccgacgagaacctgttcaactacggcaagggcggcacccactccggcgagttcaacaagatcggcgagctcaacagcggcaactacaacgagatgaagccagacgccaacgactaccagtacttcgacaacgaggacatcctggagggcgacgaggacctgaccaacatctggaacaagaacatgcaaaacttcgagccaagcaccctcctgaccttcgagatccagggcaactccgaggagtacctcttcgaggaagtgaccagcctgaacacctacttccgcggcgtcttctactccaacaacgagagcgacgacaacaagatcctgttcttcatcaccgacccagacggcgaggtcatctacaagaaggaagcctccgagggcatcttctacttctacacccaaaagatcggcgtgtacaccatcaccctcaagaacagcaagtggatgggcaagaagctgaccaccgtggctctgggcctgggcgagtccccaagcctcaagagcgagcacatcaaggacttcaccaactacatcgacaagatcgtcgccgagacgaagaggctgaagaacgagctcaagtacctgtccagcaagcacatgacccacatcgagaagatgaagaagatcaccaacaaggccttcctctactgcttcatcaagctcttcgtgctggtcttcctctccctgttcaccatctactacatcaagaacctcgtgagcaacaagcgcgtcctgcaccaccaccaccaccactga

SEQ ID NO: 91 - protein, 501 aa, Plasmodium vivax
MNNHQAVKQQMNPKGSKEQNRMVAPNSNMPGGMRDLAYHRNNGNNEMGKMNMNANGQQHNAGSSNTYNSNSINNNNYSLGLYIDNPQNAFVFDENDLKTLFSHYKGAKNIRILNDKAAAQITFNDKNMIQQVRKDINGLTITDIGTIRCIILNEGKIVEQFLPFSANDPASAQQKGGSNQSGDSTVDMLKKLANLLQPERAMDSSMAPKMGDNGGLSATGSVNMGASIATNVGMGGNMPTNANMGGVITTNANVSANVSANVSANPMPGKNQVKNKMGNHAIYNNGGSHFNQAHMNKGEPGENNPYATKRLSRIELIDIFGFPVEFDVMKKILGKNNSNISYIKEQTNNSVSIEIKGKPFNEAPIVERMHVSVSSDDLIGYKKATELIVKLLNSIFEEFYDFCYEKNYPVPENLSFKRHEYMYNPDGSTKYVGFKDKWHVMKDSYRTDYSFRKNKGLQKNDKDKRMHGGAFGGHPNLSIGYANQNAPQGDFKEMNHHHHHH

SEQ ID NO: 92 - DNA, 1506 bp, Plasmodium vivax
  source 1..1506: mol_type = genomic DNA
atgaacaaccaccaagccgtcaagcaacagatgaacccaaagggctccaaggagcagaacaggatggtggccccaaacagcaacatgccaggcggcatgagggacctcgcttaccacaggaacaacggcaacaacgagatgggcaagatgaacatgaacgccaacggccaacagcacaacgccggctccagcaacacctacaactccaactccatcaacaacaacaactactccctcggcctgtacatcgacaacccacaaaacgccttcgtcttcgacgagaacgacctcaagaccctgttcagccactacaagggcgccaagaacatcaggatcctcaacgacaaggctgccgcccagatcaccttcaacgacaagaacatgatccaacaggtcaggaaggacatcaacggcctgaccatcaccgacatcggcaccatccgctgcatcatcctcaacgagggcaagatcgtggagcaattcctgccattctccgccaacgacccggctagcgctcaacagaagggcggctccaaccaaagcggcgactccaccgtggacatgctcaagaagctcgctaacctcctgcagccagagagggccatggactccagcatggccccaaagatgggcgacaacggcggcctctccgctaccggctccgtcaacatgggcgcctccatcgccaccaacgtgggcatgggcggcaacatgccaaccaacgccaacatgggcggcgtcatcaccaccaacgccaacgtgagcgccaacgtctccgctaacgtgagcgctaacccaatgccaggcaagaaccaagtgaagaacaagatgggcaaccacgccatctacaacaacggcggctcccacttcaaccaggcccacatgaacaagggcgagccaggcgagaacaacccatacgccaccaagaggctcagccgcatcgagctgatcgacatcttcggcttcccagtcgagttcgacgtgatgaagaagatcctcggcaagaacaacagcaacatctcctacatcaaggagcaaaccaacaactccgtcagcatcgagatcaagggcaagccattcaacgaggccccaatcgtggagcgcatgcacgtgtccgtctccagcgacgacctcatcggctacaagaaggccaccgagctgatcgtcaagctcctgaacagcatcttcgaggagttctacgacttctgctacgagaagaactacccagtgccagagaacctgtccttcaagaggcacgagtacatgtacaacccagacggcagcaccaagtatgtgggcttcaaggacaagtggcacgtgatgaaggactcctacaggaccgactacagcttccgcaagaacaagggcctccagaagaacgacaaggacaagaggatgcacggcggcgctttcggcggacacccaaacctgagcatcggctacgccaaccaaaacgccccacagggcgacttcaaggagatgaaccaccaccaccaccaccactga

SEQ ID NO: 93 - protein, 185 aa, Plasmodium vivax
MREAKGSVRDGKQYVKTKSPTYTPQKKTKVIFYMPGQEQEEEEDDNDPNGSKKNGKSDTGANKGTHMGSKTDAGNSPSGLNKGSGVGSGSRPASNNYKGNAGGGINIDMSPHGDNSNKGQQGNAGLNKNQEDTLRDEYEKIRKQEEEEEERINNQRRADMKRAQRGKNKFGDDKGVQDSHHHHHH

SEQ ID NO: 94 - DNA, 558 bp, Plasmodium vivax
  source 1..558: mol_type = genomic DNA
atgcgcgaggctaagggctccgtgcgcgacggcaagcaatacgtcaagaccaagagcccaacctacaccccacagaagaagaccaaggtcatcttctacatgccaggccaagagcaagaggaagaggaagacgacaacgacccaaacggctccaagaagaacggcaagagcgacaccggcgccaacaagggcacccacatgggctccaagaccgacgctggcaactccccgagcggcctcaacaagggctccggcgtgggctccggcagcaggccagccagcaacaactacaagggcaacgccggcggcggcatcaacatcgacatgtccccacacggcgacaacagcaacaagggccaacagggcaacgccggcctcaacaagaaccaagaggacaccctgagggacgagtacgagaagatccgcaaacaagaggaagaggaagaggagcgcatcaacaaccaaaggcgcgctgacatgaagagggctcagaggggcaagaacaagttcggcgacgacaagggcgtgcaagacagccaccaccaccaccaccactga

SEQ ID NO: 95 - protein, 259 aa, Plasmodium vivax
MSSQSAVDYIEQEPLDILNLEEGDLEVTEQWKDNEWHNWKLKLEEDWDSFSTSLIRDKKDFMKIKTDELNGWLNLEENKWNNFSGYLSDGYKNYLLKKSEKWNDADWENWANTEMVAHLDKDYHLWSLNTERSVNALVRGEWNQWQHDKMSSWLSSDWKKVGAMYWDLQESRNWASYSHTDDMKEHWIKWNDRNARENIEWSKWVQNKEYFIMYARHSDIEQWKYDNYALYSTWRNDFINRWVSEKKWNSILNHHHHHH

SEQ ID NO: 96 - DNA, 780 bp, Plasmodium vivax
  source 1..780: mol_type = genomic DNA
atgtccagccaaagcgccgtggactacatcgagcaggagccactcgacatcctcaacctcgaagagggcgacctggaggtcaccgagcagtggaaggacaacgagtggcacaactggaagctcaagctcgaagaggactgggactccttcagcacctccctcatcagggacaagaaggacttcatgaagatcaagaccgacgagctgaacggctggctcaacctggaggagaacaagtggaacaacttcagcggctacctctccgacggctacaagaactacctcctgaagaagtccgagaagtggaacgacgccgactgggagaactgggccaacaccgagatggtggcccacctcgacaaggactaccacctctggagcctgaacaccgagaggtccgtgaacgctctggtccgcggcgagtggaaccaatggcagcacgacaagatgtccagctggctctccagcgactggaagaaggtcggcgccatgtactgggacctgcaggagagcaggaactgggccagctactcccacaccgacgacatgaaggagcactggatcaagtggaacgacaggaacgcccgcgagaacatcgagtggtccaagtgggtgcaaaacaaggagtacttcatcatgtacgcccgccacagcgacatcgagcagtggaagtacgacaactacgccctctactccacctggaggaacgacttcatcaaccgctgggtcagcgagaagaagtggaactccatcctgaaccaccaccaccaccaccactga

SEQ ID NO: 97 - protein, 257 aa, Plasmodium vivax
MKSSNEIERLTHVKLKDTSEWTENVEEWVKDEWHEWMDEVQMDWKEFNSSLESEKNKWFGKKEKEMMELIKSIEDKWLDFNENMHEVLNYAILKISLMWSFSEWQKWINKDGKRIIENQWERWTISNKNLYYKIIMKEWFKWKNKKIKQWLKRNWLHHEGRILENWERLPYTKILAMSEKKPWFNSNAQVINERDYFLIWIKKKEDFLVNEERDKWENWEYYKNDFFQTWMDSFLSHWLNIKKRDILHSQSHHHHHH

SEQ ID NO: 98 - DNA, 774 bp, Plasmodium vivax
  source 1..774: mol_type = genomic DNA
atgaagtccagcaacgagatcgagaggctcacccacgtgaagctgaaggacacctccgagtggaccgagaacgtggaggagtgggtcaaggacgagtggcacgagtggatggacgaggtccagatggactggaaggagttcaactccagcctggagtccgagaagaacaagtggttcggcaagaaggagaaggagatgatggagctgatcaagagcatcgaggacaagtggctcgacttcaacgagaacatgcacgaggtgctcaactacgccatcctcaagatctccctgatgtggtccttcagcgagtggcaaaagtggatcaacaaggacggcaagaggatcatcgagaaccagtgggagcgctggaccatcagcaacaagaacctgtactacaagatcatcatgaaggagtggttcaagtggaagaacaagaagatcaagcaatggctcaagaggaactggctgcaccacgagggcaggatcctggagaactgggagcgcctgccatacaccaagatcctcgccatgtccgagaagaagccatggttcaacagcaacgcccaagtgatcaacgagagggactacttcctgatctggatcaagaagaaggaagacttcctcgtcaacgaggagcgcgacaagtgggagaactgggagtactacaagaacgacttcttccaaacctggatggactccttcctcagccactggctgaacatcaagaagcgcgacatcctccactcccagagccaccaccaccaccaccactga

SEQ ID NO: 99 - protein, 118 aa, Plasmodium vivax
MRLKHDHNLLPNYANLMRDDQNGQNSENRGDNINNHNKNHNDQNNHNGNNDNSINSEYLKTSHLQNSSAMVHLNDHKITTKPARYSYIQRSKIYAFNPNNKKIENINNELHSHHHHHH

SEQ ID NO: 100 - DNA, 357 bp, Plasmodium vivax
  source 1..357: mol_type = genomic DNA
atgaggctcaagcacgaccacaacctcctgccaaactacgccaacctgatgagggacgaccaaaacggccagaactccgagaaccgcggcgacaacatcaacaaccacaacaagaaccacaacgaccaaaacaaccacaacggcaacaacgacaactccatcaacagcgagtacctcaagaccagccacctgcagaactccagcgccatggtgcacctcaacgaccacaagatcaccaccaagccagccaggtactcctacatccaacgcagcaagatctacgccttcaacccaaacaacaagaagatcgagaacatcaacaacgagctgcactcccaccaccaccaccaccactga

SEQ ID NO: 101 - protein, 651 aa, Plasmodium vivax
MSMEQGTPIVFPHKEGTILTKGTNNLAVAHKEEVHRSEEETTLKGLKEELPHEHTLAIQKYDPSFGRGGSPGSGSTEHTNGSFSNSYETILYNKSNDVVKNLKEIKKGAPFGGVISDAVSCPASSSSNTGGNKNLCFSNMMKLSKKILGFPLLTDFERGMSTNQPCLPLSDHLKRLSVCTVCYSKHNDLAKAIICRVTKMHFEANYNDGLGDEDMFKTSSECIQSVIRELANTIKEYRKRELSGAYVQELARSGSSSYRSCSSSSYSSRGGSCAGSRGDGLAGSHGEIHAVIAGPPLTDDHNDIGAEAHSPSSSLKLPPQKPFYGMMSDPPCSDRRPGDTNNPFENNTPPLLWDNKVNYTDDYTCKRGEVNSTLGKRPHEEDNKGSSQKKSKLRTKPSNDTIGGENGDSLKGGTDEGKTHEGGGNVGSCTAQGGADQLPRSDLCRDPRGDPCVDPLPEQHAHRSKDENQKGDKNDIHFAGEKLDEIEAPGDQKGNYVTLENISKASNFIPLLGVELGSTKIQREFTNGTYVGTVTEQIKDEHGNPFFVVTYEDGDAEWMTPCFLFQELLKQSTNSVDYPLATTFKEVFNPEFKKDLKLSNCSLELKIERRKRKSNCESASNNNSVSKRQKHAQEENSSRKKKQRFHHHHHH

SEQ ID NO: 102 - DNA, 1956 bp, Plasmodium vivax
  source 1..1956: mol_type = genomic DNA
atgtccatggagcaaggcaccccaatcgtgttcccacacaaggaaggcaccatcctcaccaagggcaccaacaacctggccgtggcccacaaggaagaggtgcacaggagcgaggaagagacgaccctcaagggcctgaaggaagagctcccacacgagcacaccctggccatccagaagtacgacccaagcttcggccgcggcggctccccaggcagcggcagcaccgagcacaccaacggctccttcagcaactcctacgagacgatcctctacaacaagtccaacgacgtggtcaagaacctgaaggagatcaagaagggcgctccattcggcggcgtgatctccgacgccgtctcctgcccggcctccagctccagcaacaccggcggcaacaagaacctctgcttcagcaacatgatgaagctctccaagaagatcctgggcttcccactcctgaccgacttcgagaggggcatgagcaccaaccaaccatgcctcccactgagcgaccacctcaagcgcctgtccgtgtgcaccgtctgctacagcaagcacaacgacctggccaaggccatcatctgcagggtgaccaagatgcacttcgaggccaactacaacgacggcctcggcgacgaggacatgttcaagacctccagcgagtgcatccaatccgtgatccgcgagctggccaacaccatcaaggagtacaggaagcgcgagctgtccggcgcctacgtccaagagctcgctaggtccggctccagctcctacaggagctgcagctccagctcctacagctccaggggcggcagctgcgctggctcccgcggcgacggcctcgccggctcccacggcgagatccacgccgtcatcgctggcccaccactgaccgacgaccacaacgacatcggcgctgaggctcacagcccaagctccagcctcaagctgccaccacaaaagccattctacggcatgatgtccgacccaccatgctccgacaggcgcccaggcgacaccaacaacccattcgagaacaacaccccaccactcctgtgggacaacaaggtgaactacaccgacgactacacctgcaagaggggcgaggtcaactccaccctcggcaagcgcccacacgaggaagacaacaagggctccagccagaagaagtccaagctcaggaccaagccaagcaacgacaccatcggcggcgagaacggcgacagcctgaagggcggcaccgacgagggcaagacccacgagggcggcggcaacgtgggctcctgcaccgcccaaggcggcgccgaccagctcccaaggtccgacctgtgcagggacccacgcggcgacccatgcgtcgacccactcccagagcaacacgcccaccgctccaaggacgagaaccagaagggcgacaagaacgacatccacttcgccggcgagaagctcgacgagatcgaggccccaggcgaccaaaagggcaactacgtgaccctggagaacatcagcaaggcctccaacttcatcccgctcctgggcgtggagctgggcagcaccaagatccaacgcgagttcaccaacggcacctacgtgggcaccgtcaccgagcagatcaaggacgagcacggcaacccattcttcgtggtcacctacgaggacggcgacgctgagtggatgaccccatgcttcctcttccaagagctcctgaagcagagcaccaactccgtggactacccactggccaccaccttcaaggaagtgttcaacccagagttcaagaaggacctcaagctgagcaactgctccctggagctgaagatcgagaggcgcaagaggaagtccaactgcgagagcgcctccaacaacaacagcgtgtccaagcgccaaaagcacgcccaagaggagaactcctccaggaagaagaagcagcgcttccaccaccaccaccaccactga

SEQ ID NO: 103 - protein, 793 aa, Plasmodium vivax
MTFNDGSDEISTAQKYKTDVEGIIDKLNVIDETINGINSTLDELLELGNNCQLHRTFLISSSLNNKIAKFLVEIREQKENTKKCFQYVKRNHQHLANFVSELHKTQGGIFENVNLVDNTPDADKYYHEFMEIEQEATKIVKDIKKEIYHLNDDVDEPVLEKRIKDVINTYNKLKTKKVQMDQSYKNMYITKLREVEGSHDLFNQVAQLIRGETDKKGKALSERENNLHSIYNFVKLHETELHNLYAKYTPEYMEKINKIFDDINARMIAVDLNDDHSSEYSDVKRHEHEAMLLMDATNNLSKEVEMMQNESGGKNDGINGGKSQLVEDYTNTMSEFTEQAKTVAKKIHDSKGDYANMFDHIRENEAMLERIDLKKKDIKEILAHLNRMKEYLLKKLSEEEKLHHMREKLEEVNTSTDEIVKKFRTYDQMVDISQNIDIKNVQSKRYDSVDEIDKEMSYIKTHNKDLIDSKFIVERALENDKRKKSEMAQIFSTISRDNSSMYEYAKSFFDSVLKEIEKLTQMIRNMDKLINENEAVMEKLKDQRRELQNVENASTDLGKLEEVDKMAQTKSETELSERNDSRNAKDGATYSTLMDDKETDSVNGEETKQENVVVKKGLPPQTDIYTSVVLKNDRNDQKSEKIGEKKSNKPVGTEENIQHSSYLNNDNSNNDIDVGTLYTLGGYNAPNDNYNTNESGDDINEEAKKKRNAVLFVYVGGLFSALFICIGAVFYLLHRKIGIEGVGKSDHEKKPTIEDTKIEVFEETNGSKRNVKDEVIDVPFVDMEDNLHHHHHH

SEQ ID NO: 104 - DNA, 2382 bp, Plasmodium vivax
  source 1..2382: mol_type = genomic DNA
atgaccttcaacgacggcagcgacgagatctccaccgcccaaaagtacaagaccgacgtggagggcatcatcgacaagctgaacgtcatcgacgagacgatcaacggcatcaacagcaccctggacgagctcctggagctcggcaacaactgccaactccacaggaccttcctgatctccagctccctcaacaacaagatcgccaagttcctcgtggagatcagggagcagaaggagaacaccaagaagtgcttccaatacgtgaagcgcaaccaccagcacctggccaacttcgtctccgagctccacaagacccaaggcggcatcttcgagaacgtcaacctggtggacaacaccccagacgccgacaagtactaccacgagttcatggagatcgagcaagaggccaccaagatcgtcaaggacatcaagaaggagatctaccacctgaacgacgacgtggacgagccagtcctggagaagaggatcaaggacgtgatcaacacctacaacaagctgaagaccaagaaggtccagatggaccagtcctacaagaacatgtacatcaccaagctgagggaggtggagggcagccacgacctgttcaaccaagtcgcccagctcatcaggggcgagacggacaagaagggcaaggccctgtccgagcgcgagaacaacctccacagcatctacaacttcgtgaagctgcacgagacggagctccacaacctgtacgccaagtacaccccagagtacatggagaagatcaacaagatcttcgacgacatcaacgccaggatgatcgccgtggacctcaacgacgaccacagctccgagtacagcgacgtcaagcgccacgagcacgaggccatgctcctgatggacgccaccaacaacctgtccaaggaagtggagatgatgcagaacgagagcggcggcaagaacgacggcatcaacggcggcaagtcccaactcgtggaggactacaccaacaccatgagcgagttcaccgagcaggccaagaccgtcgccaagaagatccacgactccaagggcgactacgccaacatgttcgaccacatcagggagaacgaggccatgctggagcgcatcgacctcaagaagaaggacatcaaggagatcctcgcccacctgaacaggatgaaggagtacctcctgaagaagctgtccgaggaagagaagctccaccacatgcgcgagaagctcgaagaggtgaacacgagcaccgacgagatcgtcaagaagttccgcacctacgaccaaatggtggacatctcccagaacatcgacatcaagaacgtgcaaagcaagcgctacgactccgtcgacgagatcgacaaggagatgtcctacatcaagacccacaacaaggacctgatcgacagcaagttcatcgtcgagagggccctggagaacgacaagcgcaagaagagcgagatggcccaaatcttcagcaccatctccagggacaacagctccatgtacgagtacgccaagagcttcttcgactccgtgctgaaggagatcgagaagctcacccagatgatccgcaacatggacaagctcatcaacgagaacgaggccgtcatggagaagctgaaggaccaaaggcgcgagctccagaacgtggagaacgcctccaccgacctcggcaagctcgaagaggtggacaagatggcccagaccaagagcgagacggagctgtccgagaggaacgacagccgcaacgctaaggacggcgctacctactccaccctcatggacgacaaggagacggacagcgtgaacggcgaggagacgaagcaagagaacgtggtcgtgaagaagggcctgccaccacagaccgacatctacaccagcgtcgtgctcaagaacgacaggaacgaccaaaagtccgagaagatcggcgagaagaagagcaacaagccagtgggcaccgaggagaacatccagcacagctcctacctcaacaacgacaactccaacaacgacatcgacgtgggcaccctctacaccctgggcggctacaacgccccaaacgacaactacaacaccaacgagagcggcgacgacatcaacgaggaagccaagaagaagaggaacgccgtgctcttcgtctacgtgggcggcctcttctccgccctgttcatctgcatcggcgccgtgttctacctcctgcaccgcaagatcggcatcgagggcgtcggcaagagcgaccacgagaagaagccaaccatcgaggacaccaagatcgaggtgttcgaggagacgaacggctccaagcgcaacgtcaaggacgaggtcatcgacgtgccattcgtcgacatggaggacaacctccaccaccaccaccaccactga

SEQ ID NO: 105 - protein, 396 aa, Plasmodium vivax
MGEHKTDSKTDNGKGANNLVMLDYETSSNGQPAGTLDNVLEFVTGHEGNSRKNSSNGGNPYDIDHKKTISSAIINHAFLQNTVMKNCNYKRKRRERDWDCNTKKDVCIPDRRYQLCMKELTNLVNNTDTNFHRDITFRKLYLKRKLIYDAAVEGDLLLKLNNYRYNKDFCKDIRWSLGDFGDIIMGTDMEGIGYSKVVENNLRSIFGTDEKAQQRRKQWWNESKAQIWTAMMYSVKKRLKGNFIWICKLNVAVNIEPQIYRWIREWGRDYVSELPTEVQKLKEKCDGKINYTDKKVCKVPPCQNACKSYDQWITRKKNQWDVLSNKFISVKNAEKVQTAGIVTPYDILKQELDEFNEVAFENEINKRDGAYIELCVCSVEEAKKNTQEVVHHHHHH

SEQ ID NO: 106 - DNA, 1191 bp, Plasmodium vivax
  source 1..1191: mol_type = genomic DNA
atgggcgagcacaagaccgactccaagaccgacaacggcaagggcgccaacaacctggtcatgctcgactacgagacgtcctccaacggccagccagctggcaccctggacaacgtgctggagttcgtcaccggccacgagggcaacagcaggaagaactccagcaacggcggcaacccatacgacatcgaccacaagaagaccatctccagcgccatcatcaaccacgccttcctgcagaacaccgtgatgaagaactgcaactacaagaggaagaggcgcgagcgcgactgggactgcaacaccaagaaggacgtctgcatcccagacaggcgctaccaactctgcatgaaggagctgaccaacctcgtgaacaacaccgacaccaacttccacagggacatcaccttccgcaagctgtacctcaagaggaagctgatctacgacgctgctgtggagggcgacctcctgctcaagctcaacaactacaggtacaacaaggacttctgcaaggacatccgctggtccctgggcgacttcggcgacatcatcatgggcaccgacatggagggcatcggctactccaaggtggtcgagaacaacctccgcagcatcttcggcaccgacgagaaggcccaacagaggcgcaagcaatggtggaacgagtccaaggcccagatctggaccgccatgatgtacagcgtgaagaagaggctgaagggcaacttcatctggatctgcaagctcaacgtggccgtcaacatcgagccacagatctacaggtggatcagggagtggggcagggactacgtctccgagctgccaaccgaggtgcaaaagctcaaggagaagtgcgacggcaagatcaactacaccgacaagaaggtgtgcaaggtcccaccatgccaaaacgcctgcaagagctacgaccagtggatcaccaggaagaagaaccaatgggacgtcctgtccaacaagttcatcagcgtgaagaacgccgagaaggtccagaccgccggcatcgtgaccccatacgacatcctgaagcaagagctcgacgagttcaacgaggtggccttcgagaacgagatcaacaagcgcgacggcgcctacatcgagctctgcgtgtgcagcgtcgaggaagccaagaagaacacccaagaggtggtccaccaccaccaccaccactga

SEQ ID NO: 107 - protein, 835 aa, Plasmodium vivax
MVIGGSPNNEAPNSSRHHLRNGFPGKNDSLPHEEPNNLEGKNESSDQCDTINLGQVTEKEKKTIEQASVQAQDATKPEANNAEQIQAELQKVKTAKDESATAAKDAETAKKNAVDAGKGLDAAKGAIKKAEEAAAEAKKQAGIAEKAEKDAEAAGKKDKLEDVNSQVQIAVEASTKAKDKKTEAEIAVEIVKAVVAKEEAQKASDEAQKACEKAQKAHAKAQKASDTTKTVETFKTNAEAAAKNAKEKAGNANKAATEAESANELSVAKQKAKDAEEAAKEAKKEQVKAEIAAEVAKAKVAKEEADAAQKKAEAAKKIVDKIAQDTKVPEAQREAKLATQTASKATEAATEAGKKAQEAEESSKEAEEKAETSDAVKGKADAAEKAAGEAKKASIETEIAIEVAKAEVLNAEVKKTAQEAEKDATEAKEQAEKAKAAAEEAKTHGEKAEKVGESTKAHSDEAQQENKNAKDASEEAENRAVDALEEAYAVEAHLARTKNAAESAKSATDMSELEKAKEEAIDAANIAHQKWLKATQAATIAKEKKEAAKVAAEKAQTAANVVKDKAAKAEAKKAETEAVKAAVEARAAAEEAKQEAAKVGASKEPQETKNKANVEAEATGNEAKKAEDAAEEAKEAAKKANEATDANVARSEADKAIAAAKKAKKAREKAAYGLLKTKNQYVLEPLDISPESADNITSKEEQVKEEMEDQGDEDSNEAEVEEALPNGSGAQEEDVNLEMDDEEEVEEVEENVATNQQTGGKREKRNTNDTVDDTNADKQFGDEFDTYNDIKKVTEALVKSMTSLVSDDPSVGDTINEFLSDMNHLFLSWHHHHHH

SEQ ID NO: 108 - DNA, 2508 bp, Plasmodium vivax
  source 1..2508: mol_type = genomic DNA
atggtcatcggcggctccccaaacaacgaggccccaaactccagcaggcaccacctccgcaacggcttcccaggcaagaacgactccctcccacacgaggagccaaacaacctggagggcaagaacgagtccagcgaccaatgcgacaccatcaacctgggccaggtgaccgagaaggagaagaagaccatcgagcaagctagcgtccaagctcaggacgctaccaagccagaggccaacaacgccgagcaaatccaggccgagctccaaaaggtgaagaccgctaaggacgagtccgctaccgctgctaaggacgctgagacggccaagaagaacgctgtggacgctggcaagggcctggacgccgccaagggcgccatcaagaaggctgaggaagccgccgccgaggccaagaagcaggctggcatcgccgagaaggctgagaaggacgctgaggctgctggcaagaaggacaagctggaggacgtgaacagccaagtccagatcgccgtggaggcctccaccaaggccaaggacaagaagaccgaggccgagatcgccgtggagatcgtcaaggccgtggtcgccaaggaagaggcccaaaaggctagcgacgaggctcagaaggcttgcgagaaggcccaaaaggctcacgctaaggctcagaaggcttccgacaccaccaagaccgtggagacgttcaagaccaacgccgaggctgccgccaagaacgccaaggagaaggctggcaacgctaacaaggctgctaccgaggctgagagcgctaacgagctctccgtggccaagcagaaggccaaggacgccgaggaagccgccaaggaagccaagaaggagcaagtcaaggctgagatcgctgctgaggtggctaaggctaaggtggctaaggaagaggccgacgctgctcagaagaaggctgaggccgccaagaagatcgtggacaagatcgcccaagacaccaaggtgccggaggctcagagggaggctaagctggctacccagaccgctagcaaggctaccgaggccgccaccgaggctggcaagaaggctcaagaggccgaggagtccagcaaggaagccgaggagaaggctgagacgagcgacgctgtgaagggcaaggctgacgctgctgagaaggctgctggcgaggccaagaaggcttccatcgagacggagatcgccatcgaggtcgccaaggccgaggtgctcaacgccgaggtcaagaagaccgctcaagaggccgagaaggacgctaccgaggccaaggagcaagccgagaaggccaaggctgccgccgaggaagccaagacccacggcgagaaggctgagaaggtgggcgagagcaccaaggcccactccgacgaggcccaacaggagaacaagaacgccaaggacgccagcgaggaagccgagaacagggctgtggacgctctcgaagaggcctacgctgtggaggctcacctggctaggaccaagaacgctgctgagtccgctaagagcgctaccgacatgtccgagctggagaaggccaaggaagaggccatcgacgccgccaacatcgcccaccaaaagtggctcaaggctacccaggctgctaccatcgctaaggagaagaaggaagccgccaaggtggctgctgagaaggctcagaccgctgccaacgtggtcaaggacaaggctgctaaggctgaggccaagaaggctgagacggaggccgtcaaggctgctgtggaggccagggccgccgccgaggaagccaaacaagaggccgctaaggtcggcgctagcaaggagccacaagagacgaagaacaaggctaacgtggaggctgaggctaccggcaacgaggccaagaaggccgaggacgctgctgaggaagccaaggaagccgccaagaaggctaacgaggctaccgacgctaacgtggctaggtccgaggctgacaaggctatcgccgccgccaagaaggccaagaaggcccgcgagaaggctgcttacggcctcctgaagaccaagaaccaatacgtgctggagccactggacatctccccagagagcgccgacaacatcacctccaaggaagagcaggtgaaggaagagatggaggaccaaggcgacgaggacagcaacgaggccgaggtggaggaagccctgccaaacggctccggcgctcaagaggaagacgtcaacctggagatggacgacgaggaagaggtggaggaagtggaggagaacgtggccaccaaccaacagaccggcggcaagagggagaagcgcaacaccaacgacaccgtcgacgacaccaacgccgacaagcaattcggcgacgagttcgacacctacaacgacatcaagaaggtgaccgaggccctcgtcaagtccatgaccagcctggtgtccgacgacccatccgtgggcgacaccatcaacgagttcctcagcgacatgaaccacctcttcctgtcctggcaccaccaccaccaccactga

SEQ ID NO: 109 - protein, 117 aa, Plasmodium vivax
MENNKIKGGKVPPPSVPTGNNSDNNVPKKDGGENNPPPDAENALQELKNFTKNLEKKTTTNRNIIISTTVINMVLLVLLSGLIGYNTKKGFKKGQMGSVKEVTPEAQKGKLHHHHHH

SEQ ID NO: 110 - DNA, 354 bp, Plasmodium vivax
  source 1..354: mol_type = genomic DNA
atggagaacaacaagatcaagggcggcaaggtgccaccaccatccgtcccaaccggcaacaactccgacaacaacgtgccaaagaaggacggcggcgagaacaacccaccaccagacgccgagaacgccctccaagagctgaagaacttcaccaagaacctggagaagaagaccaccaccaacaggaacatcatcatctccaccaccgtcatcaacatggtgctcctggtcctcctgagcggcctgatcggctacaacaccaagaagggcttcaagaagggccaaatgggctccgtgaaggaagtgaccccagaggcccagaagggcaagctccaccaccaccaccaccactga

SEQ ID NO: 111 - protein, 614 aa, Plasmodium vivax
ENPVRHSVDIKSEDFVVLISLQNLQTFIMIGYTAVNKDHLNFDFSYLWALCIGTGLFIYSLISFVLIRSLALSKIDIGKYVLELLFSLSIIATCSLSIIIDSFKIANMQLLFFSFALTGYAYYNLMSLFFFCTLVGMTIQYNLSFTGFRAHSTSFFFLDMLSYLVQMIGGNILYFRMYELCTLIVISKRNPCKYVVASKEVKQVEKQIFSSLFNSYMCIKSKTYSDLTCTNDLLNKDSQSVVGRDTNPKWNSPIGTSYQDKVNHTKKLLLRRGKRDKRYPKGGGGARLTCAKHSAYHNSRSLANCASKNTPICTTNFRISNTLSLKNHFNPNLTLEASPPVCKKCVSEKNSHKDNEYKNGEERKKAKRGIKSGTANKSNQLGNHGGDATQVANPTYRTTSHGGDATQVAYPTYRTTSHGGDATQVDSPTHPTTSHGGNNSSSGHPQDDEVLIPIRGTNATNDAAATYNSNASWIKTAAVIDVSVEGKQKKGGHQTFAGNPVNSSANFPSDKKPSYNSHRNGGTPPPNEQLRYYACPCYQTHSSGSSLSEVPSGQTTKRKNSAHNSVEGGNPKMDNQQSRRVSNKRVDGATGEEHDHPSDPPADNPNGNSNTYHC

SEQ ID NO: 112 - protein, 365 aa, Plasmodium vivax
ELSHSLSVKNAPDASALNIEVEKDKKKICKNAFQYINVAELLSPREEETYVQKCEEVLDTIKNDSPDESAEAEINEFILSLLHARSKYTIINDSDEEVLSKLLRSINGSISEEAALKRAKQLITFNRFIKDKAKVKNVQEMLVISSKADDFMNEPKQKMLQKIIDSFELYNDYLVILGSNINIAKRYSSETFLSIKNEKFCSDHIHLCQKFYEQSIIYYRLKVIFDNLVTYVDQNSKHFKKEKLLELLNMDYRVNRESKVHENYVLEDETVIPTMRITDIYDQDRLIVEVVQDGNSKLMHGRDIEKREISERYIVTVKNLRKDLNDEGLYADLMKTVKNYVLSITQIDNDISNLVRELDHEDVEK

SEQ ID NO: 113 - protein, 162 aa, Plasmodium vivax
LPWTKKRKAVNQMGIIKDMSQELRTKAEQLPTPEDISAKIHRVDKEVIDKLNKDIIEEENLDKHKPHVCQEPAYERDYSYLCPEDWVKNSNDQCWGIDYDGHCEALKYFQDYSVEEKKEFEMNCCVLWPKLKNEGMKGAHKKDLLRGSISSNNGLIIKPKYL

SEQ ID NO: 114 - protein, 315 aa, Plasmodium vivax
ELKKNNAALTSQRSSSRTTSTRSYKNAPKNSTSFLSRLSILIFALSCAIFVNTASGAAANRPNANGFVSPTLIGFGELSIQESEEFKRMAWNNWMLRLESDWKHFNDSVEEAKTKWLHERDSAWSDWLRSLQSKWSHYSEKMLKEHKSNVMEKSANWNDTQWGNWIKTEGRKILEAQWEKWIKKGDDQLQKLILDKWVQWKNDKIRSWLSSEWKTEEDYYWANVERATTAKWLQEAEKMHWLKWKERINRESEQWVNWVQMKESVYINVEWKKWPKWKNDKKILFNKWSTNLVYKWTLKKQWNVWIKEANTAPQV

SEQ ID NO: 115 - protein, 529 aa, Plasmodium vivax
KGVTLSCVFSHASEEREGGTGTFALSNEPIYYAPSGGLAPCALISRGLSGDEEGSGEDGGEDGDGDGGEDSAEDNAEDGDDDGGEDGGLPGGRFPYEEGKKSSLVSDAPSDLLDGDADEHAAEDGGAKRKMSKKEEEAEDNKIDKLVNAEMKKLEAGEEANKDPDAEPEKEDQGSGQGQRAKLRCSNKLNYIQVTANGQREGDLFGENDGESAPAFVEIPHEVEEESGGVPTKHDEAGEAAAAEEPHNRVDRAEKENNAKDLKFVEGERERQRSSPPSNGYSQNSFVELKGVPDKLPPNFTNSLGSSPTHSNLEKPVYKHLPWSILASDSGSNTGSWADVNSSTYNVSPFSFTSIRSGNSLHLLPMNFQIQNSIVKVTDEEYDKLKLKNSVKVYDKNALVDYKYEIFEVKEGEEYNDGNDPYEERNGEEGDAGGEGGSDGEGDADSKSYQNNKSDGRGFFDGTLVTYTIIILAGVIILLLSFVIYYYDIINKVKRRMSAKRKNNKSMAIANDTSAGMYMGDTYMENPHV

SEQ ID NO: 116 - protein, 334 aa, Plasmodium vivax
SQGCSGYRLPPPKRWFTFTSRPYCKTAAYYELKHMPYYVDAVSASENVKHEKWNNWLKEMKISLTEKLEKESQEYMEKLEQQWDEFMKNSEDKWRHYNPQMEEEYQCSVYPLGLKWDDEKWTAWFYEKGLWCLKKSFKTWLTDSKKGYNTYMKNLLQEFGKQFYEDWCRRPEKRREDKICKRWGQKGLRNDNYYSLKWMQWRNWKNRNHDQKHVWVTLMKDALKEYTGPEFKLWTEFRKEKIDFYKQWMQAFAEQWTQDKQWNTWTEERNEYMKKKKEEEAKKKAASKKKAASKKGGAAKKAPAKKAPTKKAAPGTKAPAKKAAPKKVAAPNAA

SEQ ID NO: 117 - protein, 269 aa, Plasmodium vivax
KEAVKKGSKKAMKQPMHKPNLLEEEDFEEKESFSDDEMNGFMEESMDASKLDAKKAKTTLRSSEKKKTPTSGMSGMSGSGATSAATEAATNMNATAMNAAAKGNSEASKKQTDLSNEDLFNDELTEEVIADSYEEGGNVGSEEAESLTNAFDDKLLDQGVNENTLLNDNMIYNVNMVPHKKRELYISPHKHTSAASSKNGKHHAADADALDKKLRAHELLELENGEGSNSVIVETEEVDVDLNGGKSSGSVSFLSSVVFLLIGLLCFTN

SEQ ID NO: 118 - protein, 771 aa, Plasmodium vivax
NLSNDCKKGANNSFKLIVHTSDDILTLKWKVTGEGAAPGNKADVKKYKLPTLERPFTSVQVHSANAKSKIIESKFYDIGSGMPAQCSAIATNCFLSGSLEIEHCYHCTLLEKKLAQDSECFKYVSSEAKELIEKDTPIKAQEEDANSADHKLIESIDVILKAVYKSDKDEEKKELITPEEVDENLKKELANYCTLLKEVDTSGTLNNHQMANEEETFRNLTRLLRMHSEENVVTLQDKLRNAAICIKHIDKWILNKRGLTLPEEGYPSEGYPPEEYPPEELLKEIEKEKSALNDEAFAKDTNGVIHLDKPPNEMKFKSPYFKKSKYCNNEYCDRWKDKTSCMSNIEVEEQGDCGLCWIFASKLHLETIRCMRGYGHFRSSALFVANCSKRKPEDRCNVGSNPTEFLQIVKDTGFLPLESDLPYSYSDAGNSCPNKRNKWTNLWGDTKLLYHKRPNQFAQTLGYVSYESSRFEHSIDLFIDILKREIQNKGSVIIYIKTNNVIDYDFNGRVVHSLCGHKDADHAANLIGYGNYISAGGEKRSYWIVRNSWGYYWGDEGNFKVDMYGPEGCKRNFIHTAVVFKIDLGIVEVPKKDEGSIYSYFVQYVPNFLHSLFYVSYGKGADKGAAVVTGQAGGAVVTGQTETPTPEAAKNGDQPGAQGSEAEVAEGGQAGNEAPGGLQESAVSSQTSEVTPQSSITAPQIGAVAPQIGAAAPQIDVAAPQIDVVAPQTRSVDAPQTSSVAAHPPNVTPQNVTLGEGQHAGGVGSLIPADN

SEQ ID NO: 119 - protein, 219 aa, Plasmodium vivax
ETLLDSETLKNYEKETNEYIRKKKVEKLFDVILKNVLVNKPENVYLYIYKNIYSFLLNKIFVIGPPLLKITPTLCSAIASCFSYYHLSASHMIESYTTGEVDDAAESSTSKKLVSDDLICSIVKSNINQLNAKQKRGYVVEGFPGTNLQADSCLRHLPSYVFVLYADEEYIYDKYEQENNVKIRSDMNSQTFDENTQLFEVAEFNTNPLKDEVKVYLRN

SEQ ID NO: 120 - protein, 113 aa, Plasmodium vivax
YPKKNFDKPDPTSPYQGQYGESEEQRQGYGIPPNPTMINLTGNQDQRPNVLQQFGINNKNVMQFLINMFVYVAAILVSLKIWDYMSYSKCDYYKDLLLRIVRYQSHMNDGKMA

SEQ ID NO: 121 - protein, 666 aa, Plasmodium vivax
SRIDKQPIQSSYLFQDNAVPPVRFSAVDADLFSIGVVHTEEQIFMDDANWVISSVPSKYLNLHLLKTGSRPHFSHFSVSMNTGCNLFIASPVGETFPLSPSKDGATWKAFETDDSVEVIHRETKEKRIYKLKFIPLKSGALLKVDVLKGIPFWVISQGRKILPTICSGDEEVLSNPQNEVFKECTSSSSLSPEFDCLAGLSTYHRDKKNHTWKTSSGSIGQFIKIFFNKPVQITKFRFKPRDDLLSWPSEVALQFDTDEEVIIPILHTHNMGQNTTRLEHPIITTSVKVEVRDMYERASENTGGSFEVIGSTCQMMEDDYMTHHAVIDITECDRRLESLPDVMPLTKGSKFLAICPRPCLSSSNGGVIYGSDVYSTDSAVCGAAVHAGVCSREGEGSCHFLVVVRGGRANFVGALQNNVLSLSRGGGGSGSGSSTSSDGDGDSDSSTSRANFSFSLSSASGFGGGPRGAHAEAAPSSYSIVFKPRDHLAPTNGFLVDSGREFTSYGSVAYGWKREVSPSSSFSSPSPSYTSPPLEEPTLLRGDSSSFNGIYSGGIEFPPASASQNCISQLDCQTNFWKFQMQENGTYFVQVLVGNKTSPEKQKAFVELNGVPIIKGVDLGPDEVFVATDRVQVTNRALVLTSTCLGGESACSRARVSIMAVQIVKT

SEQ ID NO: 122 - protein, 582 aa, Plasmodium vivax
NGMNKDKDAEITPPPFIVLPGGKKIHMLQSEYEYDVLRDMYRTDEANGGSGEKESHPSGDGAIRRNEFFKLFHHREGHYKFVIKNVPTKLSDLLQKGGNEQETDLFPLLYRSLQFACSADGTWPYARREVAFFKNGSVHCEAEFQNELSVRRTPRSGKKSFGRFPRGTLIKSSDLRSKIVEGNSYDKRAAPLKSEKKKKALFLHPESVLYKMEEIFFYENPSVKSEIVGFVLFHDVCTVTSLGHGAHPVNSPFLGSDLLEMIFGYCILHGFKKIRVKSESLNYETGIRTSFIEILLNGKTALEHLGLRLTNVAKFSKELYYVITGYTWKSDLVLSPIVRFEHDLYVHHDIEERFFLYVNKMYRNMLHDLSFSCDENYYPYKNCYDIYPSVRRSQNNLCLFELNPIYEELKELFPDSCNIGQRVRKCYEEIKKNVVCTHNGEGGEDGCKYYQFIVNTFIKPRRKTSFFIYHNMYVQEYLSKKSYPYYLLLSEVIKNEENNFLEKGNYDLVADAQTHLFLNYVLQNSTFFIFWNFSTEFWKRFRYIQAGPTGATSTPQKGQAVFCPMAYAYEFVEHLDTFYVRG

SEQ ID NO: 123 - protein, 392 aa, Plasmodium vivax
SVEEAKKNTQEVVTNVDNAAKSQATNSNPISQPVDSSKAEKVPGDSTHGNVNSGQDSSTTGKAVTGDGQNGNQTPAESDVQRSDIAESVSAKNVDPQKSVSKRSDDTASVTGIAEAGKENLGASNSRPSESTVEANSPGDDTVNSASIPVVSGENPLVTPYNGLRHSKDNSDSDGPAESMANPDSNSKGETGKGQDNDMAKATKDSSNSSDGTSSATGDTTDAVDREINKGVPEDRDKTVGSKDGGGEDNSANKDAATVVGEDRIRENSAGGSTNDRSKNDTEKNGASTPDSKQSEDATALSKTESLESTESGDRTTNDTTNSLENKNGGKEKDLQKHDFKSNDTPNEEPNSDQTTDAEGHDRDSIKNDKAERRKHMNKDTFTKNTNSHHLN

SEQ ID NO: 124 - protein, 530 aa, Plasmodium vivax
IRNGNNPQALVPEKGADPSGGQNNRSGENQDTCEIQKMAEEMMEKMMKEKDVFSSIMEPLQSKLTDDHLCSKMKYTNICLHEKDKTPLTFPCTSPQYEQLIHRFTYKKLCNSKVAFSNVLLKSFIDKKNEENTFNTIIQNYKVLSTCIDDDLKDIYNASIELFSDIRTSVTEITEKLWSKNMIEVLKTREQTIAGILCELRNGNNSPLVSNSFSYENFGILKVNYEGLLNQAYAAFSDYYSYFPAFAISMLEKGGLVDRLVAIHESLTNYRTRNILKKINEKSKNEVLNNEEIMHSLSSYKHHAGGTRGAFLQSRDVREVTQGDVSVDEKGDRATTAGGNQSASVAAAAPKDAGPTVAAPNTAATLKTAASPNAAATNTAAPPNMGATSPLSNPLYGTSSLQPKDVAVLVRDLLKNTNIIKFENNEPTSQMDDEEIKKLIESSFFDLSDNTMLMRLLIKPQAAILLIIESFIMMTPSPTRDAKTYCKKALVNGQLIETSDLNAATEEDDLINEFSSRYNLFYERLKLEEL

SEQ ID NO: 125 - protein, 524 aa, Plasmodium vivax
KEYCDQLSFCDVGLTHHFDTYCKNDQYLFVHYTCEDLCKTCGPNSSCYGNKYKHKCLCNSPFESKKNHSICEARGSCDAQVCGKNQICKMVDAKATCTCADKYQNVNGVCLPEDKCDLLCPSNKSCLLENGKKICKCINGLTLQNGECVCSDSSQIEEGHLCVPKNKCKRKEYQQLCTNEKEHCVYDEQTDIVRCDCVDHFKRNERGICIPVDYCKNVTCKENEICKVVNNTPTCECKENLKRNSNNECVFNNMCLVNKGNCPIDSECIYHEKKRHQCLCHKKGLVAINGKCVMQDMCRSDQNKCSENSICVNQVNKEPLCICLFNYVKSRSGDSPEGGQTCVVDNPCLAHNGGCSPNEVCTFKNGKVSCACGENYRPRGKDSPTGQAVKRGEATKRGDAGQPGQAHSANENACLPKTSEADQTFTFQYNDDAAIILGSCGIIQFVQKSDQVIWKINSNNHFYIFNYDYPSEGQLSAQVVNKQESSILYLKKTHAGKVFYADFELGHQGCSYGNMFLYAHREEA

SEQ ID NO: 126 - protein, 340 aa, Plasmodium vivax
SKNIIILNDEITTIKSPIHCITDIYFLFRNELYKTCIQHVIKGRTEIHVLVQKKINSAWETQTTLFKDHMWFELPSVFNFIHNDEIIIVICRYKQRSKREGTICKRWNSVTGTIYQKEDVQIDKEAFANKNLESYQSVPLTVKNKKFLLICGILSYEYKTANKDNFISCVASEDKGRTWGTKILINYEELQKGVPYFYLRPIIFGDEFGFYFYSRISTNNTARGGNYMTCTLDVTNEGKKEYKFKCKHVSLIKPDKSLQNVAKLNGYYITSYVKKDNFNECYLYYTEQNAIVVKPKVQNDDLNGCYGGSFVKLDESKALFIYSTGYGVQNIHTLYYTRYD

SEQ ID NO: 127 - DNA, 1842 bp, Plasmodium vivax
  source 1..1842: mol_type = genomic DNA
gagaaccccgtgaggcactcggtggacataaagtcggaagacttcgtcgtcctgatttcgctccaaaacctgcagaccttcatcatgatagggtacacagccgtgaacaaagaccacctgaatttcgacttctcctacttatgggccctctgcatcgggacgggcctcttcatatactccctcatcagctttgtactcataagatccctagcactgtcaaaaatagacataggcaaatacgtcctggagctgctattcagtttgagtataatcgccacatgttcactctccataataattgactctttcaaaatagccaacatgcagttgctttttttttcgttcgctttaacgggctatgcctactacaatttgatgagcctcttctttttctgcacactggtaggaatgaccattcagtacaatttaagtttcactgggttcagagcgcattcgacttctttcttctttttagatatgctatcttacctagtgcaaatgataggagggaacatcctctactttcgcatgtacgagctgtgtaccctaatcgtcatttcgaagaggaacccctgcaagtatgttgtcgcatcgaaggaagtgaaacaagtggagaagcaaattttctcttctttatttaattcttacatgtgcatcaagtccaaaacttattcagatttaacctgcactaatgatctgttaaataaagacagtcaatctgttgtcggtagggatacgaaccctaagtggaactcccccattggtacttcctaccaggataaggtcaatcatacgaagaagttactccttcggaggggaaaacgggacaaacgctaccccaaagggggagggggagctcgactaacatgtgcaaaacatagtgcctaccataatagccgaagtcttgccaactgtgccagtaagaatacccccatttgcacaactaactttaggatatctaacaccctttcacttaaaaatcatttcaaccctaacctaaccttagaagcgtctccccccgtttgtaaaaaatgcgtttcggaaaagaatagccataaggataatgagtacaaaaacggggaagagagaaaaaaagcaaaacgtggtatcaagtcgggcactgcaaacaagtctaaccagttgggcaaccacgggggggacgctacgcaggtggctaatcctacctacagaactacttcccacgggggggacgcaacccaggtggcttatcctacctacagaactacttcccacgggggggacgcaacgcaggtggatagtcctacccacccaactacctcccatggggggaacaactcgtcgagcgggcacccccaagacgacgaagtgctcatccccattaggggaaccaacgccactaacgatgcagccgccacctacaactcgaacgctagttggatcaaaaccgctgcggttattgacgtgtctgtggaggggaagcagaaaaaggggggacatcaaacgttcgcgggcaatcccgtaaattcatccgctaatttcccatcggacaagaaaccttcctacaactcgcaccgcaacggaggtactccccccccaaatgaacaactcaggtactacgcctgcccctgctaccagacccactccagcggatcgtccctcagtgaggtgccctcgggacaaacgacgaagcggaaaaatagtgcgcacaactcggttgaagggggaaaccccaaaatggataatcagcaaagtcgccgcgtgagtaacaagcgggtagatggcgcaacgggtgaggaacatgaccacccaagtgacccccccgcagataacccaaatggaaactccaacacctaccactgc

SEQ ID NO: 128 - DNA, 1095 bp, Plasmodium vivax
  source 1..1095: mol_type = genomic DNA
gagctgagccacagcttgtccgtgaagaacgcgccggacgcgagcgcgctgaacatcgaggtggagaaggacaaaaagaagatctgcaaaaacgcattccaatacataaacgtagctgagctgttgtccccaagggaggaagaaacctacgtgcagaaatgtgaagaggtcctagacacaataaagaatgacagtccagatgaatcggcagaagcagagataaacgaatttatactgagcttactgcacgctcgttctaagtataccataataaatgactcagatgaggaggtactgagcaagctcctgaggagtatcaacggatcgataagtgaagaggcagcgttgaagagagccaaacagctaatcacattcaatcggtttataaaagacaaagcgaaggtaaaaaatgtgcaagagatgctagtaataagtagcaaagcagatgacttcatgaatgagccgaagcaaaaaatgctccaaaaaattatagattcgtttgaactgtataatgattacctagtcattttagggtcaaatattaacatcgccaagaggtactcctcagaaacgtttctttctattaaaaatgaaaagttctgctcagaccacatccacttatgccagaagttctacgagcagtctatcatttactacagattgaaggttatttttgataacctggtgacttatgtagatcaaaattccaagcattttaaaaaggaaaagttgctggagcttctaaatatggattatagggtcaatcgagagtcgaaggtgcatgaaaattacgtgctggaggatgagacggtcatccccacgatgcgcattacagacatttacgatcaagataggctaattgttgaggtcgttcaggatggaaatagcaagctgatgcacggcagggatattgagaagagggaaatcagcgagaggtacatcgtcaccgtgaagaacctgcgcaaggacctcaacgacgaggggctctacgccgacttgatgaagaccgtcaagaactacgtgctctccatcacgcagatcgacaacgacatttccaacctcgtgcgcgagctcgaccacgaggatgtggagaag

SEQ ID NO: 129 - DNA, 486 bp, Plasmodium vivax
  source 1..486: mol_type = genomic DNA
ctaccatggacgaagaaaagaaaggcggtgaaccaaatgggcatcataaaagatatgtcgcaggagcttaggactaaggccgaacagcttccaacccccgaggatatatcagccaaaattcacagagtagataaagaggtcatcgataagttaaacaaagacatcatagaggaagaaaatttagacaagcacaaaccgcacgtctgccaggagccagcatacgagagggactattcgtacctatgtcccgaagactgggtgaagaactccaacgatcagtgctggggcatagactacgatggtcactgtgaagcgctaaaatatttccaagattattctgtagaggagaaaaaagaatttgaaatgaactgctgcgtcttgtggcctaagctaaaaaatgaaggcatgaaaggagcgcacaagaaggacctcctaaggggatcgataagttcaaacaatgggttaataataaagccgaaatatttg

SEQ ID NO: 130 - DNA, 945 bp, Plasmodium vivax
  source 1..945: mol_type = genomic DNA
gaattgaagaagaacaatgccgcgttgacctcacaaaggtcatcttctagaaccacatccacaaggagctacaaaaatgccccaaaaaattccacttcattcctttctcgtttatctattctgatatttgccttatcatgtgctatttttgtaaatactgcatcaggggcggcagctaatagaccaaacgcgaatggctttgtgtcacctactttaataggatttggcgaattaagcatccaagaatcagaagaattcaaaagaatggcttggaataattggatgttgcgattggagtccgactggaaacattttaacgattctgttgaagaagccaaaaccaaatggcttcatgaaagagactcagcttggtctgattggcttcgttccttgcaaagtaaatggtctcactatagtgaaaaaatgcttaaagaacacaaaagtaatgttatggaaaaatcagccaactggaatgacacgcaatggggaaattggataaaaactgaaggaagaaaaattctagaagcgcaatgggaaaaatggattaaaaaaggtgatgaccaattacaaaagttaattttagataaatgggttcaatggaaaaatgataagatccgatcctggttatccagtgaatggaaaaccgaagaagattactactgggcaaatgtagagcgcgctacaacagcaaaatggttgcaagaagcagagaaaatgcattggcttaaatggaaagaaagaattaacagagagtctgaacaatgggtgaactgggtccaaatgaaagaaagcgtttacatcaatgtagaatggaaaaaatggcccaaatggaaaaatgataaaaaaattctatttaacaaatggtcaactaaccttgtctacaaatggacactgaaaaagcagtggaacgtttggattaaggaagcaaatactgcaccccaagtt

SEQ ID NO: 131 - DNA, 1587 bp, Plasmodium vivax
  source 1..1587: mol_type = genomic DNA
aagggtgtcaccttgagttgcgttttttcccatgcgagtgaggaacgtgagggtggcacagggacatttgctttgagcaatgagccgatttattacgcccctagtggggggctggcgccgtgcgcgctcatcagcagagggttaagcggggatgaggagggtagcggcgaggacggcggtgaagatggcgacggagatggtggtgaagacagcgctgaggacaacgctgaggatggagacgatgatggtggcgaagatggcggcttgcccgggggacgcttcccatacgaagaaggaaaaaagagtagccttgtgagcgacgcacccagcgacctcctggatggagatgcggatgaacatgccgccgaagatgggggagcgaagcgaaagatgagtaagaaggaggaagaggcggaggataacaaaattgacaagttggtaaatgcggaaatgaaaaagctcgaggcaggggaagaggcgaacaaggatcccgacgcagaaccagaaaaagaggaccagggaagtggccaaggacaaagggcgaagctgaggtgctcaaacaagctaaattacatacaggtgacggcgaatggccaaagggagggcgacctctttggcgagaacgacggggagagcgccccagctttcgtggagataccccacgaggttgaggaggaaagcggcggtgtgcccacaaagcatgacgaagcgggggaagcagctgcggcggaggaaccacataaccgcgtcgaccgagcggaaaaagaaaacaacgcgaaggacttaaaatttgtggagggggagcgagaaagacaaaggagcagccccccctcgaatggatattcccaaaacagctttgtcgaactgaaaggtgtgcccgataaattgccccctaattttaccaactcgcttggtagctccccaacgcacagtaatttggagaaaccagtttataagcacttaccctggtctatcctggcatccgactctggttcgaacaccgggtcctgggcagacgtcaacagtagtacctacaatgtgagtccattcagtttcacctcaatacgtagtggtaactctctgcatctactgccgatgaatttccaaatccaaaactccatcgtgaaagtaactgatgaggagtatgacaaattgaagcttaaaaacagcgtcaaagtgtatgacaaaaatgccctggtagattataagtatgaaatttttgaggtgaaggaaggggaggaatataatgatgggaatgacccttatgaggaaaggaatggggaagaaggggatgcaggtggagaggggggttccgatggggagggagatgcagattctaaatcatatcaaaataacaaatcggatggacgtgggttcttcgatgggaccttagtaacctacaccattatcattttagctggtgttataattctgctgctaagttttgtcatttattactacgatataataaataaggtgaagaggcgaatgagtgccaagcggaagaacaacaaatctatggccatcgcgaatgatacatccgcggggatgtacatgggcgacacctacatggagaatccccacgtt

SEQ ID NO: 132 - DNA, 1002 bp, Plasmodium vivax
  source 1..1002: mol_type = genomic DNA
tcacaaggatgttcaggataccgtttaccaccaccaaaaagatggtttaccttcacttctcgaccatactgtaaaacagctgcatattatgaacttaaacatatgccatattatgtagatgcagttagtgcatcagaaaacgtaaaacatgagaaatggaataactggttaaaagaaatgaaaatatcattaactgaaaaattagaaaaagaatcacaagaatatatggaaaaattggaacagcaatgggatgaatttatgaaaaattcagaagataaatggaggcattataatccccaaatggaagaagaatatcaatgtagtgtttatccacttggattaaaatgggatgatgaaaagtggactgcatggttttatgaaaaaggattatggtgtttgaagaaaagctttaaaacatggctcactgattctaaaaaaggttacaacacctacatgaaaaatcttttacaggaatttggtaaacaattttatgaagattggtgtcgtagacctgaaaaacgtcgtgaagataaaatttgcaagagatggggacaaaaaggattacgtaatgacaattactattcgttaaagtggatgcagtggagaaattggaaaaacagaaaccacgatcaaaaacatgtgtgggtaactcttatgaaggatgcgctaaaggaatatacggggcccgaattcaaattatggactgagtttagaaaagaaaagatagacttttacaagcaatggatgcaagctttcgccgaacagtggacacaagacaaacaatggaatacgtggactgaagaaagaaatgaatatatgaaaaagaaaaaagaagaagaagcaaaaaaaaaagcagcatcaaaaaaaaaagcagcatcaaaaaaaggaggagcagcaaaaaaggcaccagcaaaaaaggcaccaacaaaaaaagccgcaccaggaacaaaggcaccagcaaaaaaagcagcacctaaaaaagttgcagcaccaaatgcagca

SEQ ID NO: 133 - DNA, 807 bp, Plasmodium vivax
  source 1..807: mol_type = genomic DNA
aaggaggcagtgaagaaggggtccaagaaggcaatgaagcagcccatgcacaagccgaaccttcttgaagaggaagactttgaggagaaagaatccttttcggatgacgagatgaatgggttcatggaggagagcatggatgcttctaagttggatgcgaagaaggccaagacgaccctcaggagctcggagaagaagaagactccaacgagcggaatgagtggaatgagtggaagcggcgccaccagcgcagccaccgaggcagccacgaacatgaacgccaccgccatgaacgccgctgctaagggcaacagcgaggcgagcaaaaagcaaaccgacttgtccaacgaagacctgttcaacgacgagctcacagaagaggtcattgcagattcgtacgaagagggaggaaacgtgggaagcgaggaagccgaaagcctcacaaatgcatttgacgacaagctactagaccaaggagtgaatgaaaatactctgctgaacgacaacatgatttacaacgtcaatatggttccacataagaagcgagaattatacatctccccacacaagcatacctctgcagcaagcagtaaaaatggcaaacatcatgcggcggacgcggacgctttggacaaaaaactgagggctcacgagctgctcgagctggaaaacggagaaggcagcaactcagtcattgtcgaaacggaagaagtggatgttgacctaaacggaggaaagtcaagcggctccgtgtccttcctcagctccgtagtcttcttgctcatcggattgttatgtttcaccaat

SEQ ID NO: 134 - DNA, 2313 bp, Plasmodium vivax
  source 1..2313: mol_type = genomic DNA
aacctgagcaacgattgcaaaaaaggagccaacaacagctttaagttaatcgtgcacaccagcgatgatattttgacactcaagtggaaggtcactggggaaggggcagctccaggcaacaaagcagatgtaaagaagtacaaactccctaccctagagaggcctttcacttccgtgcaagtgcattcagccaacgccaagtcgaagataatcgaaagcaaattttacgacattggcagcggcatgccagcccagtgcagcgcgatcgccacgaactgcttcctcagcggcagcctcgaaatcgagcactgctaccactgcaccctgttggagaagaagctggcccaagacagcgagtgcttcaagtacgtctcgagtgaagcgaaggagttgatcgagaaagacacgccgattaaagctcaagaagaagacgccaactctgcagaccacaaactgatcgagtccatagacgtgatactaaaggcagtgtacaaatcagataaagatgaggaaaagaaggagctcatcaccccggaggaagtggacgaaaatttgaagaaagagctagccaattattgtaccctactgaaggaggtagacacaagtggcactcttaacaaccaccagatggcaaacgaagaggaaacgttcagaaatttgactcgactgttgcgaatgcatagcgaagaaaacgtggtgacccttcaggacaaactgagaaacgcagccatatgcatcaagcacatcgacaagtggattcttaacaagagggggttgaccctaccggaagaagggtacccatcggaagggtaccccccagaagagtaccccccggaggaactcctcaaagaaatcgagaaggaaaaaagcgctctgaatgatgaagcgttcgctaaagataccaacggagtcatccacctggataagcctcccaacgaaatgaaatttaaatccccctattttaaaaagagcaaatactgtaacaatgagtactgtgataggtggaaagataaaacgagttgcatgtcaaatatagaagtggaagagcaaggggattgcgggctctgttggattttcgcctctaagttacacttagaaacgatcaggtgcatgagagggtatggccacttccgcagctccgctctgtttgtggccaactgctcgaagaggaagccagaagatagatgcaacgtgggttctaaccctacagagtttcttcaaattgttaaggacacgggatttttacctctagagtccgatctcccctacagctatagcgacgcggggaactcctgccccaataaaagaaacaagtggaccaacctgtggggggataccaaactgctgtatcataagagacccaatcagtttgcacaaacactcgggtacgtttcctacgaaagcagtcgctttgagcacagcatcgacctcttcatagacatcctcaaaagggaaattcaaaacaaaggctccgttatcatttacataaaaaccaacaatgtcatcgattatgactttaatggaagagtcgtccacagcctatgtggccataaggatgcagatcatgccgctaacctgatcggttatggtaactacatcagtgctggtggggagaagaggtcctattggattgtgcgaaacagctgggggtactactggggagatgaaggcaactttaaggttgacatgtacggcccggagggatgcaaacggaacttcatccacacggctgttgtgtttaagatagacctgggcatcgtcgaagtcccgaagaaggacgaggggtccatttatagctacttcgttcagtacgtccccaactttttgcacagccttttctacgtgagttacggtaagggtgctgataagggagcggcggtggtgacagggcaggcgggaggagcggtagtcacaggacagactgaaacgcccactccggaggccgctaaaaatggggatcagccaggagcacagggtagcgaggcagaagtcgcggagggtggccaggcaggaaatgaagccccgggagggttgcaagagagtgctgtttcgtcgcaaacgagtgaggttacgccgcaatctagtataactgctccgcaaatcggtgcagttgccccacaaatcggtgcagctgccccacaaatcgatgtagccgccccacaaatcgatgtagtcgccccacaaacgaggtccgttgacgccccccaaacgagctcggttgccgcccaccccccaaacgtgacgccgcagaacgtgacgcttggggagggccagcacgcggggggtgtaggctccctcatccccgcggacaac

SEQ ID NO: 135 - DNA, 657 bp, Plasmodium vivax
  source 1..657: mol_type = genomic DNA
gaaaccctgctagacagcgaaacgttaaagaactacgaaaaggaaacgaacgaatacattcgcaaaaaaaaagtggagaaactgttcgatgttattttaaaaaatgttctggtaaacaaaccggaaaatgtatacctgtacatatacaagaacatttattccttccttttgaacaaaatttttgtgatcggccctcctttgctgaaaattactcccaccttatgttctgcgattgccagctgctttagctactaccacctcagcgcctcgcacatgatcgagtcttacactactggtgaagtagatgacgctgcagagagttccacaagcaaaaagttagtcagtgacgacttaatctgctccatcgttaaaagcaacataaaccagctgaacgcgaagcaaaagcgggggtatgtagtcgaagggttccccggcaccaatcttcaggcagacagttgcctacggcatttgccatcttacgtttttgtcctgtacgccgacgaagagtacatttatgacaagtacgaacaagagaacaacgtaaaaattcgttcagacatgaacagccaaacttttgatgaaaacacacagttgttcgaagtggccgagttcaacacgaatccgctgaaggatgaggtaaaggtctacttaaggaac

SEQ ID NO: 136 - DNA, 339 bp, Plasmodium vivax
  source 1..339: mol_type = genomic DNA
tatccaaaaaagaacttcgacaaacccgacccaacttccccataccaaggacaatatggagagtctgaggaacaaagacaaggttatggaatcccccccaacccaaccatgattaaccttactggtaaccaagaccaacgaccaaatgtattgcaacaatttggaataaacaacaaaaatgtaatgcagtttttaataaacatgtttgtgtacgttgctgctatattagttagtttaaaaatatgggactacatgtcttacagcaaatgtgattattacaaagatttattattaagaattgtaagataccaatcacacatgaatgatggtaagatggcc

SEQ ID NO: 137 - DNA, 1998 bp, Plasmodium vivax
  source 1..1998: mol_type = genomic DNA
agccgcatcgacaagcagcccatccagagcagctacctcttccaggataacgcagtcccgcctgttcgattctccgcagtagatgcagacctgttttccattggagtagttcacacagaggagcaaatatttatggacgacgccaactgggtgattagcagcgtgcccagtaagtacctgaacttgcatctactcaaaacgggttctagaccccatttttcgcacttctccgtatctatgaacacgggttgcaacctattcatcgcttcaccggtgggggaaaccttccccttgagtccctccaaagatggagcgacgtggaaagcatttgaaacggacgacagtgtagaggtgattcacagagagacgaaggaaaagagaatctataagctcaagttcattcctctgaagagtggggctctcctaaaggttgacgttttgaagggaattcccttttgggttatctcacaagggaggaaaatcctaccaacgatttgttctggagatgaggaggtgctatcaaacccacagaatgaggtcttcaaagagtgcacatcgtcgagtagtctctctcccgaatttgattgtctagccgggctgagcacctaccatagggataagaagaaccacacgtggaaaacgtctagcggatctataggtcagtttataaagatcttcttcaataagcccgtacaaattaccaagtttaggtttaagcccagagacgacctgctgtcttggccctccgaagtagctctccaattcgataccgatgaggaggtgatcataccaattctgcatacgcacaatatggggcagaacacgactaggctagaacacccaatcatcaccacctctgttaaggtagaagtgagagacatgtacgaacgggcaagtgaaaatacaggaggttctttcgaggtaattggaagcacatgccagatgatggaagacgactacatgacgcaccatgctgttatagacatcaccgagtgtgatcgtaggttggagtccctcccagatgttatgcccttaacgaaggggagcaaatttctggccatttgtccccgcccctgcttgagcagctccaatgggggagtcatttacgggtcagatgtttattccacagattctgccgtatgtggggcggccgtacacgcgggggtgtgcagccgtgagggggagggcagctgccacttcctcgttgtggtgcgcggcgggcgggccaacttcgtgggggctctccagaacaacgtcctgtctctcagtcggggtggtggcggtagcggtagcggtagctccaccagtagcgatggcgatggcgatagcgatagctccaccagtagggccaacttctcattttccctctccagtgcgtcagggttcggggggggtccgcgcggggcccacgcagaagccgcgccaagcagctactccattgtgttcaagccgagggaccatttggctccaacgaacggctttctagtagactcagggagagagttcaccagctacggaagcgttgcctacggatggaagagggaggtttctccttcgtcctctttttcctctccttctcctagctacacttcccccccgttggaagaaccgacgctgcttaggggggactcctcctcattcaatgggatttactccggggggatagaattcccccccgcctcggctagccaaaattgcatttcccaactggattgccagaccaacttctggaagtttcagatgcaagaaaatggcacctactttgtgcaggtgctagtggggaataaaacttcccctgagaagcagaaggccttcgtcgagctgaatggcgttcccatcataaagggggtggaccttggcccagacgaggtcttcgtcgccactgaccgcgtgcaggtgacgaaccgggccctcgtcctcacgtccacttgcctgggcggcgagagtgcctgctcgcgggcgcgcgtcagcatcatggcggtccagattgtgaagacg

SEQ ID NO: 138 - DNA, 1746 bp, Plasmodium vivax
  source 1..1746: mol_type = genomic DNA
aacggtatgaataaagacaaagacgcagagattactccccctccgttcatcgtcttgccgggtggaaaaaaaatccacatgctgcaaagcgaatacgagtatgacgttctgcgggatatgtaccgaacggatgaggcgaatgggggaagtggtgagaaggagagtcacccctctggggatggtgcaatcagaagaaacgaattttttaaactttttcaccacagggagggtcattataagtttgttatcaaaaatgttcccaccaaattgagcgaccttttgcagaaaggtggcaacgaacaggagacagacctatttcctcttttatacaggagtctgcaattcgcatgcagcgcagacgggacgtggccatatgccagaagagaggtggccttttttaaaaacgggagcgtccactgcgaagcggaatttcaaaacgagttatcagtgaggagaaccccccgaagtgggaagaaatcatttggacgttttccaagggggacactaataaaaagtagcgacctgaggagcaaaattgtggaggggaattcttatgataaaagggccgcacccctgaagagtgaaaaaaaaaagaaggctctctttttacacccagaaagtgtgctatacaaaatggaagaaatatttttttatgaaaatccaagtgtcaaaagtgaaattgtcgggtttgttctttttcatgatgtgtgcacagtaacgtccttaggacatggagcacatcccgttaactccccctttttgggaagcgacctgctggagatgatatttggctactgcattttacacgggtttaaaaaaatcagagtgaaaagcgaatccttaaattacgaaactgggataaggacctcattcattgagattttactcaacggaaaaacagcacttgaacatttagggttaagacttacaaacgtagcgaagttttctaaagaactgtattatgtaatcactgggtatacgtggaaaagtgatttggtgctatcacccatagtaaggtttgaacatgatttatacgtgcatcacgacatagaggagcgatttttcctttacgtgaataaaatgtataggaatatgctccacgatttgtccttctcttgtgatgaaaattattatccttataaaaattgttatgacatctacccctccgtgagaaggagtcaaaataatctttgtctcttcgaactgaatcccatatatgaagaattgaaggagctctttccagactcttgtaatattggccaacgcgttagaaaatgctatgaggagataaaaaaaaacgttgtctgcacacataacggtgaaggaggagaagacggatgtaagtactaccaatttattgtaaatacattcataaagccgaggaggaaaacgtccttttttatttatcacaatatgtatgtacaggaatatctttcaaagaaatcctacccctattacttgctactcagtgaggttataaaaaatgaagaaaataactttctcgaaaaaggcaactacgacttagtggccgatgcacagacgcacctcttcttaaattacgttttgcaaaattctaccttttttatcttttggaatttctctaccgaattttggaaaaggtttcggtacatccaggctggcccaaccggggccacttccacaccgcagaaggggcaagctgtgttttgccccatggcctatgcgtacgaatttgtggagcacctcgacacgttttatgtgaggggg

SEQ ID NO: 139 - DNA, 1176 bp, Plasmodium vivax
  source 1..1176: mol_type = genomic DNA
tccgttgaagaggctaaaaaaaatactcaggaagttgtgacaaatgtggacaatgctgctaaatctcaggccaccaattcaaatccgataagtcagcctgtagatagtagtaaagcggagaaggttccaggagattctacgcatggaaatgttaacagtggccaagatagttctaccacaggtaaagctgttacgggggatggtcaaaatggaaatcagacacctgcagaaagcgatgtacagcgaagtgatattgccgaaagtgtaagtgctaaaaatgttgatccgcagaaatctgtaagtaaaagaagtgacgacactgcaagcgttacaggtattgccgaagctggaaaggaaaacttaggcgcatcaaatagtcgaccttctgagtccaccgttgaagcaaatagcccaggtgatgatactgtgaacagtgcatctatacctgtagtgagtggtgaaaacccattggtaaccccctataatggtttgaggcattcgaaagacaatagtgatagcgatggacctgcggaatcaatggcgaatcctgattcaaatagtaaaggtgagacgggaaaggggcaagataatgatatggcgaaggctactaaagatagtagtaatagttcagatggtaccagctctgctacgggtgatactactgatgcagttgatagggaaattaataaaggtgttcctgaggatagggataaaactgtaggaagtaaagatggagggggggaagataactctgcaaataaggatgcagcgactgtagttggtgaggatagaattcgtgagaacagcgctggtggtagcactaatgatagatcaaaaaatgacacggaaaagaacggggcctctacccctgacagtaaacaaagtgaggatgcaactgcgctaagtaaaaccgaaagtttagaatcaacagaaagtggagatagaactactaatgatacaactaacagtttagaaaataaaaatggaggaaaagaaaaggatttacaaaagcatgattttaaaagtaatgatacgccgaatgaagaaccaaattctgatcaaactacagatgcagaaggacatgacagggatagcatcaaaaatgataaagcagaaaggagaaagcatatgaataaagatacttttacgaaaaatacaaatagtcaccatttaaat

SEQ ID NO: 140 - DNA, 1590 bp, Plasmodium vivax
  source 1..1590: mol_type = genomic DNA
atacggaatggaaacaacccgcaggcattagttcctgaaaagggcgctgacccgagtgggggccagaacaaccgctccggagaaaaccaagacacgtgcgaaattcaaaagatggccgaagaaatgatggaaaaaatgatgaaggaaaaagacgtgtttagctccatcatggaacctctccagagcaaattaactgacgatcatctgtgttcaaaaatgaaatatacgaacatttgtcttcacgaaaaggacaaaactcccttgaccttcccctgcacaagtccgcagtacgaacagctaattcatcgcttcacttataaaaagttgtgcaactccaaggtggcctttagcaacgtcttgctcaaatccttcatcgataaaaaaaatgaagaaaacacatttaacacgatcatacagaattacaaagttctgtccacttgcattgacgatgatttgaaggacatttataatgcatccatagagttattctccgacataagaacctccgtcacagaaattaccgaaaagttgtggtccaaaaatatgatcgaagttttaaagacaagagagcaaaccattgcaggcattttatgtgagttaagaaatggaaataattctcccctagtatcgaacagtttttcctatgaaaattttggaattctcaaggttaattatgagggattactaaaccaggcgtatgcggccttttcagactactattcatactttcccgcttttgccattagcatgttagaaaagggagggttggtcgaccgcttggtcgccatccatgagagcttgaccaactacaggacgagaaatattctcaagaagatcaatgagaagtccaaaaatgaggtcctcaataatgaagaaattatgcacagcttgagcagttacaagcaccatgccgggggcacgcgtggcgccttcctgcagtccagagatgtgcgcgaagttacgcaaggagatgtgagcgttgatgagaagggcgaccgggccaccaccgcggggggcaaccaaagcgcaagcgtggctgcggcggccccgaaggatgcgggcccaaccgtggctgctcctaacactgctgctacgctcaaaacggctgcttcccccaacgcggctgctactaacactgctgctccccccaacatgggtgccacctccccgctgagcaaccccctgtacggcaccagctccctgcagccaaaggacgtcgcggtgctggtcagagatctgctcaagaacacgaacatcatcaagttcgagaataacgaaccgactagccaaatggacgatgaagaaattaagaagctcattgagagctcctttttcgacttgagcgacaacaccatgttaatgcggttgctcataaagccgcaggcggccatcttactaatcattgagtccttcattatgatgacgccctcccccacgagggacgccaagacctattgcaagaaagccctagttaatggccagctaatcgaaacctcagatttaaacgcggcgacggaggaagacgacctcataaacgagttttccagcaggtacaatttattctacgagaggctcaagctggaggagttg

SEQ ID NO: 141 - DNA, 1572 bp, Plasmodium vivax
  source 1..1572: mol_type = genomic DNA
aaggagtactgcgaccagcttagcttttgcgatgtgggattgacacaccactttgatacgtattgtaagaatgaccagtacctgttcgttcactacacttgtgaggacctctgcaaaacgtgtggccctaattcgtcctgctacggaaacaagtacaaacataagtgcctgtgcaatagccccttcgagagtaaaaagaaccattccatttgcgaagcacgaggtagctgcgatgcacaggtatgcggcaagaatcaaatttgcaaaatggtagacgctaaagcaacatgcacatgtgcagataaataccaaaatgtgaatggggtgtgtctaccggaagataagtgcgaccttctgtgcccctcaaacaaatcgtgcctgctggaaaatgggaaaaaaatatgcaagtgcattaatgggttgactctacagaacggcgagtgcgtctgctcggatagcagccaaattgaagaaggacacctctgtgtgcccaagaataaatgtaaacggaaggagtaccaacagctctgcaccaatgagaaggaacactgtgtgtatgatgagcagacggacattgtgcggtgcgactgcgtggaccacttcaagcggaacgaacggggaatttgcatcccagtcgactactgcaaaaatgtcacctgcaaggaaaatgagatttgcaaagttgttaataatacacccacatgtgagtgtaaagaaaatttaaaaagaaatagtaacaatgaatgtgtattcaataacatgtgtcttgttaataaagggaactgccccattgattcggagtgcatttatcacgagaaaaaaaggcatcagtgtttgtgccataagaagggcctcgtcgccattaatggcaagtgcgtcatgcaggacatgtgcaggagcgatcagaacaaatgctccgaaaattccatttgtgtaaatcaagtgaataaagaaccgctgtgcatatgtttgtttaattatgtgaagagtcggtcgggcgactcgcccgagggtggacagacgtgcgtggtggacaatccctgcctcgcgcacaacgggggctgctcgccaaacgaggtttgcacgttcaaaaatggaaaggtaagttgcgcctgcggggagaactaccgccccagggggaaggacagcccaacgggacaagcggtcaaacggggggaagcgaccaaacggggtgacgcgggtcagcccgggcaggcgcactcagcaaatgagaacgcgtgcctgcccaagacgtccgaggcggaccaaaccttcaccttccagtacaacgacgacgcggccatcattctcgggtcctgcggaattatacagtttgtgcaaaagagcgatcaggtcatttggaaaattaacagcaacaatcacttttacatttttaattatgactatccatctgagggtcagctgtcggcacaagtcgtgaacaagcaggagagcagcattttgtacttaaagaaaacccacgcggggaaagtcttttacgccgactttgagttgggtcatcagggatgctcctacggaaacatgtttctctacgcccaccgggaggaggct

SEQ ID NO: 142 - DNA, 1020 bp, Plasmodium vivax
  source 1..1020: mol_type = genomic DNA
agcaaaaacattattattctgaacgatgaaattaccaccattaaaagcccgattcattgcattaccgatatttattttctgtttcgcaacgaactgtataaaacctgcattcagcatgtgattaaaggccgcaccgaaattcatgtgctggtgcagaaaaaaattaacagcgcgtgggaaacccagaccaccctgtttaaagatcatatgtggtttgaactgccgagcgtgtttaactttattcataacgatgaaattattattgtgatttgccgctataaacagcgcagcaaacgcgaaggcaccatttgcaaacgctggaacagcgtgaccggcaccatttatcagaaagaagatgtgcagattgataaagaagcgtttgcgaacaaaaacctggaaagctatcagagcgtgccgctgaccgtgaaaaacaaaaaatttctgctgatttgcggcattctgagctatgaatataaaaccgcgaacaaagataactttattagctgcgtggcgagcgaagataaaggccgcacctggggcaccaaaattctgattaactatgaagaactgcagaaaggcgtgccgtatttttatctgcgcccgattatttttggcgatgaatttggcttttatttttatagccgcattagcaccaacaacaccgcgcgcggcggcaactatatgacctgcaccctggatgtgaccaacgaaggcaaaaaagaatataaatttaaatgcaaacatgtgagcctgattaaaccggataaaagcctgcagaacgtggcgaaactgaacggctattatattaccagctatgtgaaaaaagataactttaacgaatgctatctgtattataccgaacagaacgcgattgtggtgaaaccgaaagtgcagaacgatgatctgaacggctgctatggcggcagctttgtgaaactggatgaaagcaaagcgctgtttatttatagcaccggctatggcgtgcagaacattcataccctgtattatacccgctatgat